(19) United States
(12) Patent Application Publication
(10) Pub. No.: US 2025/0109091 A1
(43) Pub. Date: Apr. 3, 2025

(54) NITROGEN-CONTAINING COMPOUND, ELECTRONIC ELEMENT, AND ELECTRONIC DEVICE

(71) Applicant: SHAANXI LIGHTE OPTOELECTRONICS MATERIAL CO., LTD., Xi'an (CN)

(72) Inventor: Wenqiang Liu, Xi'an (CN)

(21) Appl. No.: 18/839,022

(22) PCT Filed: Jun. 16, 2023
(86) PCT No.: PCT/CN2023/100810

§ 371 Date: Aug. 16, 2024

(30) Foreign Application Priority Data
Sep. 14, 2022 (CN) 202211119275.8
Nov. 3, 2022 (CN) 202211370565.X

(51) Int. Cl. C07C 211/54; C07B 59/00; C07C 211/61; C07D 209/90; C07D 307/91; C07D 333/76; C07D 405/12; C07D 407/12; C07D 409/12; H10K 50/15; H10K 85/60

(52) U.S. Cl. CPC C07C 211/54; C07B 59/004; C07C 211/61; C07D 209/90; C07D 307/91; C07D 333/76; C07D 405/12; C07D 407/12; C07D 409/12; H10K 50/15; H10K 85/615; H10K 85/631; H10K 85/6572; H10K 85/6574; H10K 85/6576; C07B 2200/05

(57) ABSTRACT

The present invention belongs to the technical field of organic materials, and provides a nitrogen-containing compound, an electronic element, and an electronic device. The structure of the nitrogen-containing compound is represented by formula 1, and the nitrogen-containing compound can improve the performance of the electronic element.

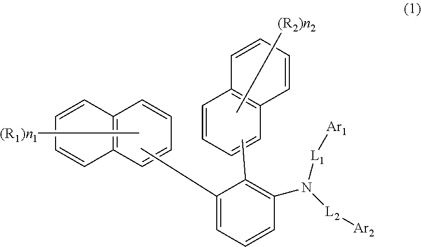

### CROSS-REFERENCE TO RELATED APPLICATIONS

The present application claims the priority of Chinese patent application No. 202211119275.8 filed on Sep. 14, 2022 and Chinese patent application No. 202211370565.X filed on Nov. 3, 2022, which are incorporated herein by references in their entirety as a part of the present application.

### FIELD OF THE INVENTION

The present application belongs to the technical field of organic electroluminescent materials, in particular relates to a nitrogen-containing compound, an electronic element, and an electronic device.

### BACKGROUND OF THE INVENTION

Organic electroluminescent device (OLED), also known as organic light-emitting device, refers to the phenomenon of light-emitting by organic light-emitting materials when excited by current under the action of an electric field. Compared with inorganic light-emitting materials, organic electroluminescent diode OLED has advantages of active light emission, large optical path range, low driving voltage, high brightness, high efficiency, low energy consumption and simple manufacturing process. Due to these advantages, organic light-emitting materials and devices have become one of the most popular scientific research topics in the scientific community and industry.

An organic electroluminescent device typically consists of an anode, a hole transport layer, an organic emissive layer, an electron transport layer and a cathode that are stacked in sequence. When a voltage is applied to the cathode and anode, the two electrodes generate an electric field. Under the action of the electric field, the electrons on the cathode side move toward the electroluminescent layer, and the holes on the anode side also move toward the emissive layer. The electrons and holes combine in the electroluminescent layer to form excitons, and the excitons release energy outwards in an excited state, thereby causing light-emitting of the organic emissive layer.

Currently, OLED display technology has been applied in smartphones, tablets and other fields, and will further expand to large-size applications such as TVs. However, compared with actual product application requirements, the current efficiency, service life and other properties of OLED devices still needs further improvement. Research on improving the performance of OLED light-emitting devices comprises: reducing the operating voltage of the device, improving the current efficiency of the device, and increasing the service life of the device. In order to continuously improve the performance of OLED devices, it not only requires innovation in the OLED device structure and manufacturing process, but also requires continuous research and innovation in OLED optoelectronic functional materials to create higher-performance OLED functional materials.

### SUMMARY OF THE INVENTION

The objective of the present application is to provide a nitrogen-containing compound, an electronic element, and an electronic device. Using the nitrogen-containing compound in an organic electroluminescent device can improve the performance of the device.

In a first aspect, the present application provides a nitrogen-containing compound, having a structure shown in Formula 1:

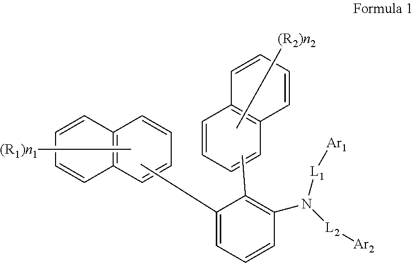

wherein Ar₁ and Ar₂ are identical or different, and are each independently selected from a substituted or unsubstituted aryl having 6 to 40 carbon atoms, or a substituted or unsubstituted heteroaryl having 3 to 40 carbon atoms;
L₁ and L₂ are identical or different, and are each independently selected from a single bond, a substituted or unsubstituted arylene having 6 to 30 carbon atoms, or a substituted or unsubstituted heteroarylene having 3 to 30 carbon atoms;
Substituent(s) of Ar₁, Ar₂, L₁ and L₂ are identical or different, and are each independently selected from deuterium, a halogen, a cyano, an alkyl having 1 to 10 carbon atoms, a haloalkyl having 1 to 10 carbon atoms, a deuterated alkyl having 1 to 10 carbon atoms, an aryl having 6 to 18 carbon atoms, a deuterated aryl having 6 to 18 carbon atoms, a heteroaryl having 5 to 18 carbon atoms, a cycloalkyl having 3 to 10 carbon atoms, or a trialkylsilyl having 3 to 12 carbon atoms, optionally, any two adjacent substituents form a saturated or unsaturated 5-membered to 15-membered ring;
R₁ and R₂ are identical or different, and are each independently selected from deuterium, a halogen, a cyano, an alkyl having 1 to 4 carbon atoms, a haloalkyl having 1 to 4 carbon atoms, a deuterated alkyl having 1 to 4 carbon atoms, a trialkylsilyl having 3 to 7 carbon atoms, an aryl having 6 to 18 carbon atoms, a deuterated aryl having 6 to 18 carbon atoms, a heteroaryl having 5 to 18 carbon atoms or a cycloalkyl having 3 to 10 carbon atoms;
n₁ represents the number of R₁, n₁ is selected from 0, 1, 2, 3, 4, 5, 6 or 7, wherein when n₁ is greater than 1, each R₁ is identical or different;
n₂ represents the number of R₂, n₂ is selected from 0, 1, 2, 3, 4, 5, 6 or 7, wherein when n₂ is greater than 1, each R₂ is identical or different.

In a second aspect, the present application provides an electronic element, comprising an anode, a cathode, and a functional layer disposed between the anode and the cathode, wherein the functional layer comprises the nitrogen-containing compound described in the first aspect of the disclosure.

In a third aspect, the present application provides an electronic device, comprising the electronic element described in the second aspect of the present application.

The nitrogen-containing compound of the present application is a triarylamine compound with a core of benzene ring. Two naphthyl structures are introduced at the adjacent positions (i.e., position 1 and position 2) of the benzene ring

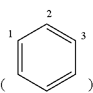

to adjust the steric configuration of the entire molecule, and increase the glass transition temperature of the compound. In addition, the two adjacent naphthyl structures enable the compound to have a deeper HOMO level and a higher LUMO level. The compound can be used as a light-emitting assisting layer (i.e., the second hole transport layer) of the OLED device, which has better interface performance when used in conjunction with the adjacent functional layer, and can also maintain high hole migration efficiency. In addition, the ortho-bis-naphthyl-substituted compound has reduced crystallinity, and improved film-forming property, resulting in higher stability of the device. When the nitrogen-containing compound of the present application is used in OLED device, it can further improve the current efficiency and service life of the devices while maintaining a lower driving voltage.

Other features and advantages of the present application will be described in detail in the subsequent Detailed Description of the Embodiments section.

### BRIEF DESCRIPTION OF THE DRAWINGS

The accompanying drawings are intended to provide a further understanding of the present application and form a part of the specification, which are used to illustrate the present application together with the following specific embodiments, but do not constitute a limitation of the present application.

FIG. 1 is a schematic structural diagram of an organic electroluminescent device according to an embodiment of the present application.

FIG. 2 is a schematic diagram of a first electronic device according to an embodiment of the present application.

FIG. 3 is a schematic structural diagram of a photoelectric conversion device according to an embodiment of the present application.

FIG. 4 is a schematic diagram of a second electronic apparatus according to an embodiment of the present application.

### LIST OF REFERENCE SIGNS

100: anode; 200: cathode; 300: functional layer;
310: hole injection layer; 320: hole transport layer;
321: first hole transport layer; 322: second hole transport layer;
330: organic emissive layer; 340: electron transport layer;
350: electron injection layer; 360: photoelectric conversion layer;
400: first electronic device; 500: second electronic device.

### DETAILED DESCRIPTION OF THE EMBODIMENTS

Specific embodiments of the present application are described in detail below with reference to the accompanying drawings. It should be appreciated that the specific embodiments described herein are intended only to illustrate and explain, rather than limiting, the present application.

The present application, in a first aspect, provides a nitrogen-containing compound, having a structure shown in Formula 1:

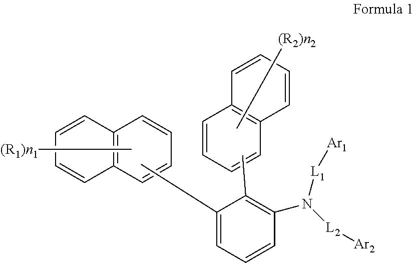

in Formula 1, Ar₁ and Ar₂ are identical or different, and are each independently selected from a substituted or unsubstituted aryl having 6 to 40 carbon atoms, or a substituted or unsubstituted heteroaryl having 3 to 40 carbon atoms;
L₁ and L₂ are identical or different, and are each independently selected from a single bond, a substituted or unsubstituted arylene having 6 to 30 carbon atoms, or a substituted or unsubstituted heteroarylene having 3 to 30 carbon atoms;
Substituent(s) of Ar₁, Ar₂, L₁ and L₂ are identical or different, and are each independently selected from deuterium, a halogen, a cyano, an alkyl having 1 to 10 carbon atoms, a haloalkyl having 1 to 10 carbon atoms, a deuterated alkyl having 1 to 10 carbon atoms, an aryl having 6 to 18 carbon atoms, a deuterated aryl having 6 to 18 carbon atoms, a heteroaryl having 5 to 18 carbon atoms, a cycloalkyl having 3 to 10 carbon atoms, or a trialkylsilyl having 3 to 12 carbon atoms; optionally, any two adjacent substituents form a saturated or unsaturated 5-membered to 15-membered ring;
R₁ and R₂ are identical or different, and are each independently selected from deuterium, a halogen, a cyano, an alkyl having 1 to 4 carbon atoms, a haloalkyl having 1 to 4 carbon atoms, a deuterated alkyl having 1 to 4 carbon atoms, a trialkylsilyl having 3 to 7 carbon atoms, an aryl having 6 to 18 carbon atoms, a deuterated aryl having 6 to 18 carbon atoms, a heteroaryl having 5 to 18 carbon atoms or a cycloalkyl having 3 to 10 carbon atoms;
n₁ represents the number of R₁, n₁ is selected from 0, 1, 2, 3, 4, 5, 6 or 7, wherein when n₁ is greater than 1, each R₁ is identical or different;
n₂ represents the number of R₂, n₂ is selected from 0, 1, 2, 3, 4, 5, 6 or 7, wherein when n₂ is greater than 1, each R₂ is identical or different.

In the present application, the expression “each . . . independently” may be used interchangeably with the expressions “. . . each independently”, and all these expressions should be interpreted in a broad sense. They can not only mean that, for same symbols in a same group, the selection of a specific option for one of the symbols and the selection of a specific option for another one of the symbols do not affect each other, but also mean that for same symbols in different groups, the selection of a specific option for one of the symbols and the selection of a specific option for another one of the symbols do not affect each other. Taking

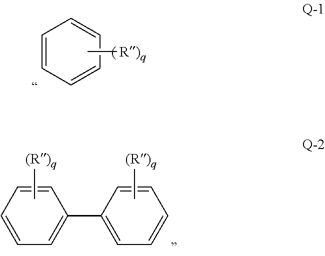

as an example, each q is independently 0, 1, 2, or 3, and each R″ is independently selected from hydrogen, deuterium, fluorine, or chlorine, which means: in Formula Q-1, there are q substituent R″(s) on the benzene ring, wherein each of the substituent R″(s) may be identical or different, with the selection of an option for one of the substituent R″(s) and the selection of an option for another one of the substituent R″(s) not affecting each other; and in Formula Q-2, there are q substituent R″(s) on each of the two benzene rings of biphenyl, wherein the number q of the substituent R″(s) on one benzene ring and the number q of the substituent R″(s) on the other benzene ring may be the same or different, and each substituent R″ may be identical or different, with the selection of an option for one of the substituent R″(s) and the selection of an option for another one of the substituent R″(s) not affecting each other.

In the present application, the term “optional” and “optionally” means that the subsequently described event or circumstance may but need not occur, and the description comprises circumstances in which the event or circumstance does or does not occur. For example, “optionally, any two adjacent substituents form a ring” means that any two adjacent substituents can form a ring but do not have to form a ring, including: the scenario where two adjacent substituents form a ring, and two adjacent substituents do not form a ring.

In the present application, the term “substituted or unsubstituted” means that the functional group defined by the term may or may not have a substituent (hereinafter referred to as Rc for ease of description). For example, “substituted or unsubstituted aryl” refers to an aryl group having a substituent Rc or an unsubstituted aryl group. The above-mentioned substituents, namely Rc, may be, for example, deuterium, halogen, cyano, heteroaryl, aryl, alkyl, haloalkyl, deuterated alkyl, cycloalkyl, trialkylsilyl, etc. In the present application, a “substituted” functional group may be substituted by one or more than two of the above substituent Rc(s), and when two substituent Rcs are connected to the same atom, the two substituent Rcs can exist independently or can be linked to each other to form a spiro ring together with the atom; and when there are two adjacent substituent Rcs on the functional group, the two adjacent substituent Rcs can exist independently or be fused to form a ring with the functional group to which they are attached.

In the present application, the term “Ring” includes a saturated ring and an unsaturated ring; the saturated ring includes cycloalkyl and heterocycloalkyl, and the unsaturated ring includes cycloalkenyl, heterocycloalkenyl, aryl and heteroaryl. In the present application, a ring system formed by n atoms is a n-membered ring. For example, phenyl is a 6-membered aryl group; fluorene ring is a 13-membered ring, cyclohexane is a 6-membered ring, and adamantane is a 10-membered ring.

In the present application, “any two adjacent substituents form a 3-membered to 15-membered saturated or unsaturated ring”, the formed ring is a saturated ring or an unsaturated ring, wherein the saturated ring may be, for example, a cyclopentane

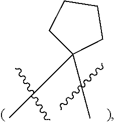 [SMILES: *C1(*)CCCC1]

a cyclohexane

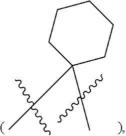 [SMILES: *C1(*)CCCCC1]

the unsaturated ring may be, for example, a benzene ring, a naphthalene ring or a fluorene ring

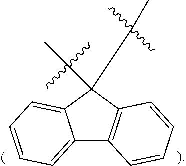 [SMILES: *C1(*)c2ccccc2-c2ccccc21]

In the present application, “aryl” refers to any functional group or substituent group derived from an aromatic carbon ring. An aryl group may be a monocyclic aryl group (e.g., phenyl) or a polycyclic aryl group. In other words, an aryl group may be a monocyclic aryl group, a fused aryl group, two or more monocyclic aryl groups linked by carbon-carbon bond conjugation, a monocyclic aryl group and a fused aryl group linked by carbon-carbon bond conjugation, or two or more fused aryl groups linked by carbon-carbon bond conjugation. That is, unless otherwise specified, two or more aromatic groups linked by carbon-carbon bond conjugation may also be regarded as an aryl group in the present application. Among them, fused aryl groups may comprise, for example, bicyclic fused aryl groups (e.g., naphthyl), tricyclic fused aryl groups (e.g., phenanthryl, fluorenyl, anthryl), etc. An aryl group does not contain a heteroatom such as B, N, O, S, P, Se, and Si. It should be noted that biphenyl and fluorenyl are considered as aryl groups in the present application. Examples of aryl groups may include, but are not limited to, phenyl, naphthyl, fluorenyl, anthryl, phenanthryl, biphenyl, terphenyl, benzo[9,10]phenanthryl, pyrenyl, benzofluoranthryl, chrysenyl, etc. In the present application, the arylene refers to a bivalent group formed by further removing one hydrogen atom from an aryl group.

In the present application, “substituted aryl” may mean that one or more than two hydrogen atoms of the aryl group are substituted by a group such as deuterium, halogen, cyano, aryl, heteroaryl, alkyl, cycloalkyl, deuterated alkyl, deuterated aryl, trialkylsilyl, etc. Specific examples of heteroaryl-substituted aryl include, but are not limited to, dibenzofuranyl-substituted phenyl, dibenzothienyl-substituted phenyl, etc. It should be appreciated that the number of carbon atoms of a substituted aryl group refers to the number of all carbon atoms of the aryl group and the substituents of the aryl group. For example, a substituted aryl having 18 carbon atoms means that the number of all carbon atoms of the aryl group and substituents thereof is 18.

In the present application, “heteroaryl” refers to a monovalent aromatic ring containing 1, 2, 3, 4, 5, or more heteroatoms or a derivative thereof. The heteroatom may be at least one of B, O, N, P, Si, Se, and S. A heteroaryl group may be a monocyclic heteroaryl group or polycyclic heteroaryl group. In other words, a heteroaryl group may be a single aromatic ring system, or a plurality of aromatic ring systems linked by carbon-carbon bond conjugation, with any of the aromatic ring systems being an aromatic monocyclic ring or a aromatic fused ring. For example, heteroaryl groups may include, but are not limited to, thiophenyl, furyl, pyrrolyl, imidazolyl, thiazolyl, oxazolyl, oxadiazolyl, triazolyl, pyridyl, bipyridyl, pyrimidyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothiophenyl, dibenzothiophenyl, thienothiophenyl, benzofuranyl, phenanthrolinyl, isoxazolyl, thiadiazolyl, phenothiazinyl, silafluorenyl, dibenzofuranyl, and N-phenylcarbazolyl, N-pyridylcarbazolyl, N-methylcarbazolyl, etc. Among them, thiophenyl, furyl, phenanthrolinyl, and the like are each a heteroaryl group of a single aromatic ring system; and N-phenylcarbazolyl and N-pyridyl are a heteroaryl group of polycyclic systems linked by carbon-carbon bond conjugation. In the present application, a heteroarylene group is a divalent group formed by further removing one or more hydrogen atoms from a heteroaryl group.

In the present application, “substituted heteroaryl” may mean that one or more than two hydrogen atoms in the heteroaryl group are substituted by a group such as deuterium, fluorine, cyano, aryl, heteroaryl, alkyl, cycloalkyl, deuterated alkyl, deuterated aryl, trialkylsilyl, etc. Specific examples of aryl-substituted heteroaryl include, but are not limited to, phenyl-substituted dibenzofuranyl, phenyl-substituted dibenzothienyl, phenyl-substituted pyridyl, etc. It should be appreciated that the number of carbon atoms of a substituted heteroaryl group is the number of all carbon atoms of the heteroaryl group and substituents of the heteroaryl group.

In the present application, a non-positional bond is single bond “ [symbol] ” extending from a ring system, and it indicates that the linkage bond can be linked at one end thereof to any position in the ring system through which the bond passes, and linked at the other end thereof to the rest of the compound molecule. For example, as shown in Formula (f) below, the naphthyl group represented by Formula (f) is linked to other positions of the molecule via two non-positional bonds passing through the two rings, which indicates any of possible linkages shown in Formulae (f-1) to (f-10):

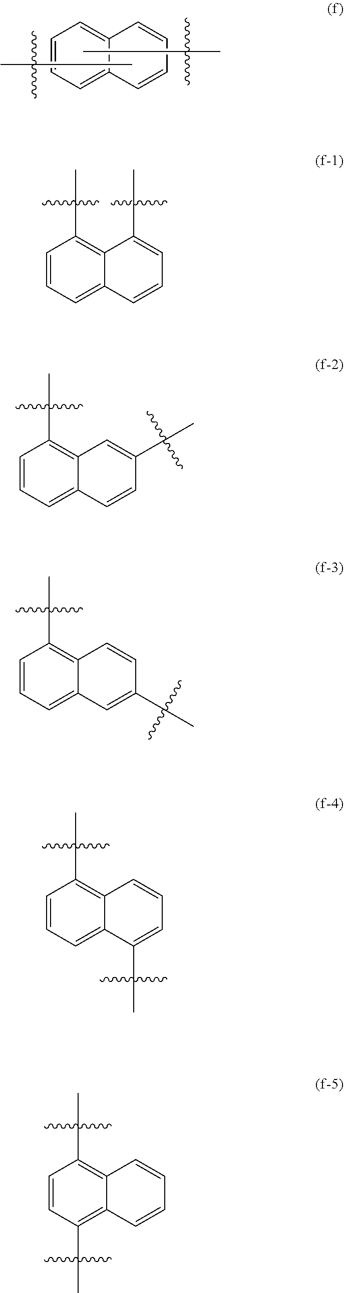

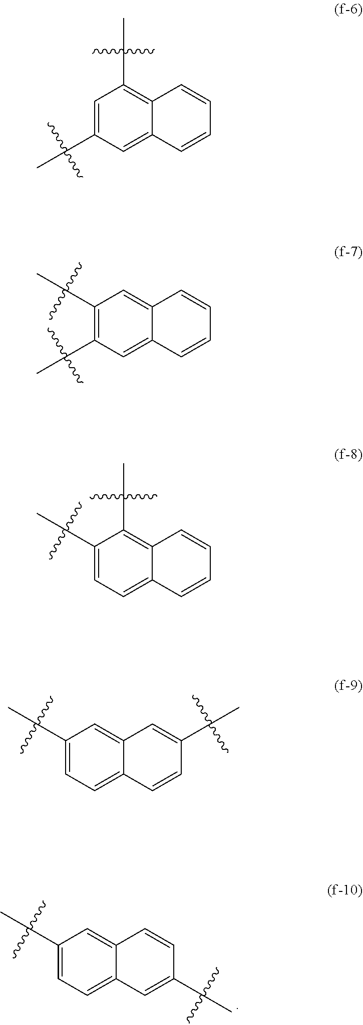 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl group represented by Formula (X′) is linked to other positions of the molecule via a non-positional bond extending from the center of benzene ring on one side, which indicates any of possible linkages shown in Formulae (X′-1) to (X′-4):

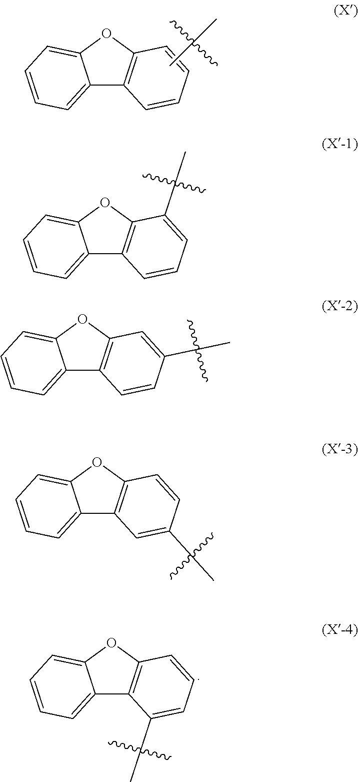

A non-positional substituent in the present application refers to a substituent linked via single bond extending from the center of a ring system, and it means that the substituent may be linked to any possible position in the ring system. For example, as shown in Formula (Y) below, the substituent R′ represented by Formula (Y) is linked to a quinoline ring via a non-positional bond, which indicates any of possible linkages shown in Formulas (Y-1) to (Y-7):

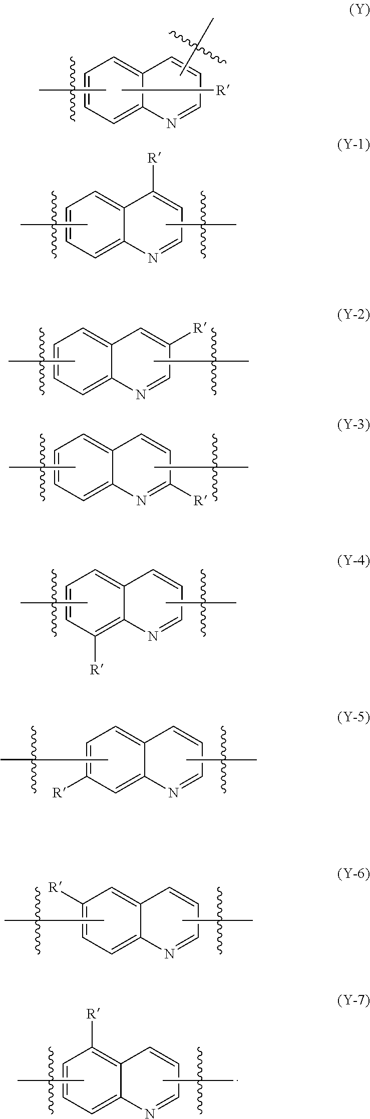

In the present application, halogen may include bromine, fluorine, chlorine, iodine, etc, preferably fluorine.

In the present application, the number of carbon atoms of an alkyl group having 1 to 10 carbon atoms may be 1, 2, 3, 4, 5, 6, 7, 8, 9, or 10. Specific examples of alkyl groups having 1 to 10 carbon atoms may include, but are not limited to, methyl, ethyl, n-propyl, isopropyl, n-butyl, isobutyl, tert-butyl, n-pentyl, isopentyl, neopentyl, cyclopenty, n-hexyl, heptyl, n-octyl, 2-ethylhexyl, nonyl, decyl, 3,7-dimethyloctyl, etc.

In the present application, the number of carbon atoms of an aryl group as a substituent may be 6 to 18, and may specifically be, for example, 6, 10, 12, 14, 18, etc. Specific examples of aryl groups as substituents include, but are not limited to, phenyl, naphthyl, biphenyl, etc.

In the present application, the number of carbon atoms of a heteroaryl group as a substituent may be 5 to 18, and may specifically be, for example, 5, 8, 9, 10, 12, 18, etc. Specific examples of heteroaryl groups as substituents include, but are not limited to, pyridyl, quinolyl, dibenzofuranyl, dibenzothienyl, carbazolyl, etc.

In the present application, the number of carbon atoms of a cycloalkyl group as a substituent may be 3 to 10, and may specifically be, for example, 3, 5, 6, 8, 10, etc. Specific examples of cycloalkyl groups include, but are not limited to, cyclopentyl, cyclohexyl, etc.

In the present application, the number of carbon atoms of a trialkylsilyl group as a substituent may be 3 to 12, and may specifically be, for example, 3, 5, 6, 8, 10, etc. Specific examples of trialkylsilyl groups include, but are not limited to, trimethylsilyl, etc.

In the present application, the number of carbon atoms of a deuterated alkyl group as a substituent may be 1 to 10, preferably 1 to 4. Specific examples of halogenated alkyl groups include, but are not limited to: trideuteratedmethyl.

In the present application, the number of carbon atoms of a haloalkyl group as a substituent may be 1 to 10, preferably 1 to 4. Specific examples of haloalkyl groups include, but are not limited to, trifluoromethyl.

In the present application, the number of carbon atoms of a deuterated aryl group as a substituent may be 6 to 18, preferably 6 to 12. Specific examples of deuterated aryl groups include, but are not limited to, pentadeuterated phenyl

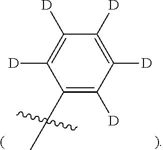 [SMILES: *c1c([2H])c([2H])c([2H])c([2H])c1[2H]]

In the present application, the structure of the nitrogen-containing compound is selected from Formula 1-1, Formula 1-2, Formula 2-1 or Formula 2-2:

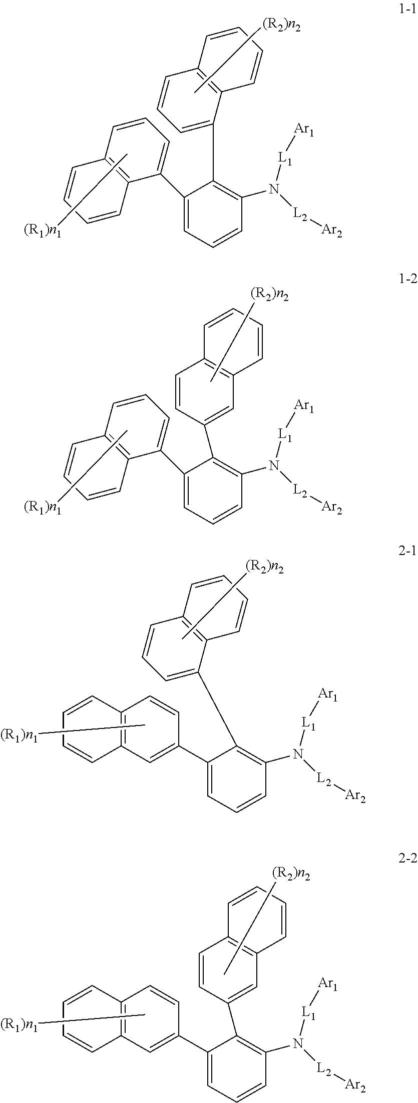

In an embodiment, R₁ and R₂ are each independently selected from deuterium, a fluorine, a cyano, a methyl, an ethyl, an isopropyl, a tert-butyl, a trifluoromethyl, a trideuteratedmethyl, a trimethylsilyl, a phenyl, a naphthyl, a biphenyl, a pentadeuterated phenyl, a dibenzofuranyl, a dibenzothienyl, a cyclopentyl or a cyclohexyl.

Further optionally, R₁ and R₂ are each independently selected from deuterium, a fluorine, a cyano, a methyl, an ethyl, an isopropyl, a tert-butyl, a trifluoromethyl, a trideuteratedmethyl or a phenyl.

In an embodiment,

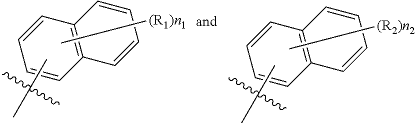

in Formula 1 are each independently selected from the group consisting of the following groups:

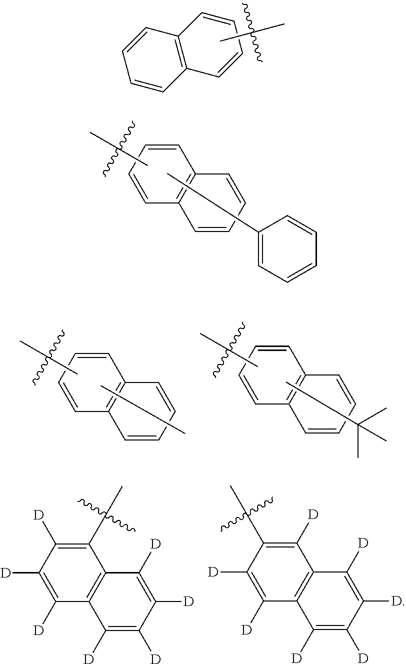

Optionally,

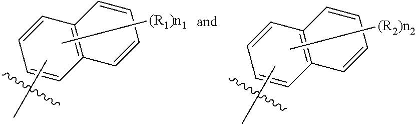

and are each independently selected from the group consisting of the following groups:

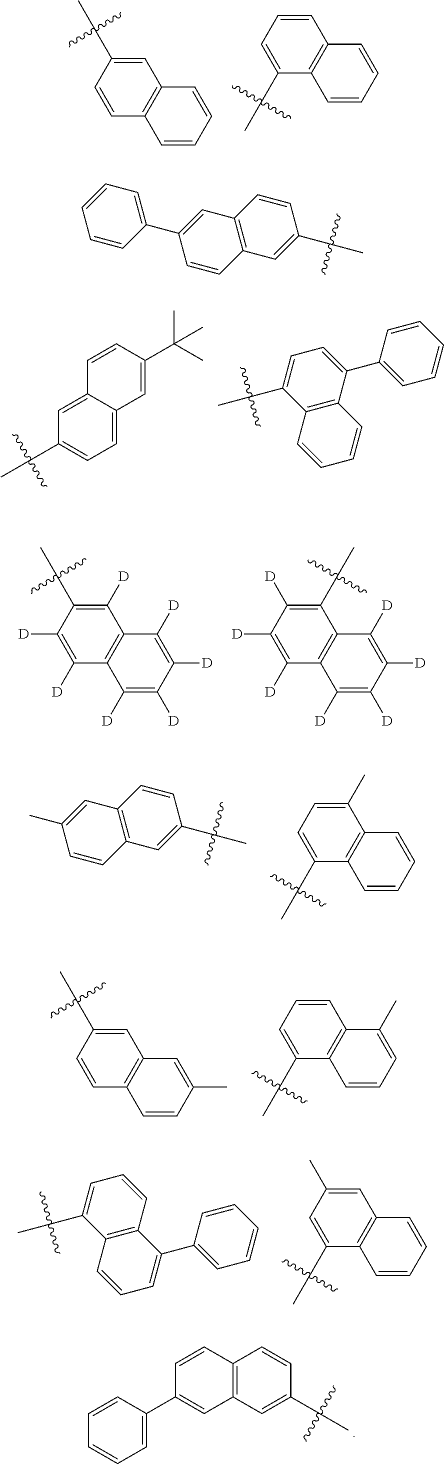

In the present application, L₁ and L₂ are each independently selected from a single bond, a substituted or unsubstituted arylene having 6, 7, 8, 9, 10, 11, 12, 13, 14, 15, 16, 17, 18, 19, 20, 21, 22, 23, 24, 25, 26, 27, 28, 29, or 30 carbon atoms, or a substituted or unsubstituted heteroarylene having 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms.

Optionally, L₁ and L₂ are each independently selected from a single bond, a substituted or unsubstituted arylene having 6 to 18 carbon atoms, or a substituted or unsubstituted heteroarylene having 12 to 18 carbon atoms.

Further optionally, L₁ and L₂ are each independently selected from a single bond, or a substituted or unsubstituted arylene having 6 to 12 carbon atoms.

In some embodiments, L₁ and L₂ are each independently selected from a single bond, a substituted or unsubstituted phenylene, a substituted or unsubstituted naphthylene, a substituted or unsubstituted biphenylene, a substituted or unsubstituted phenanthrylene, a substituted or unsubstituted fluorenylene, a substituted or unsubstituted dibenzofuranylene, a substituted or unsubstituted dibenzothienylene, or a substituted or unsubstituted carbazolylene.

Optionally, the substituent(s) of L₁ and L₂ are each independently selected from deuterium, a fluorine, a cyano, an alkyl having 1 to 4 carbon atoms, a haloalkyl having 1 to 4 carbon atoms, a deuterated alkyl having 1 to 4 carbon atoms, an aryl having 6 to 12 carbon atoms, a deuterated aryl having 6 to 12 carbon atoms, a heteroaryl having 5 to 12 carbon atoms or a trialkylsilyl having 3 to 7 carbon atoms.

Further optionally, the substituent(s) of L₁ and L₂ are each independently selected from deuterium, a fluorine, a cyano, a methyl, an ethyl, an isopropyl, a tert-butyl, a trifluoromethyl, a trideuteratedmethyl, a phenyl or a naphthyl.

In an embodiment, L₁ and L₂ are each independently selected from a single bond or the group consisting of the following groups:

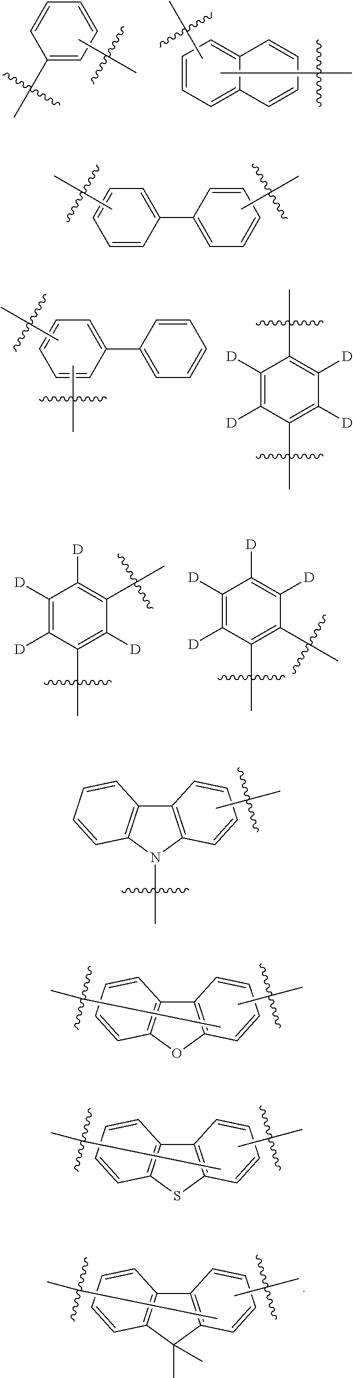

Further optionally, L₁ and L₂ are each independently selected from a single bond or the group consisting of the following groups:

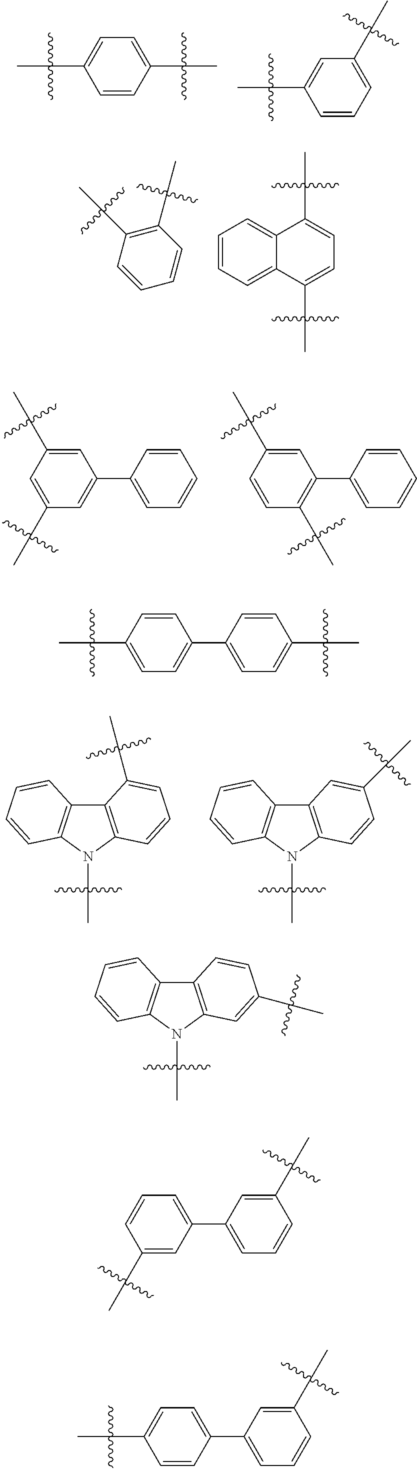

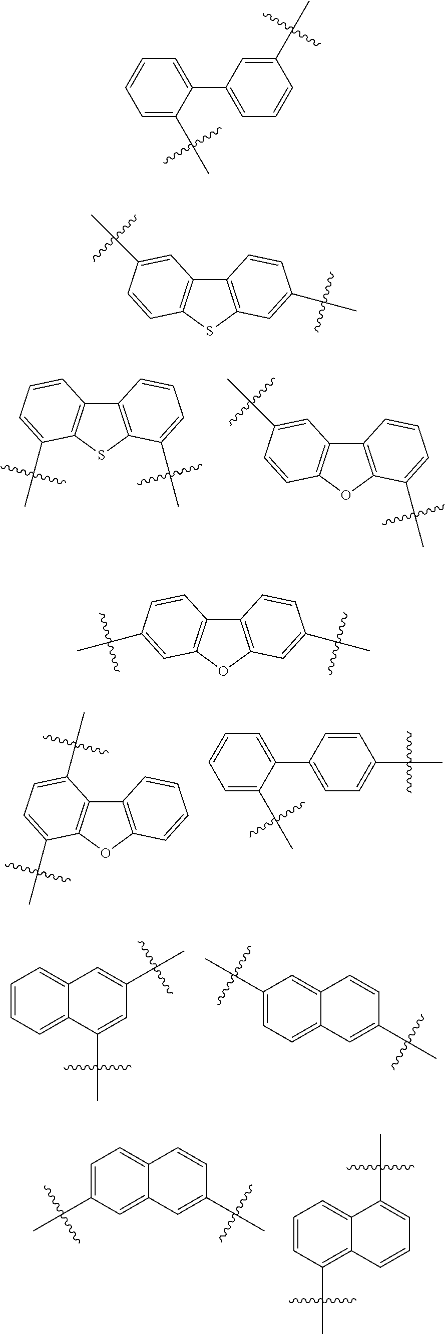

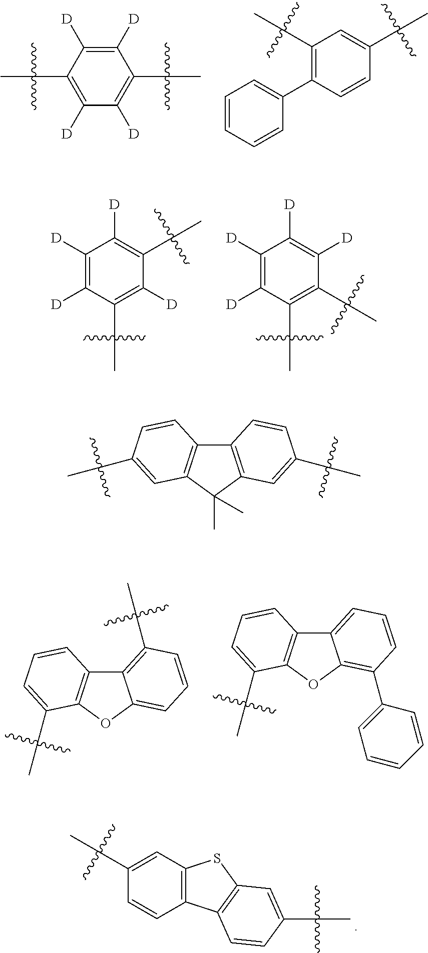

In the present application, Ar₁ and Ar₂ are each independently selected from a substituted or unsubstituted aryl having 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 carbon atoms, or a substituted or unsubstituted heteroaryl having 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 carbon atoms.

In an embodiment, Ar₁ and Ar₂ are each independently selected from a substituted or unsubstituted aryl having 6 to 25 carbon atoms, or a substituted or unsubstituted heteroaryl having 12 to 24 carbon atoms.

In an embodiment, Ar₁ and Ar₂ are each independently selected from a substituted or unsubstituted phenyl, a substituted or unsubstituted naphthyl, a substituted or unsubstituted biphenyl, a substituted or unsubstituted phenanthryl, a substituted or unsubstituted fluorenyl, a substituted or unsubstituted terphenyl, a substituted or unsubstituted triphenylene, a substituted or unsubstituted dibenzofuranyl, a substituted or unsubstituted dibenzothienyl, or a substituted or unsubstituted carbazolyl.

In an embodiment, Ar₁ is selected from a substituted or unsubstituted fluorenyl, a substituted or unsubstituted carbazolyl, a substituted or unsubstituted dibenzofuranyl, or a substituted or unsubstituted dibenzothienyl.

When Ar₁ is selected from a substituted or unsubstituted fluorenyl, a substituted or unsubstituted carbazolyl, a substituted or unsubstituted dibenzofuranyl, a substituted or unsubstituted dibenzothienyl, the molecular spatial configuration of the nitrogen-containing compound in the present application is more steric, which can increase the Ti energy level and effectively block the diffusion of excitons. When used as the second hole transport layer in an organic electroluminescent device, it can significantly increase the service life of the device.

Optionally, the substituent(s) of Ar₁ and Ar₂ are each independently selected from deuterium, a fluorine, a cyano, an alkyl having 1 to 4 carbon atoms, a haloalkyl having 1 to 4 carbon atoms, a deuterated alkyl having 1 to 4 carbon atoms, an aryl having 6 to 12 carbon atoms, a heteroaryl having 5 to 12 carbon atoms, a cycloalkyl having 5 to 10 carbon atoms or a trialkylsilyl having 3 to 7 carbon atoms; optionally, any two adjacent substituents form a saturated or unsaturated 5-membered to 15-membered ring.

Further optionally, the substituent(s) of Ar₁ and Ar₂ are each independently selected from deuterium, a fluorine, a cyano, a methyl, an ethyl, an isopropyl, a tert-butyl, a trifluoromethyl, a trideuteratedmethyl, a phenyl, a naphthyl, a dibenzofuranyl, a dibenzothienyl or a trimethylsilyl; optionally, any two adjacent substituents form a benzene ring, a cyclopentane

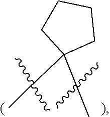 [SMILES: *C1(*)CCCC1]

a cyclohexane

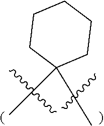 [SMILES: *C1(*)CCCCC1]

or a fluorene ring

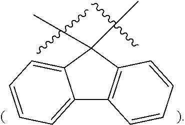 [SMILES: *C1(*)c2ccccc2-c2ccccc21]

In an embodiment, Ar₁ and Ar₂ are each independently selected from a substituted or unsubstituted group W, and the unsubstituted group W is selected from the group consisting of the following groups:

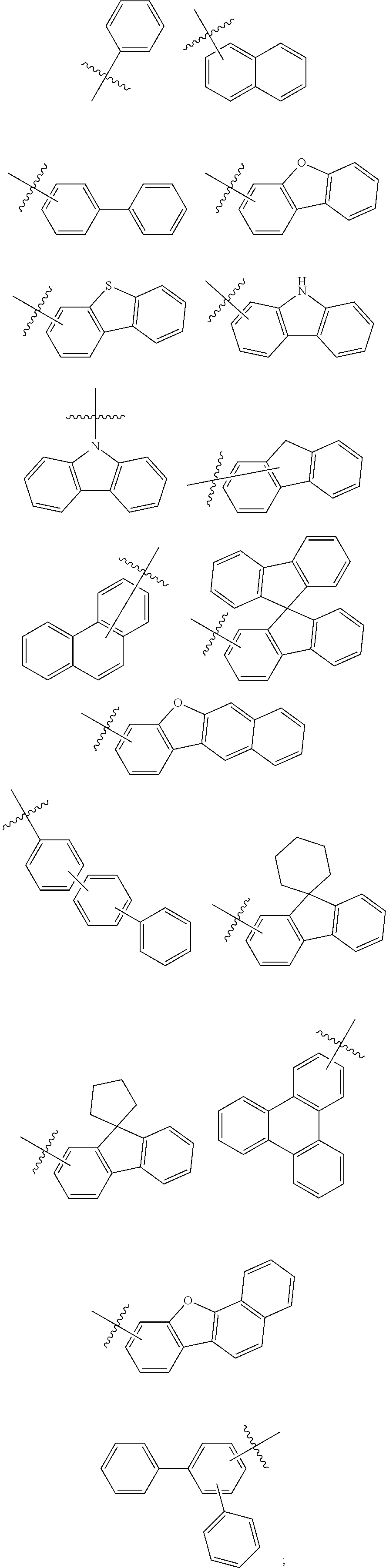

the substituent(s) of the substituted group W are each independently selected from deuterium, a fluorine, a cyano, a methyl, an ethyl, an isopropyl, a tert-butyl, a trifluoromethyl, a trideuteratedmethyl, a trimethylsilyl, a phenyl or a naphthyl, and when the number of the substituents is greater than 1, each substituent is identical or different.

Optionally, Ar₁ and Ar₂ are each independently selected from the group consisting of the following groups

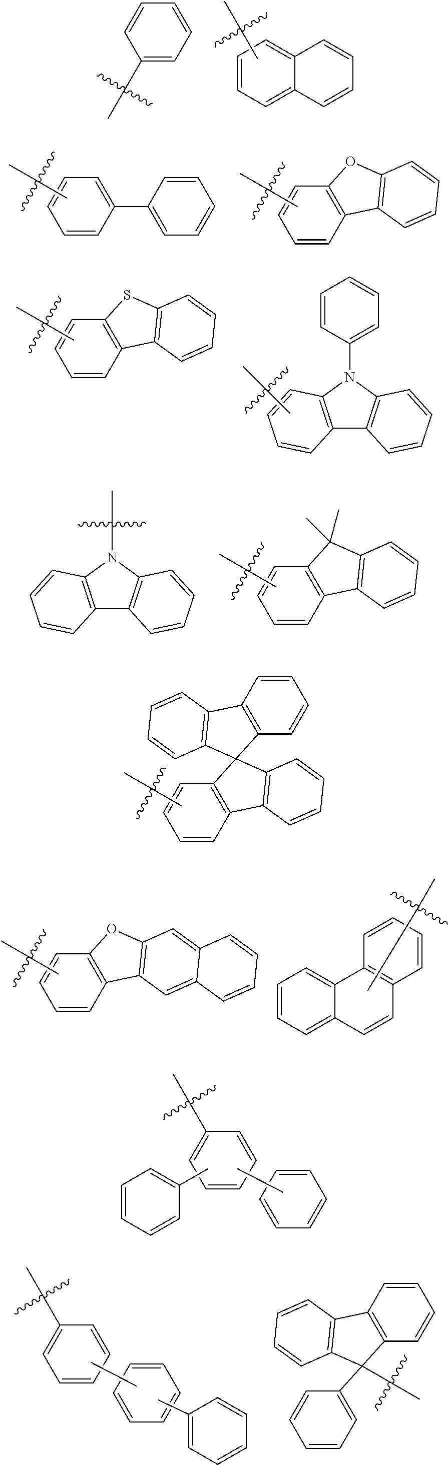

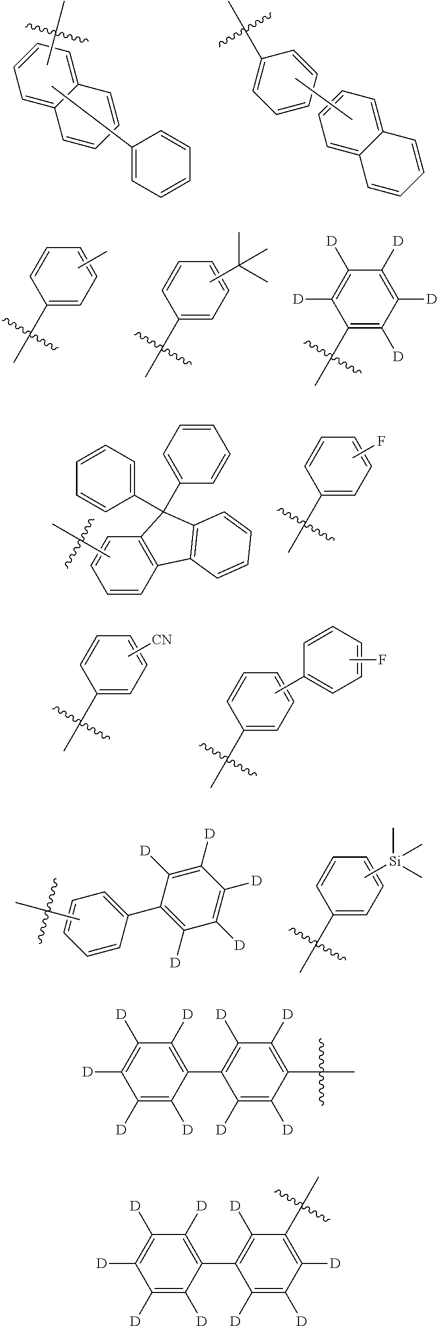

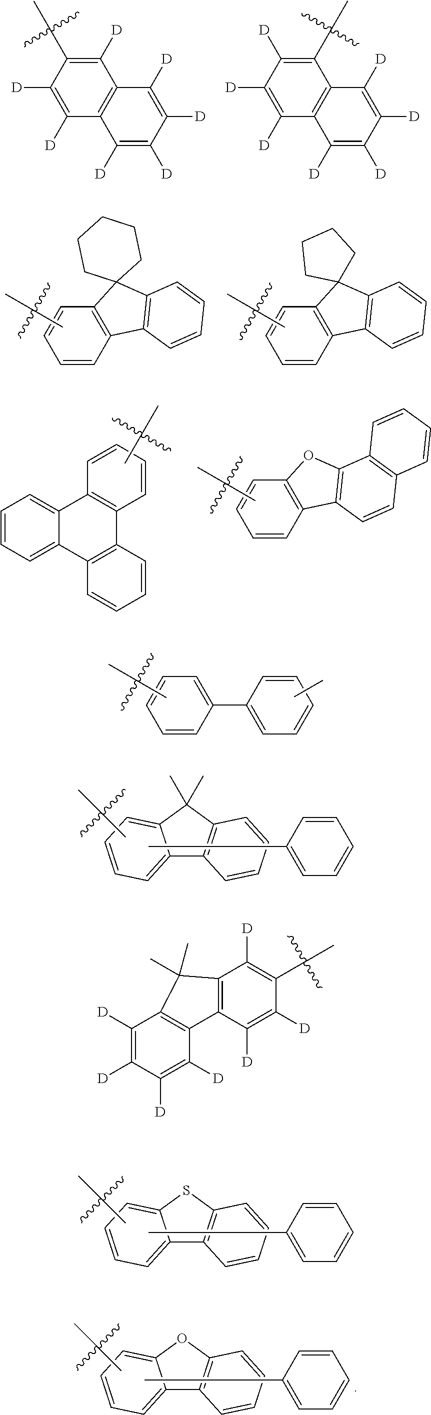

Further optionally, Ar₁ and Ar₂ are each independently selected from the group consisting of the following groups:

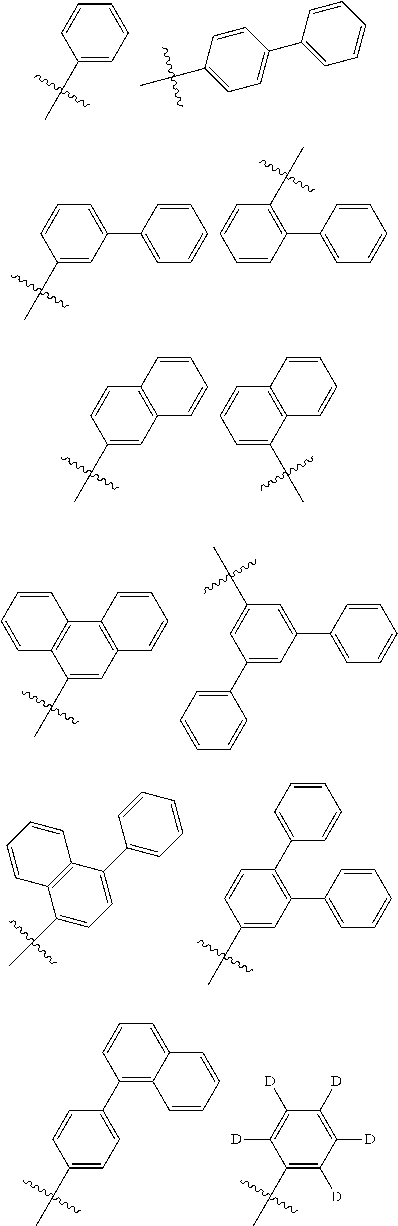

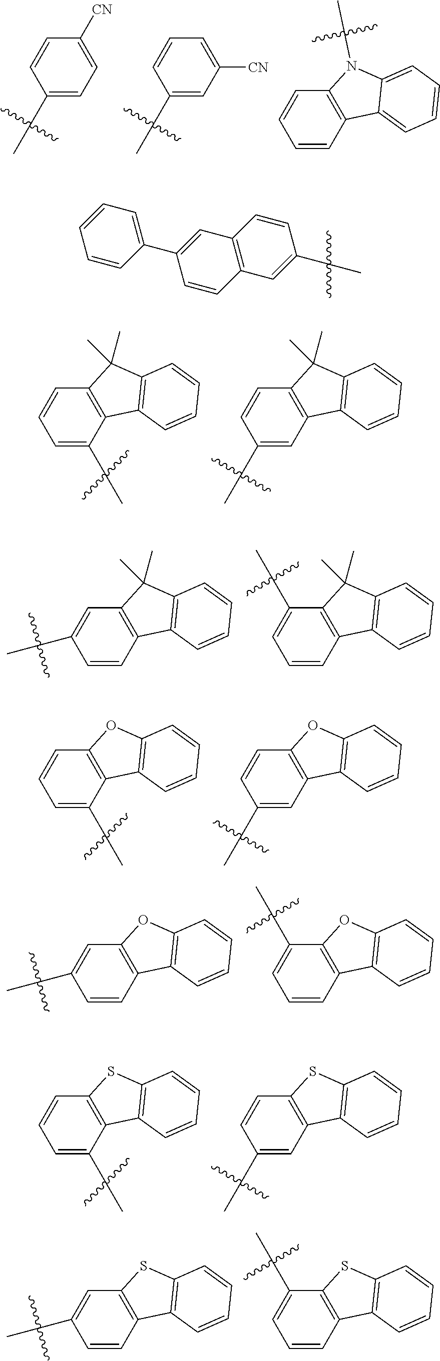

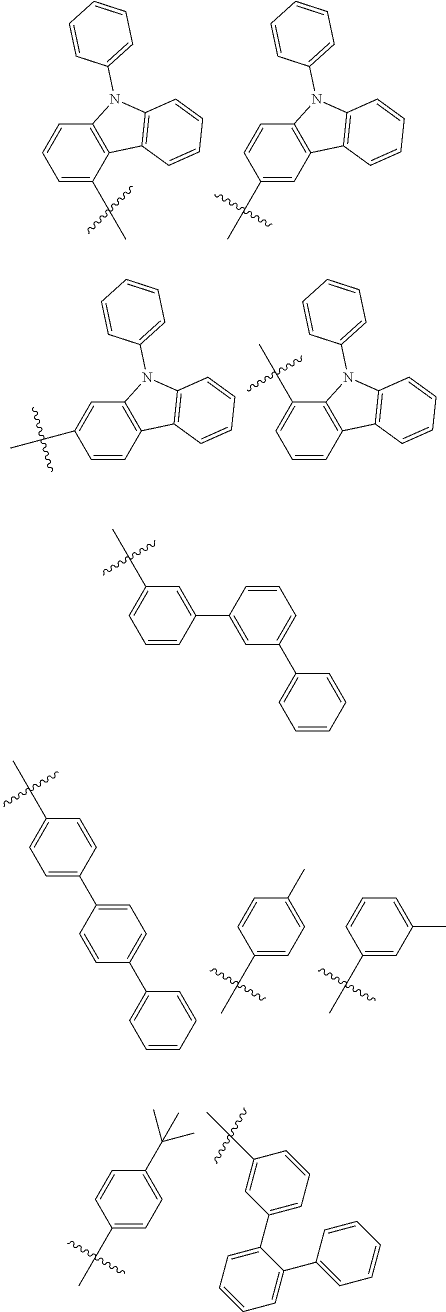

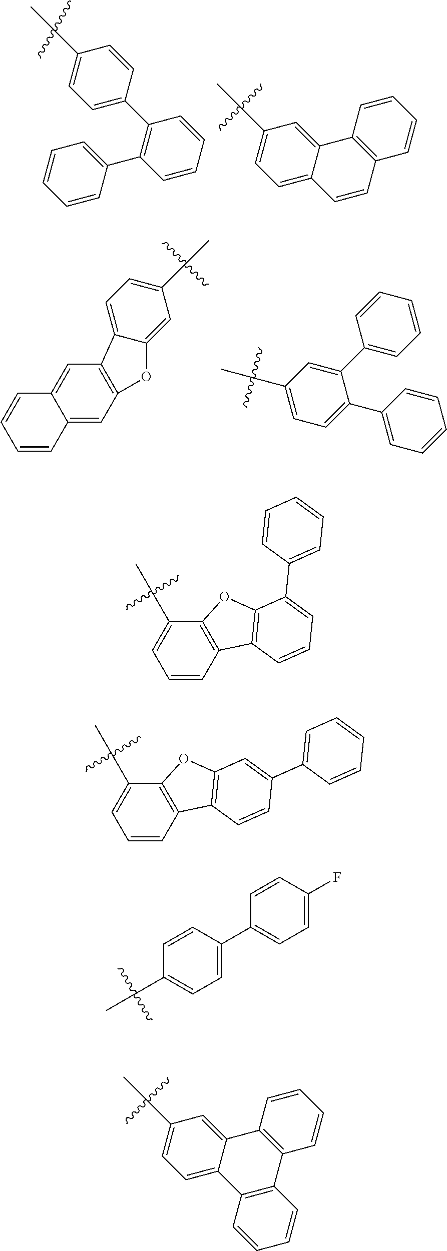

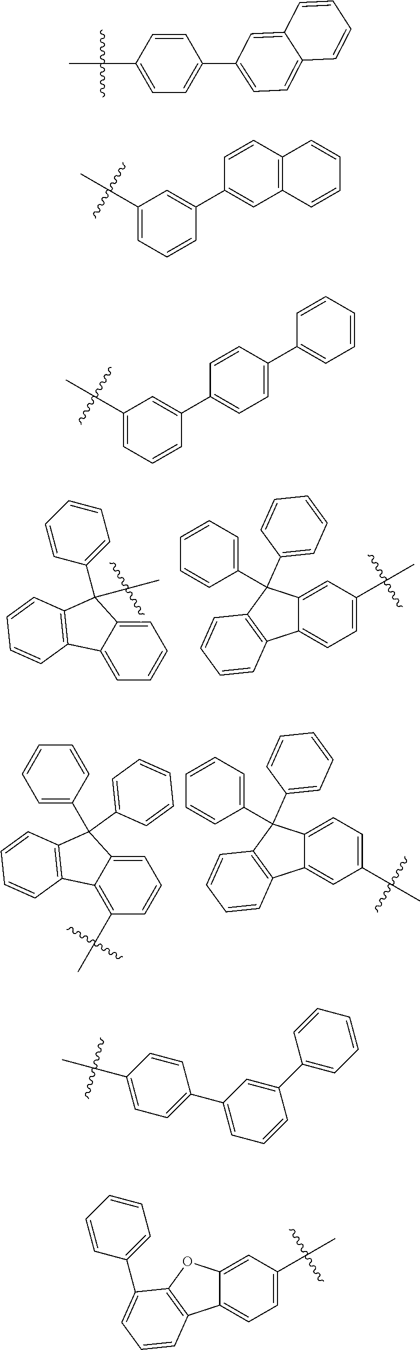

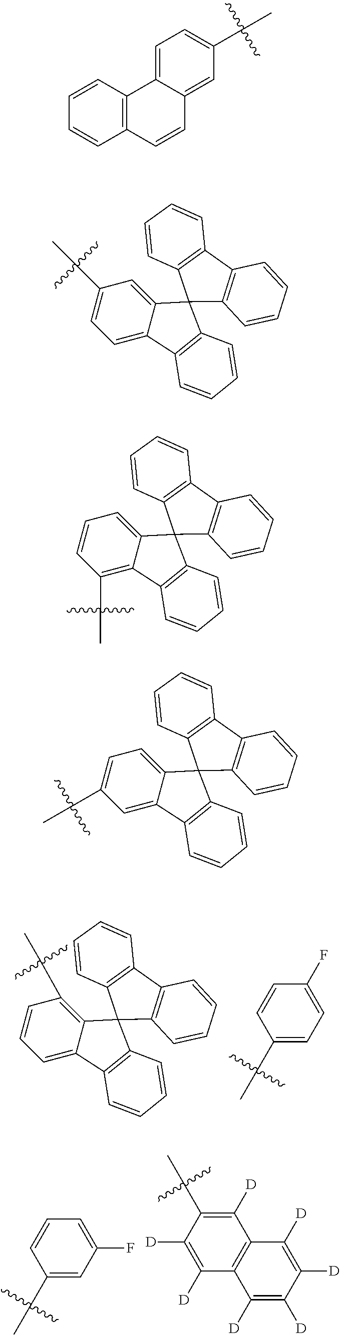

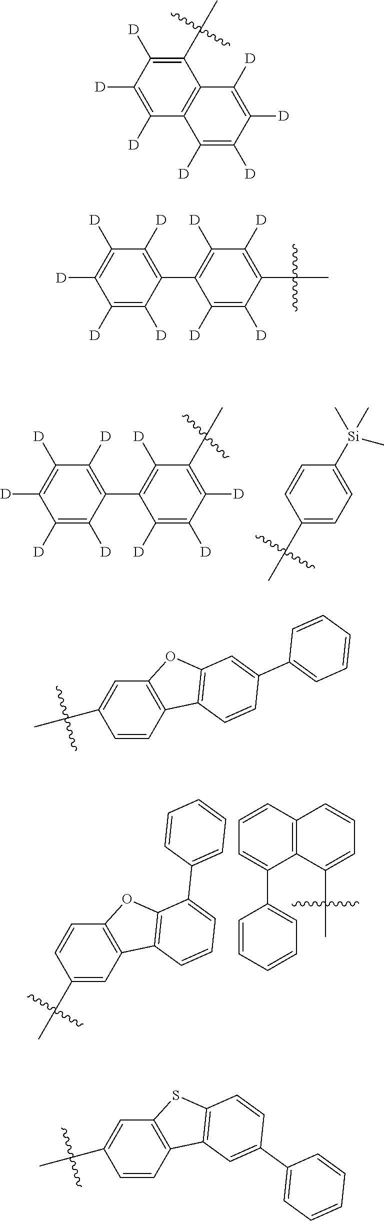

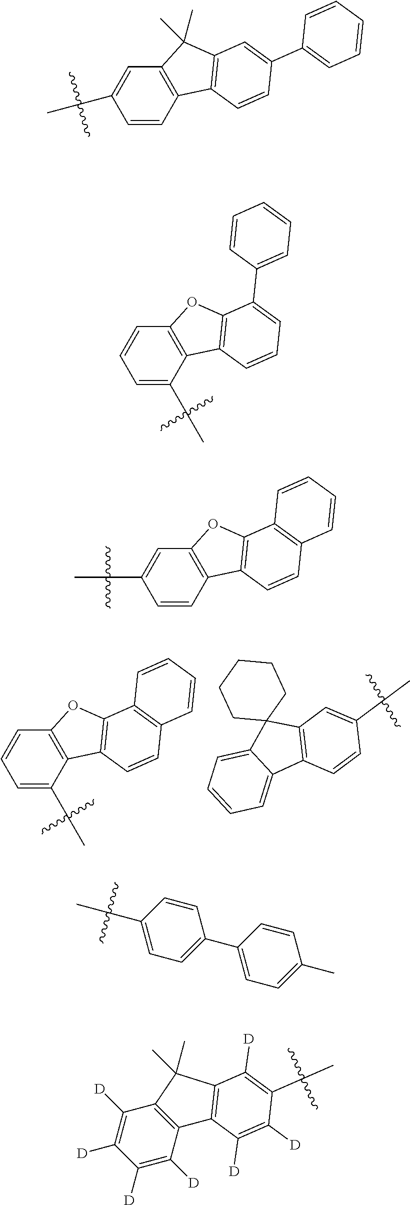

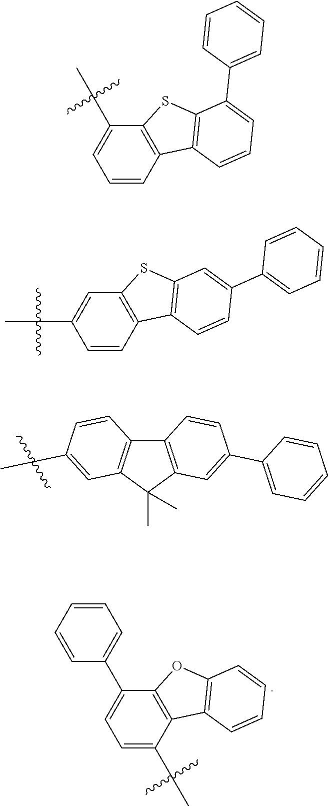 [SMILES: *c1cccc2c1sc1c(-c3ccccc3)cccc12 ; *c1ccc2c(c1)sc1cc(-c3ccccc3)ccc12 ; *c1ccc2c(c1)C(C)(C)c1cc(-c3ccccc3)ccc1-2 ; *c1ccc(-c2ccccc2)c2oc3ccccc3c12]

In a preferred embodiment, Ar₁ is selected from the following groups:

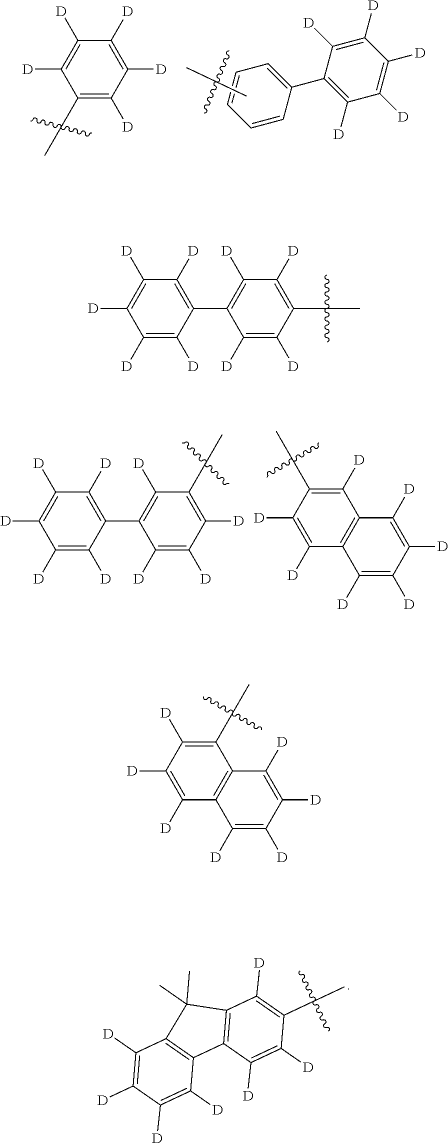

When the nitrogen-containing compound of the present application in which the Ar₁ group is selected from a deuterated aryl or a deuterated heteroaryl is used as the second hold transport layer of an organic electroluminescent device, the organic electroluminescent device has a higher lifetime.

In an embodiment,

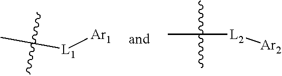

are identical or different, and each independently selected from the group consisting of the following groups:

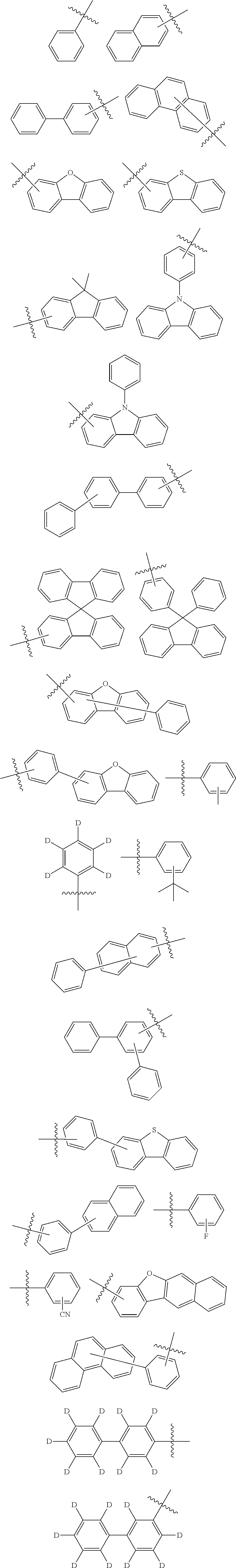

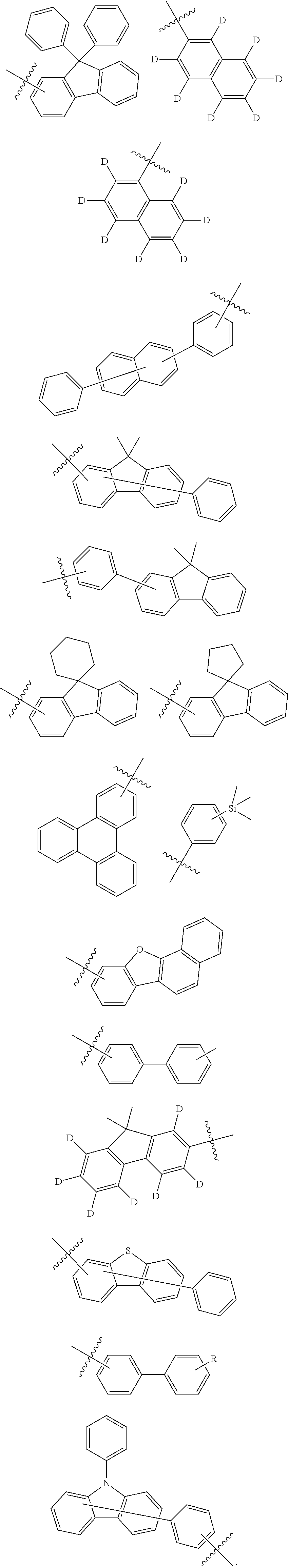

In an embodiment,

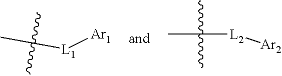

are identical or different, and each independently selected from the group consisting of the following groups:

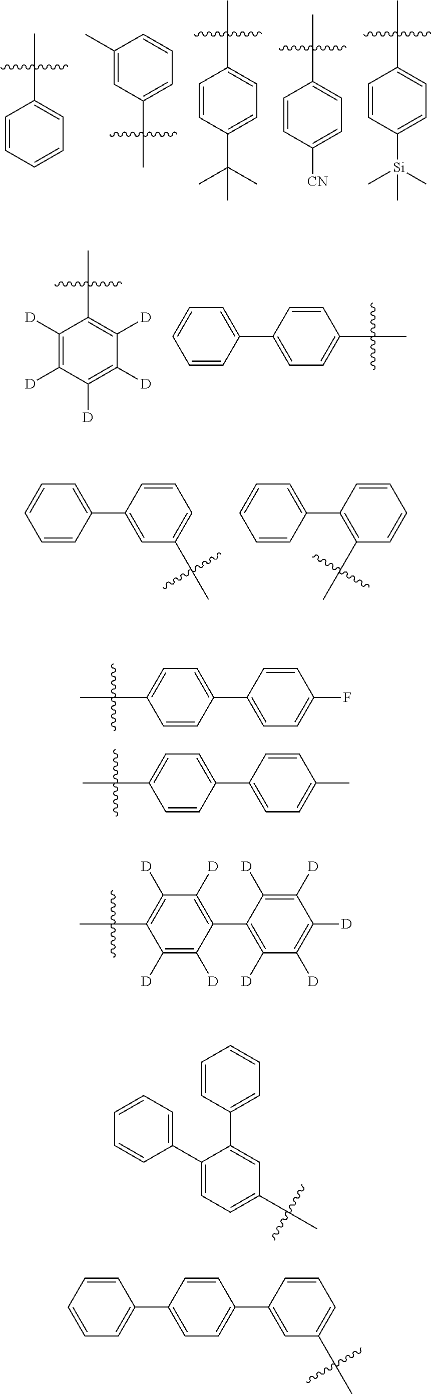

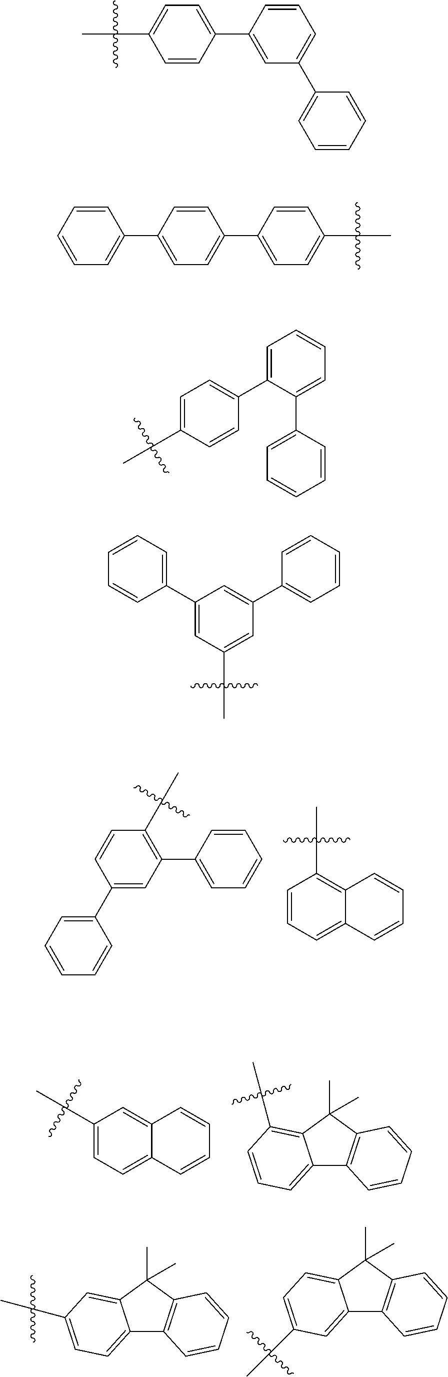

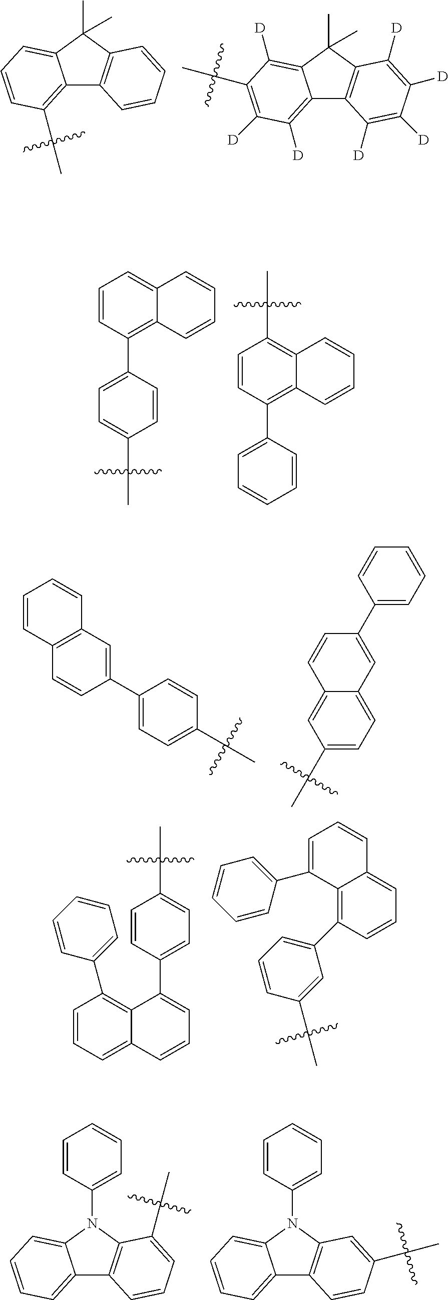

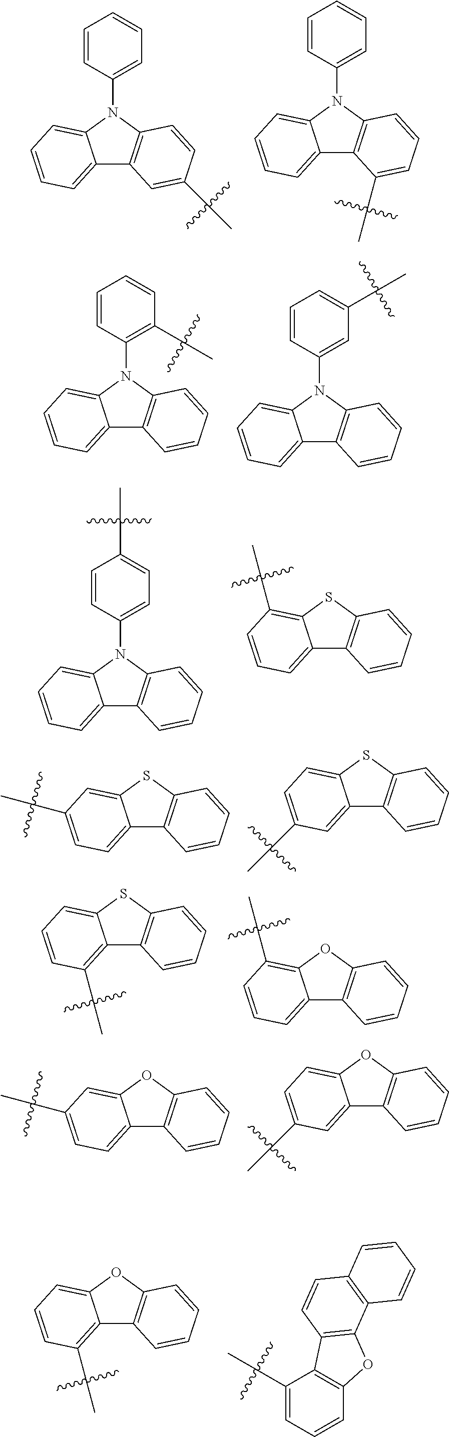

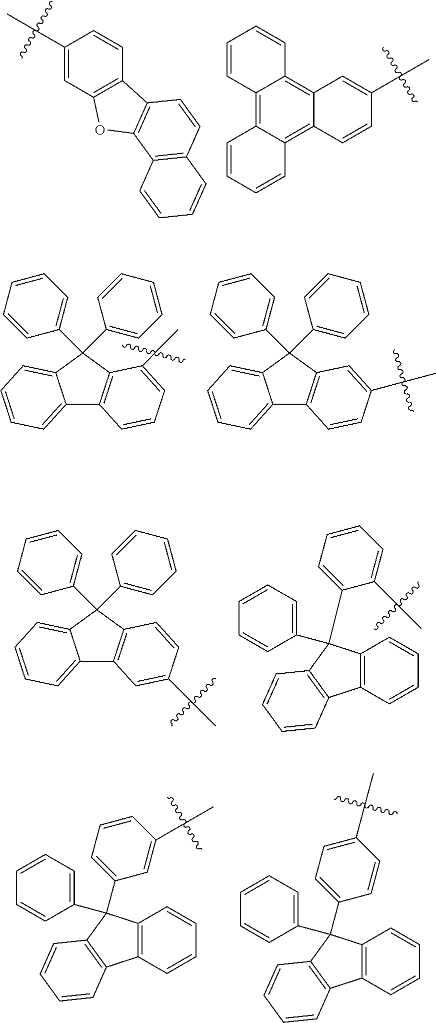

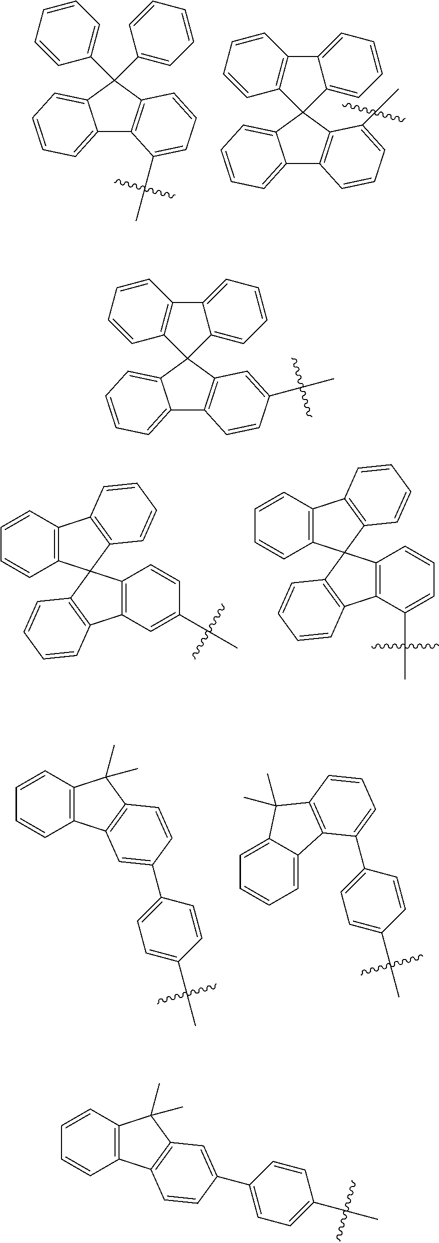

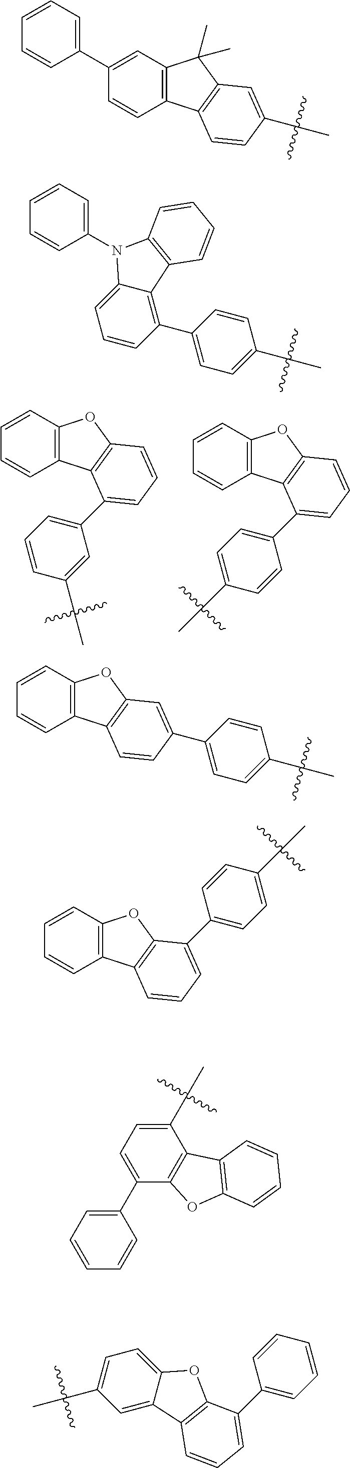

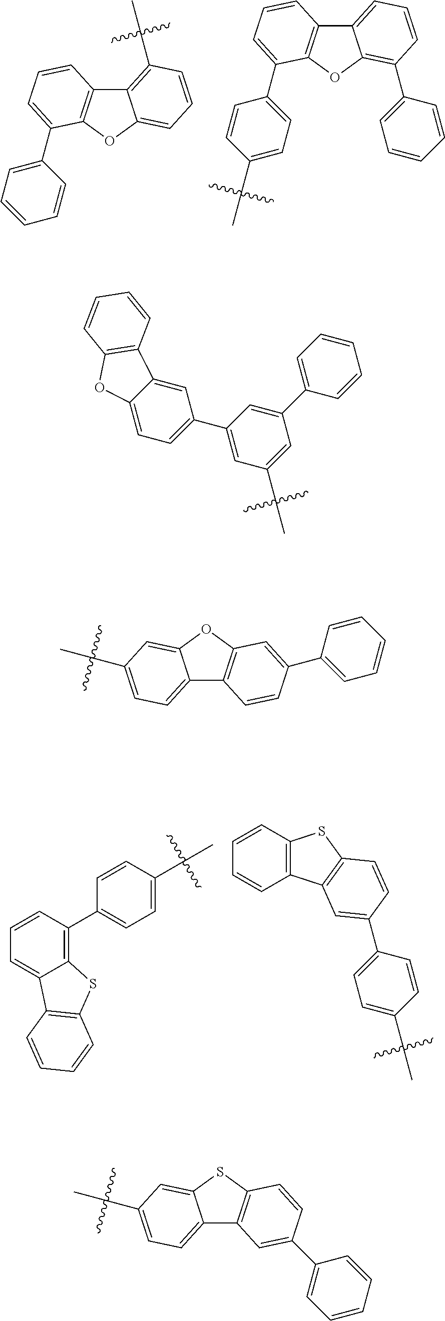

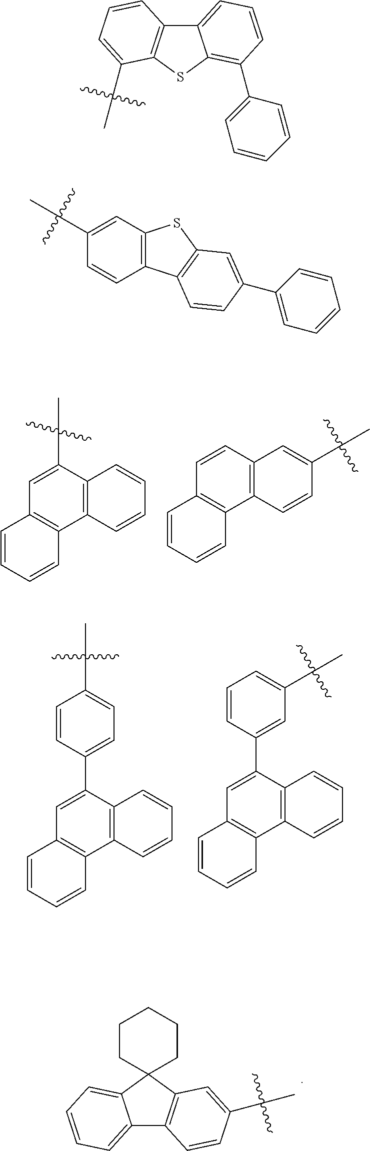

In a preferred embodiment,

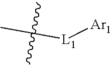

is selected from the group consisting of the following groups:

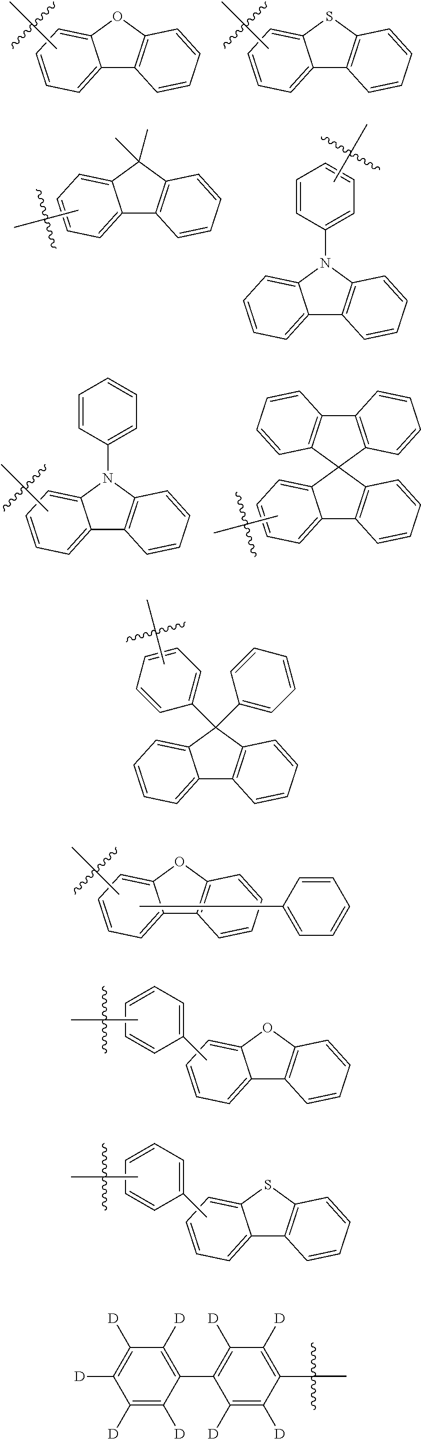

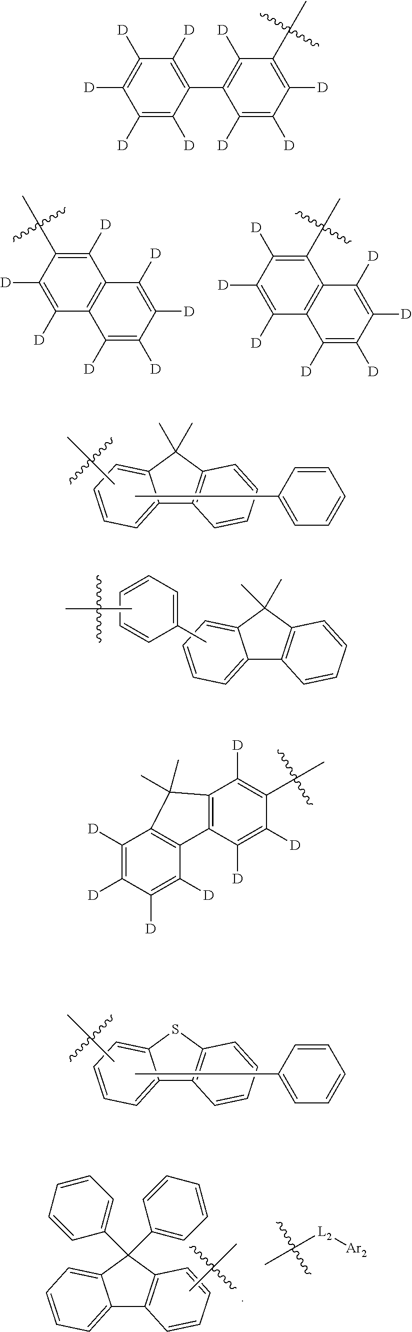

is selected from the group consisting of the following groups:

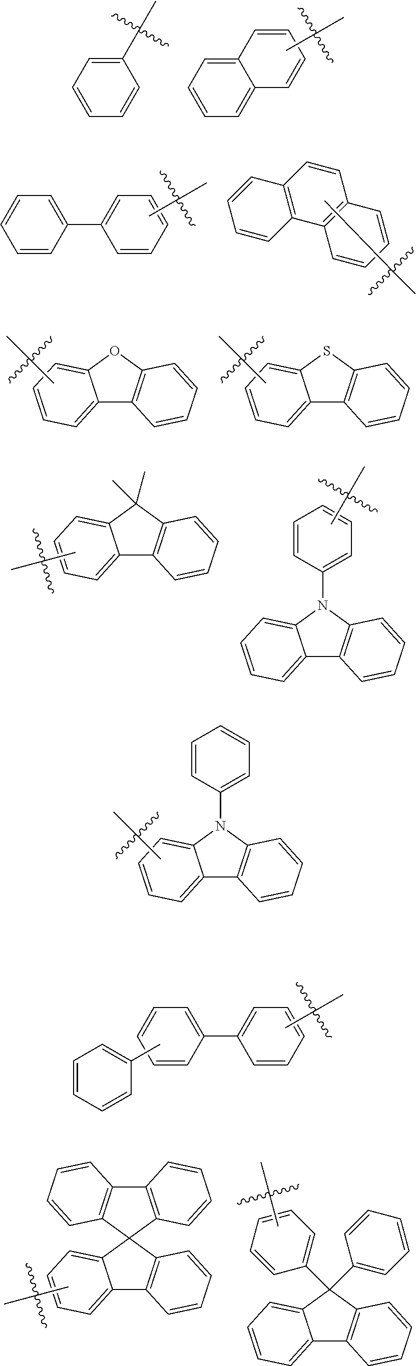

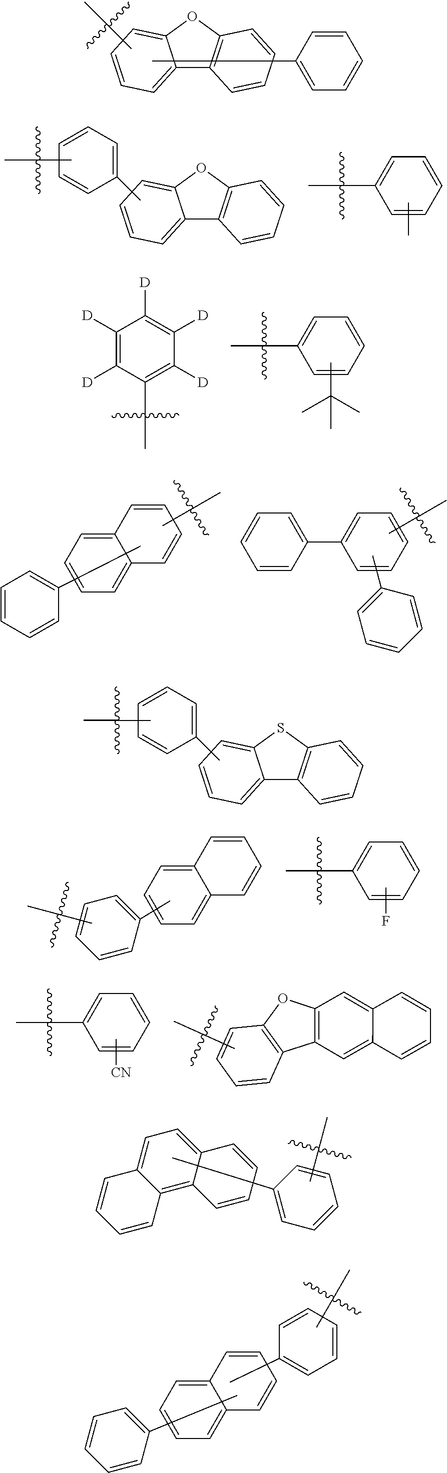

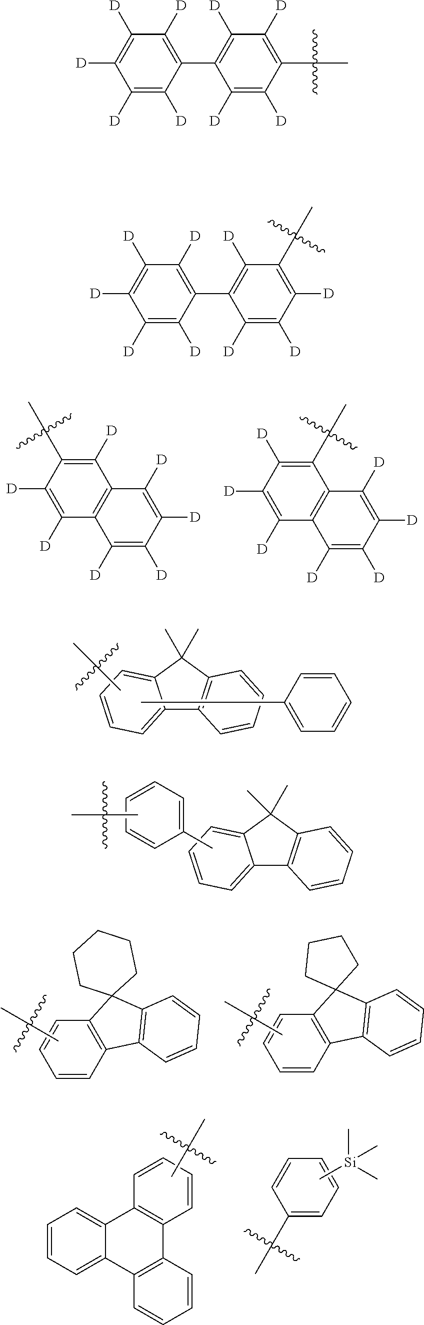

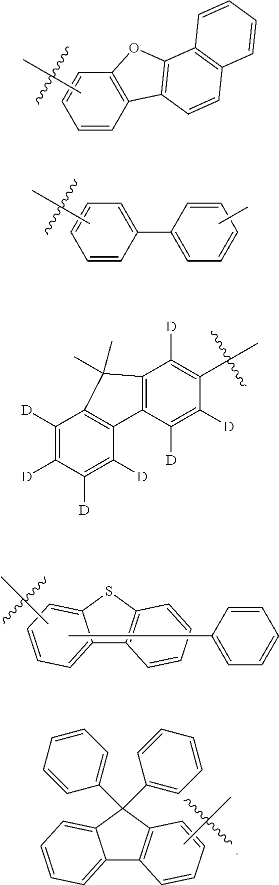

Optionally, the nitrogen-containing compound is selected from the group consisting of the following compounds:

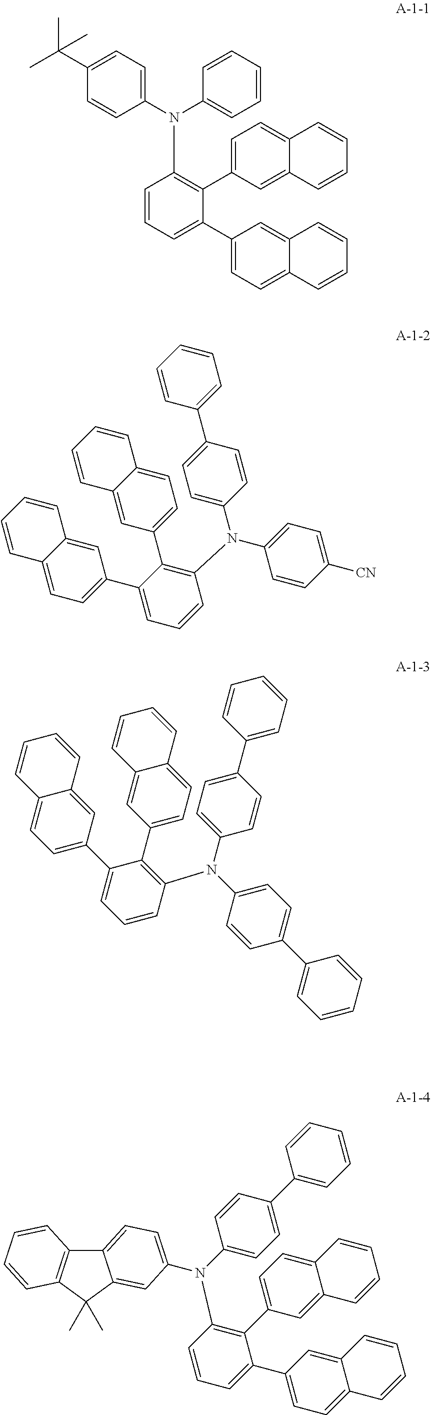 [SMILES: CC(C)(C)c1ccc(N(c2ccccc2)c2cccc(-c3ccc4ccccc4c3)c2-c2ccc3ccccc3c2)cc1 ; N#Cc1ccc(N(c2ccc(-c3ccccc3)cc2)c2cccc(-c3ccc4ccccc4c3)c2-c2ccc3ccccc3c2)cc1 ; c1ccc(-c2ccc(N(c3ccc(-c4ccccc4)cc3)c3cccc(-c4ccc5ccccc5c4)c3-c3ccc4ccccc4c3)cc2)cc1 ; CC1(C)c2ccccc2-c2ccc(N(c3ccc(-c4ccccc4)cc3)c3cccc(-c4ccc5ccccc5c4)c3-c3ccc4ccccc4c3)cc21]

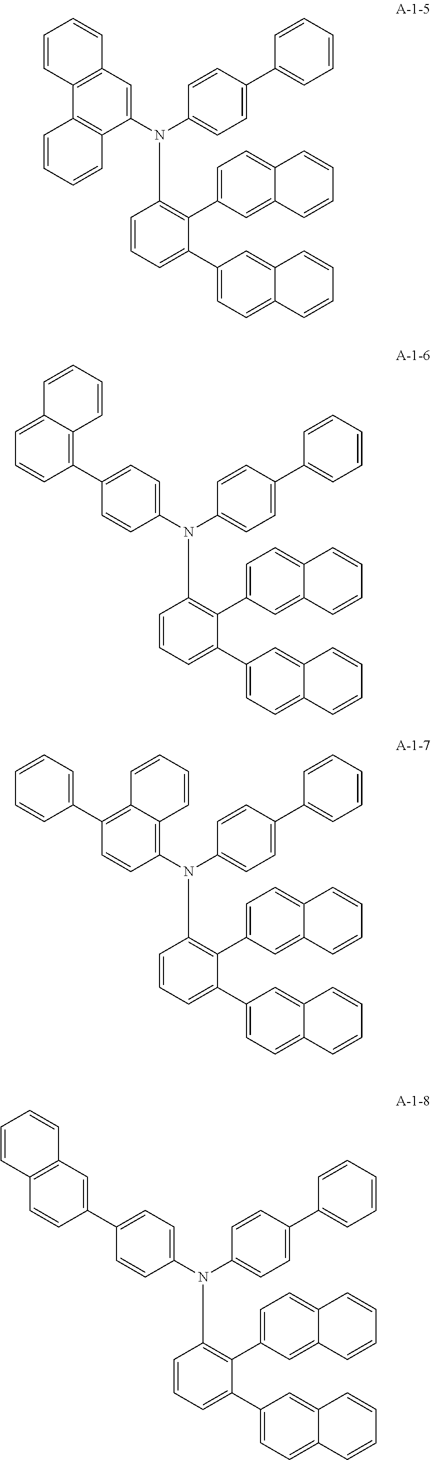

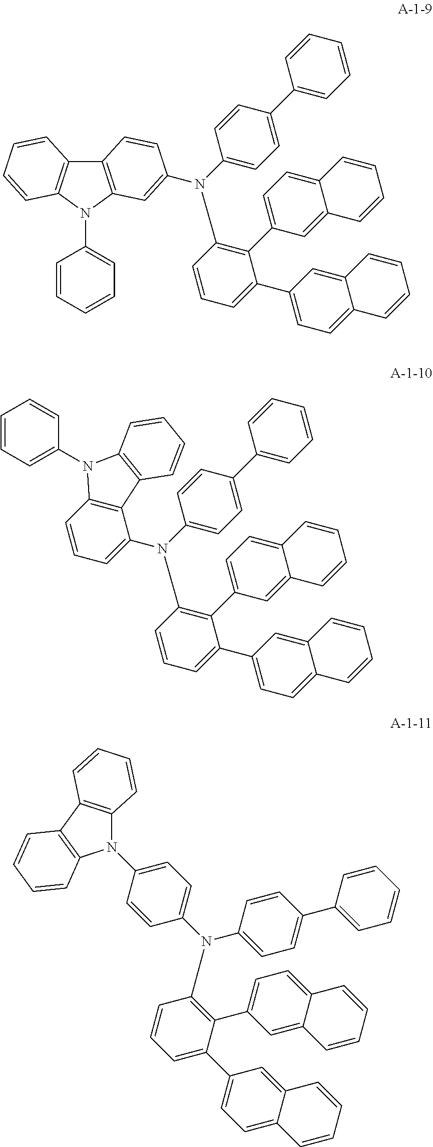 [SMILES: c1ccc(-c2ccc(N(c3ccc4c5ccccc5n(-c5ccccc5)c4c3)c3cccc(-c4ccc5ccccc5c4)c3-c3ccc4ccccc4c3)cc2)cc1 ; c1ccc(-c2ccc(N(c3cccc(-c4ccc5ccccc5c4)c3-c3ccc4ccccc4c3)c3cccc4c3c3ccccc3n4-c3ccccc3)cc2)cc1 ; c1ccc(-c2ccc(N(c3ccc(-n4c5ccccc5c5ccccc54)cc3)c3cccc(-c4ccc5ccccc5c4)c3-c3ccc4ccccc4c3)cc2)cc1]

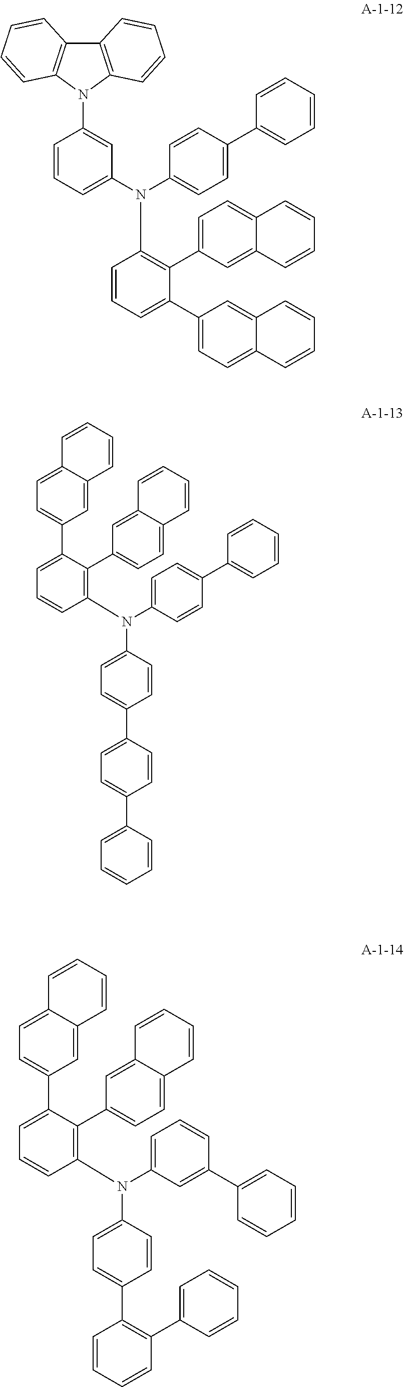 [SMILES: c1ccc(-c2ccc(N(c3cccc(-n4c5ccccc5c5ccccc54)c3)c3cccc(-c4ccc5ccccc5c4)c3-c3ccc4ccccc4c3)cc2)cc1 ; c1ccc(-c2ccc(-c3ccc(N(c4ccc(-c5ccccc5)cc4)c4cccc(-c5ccc6ccccc6c5)c4-c4ccc5ccccc5c4)cc3)cc2)cc1 ; c1ccc(-c2cccc(N(c3ccc(-c4ccccc4-c4ccccc4)cc3)c3cccc(-c4ccc5ccccc5c4)c3-c3ccc4ccccc4c3)c2)cc1]

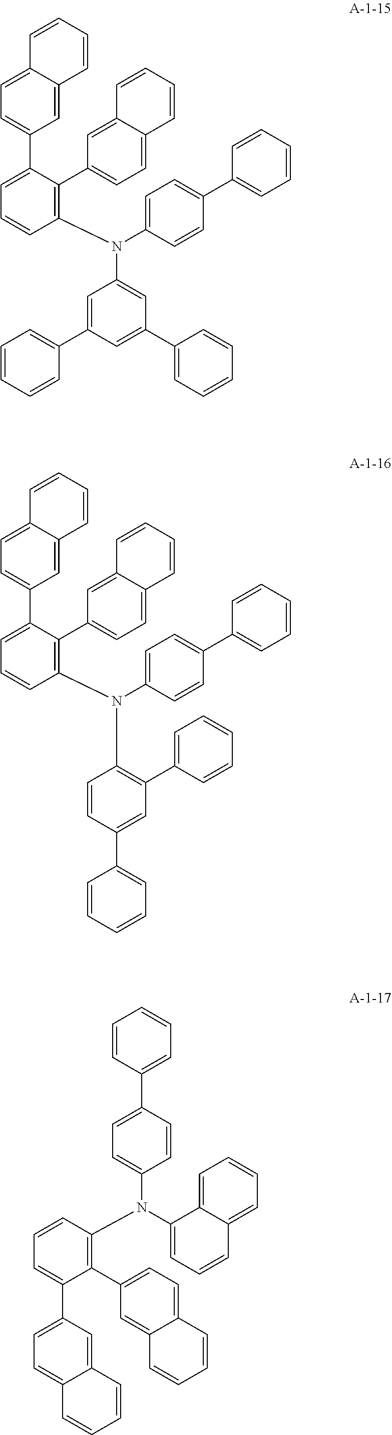 [SMILES: c1ccc(-c2ccc(N(c3cc(-c4ccccc4)cc(-c4ccccc4)c3)c3cccc(-c4ccc5ccccc5c4)c3-c3ccc4ccccc4c3)cc2)cc1 ; c1ccc(-c2ccc(N(c3ccc(-c4ccccc4)cc3-c3ccccc3)c3cccc(-c4ccc5ccccc5c4)c3-c3ccc4ccccc4c3)cc2)cc1 ; c1ccc(-c2ccc(N(c3cccc(-c4ccc5ccccc5c4)c3-c3ccc4ccccc4c3)c3cccc4ccccc34)cc2)cc1]

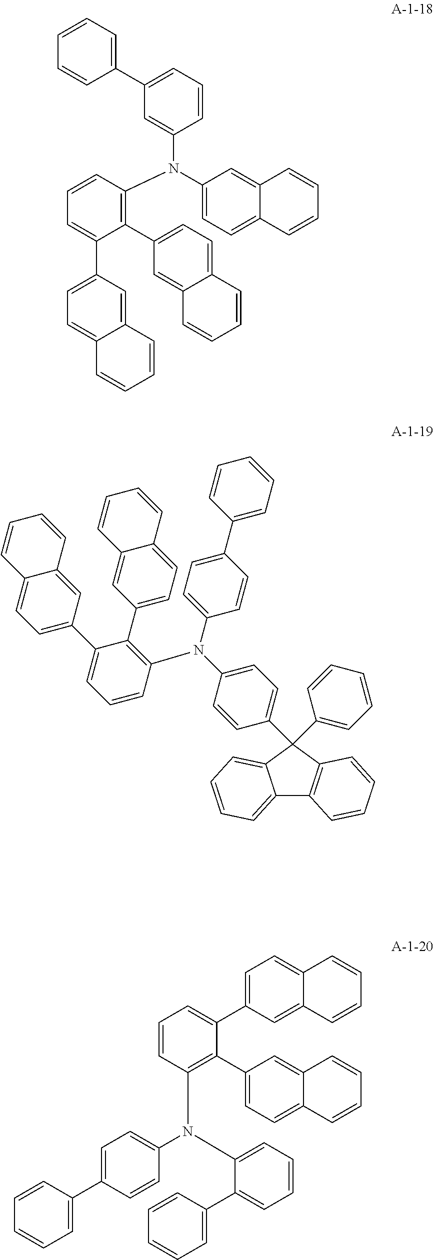 [SMILES: c1ccc(-c2cccc(N(c3ccc4ccccc4c3)c3cccc(-c4ccc5ccccc5c4)c3-c3ccc4ccccc4c3)c2)cc1 ; c1ccc(-c2ccc(N(c3ccc(C4(c5ccccc5)c5ccccc5-c5ccccc54)cc3)c3cccc(-c4ccc5ccccc5c4)c3-c3ccc4ccccc4c3)cc2)cc1 ; c1ccc(-c2ccc(N(c3ccccc3-c3ccccc3)c3cccc(-c4ccc5ccccc5c4)c3-c3ccc4ccccc4c3)cc2)cc1]

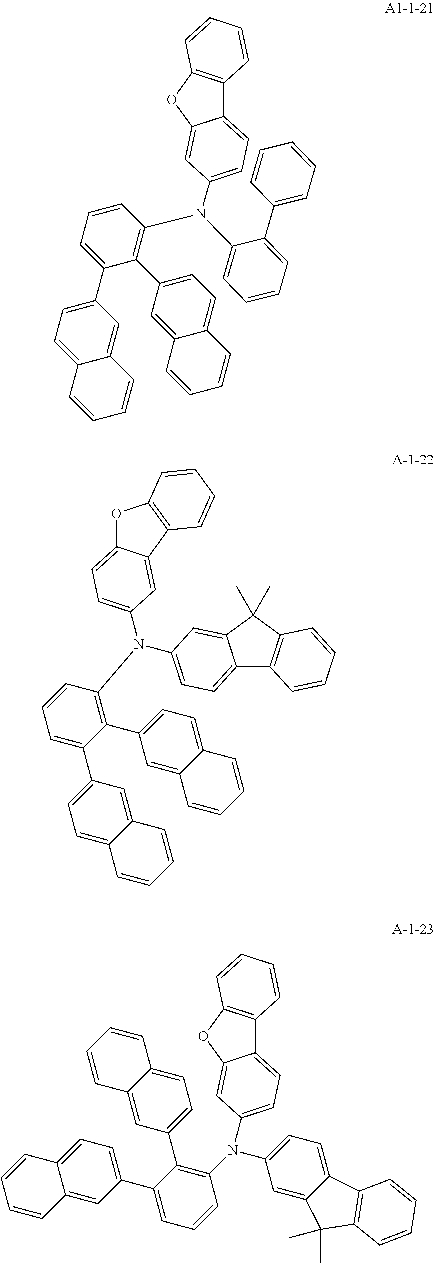 [SMILES: c1ccc(-c2ccccc2N(c2ccc3c(c2)oc2ccccc23)c2cccc(-c3ccc4ccccc4c3)c2-c2ccc3ccccc3c2)cc1 ; CC1(C)c2ccccc2-c2ccc(N(c3ccc4oc5ccccc5c4c3)c3cccc(-c4ccc5ccccc5c4)c3-c3ccc4ccccc4c3)cc21 ; CC1(C)c2ccccc2-c2ccc(N(c3ccc4c(c3)oc3ccccc34)c3cccc(-c4ccc5ccccc5c4)c3-c3ccc4ccccc4c3)cc21]

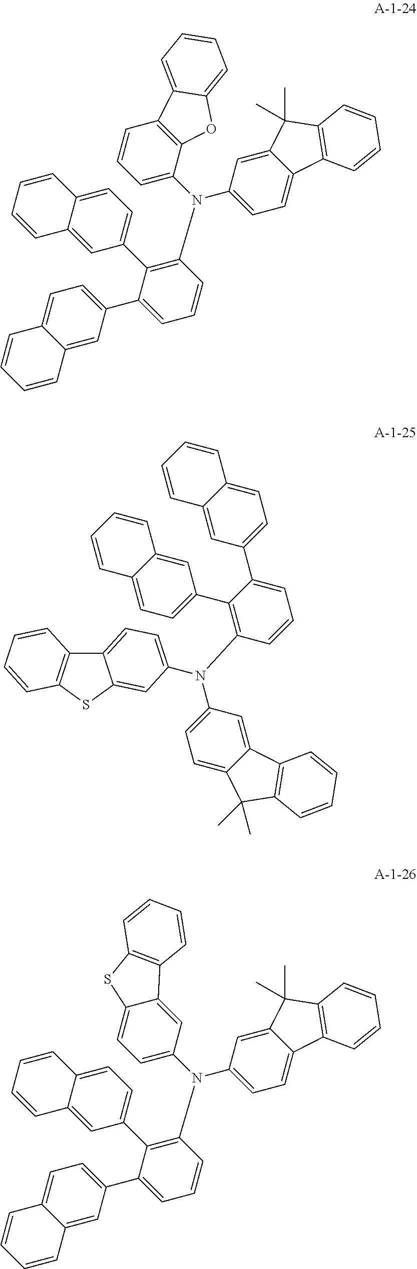 [SMILES: CC1(C)c2ccccc2-c2ccc(N(c3cccc(-c4ccc5ccccc5c4)c3-c3ccc4ccccc4c3)c3cccc4c3oc3ccccc34)cc21 ; CC1(C)c2ccccc2-c2cc(N(c3ccc4c(c3)sc3ccccc34)c3cccc(-c4ccc5ccccc5c4)c3-c3ccc4ccccc4c3)ccc21 ; CC1(C)c2ccccc2-c2ccc(N(c3ccc4sc5ccccc5c4c3)c3cccc(-c4ccc5ccccc5c4)c3-c3ccc4ccccc4c3)cc21]

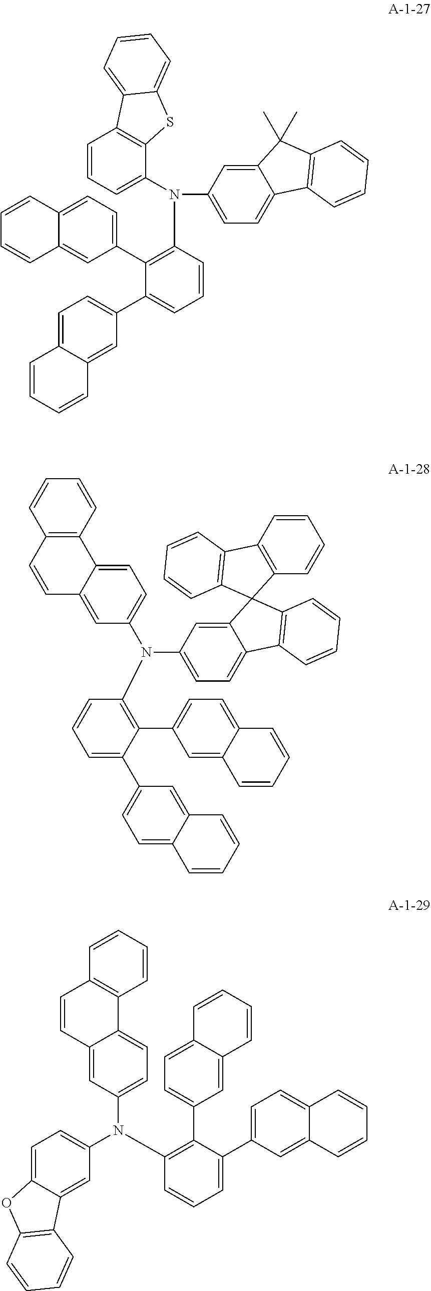 [SMILES: CC1(C)c2ccccc2-c2ccc(N(c3cccc(-c4ccc5ccccc5c4)c3-c3ccc4ccccc4c3)c3cccc4c3sc3ccccc34)cc21 ; c1ccc2c(c1)-c1ccccc1C21c2ccccc2-c2ccc(N(c3ccc4c(ccc5ccccc54)c3)c3cccc(-c4ccc5ccccc5c4)c3-c3ccc4ccccc4c3)cc21 ; c1cc(-c2ccc3ccccc3c2)c(-c2ccc3ccccc3c2)c(N(c2ccc3c(ccc4ccccc43)c2)c2ccc3oc4ccccc4c3c2)c1]

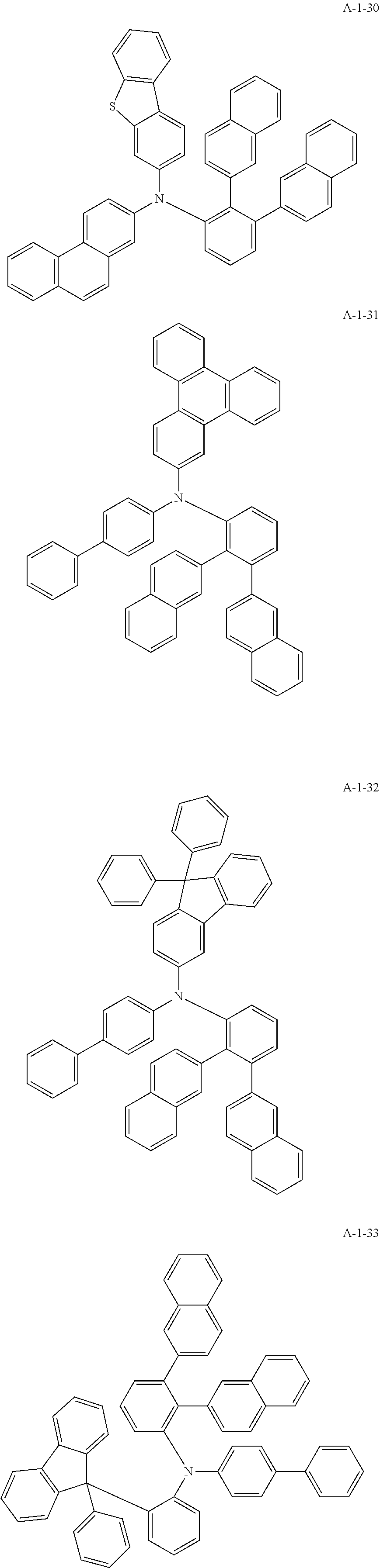 [SMILES: c1cc(-c2ccc3ccccc3c2)c(-c2ccc3ccccc3c2)c(N(c2ccc3c(ccc4ccccc43)c2)c2ccc3c(c2)sc2ccccc23)c1 ; c1ccc(-c2ccc(N(c3ccc4c5ccccc5c5ccccc5c4c3)c3cccc(-c4ccc5ccccc5c4)c3-c3ccc4ccccc4c3)cc2)cc1 ; c1ccc(-c2ccc(N(c3ccc4c(c3)-c3ccccc3C4(c3ccccc3)c3ccccc3)c3cccc(-c4ccc5ccccc5c4)c3-c3ccc4ccccc4c3)cc2)cc1 ; c1ccc(-c2ccc(N(c3ccccc3C3(c4ccccc4)c4ccccc4-c4ccccc43)c3cccc(-c4ccc5ccccc5c4)c3-c3ccc4ccccc4c3)cc2)cc1]

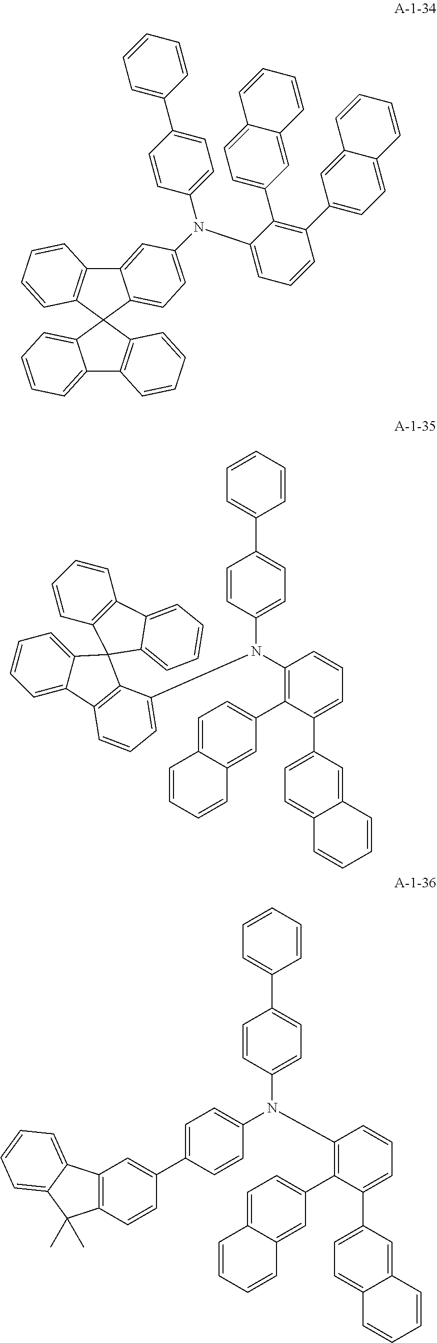 [SMILES: c1ccc(-c2ccc(N(c3ccc4c(c3)-c3ccccc3C43c4ccccc4-c4ccccc43)c3cccc(-c4ccc5ccccc5c4)c3-c3ccc4ccccc4c3)cc2)cc1 ; c1ccc(-c2ccc(N(c3cccc(-c4ccc5ccccc5c4)c3-c3ccc4ccccc4c3)c3cccc4c3C3(c5ccccc5-c5ccccc53)c3ccccc3-4)cc2)cc1 ; CC1(C)c2ccccc2-c2cc(-c3ccc(N(c4ccc(-c5ccccc5)cc4)c4cccc(-c5ccc6ccccc6c5)c4-c4ccc5ccccc5c4)cc3)ccc21]

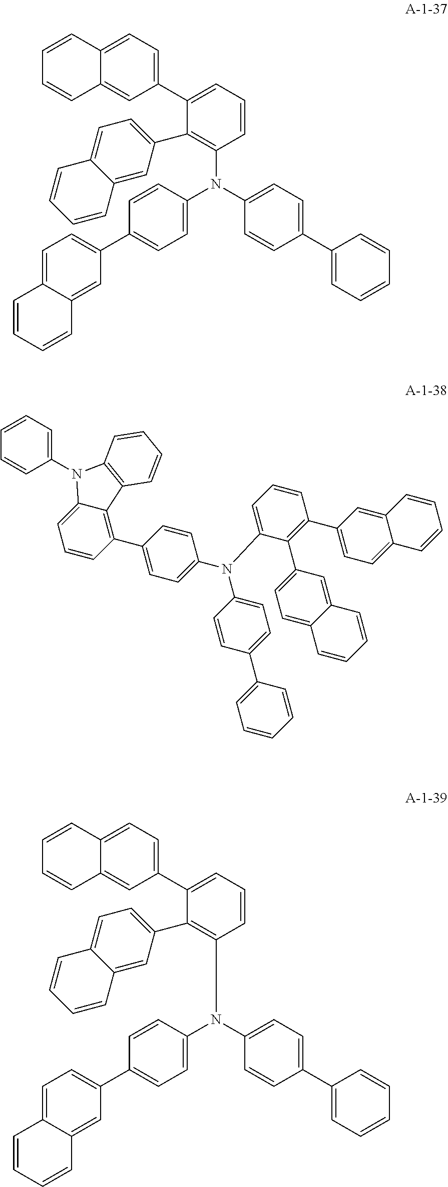 [SMILES: c1ccc(-c2ccc(N(c3ccc(-c4ccc5ccccc5c4)cc3)c3cccc(-c4ccc5ccccc5c4)c3-c3ccc4ccccc4c3)cc2)cc1 ; c1ccc(-c2ccc(N(c3ccc(-c4cccc5c4c4ccccc4n5-c4ccccc4)cc3)c3cccc(-c4ccc5ccccc5c4)c3-c3ccc4ccccc4c3)cc2)cc1 ; c1ccc(-c2ccc(N(c3ccc(-c4ccc5ccccc5c4)cc3)c3cccc(-c4ccc5ccccc5c4)c3-c3ccc4ccccc4c3)cc2)cc1]

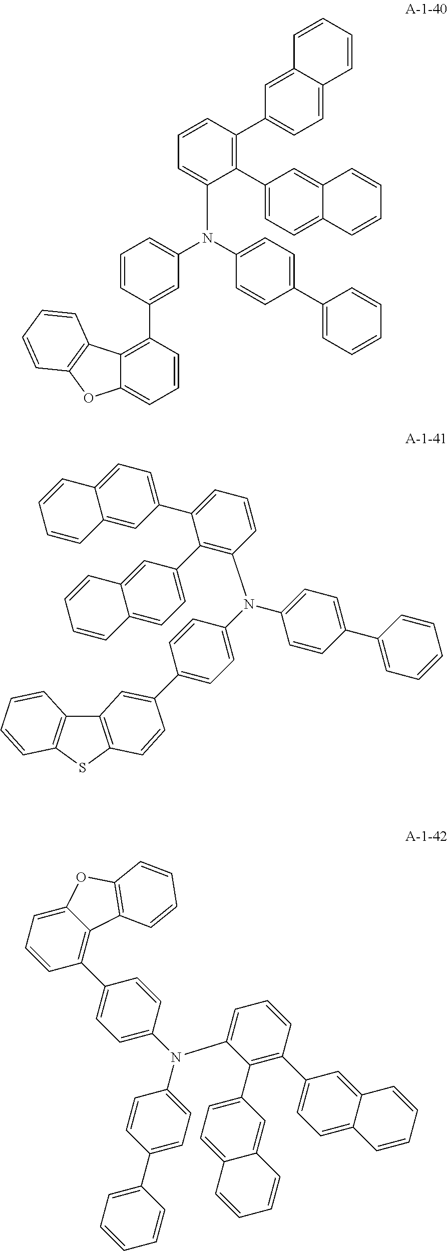 [SMILES: c1ccc(-c2ccc(N(c3cccc(-c4cccc5oc6ccccc6c45)c3)c3cccc(-c4ccc5ccccc5c4)c3-c3ccc4ccccc4c3)cc2)cc1 ; c1ccc(-c2ccc(N(c3ccc(-c4ccc5sc6ccccc6c5c4)cc3)c3cccc(-c4ccc5ccccc5c4)c3-c3ccc4ccccc4c3)cc2)cc1 ; c1ccc(-c2ccc(N(c3ccc(-c4cccc5oc6ccccc6c45)cc3)c3cccc(-c4ccc5ccccc5c4)c3-c3ccc4ccccc4c3)cc2)cc1]

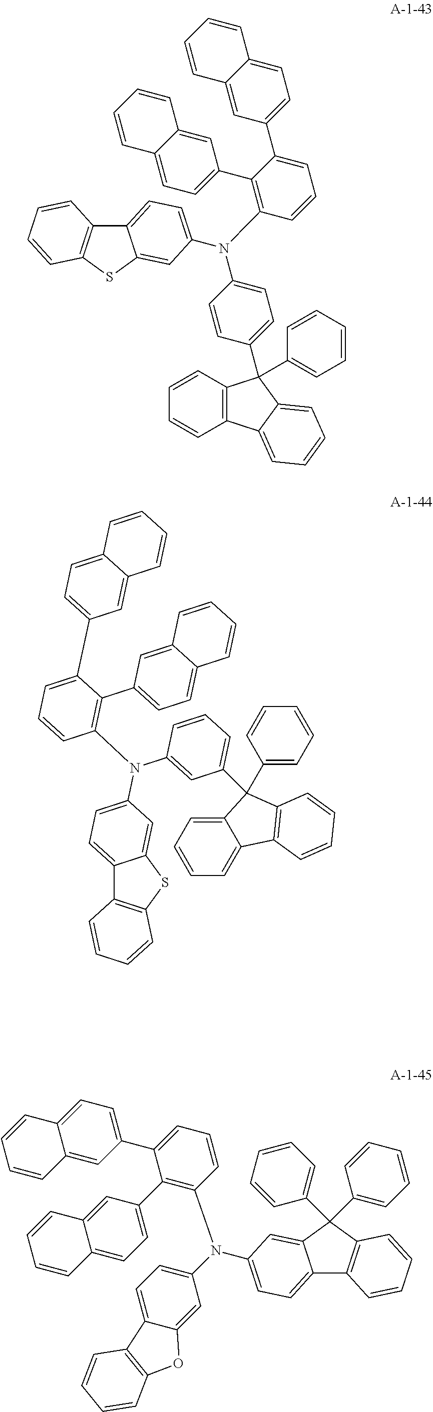 [SMILES: c1ccc(C2(c3ccc(N(c4ccc5c(c4)sc4ccccc45)c4cccc(-c5ccc6ccccc6c5)c4-c4ccc5ccccc5c4)cc3)c3ccccc3-c3ccccc32)cc1 ; c1ccc(C2(c3cccc(N(c4ccc5c(c4)sc4ccccc45)c4cccc(-c5ccc6ccccc6c5)c4-c4ccc5ccccc5c4)c3)c3ccccc3-c3ccccc32)cc1 ; c1ccc(C2(c3ccccc3)c3ccccc3-c3ccc(N(c4ccc5c(c4)oc4ccccc45)c4cccc(-c5ccc6ccccc6c5)c4-c4ccc5ccccc5c4)cc32)cc1]

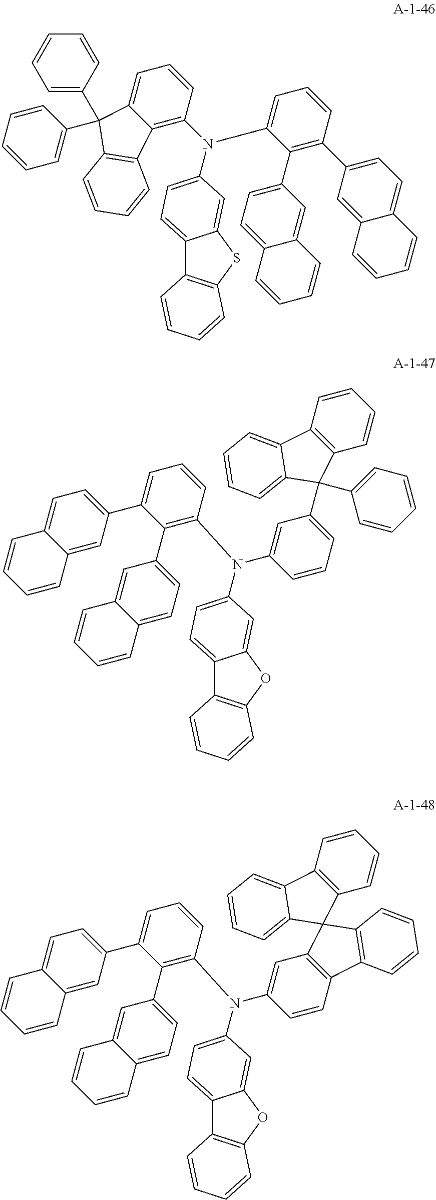 [SMILES: c1ccc(C2(c3ccccc3)c3ccccc3-c3c(N(c4ccc5c(c4)sc4ccccc45)c4cccc(-c5ccc6ccccc6c5)c4-c4ccc5ccccc5c4)cccc32)cc1 ; c1ccc(C2(c3cccc(N(c4ccc5c(c4)oc4ccccc45)c4cccc(-c5ccc6ccccc6c5)c4-c4ccc5ccccc5c4)c3)c3ccccc3-c3ccccc32)cc1 ; c1ccc2c(c1)-c1ccccc1C21c2ccccc2-c2ccc(N(c3ccc4c(c3)oc3ccccc34)c3cccc(-c4ccc5ccccc5c4)c3-c3ccc4ccccc4c3)cc21]

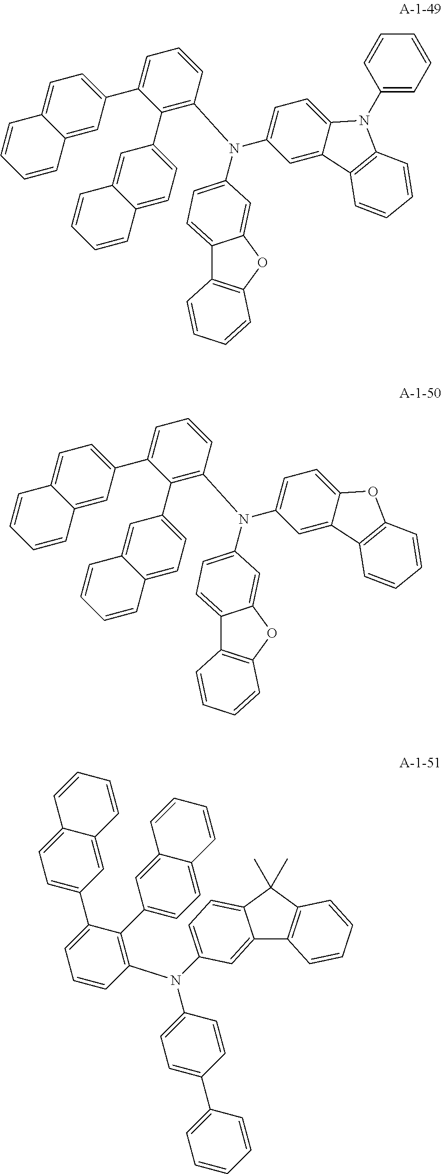 [SMILES: c1ccc(-n2c3ccccc3c3cc(N(c4ccc5c(c4)oc4ccccc45)c4cccc(-c5ccc6ccccc6c5)c4-c4ccc5ccccc5c4)ccc32)cc1 ; c1cc(-c2ccc3ccccc3c2)c(-c2ccc3ccccc3c2)c(N(c2ccc3c(c2)oc2ccccc23)c2ccc3oc4ccccc4c3c2)c1 ; CC1(C)c2ccccc2-c2cc(N(c3ccc(-c4ccccc4)cc3)c3cccc(-c4ccc5ccccc5c4)c3-c3ccc4ccccc4c3)ccc21]

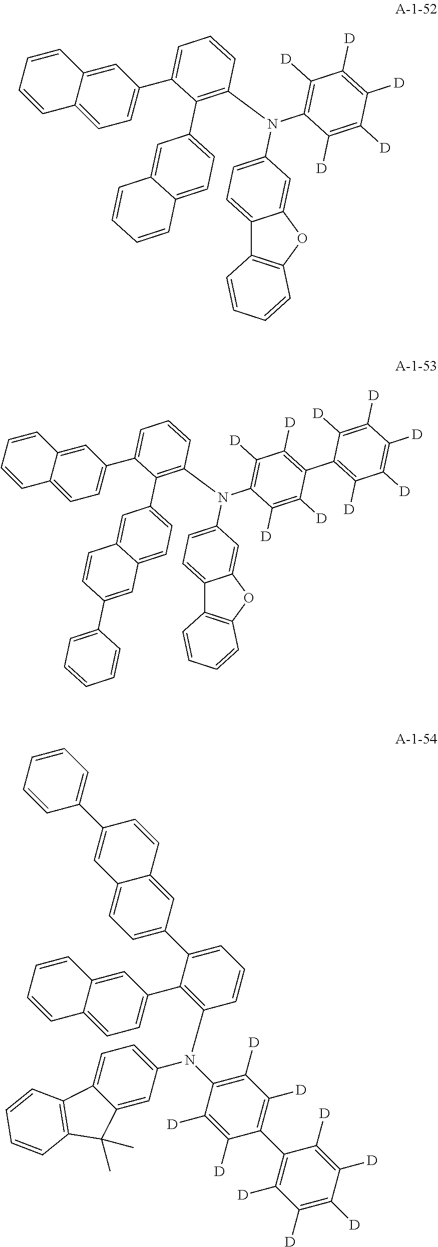 [SMILES: [2H]c1c([2H])c([2H])c(N(c2ccc3c(c2)oc2ccccc23)c2cccc(-c3ccc4ccccc4c3)c2-c2ccc3ccccc3c2)c([2H])c1[2H] ; [2H]c1c([2H])c([2H])c(-c2c([2H])c([2H])c(N(c3ccc4c(c3)oc3ccccc34)c3cccc(-c4ccc5ccccc5c4)c3-c3ccc4cc(-c5ccccc5)ccc4c3)c([2H])c2[2H])c([2H])c1[2H] ; [2H]c1c([2H])c([2H])c(-c2c([2H])c([2H])c(N(c3ccc4c(c3)C(C)(C)c3ccccc3-4)c3cccc(-c4ccc5cc(-c6ccccc6)ccc5c4)c3-c3ccc4ccccc4c3)c([2H])c2[2H])c([2H])c1[2H]]

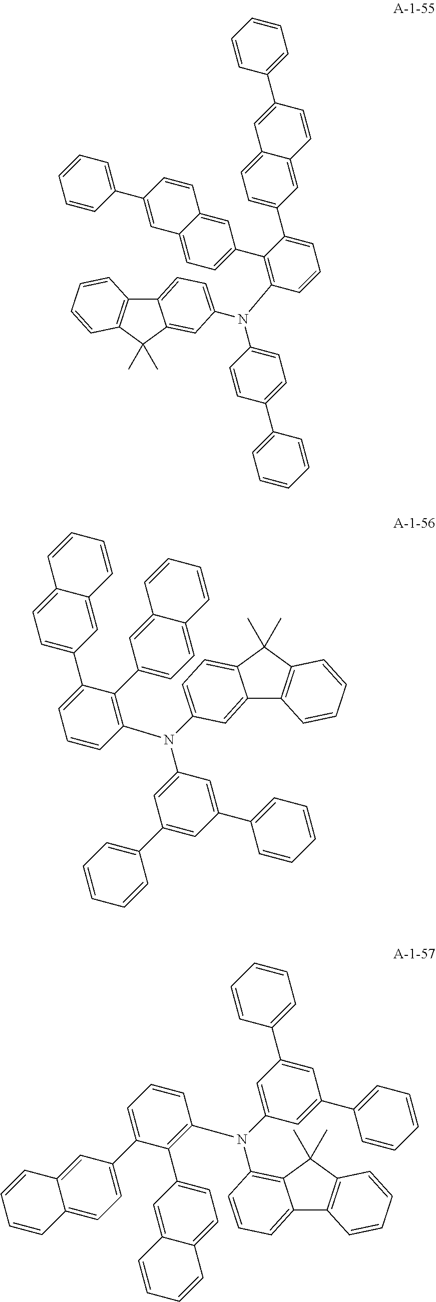 [SMILES: CC1(C)c2ccccc2-c2ccc(N(c3ccc(-c4ccccc4)cc3)c3cccc(-c4ccc5cc(-c6ccccc6)ccc5c4)c3-c3ccc4cc(-c5ccccc5)ccc4c3)cc21 ; CC1(C)c2ccccc2-c2cc(N(c3cc(-c4ccccc4)cc(-c4ccccc4)c3)c3cccc(-c4ccc5ccccc5c4)c3-c3ccc4ccccc4c3)ccc21 ; CC1(C)c2ccccc2-c2cccc(N(c3cc(-c4ccccc4)cc(-c4ccccc4)c3)c3cccc(-c4ccc5ccccc5c4)c3-c3ccc4ccccc4c3)c21]

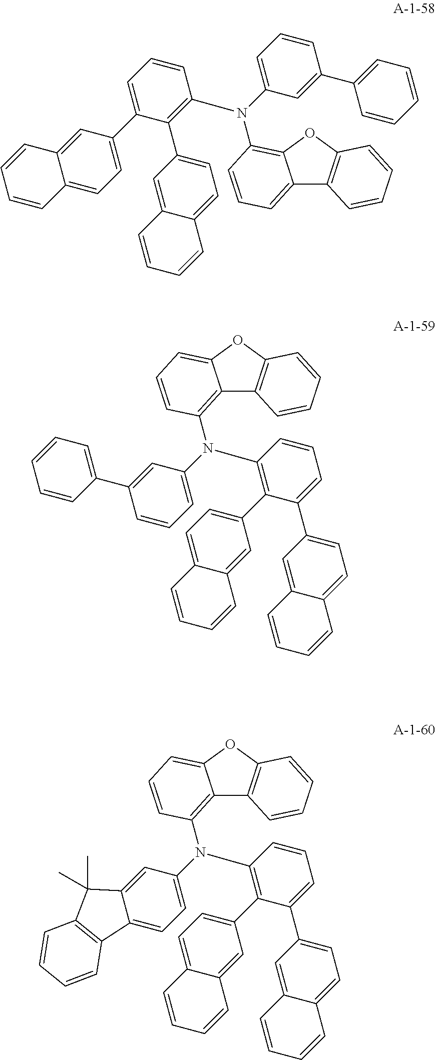 [SMILES: c1ccc(-c2cccc(N(c3cccc(-c4ccc5ccccc5c4)c3-c3ccc4ccccc4c3)c3cccc4c3oc3ccccc34)c2)cc1 ; c1ccc(-c2cccc(N(c3cccc(-c4ccc5ccccc5c4)c3-c3ccc4ccccc4c3)c3cccc4oc5ccccc5c34)c2)cc1 ; CC1(C)c2ccccc2-c2ccc(N(c3cccc(-c4ccc5ccccc5c4)c3-c3ccc4ccccc4c3)c3cccc4oc5ccccc5c34)cc21]

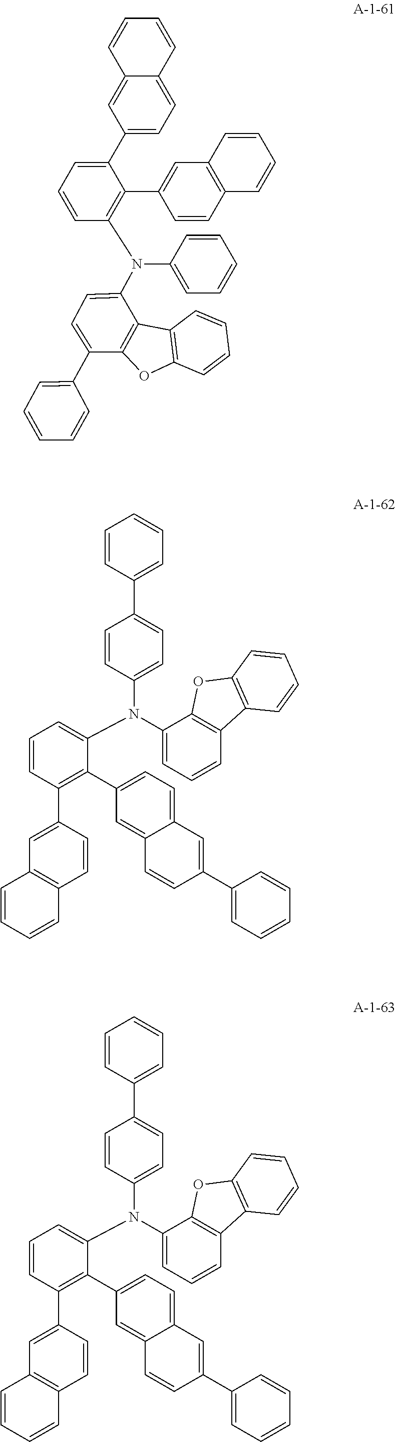 [SMILES: c1ccc(-c2ccc(N(c3ccccc3)c3cccc(-c4ccc5ccccc5c4)c3-c3ccc4ccccc4c3)c3c2oc2ccccc23)cc1 ; c1ccc(-c2ccc(N(c3cccc(-c4ccc5ccccc5c4)c3-c3ccc4cc(-c5ccccc5)ccc4c3)c3cccc4c3oc3ccccc34)cc2)cc1 ; c1ccc(-c2ccc(N(c3cccc(-c4ccc5ccccc5c4)c3-c3ccc4cc(-c5ccccc5)ccc4c3)c3cccc4c3oc3ccccc34)cc2)cc1]

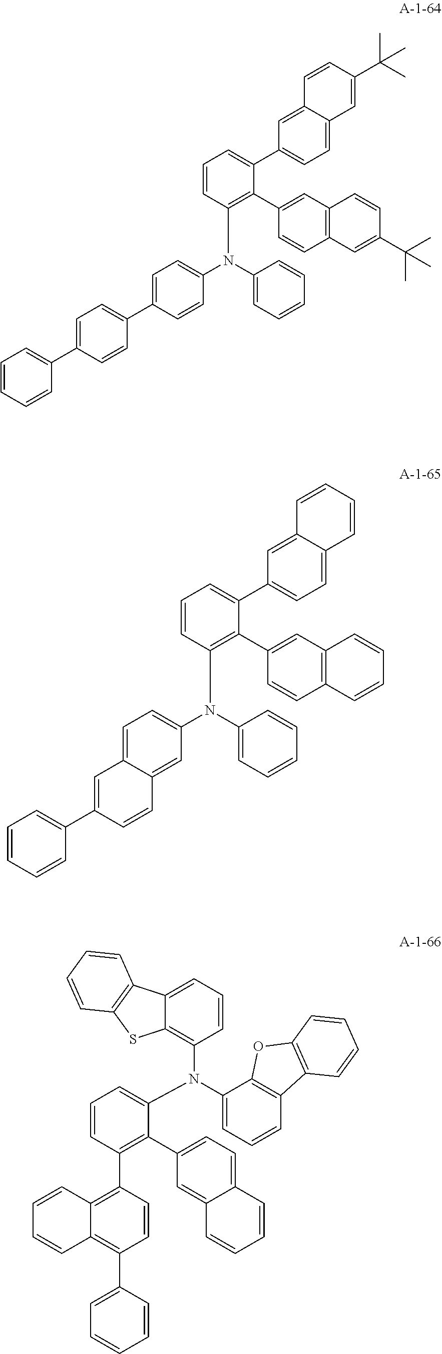 [SMILES: CC(C)(C)c1ccc2cc(-c3cccc(N(c4ccccc4)c4ccc(-c5ccc(-c6ccccc6)cc5)cc4)c3-c3ccc4cc(C(C)(C)C)ccc4c3)ccc2c1 ; c1ccc(-c2ccc3cc(N(c4ccccc4)c4cccc(-c5ccc6ccccc6c5)c4-c4ccc5ccccc5c4)ccc3c2)cc1 ; c1ccc(-c2ccc(-c3cccc(N(c4cccc5c4oc4ccccc45)c4cccc5c4sc4ccccc45)c3-c3ccc4ccccc4c3)c3ccccc23)cc1]

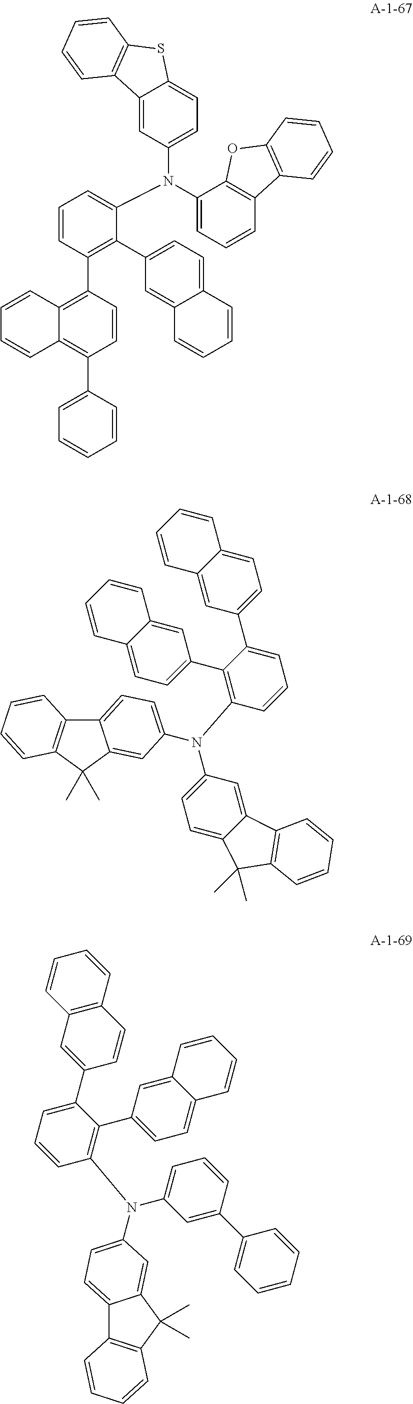 [SMILES: c1ccc(-c2ccc(-c3cccc(N(c4ccc5sc6ccccc6c5c4)c4cccc5c4oc4ccccc45)c3-c3ccc4ccccc4c3)c3ccccc23)cc1 ; CC1(C)c2ccccc2-c2cc(N(c3ccc4c(c3)C(C)(C)c3ccccc3-4)c3cccc(-c4ccc5ccccc5c4)c3-c3ccc4ccccc4c3)ccc21 ; CC1(C)c2ccccc2-c2ccc(N(c3cccc(-c4ccccc4)c3)c3cccc(-c4ccc5ccccc5c4)c3-c3ccc4ccccc4c3)cc21]

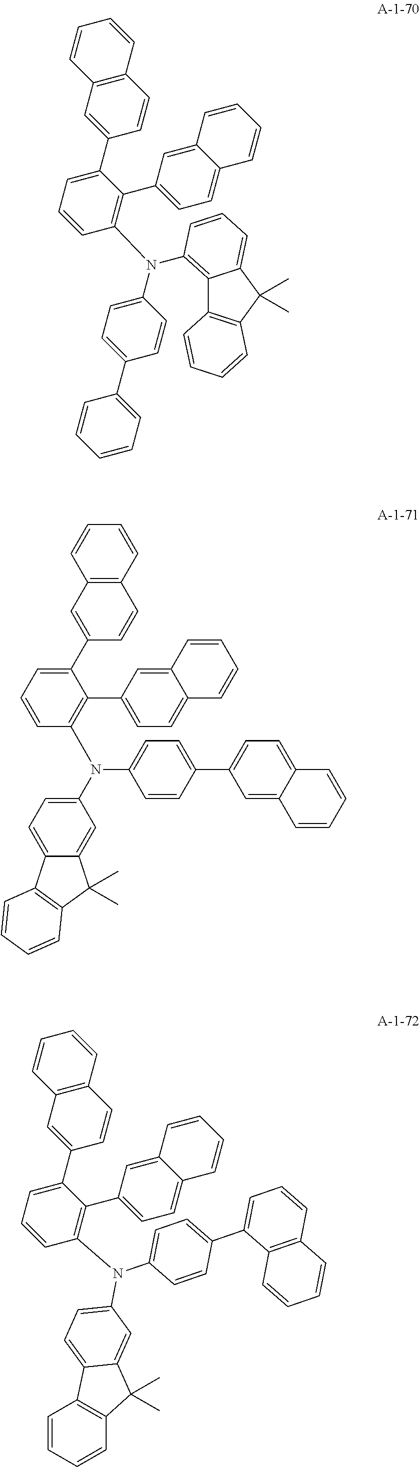 [SMILES: CC1(C)c2ccccc2-c2c(N(c3ccc(-c4ccccc4)cc3)c3cccc(-c4ccc5ccccc5c4)c3-c3ccc4ccccc4c3)cccc21 ; CC1(C)c2ccccc2-c2ccc(N(c3ccc(-c4ccc5ccccc5c4)cc3)c3cccc(-c4ccc5ccccc5c4)c3-c3ccc4ccccc4c3)cc21 ; CC1(C)c2ccccc2-c2ccc(N(c3ccc(-c4cccc5ccccc45)cc3)c3cccc(-c4ccc5ccccc5c4)c3-c3ccc4ccccc4c3)cc21]

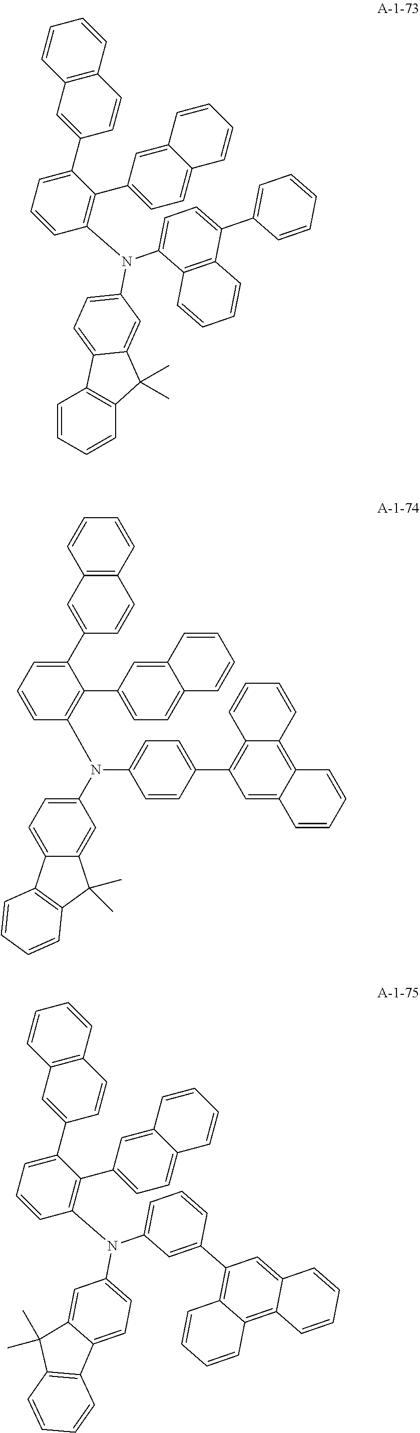 [SMILES: CC1(C)c2ccccc2-c2ccc(N(c3cccc(-c4ccc5ccccc5c4)c3-c3ccc4ccccc4c3)c3ccc(-c4ccccc4)c4ccccc34)cc21 ; CC1(C)c2ccccc2-c2ccc(N(c3ccc(-c4cc5ccccc5c5ccccc45)cc3)c3cccc(-c4ccc5ccccc5c4)c3-c3ccc4ccccc4c3)cc21 ; CC1(C)c2ccccc2-c2ccc(N(c3cccc(-c4cc5ccccc5c5ccccc45)c3)c3cccc(-c4ccc5ccccc5c4)c3-c3ccc4ccccc4c3)cc21]

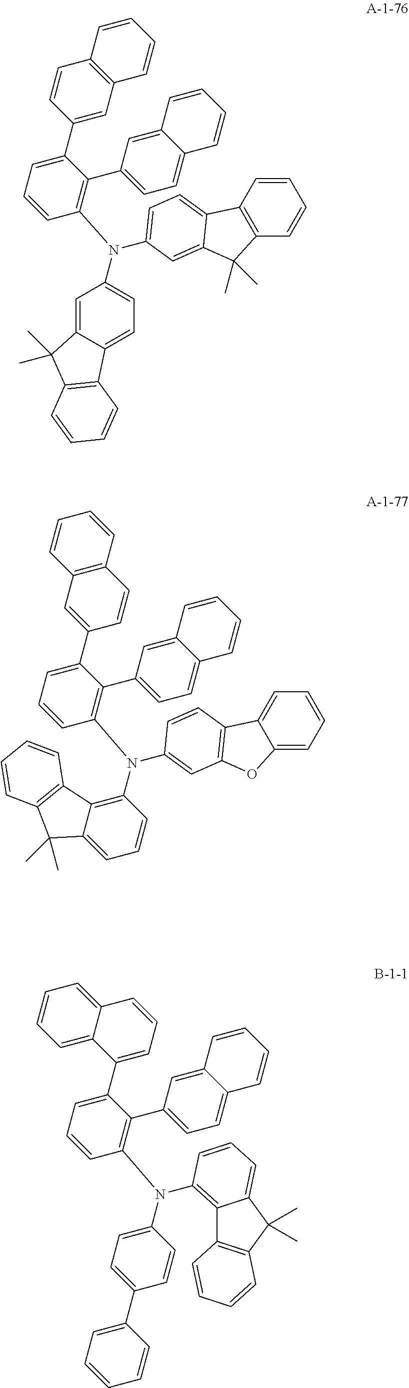 [SMILES: CC1(C)c2ccccc2-c2ccc(N(c3ccc4c(c3)C(C)(C)c3ccccc3-4)c3cccc(-c4ccc5ccccc5c4)c3-c3ccc4ccccc4c3)cc21 ; CC1(C)c2ccccc2-c2c(N(c3ccc4c(c3)oc3ccccc34)c3cccc(-c4ccc5ccccc5c4)c3-c3ccc4ccccc4c3)cccc21 ; CC1(C)c2ccccc2-c2c(N(c3ccc(-c4ccccc4)cc3)c3cccc(-c4cccc5ccccc45)c3-c3ccc4ccccc4c3)cccc21]

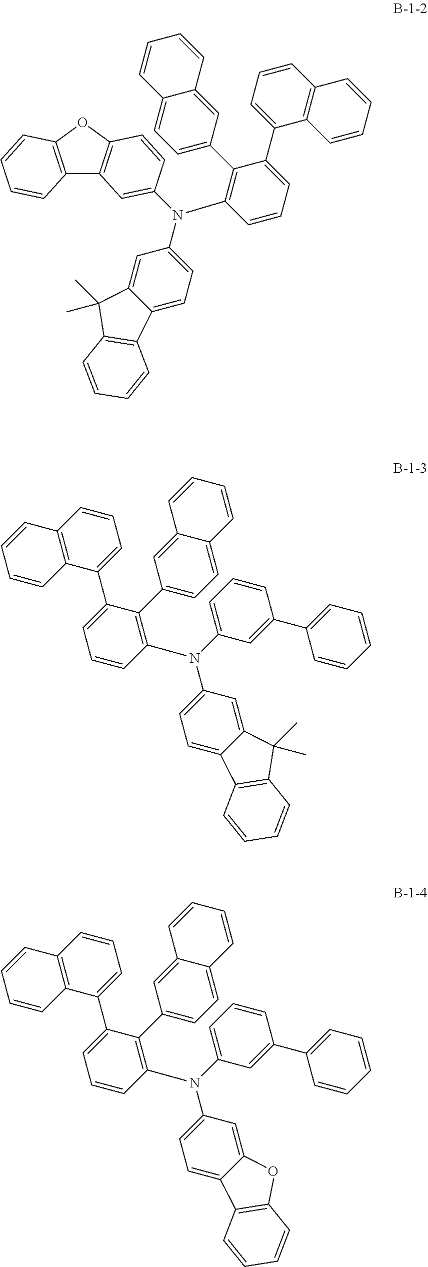 [SMILES: CC1(C)c2ccccc2-c2ccc(N(c3ccc4oc5ccccc5c4c3)c3cccc(-c4cccc5ccccc45)c3-c3ccc4ccccc4c3)cc21 ; CC1(C)c2ccccc2-c2ccc(N(c3cccc(-c4ccccc4)c3)c3cccc(-c4cccc5ccccc45)c3-c3ccc4ccccc4c3)cc21 ; c1ccc(-c2cccc(N(c3ccc4c(c3)oc3ccccc34)c3cccc(-c4cccc5ccccc45)c3-c3ccc4ccccc4c3)c2)cc1]

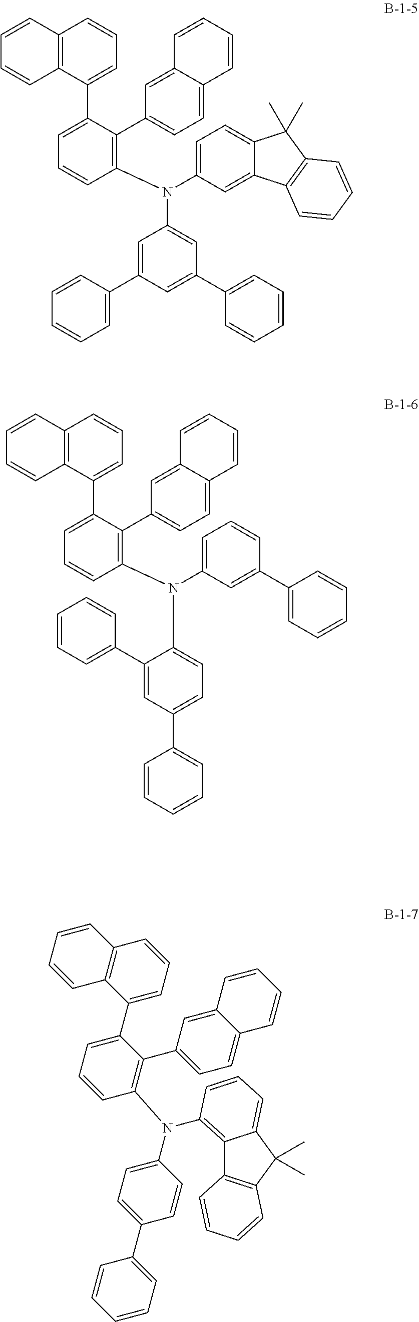 [SMILES: CC1(C)c2ccccc2-c2cc(N(c3cc(-c4ccccc4)cc(-c4ccccc4)c3)c3cccc(-c4cccc5ccccc45)c3-c3ccc4ccccc4c3)ccc21 ; c1ccc(-c2cccc(N(c3ccc(-c4ccccc4)cc3-c3ccccc3)c3cccc(-c4cccc5ccccc45)c3-c3ccc4ccccc4c3)c2)cc1 ; CC1(C)c2ccccc2-c2c(N(c3ccc(-c4ccccc4)cc3)c3cccc(-c4cccc5ccccc45)c3-c3ccc4ccccc4c3)cccc21]

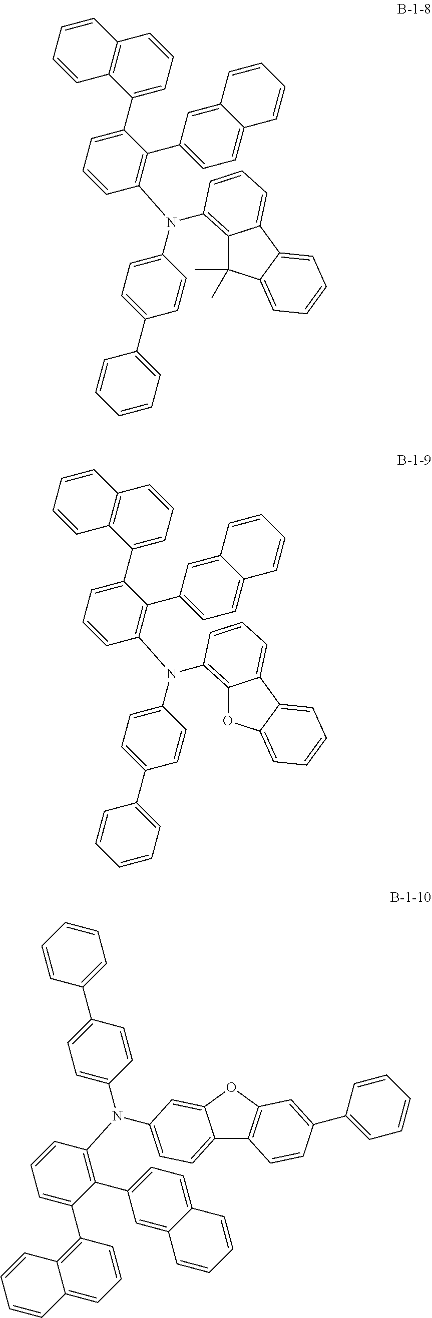 [SMILES: CC1(C)c2ccccc2-c2cccc(N(c3ccc(-c4ccccc4)cc3)c3cccc(-c4cccc5ccccc45)c3-c3ccc4ccccc4c3)c21 ; c1ccc(-c2ccc(N(c3cccc(-c4cccc5ccccc45)c3-c3ccc4ccccc4c3)c3cccc4c3oc3ccccc34)cc2)cc1 ; c1ccc(-c2ccc(N(c3ccc4c(c3)oc3cc(-c5ccccc5)ccc34)c3cccc(-c4cccc5ccccc45)c3-c3ccc4ccccc4c3)cc2)cc1]

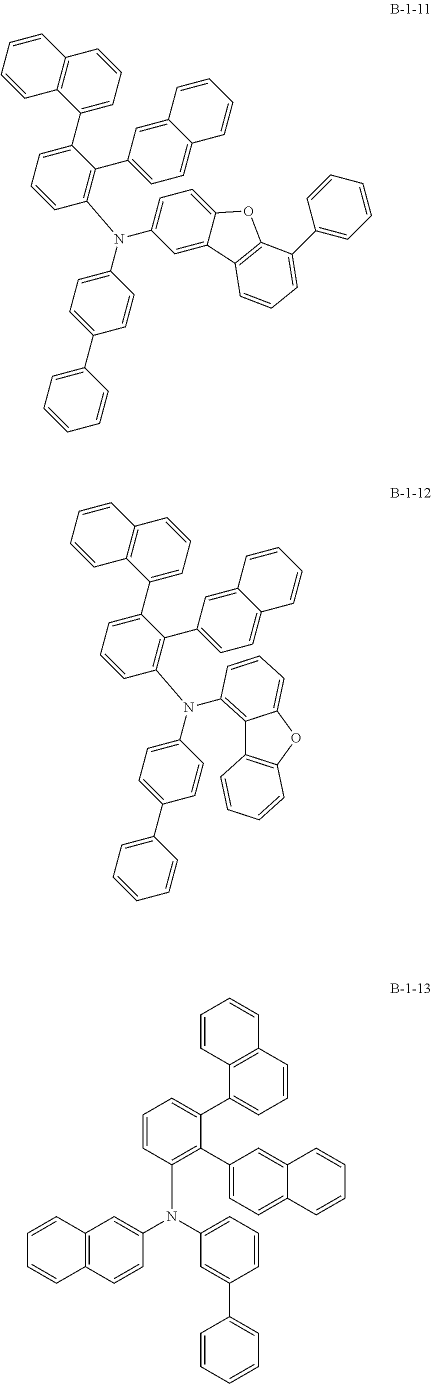 [SMILES: c1ccc(-c2ccc(N(c3ccc4oc5c(-c6ccccc6)cccc5c4c3)c3cccc(-c4cccc5ccccc45)c3-c3ccc4ccccc4c3)cc2)cc1 ; c1ccc(-c2ccc(N(c3cccc(-c4cccc5ccccc45)c3-c3ccc4ccccc4c3)c3cccc4oc5ccccc5c34)cc2)cc1 ; c1ccc(-c2cccc(N(c3ccc4ccccc4c3)c3cccc(-c4cccc5ccccc45)c3-c3ccc4ccccc4c3)c2)cc1]

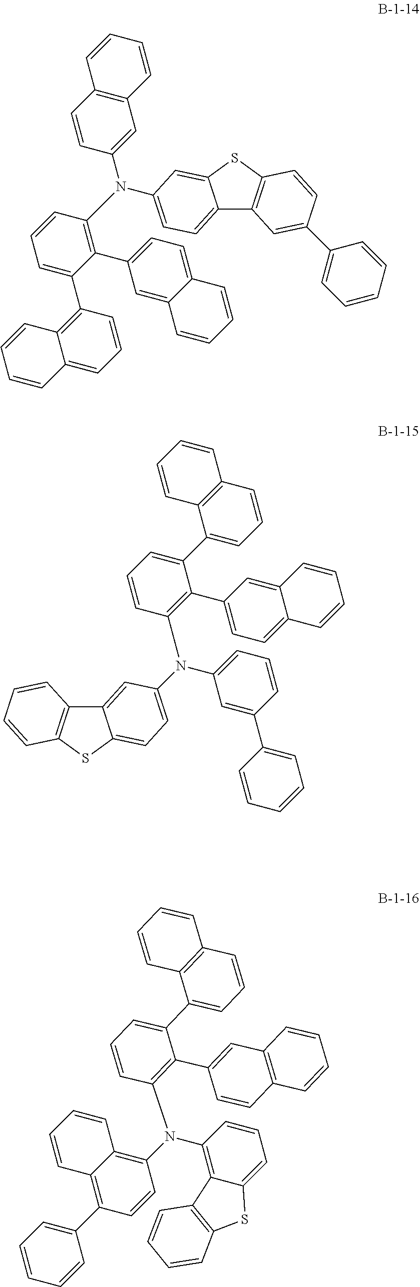 [SMILES: c1ccc(-c2ccc3sc4cc(N(c5ccc6ccccc6c5)c5cccc(-c6cccc7ccccc67)c5-c5ccc6ccccc6c5)ccc4c3c2)cc1 ; c1ccc(-c2cccc(N(c3ccc4sc5ccccc5c4c3)c3cccc(-c4cccc5ccccc45)c3-c3ccc4ccccc4c3)c2)cc1 ; c1ccc(-c2ccc(N(c3cccc(-c4cccc5ccccc45)c3-c3ccc4ccccc4c3)c3cccc4sc5ccccc5c34)c3ccccc23)cc1]

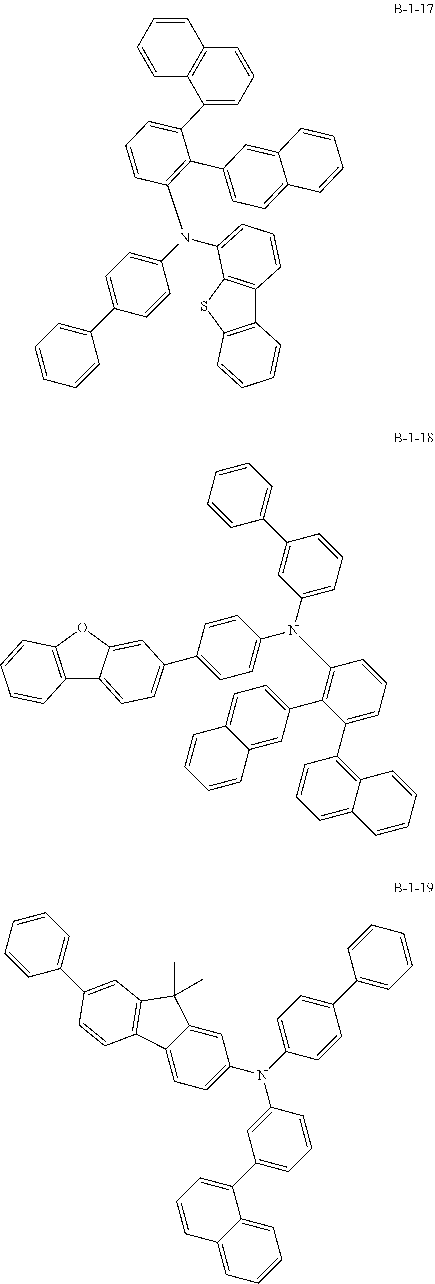 [SMILES: c1ccc(-c2ccc(N(c3cccc(-c4cccc5ccccc45)c3-c3ccc4ccccc4c3)c3cccc4c3sc3ccccc34)cc2)cc1 ; c1ccc(-c2cccc(N(c3ccc(-c4ccc5c(c4)oc4ccccc45)cc3)c3cccc(-c4cccc5ccccc45)c3-c3ccc4ccccc4c3)c2)cc1 ; CC1(C)c2cc(-c3ccccc3)ccc2-c2ccc(N(c3ccc(-c4ccccc4)cc3)c3cccc(-c4cccc5ccccc45)c3)cc21]

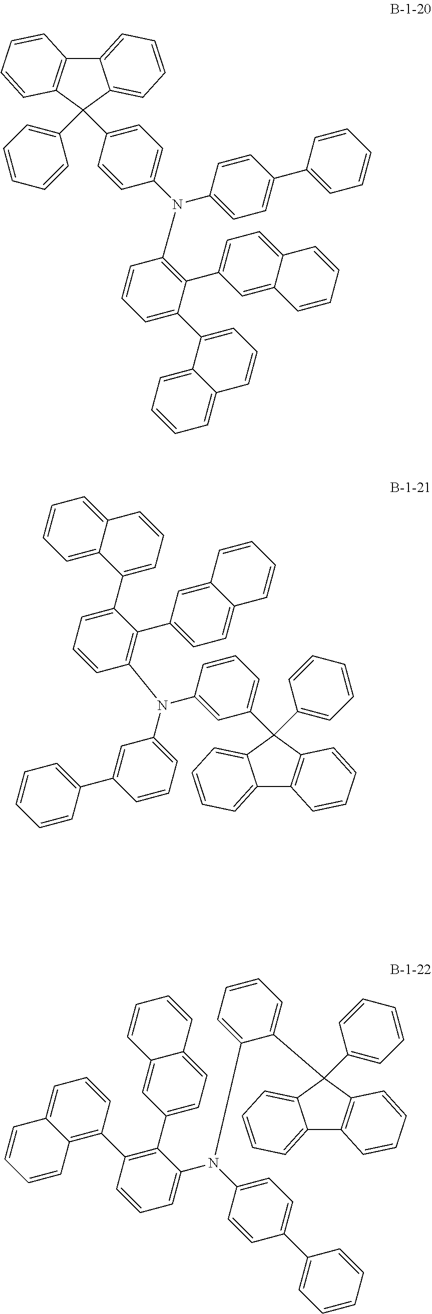 [SMILES: c1ccc(-c2ccc(N(c3ccc(C4(c5ccccc5)c5ccccc5-c5ccccc54)cc3)c3cccc(-c4cccc5ccccc45)c3-c3ccc4ccccc4c3)cc2)cc1 ; c1ccc(-c2cccc(N(c3cccc(C4(c5ccccc5)c5ccccc5-c5ccccc54)c3)c3cccc(-c4cccc5ccccc45)c3-c3ccc4ccccc4c3)c2)cc1 ; c1ccc(-c2ccc(N(c3ccccc3C3(c4ccccc4)c4ccccc4-c4ccccc43)c3cccc(-c4cccc5ccccc45)c3-c3ccc4ccccc4c3)cc2)cc1]

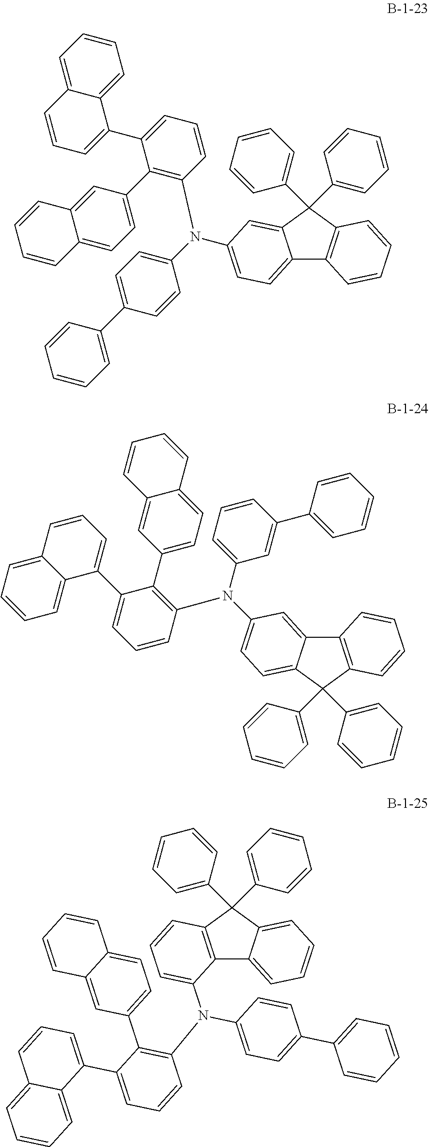 [SMILES: c1ccc(-c2ccc(N(c3ccc4c(c3)C(c3ccccc3)(c3ccccc3)c3ccccc3-4)c3cccc(-c4cccc5ccccc45)c3-c3ccc4ccccc4c3)cc2)cc1 ; c1ccc(-c2cccc(N(c3ccc4c(c3)-c3ccccc3C4(c3ccccc3)c3ccccc3)c3cccc(-c4cccc5ccccc45)c3-c3ccc4ccccc4c3)c2)cc1 ; c1ccc(-c2ccc(N(c3cccc(-c4cccc5ccccc45)c3-c3ccc4ccccc4c3)c3cccc4c3-c3ccccc3C4(c3ccccc3)c3ccccc3)cc2)cc1]

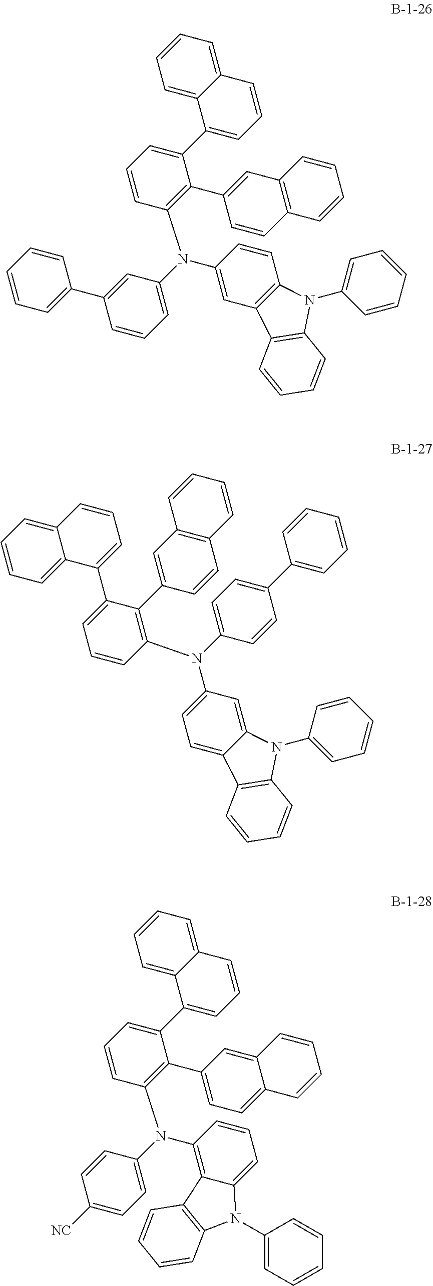 [SMILES: c1ccc(-c2cccc(N(c3ccc4c(c3)c3ccccc3n4-c3ccccc3)c3cccc(-c4cccc5ccccc45)c3-c3ccc4ccccc4c3)c2)cc1 ; c1ccc(-c2ccc(N(c3ccc4c5ccccc5n(-c5ccccc5)c4c3)c3cccc(-c4cccc5ccccc45)c3-c3ccc4ccccc4c3)cc2)cc1 ; N#Cc1ccc(N(c2cccc(-c3cccc4ccccc34)c2-c2ccc3ccccc3c2)c2cccc3c2c2ccccc2n3-c2ccccc2)cc1]

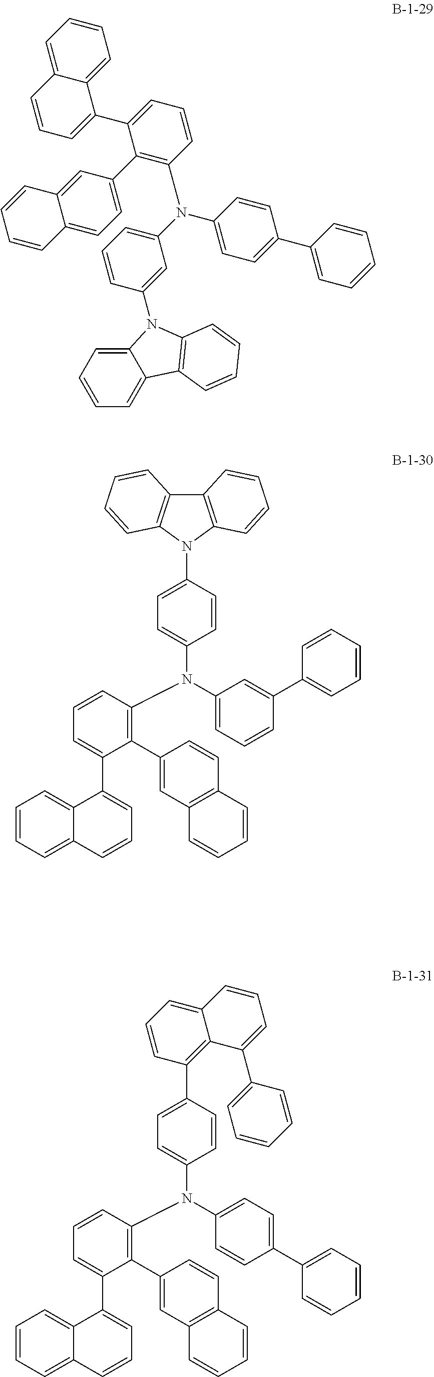 [SMILES: c1ccc(-c2ccc(N(c3cccc(-n4c5ccccc5c5ccccc54)c3)c3cccc(-c4cccc5ccccc45)c3-c3ccc4ccccc4c3)cc2)cc1 ; c1ccc(-c2cccc(N(c3ccc(-n4c5ccccc5c5ccccc54)cc3)c3cccc(-c4cccc5ccccc45)c3-c3ccc4ccccc4c3)c2)cc1 ; c1ccc(-c2ccc(N(c3ccc(-c4cccc5cccc(-c6ccccc6)c45)cc3)c3cccc(-c4cccc5ccccc45)c3-c3ccc4ccccc4c3)cc2)cc1]

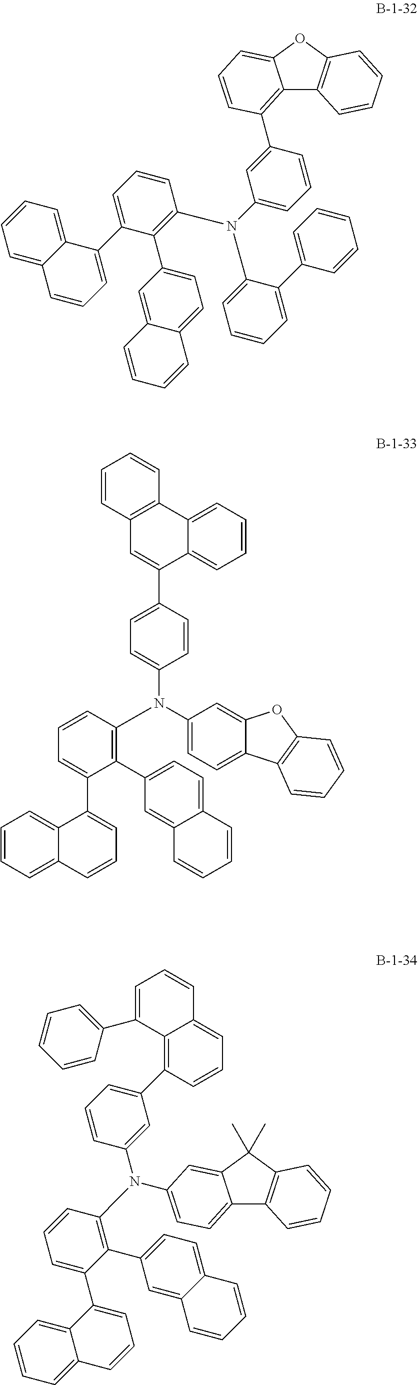 [SMILES: c1ccc(-c2ccccc2N(c2cccc(-c3cccc4oc5ccccc5c34)c2)c2cccc(-c3cccc4ccccc34)c2-c2ccc3ccccc3c2)cc1 ; c1cc(-c2cccc3ccccc23)c(-c2ccc3ccccc3c2)c(N(c2ccc(-c3cc4ccccc4c4ccccc34)cc2)c2ccc3c(c2)oc2ccccc23)c1 ; CC1(C)c2ccccc2-c2ccc(N(c3cccc(-c4cccc5cccc(-c6ccccc6)c45)c3)c3cccc(-c4cccc5ccccc45)c3-c3ccc4ccccc4c3)cc21]

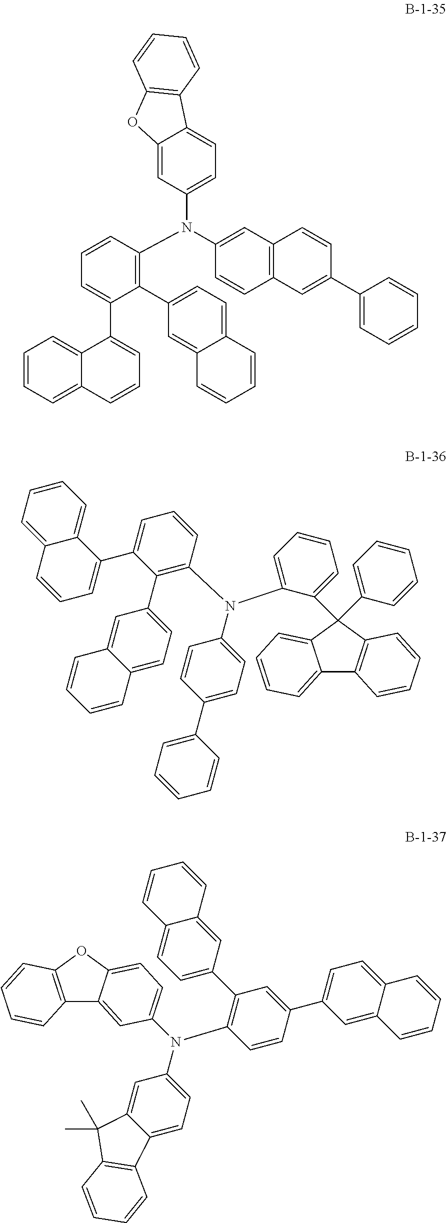 [SMILES: c1ccc(-c2ccc3cc(N(c4ccc5c(c4)oc4ccccc45)c4cccc(-c5cccc6ccccc56)c4-c4ccc5ccccc5c4)ccc3c2)cc1 ; c1ccc(-c2ccc(N(c3ccccc3C3(c4ccccc4)c4ccccc4-c4ccccc43)c3cccc(-c4cccc5ccccc45)c3-c3ccc4ccccc4c3)cc2)cc1 ; CC1(C)c2ccccc2-c2ccc(N(c3ccc4oc5ccccc5c4c3)c3ccc(-c4ccc5ccccc5c4)cc3-c3ccc4ccccc4c3)cc21]

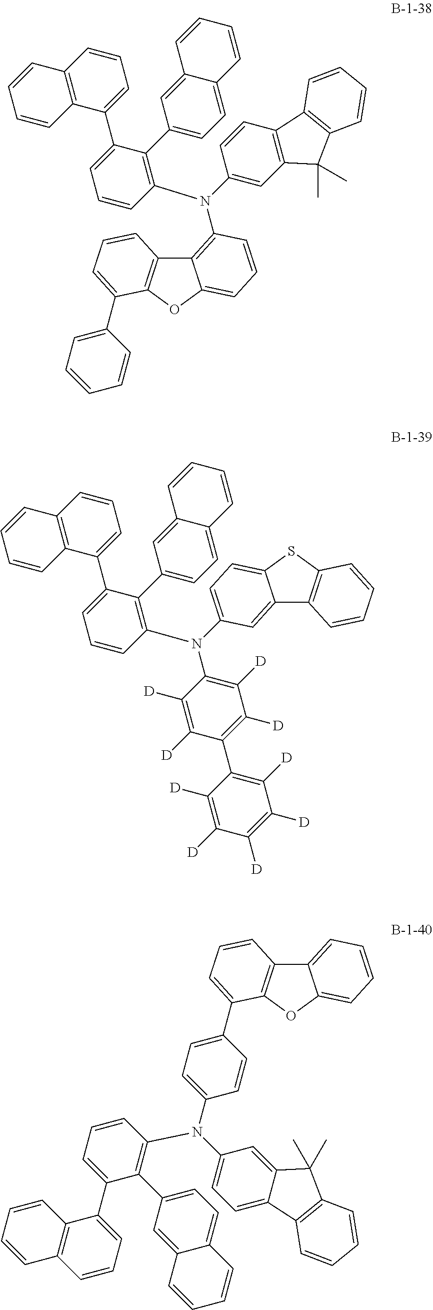 [SMILES: CC1(C)c2ccccc2-c2ccc(N(c3cccc(-c4cccc5ccccc45)c3-c3ccc4ccccc4c3)c3cccc4oc5c(-c6ccccc6)cccc5c34)cc21 ; [2H]c1c([2H])c([2H])c(-c2c([2H])c([2H])c(N(c3ccc4sc5ccccc5c4c3)c3cccc(-c4cccc5ccccc45)c3-c3ccc4ccccc4c3)c([2H])c2[2H])c([2H])c1[2H] ; CC1(C)c2ccccc2-c2ccc(N(c3ccc(-c4cccc5c4oc4ccccc45)cc3)c3cccc(-c4cccc5ccccc45)c3-c3ccc4ccccc4c3)cc21]

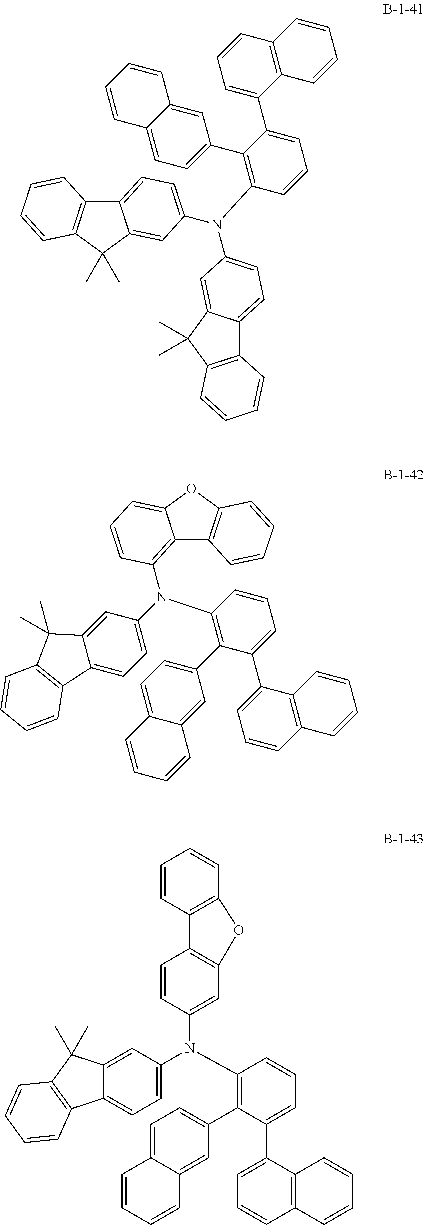 [SMILES: CC1(C)c2ccccc2-c2ccc(N(c3ccc4c(c3)C(C)(C)c3ccccc3-4)c3cccc(-c4cccc5ccccc45)c3-c3ccc4ccccc4c3)cc21 ; CC1(C)c2ccccc2-c2ccc(N(c3cccc(-c4cccc5ccccc45)c3-c3ccc4ccccc4c3)c3cccc4oc5ccccc5c34)cc21 ; CC1(C)c2ccccc2-c2ccc(N(c3ccc4c(c3)oc3ccccc34)c3cccc(-c4cccc5ccccc45)c3-c3ccc4ccccc4c3)cc21]

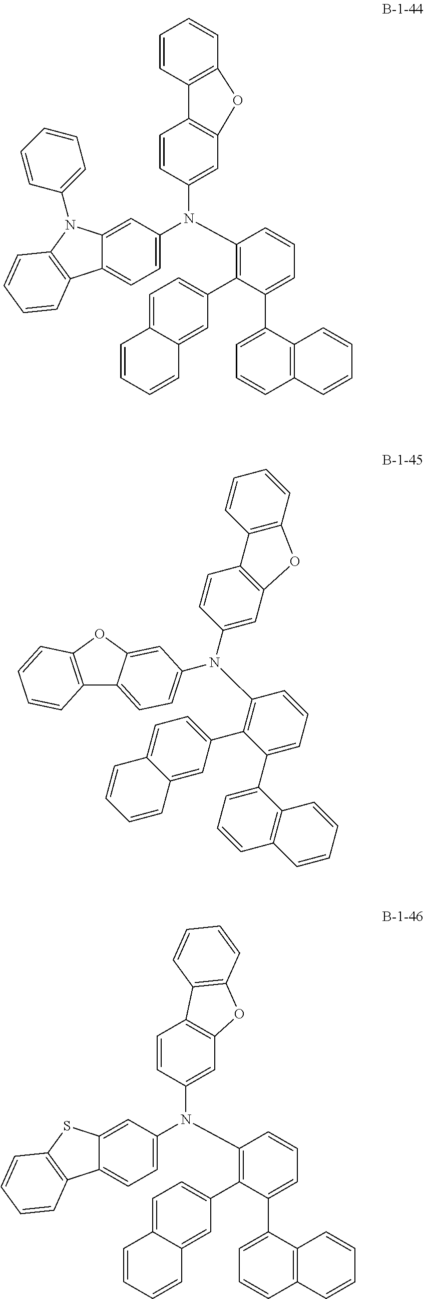 [SMILES: c1ccc(-n2c3ccccc3c3ccc(N(c4ccc5c(c4)oc4ccccc45)c4cccc(-c5cccc6ccccc56)c4-c4ccc5ccccc5c4)cc32)cc1 ; c1cc(-c2cccc3ccccc23)c(-c2ccc3ccccc3c2)c(N(c2ccc3c(c2)oc2ccccc23)c2ccc3c(c2)oc2ccccc23)c1 ; c1cc(-c2cccc3ccccc23)c(-c2ccc3ccccc3c2)c(N(c2ccc3c(c2)oc2ccccc23)c2ccc3c(c2)sc2ccccc23)c1]

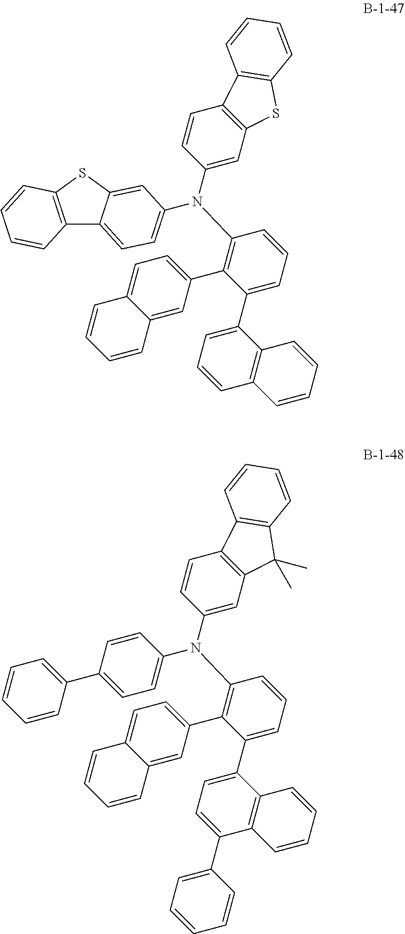 [SMILES: c1cc(-c2cccc3ccccc23)c(-c2ccc3ccccc3c2)c(N(c2ccc3c(c2)sc2ccccc23)c2ccc3c(c2)sc2ccccc23)c1 ; CC1(C)c2ccccc2-c2ccc(N(c3ccc(-c4ccccc4)cc3)c3cccc(-c4ccc(-c5ccccc5)c5ccccc45)c3-c3ccc4ccccc4c3)cc21]

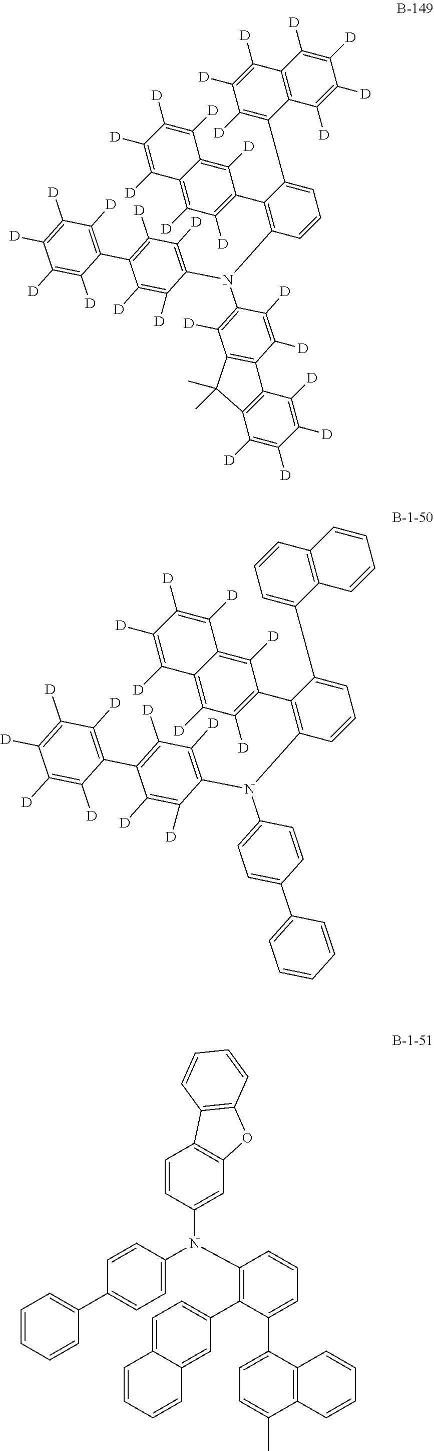

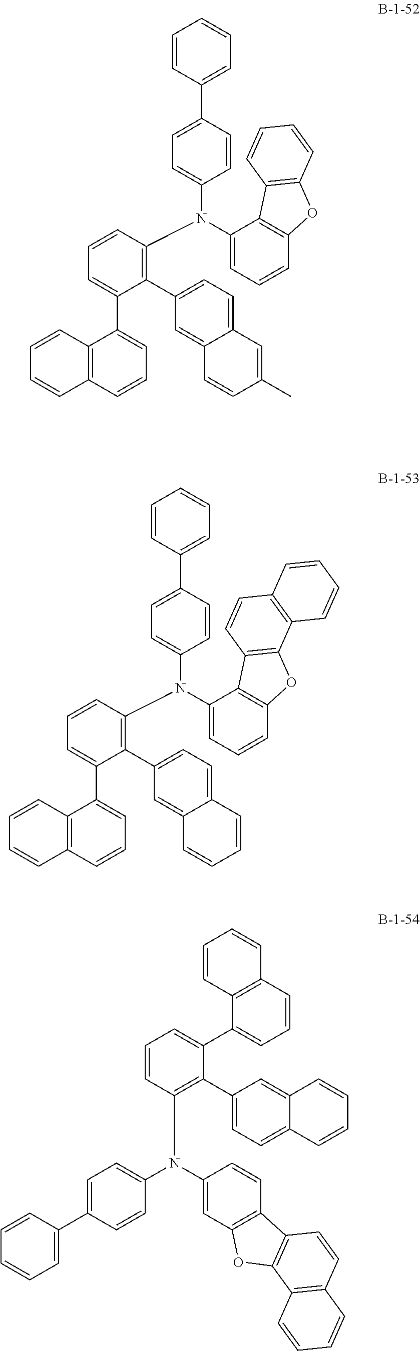 [SMILES: Cc1ccc2cc(-c3c(-c4cccc5ccccc45)cccc3N(c3ccc(-c4ccccc4)cc3)c3cccc4oc5ccccc5c34)ccc2c1 ; c1ccc(-c2ccc(N(c3cccc(-c4cccc5ccccc45)c3-c3ccc4ccccc4c3)c3cccc4oc5c6ccccc6ccc5c34)cc2)cc1 ; c1ccc(-c2ccc(N(c3ccc4c(c3)oc3c5ccccc5ccc43)c3cccc(-c4cccc5ccccc45)c3-c3ccc4ccccc4c3)cc2)cc1]

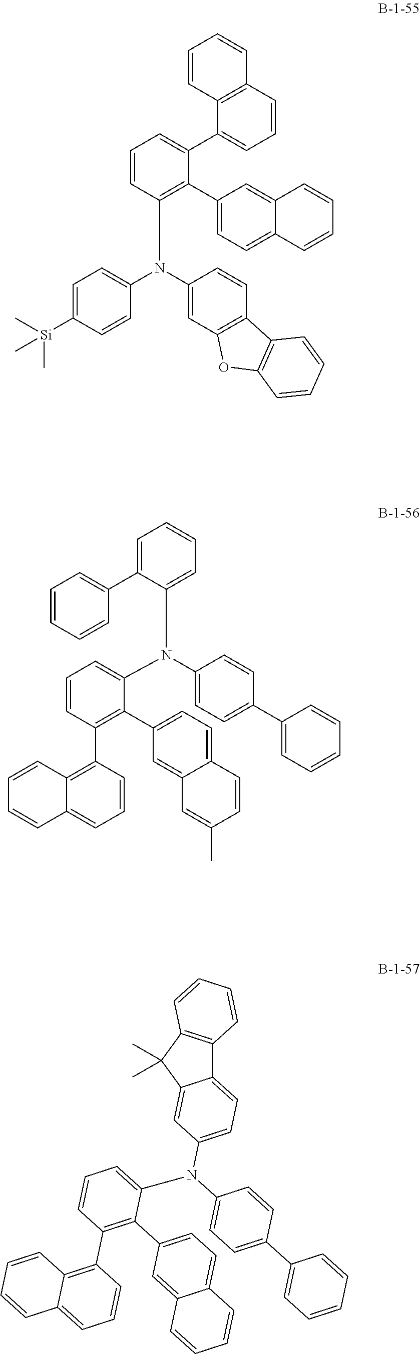 [SMILES: C[Si](C)(C)c1ccc(N(c2ccc3c(c2)oc2ccccc23)c2cccc(-c3cccc4ccccc34)c2-c2ccc3ccccc3c2)cc1 ; Cc1ccc2ccc(-c3c(-c4cccc5ccccc45)cccc3N(c3ccc(-c4ccccc4)cc3)c3ccccc3-c3ccccc3)cc2c1 ; CC1(C)c2ccccc2-c2ccc(N(c3ccc(-c4ccccc4)cc3)c3cccc(-c4cccc5ccccc45)c3-c3ccc4ccccc4c3)cc21]

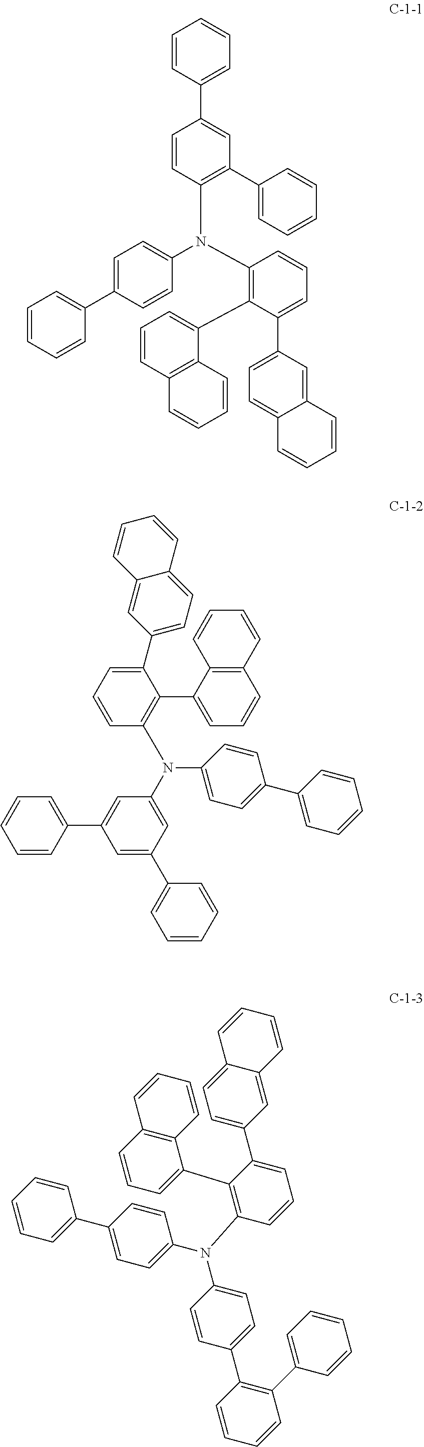 [SMILES: c1ccc(-c2ccc(N(c3ccc(-c4ccccc4)cc3-c3ccccc3)c3cccc(-c4ccc5ccccc5c4)c3-c3cccc4ccccc34)cc2)cc1 ; c1ccc(-c2ccc(N(c3cc(-c4ccccc4)cc(-c4ccccc4)c3)c3cccc(-c4ccc5ccccc5c4)c3-c3cccc4ccccc34)cc2)cc1 ; c1ccc(-c2ccc(N(c3ccc(-c4ccccc4-c4ccccc4)cc3)c3cccc(-c4ccc5ccccc5c4)c3-c3cccc4ccccc34)cc2)cc1]

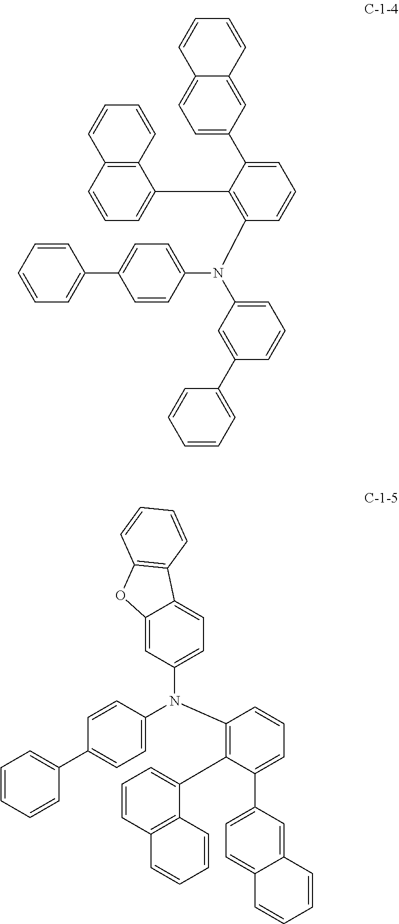 [SMILES: c1ccc(-c2ccc(N(c3cccc(-c4ccccc4)c3)c3cccc(-c4ccc5ccccc5c4)c3-c3cccc4ccccc34)cc2)cc1 ; c1ccc(-c2ccc(N(c3ccc4c(c3)oc3ccccc34)c3cccc(-c4ccc5ccccc5c4)c3-c3cccc4ccccc34)cc2)cc1]

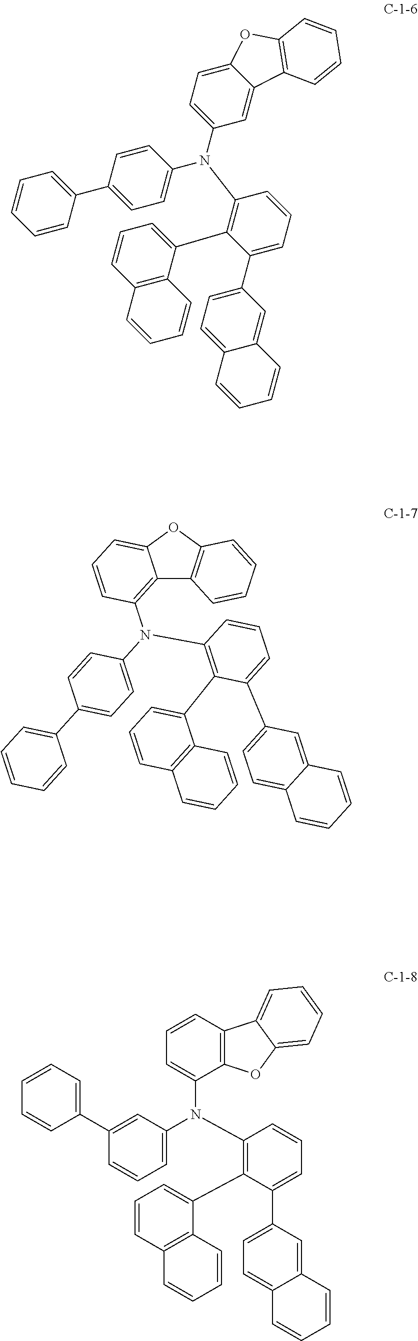 [SMILES: c1ccc(-c2ccc(N(c3ccc4oc5ccccc5c4c3)c3cccc(-c4ccc5ccccc5c4)c3-c3cccc4ccccc34)cc2)cc1 ; c1ccc(-c2ccc(N(c3cccc(-c4ccc5ccccc5c4)c3-c3cccc4ccccc34)c3cccc4oc5ccccc5c34)cc2)cc1 ; c1ccc(-c2cccc(N(c3cccc(-c4ccc5ccccc5c4)c3-c3cccc4ccccc34)c3cccc4c3oc3ccccc34)c2)cc1]

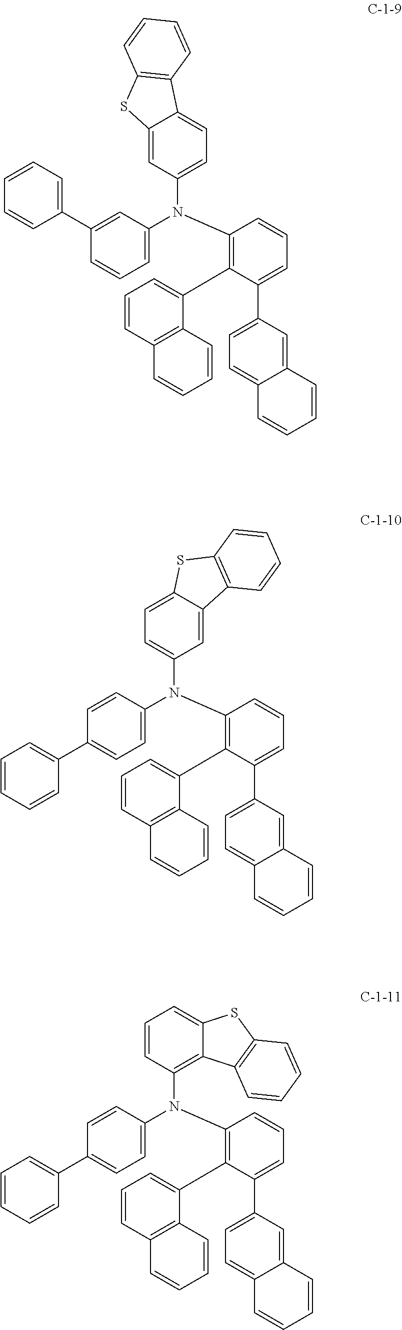 [SMILES: c1ccc(-c2cccc(N(c3ccc4c(c3)sc3ccccc34)c3cccc(-c4ccc5ccccc5c4)c3-c3cccc4ccccc34)c2)cc1 ; c1ccc(-c2ccc(N(c3ccc4sc5ccccc5c4c3)c3cccc(-c4ccc5ccccc5c4)c3-c3cccc4ccccc34)cc2)cc1 ; c1ccc(-c2ccc(N(c3cccc(-c4ccc5ccccc5c4)c3-c3cccc4ccccc34)c3cccc4sc5ccccc5c34)cc2)cc1]

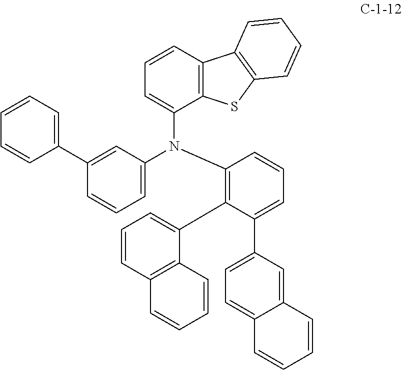 [SMILES: c1ccc(-c2cccc(N(c3cccc(-c4ccc5ccccc5c4)c3-c3cccc4ccccc34)c3cccc4c3sc3ccccc34)c2)cc1]

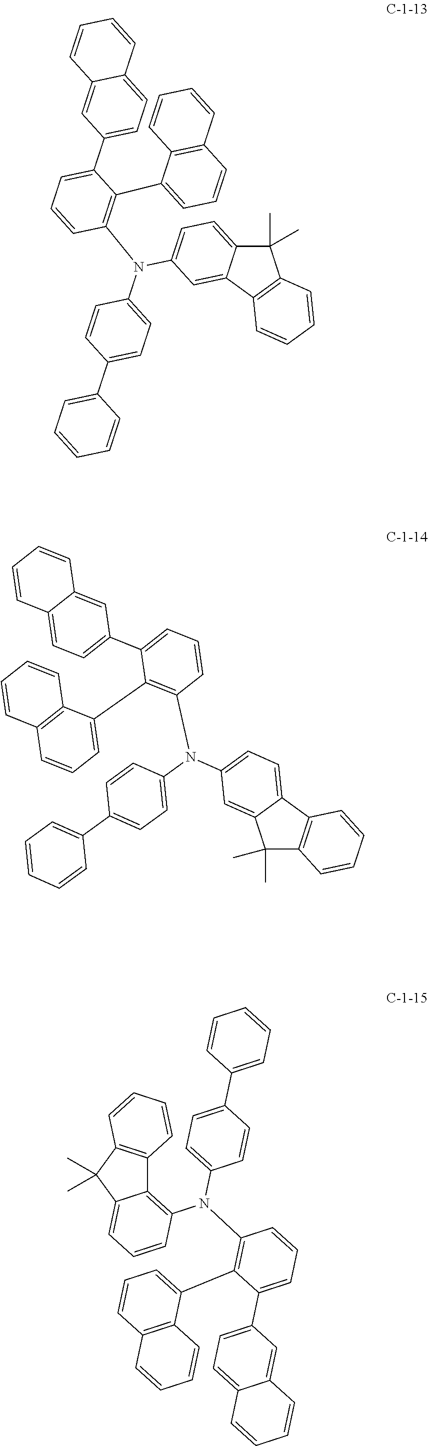 [SMILES: CC1(C)c2ccccc2-c2cc(N(c3ccc(-c4ccccc4)cc3)c3cccc(-c4ccc5ccccc5c4)c3-c3cccc4ccccc34)ccc21 ; CC1(C)c2ccccc2-c2ccc(N(c3ccc(-c4ccccc4)cc3)c3cccc(-c4ccc5ccccc5c4)c3-c3cccc4ccccc34)cc21 ; CC1(C)c2ccccc2-c2c(N(c3ccc(-c4ccccc4)cc3)c3cccc(-c4ccc5ccccc5c4)c3-c3cccc4ccccc34)cccc21]

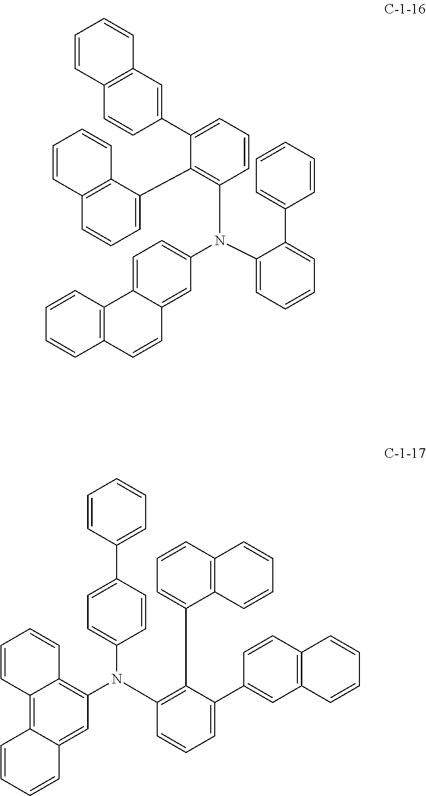 [SMILES: c1ccc(-c2ccccc2N(c2ccc3c(ccc4ccccc43)c2)c2cccc(-c3ccc4ccccc4c3)c2-c2cccc3ccccc23)cc1 ; c1ccc(-c2ccc(N(c3cccc(-c4ccc5ccccc5c4)c3-c3cccc4ccccc34)c3cc4ccccc4c4ccccc34)cc2)cc1]

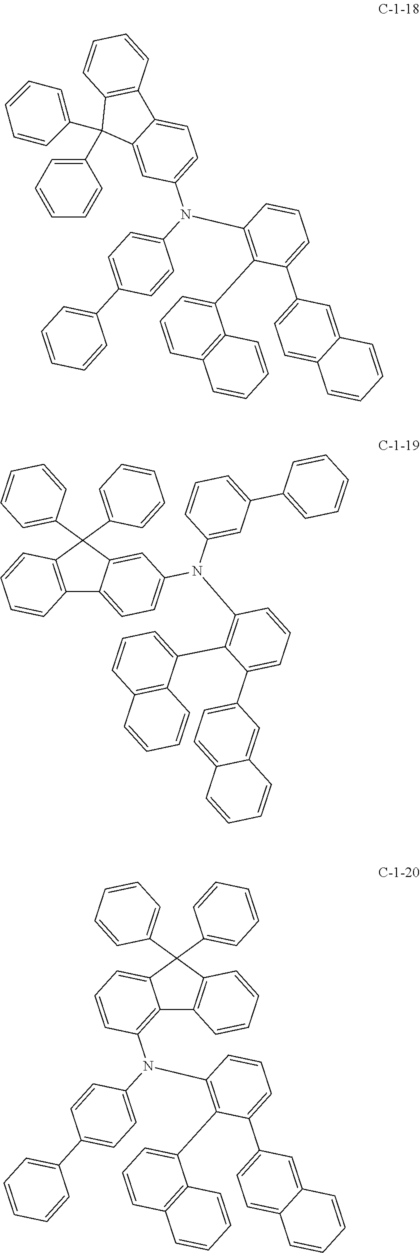 [SMILES: c1ccc(-c2ccc(N(c3ccc4c(c3)C(c3ccccc3)(c3ccccc3)c3ccccc3-4)c3cccc(-c4ccc5ccccc5c4)c3-c3cccc4ccccc34)cc2)cc1 ; c1ccc(-c2cccc(N(c3ccc4c(c3)C(c3ccccc3)(c3ccccc3)c3ccccc3-4)c3cccc(-c4ccc5ccccc5c4)c3-c3cccc4ccccc34)c2)cc1 ; c1ccc(-c2ccc(N(c3cccc(-c4ccc5ccccc5c4)c3-c3cccc4ccccc34)c3cccc4c3-c3ccccc3C4(c3ccccc3)c3ccccc3)cc2)cc1]

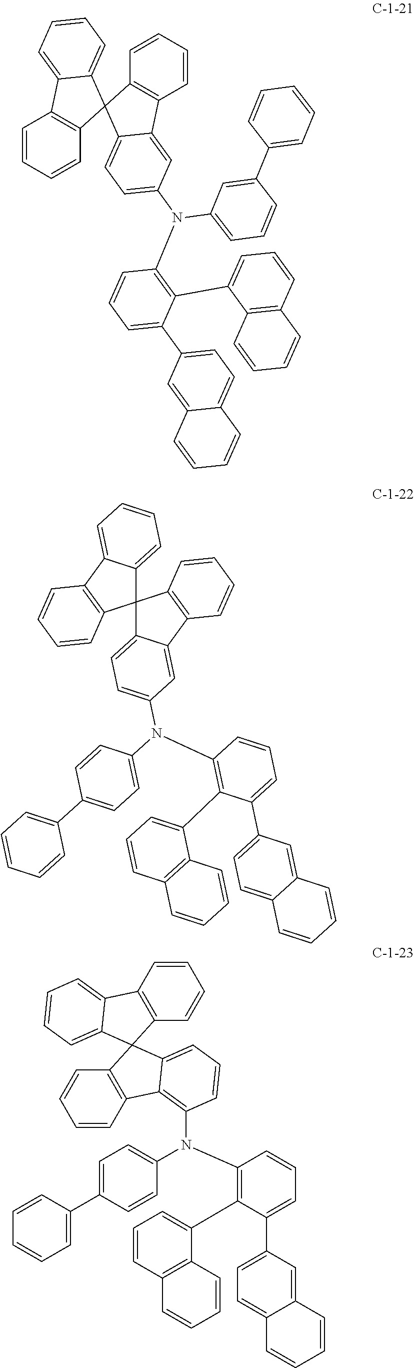

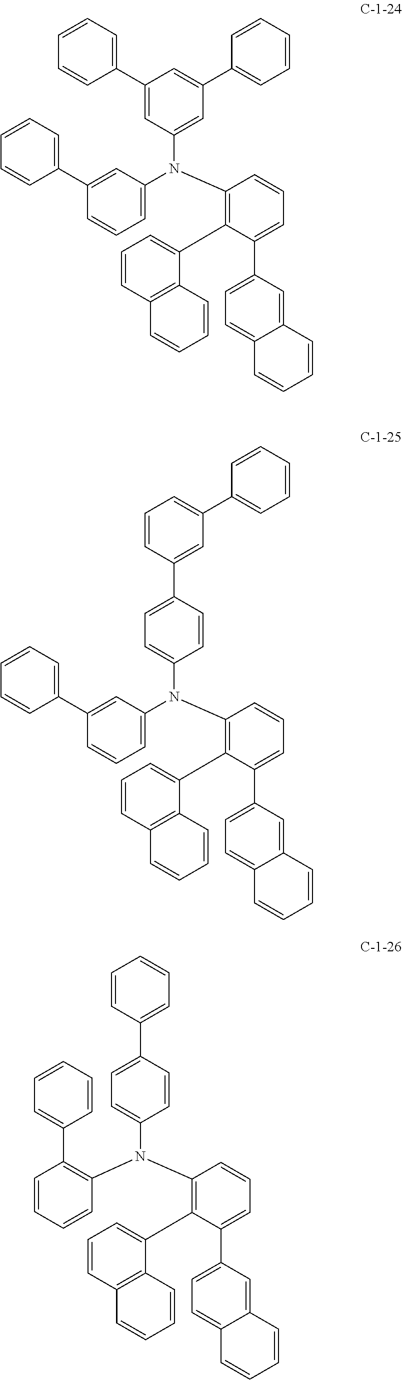 [SMILES: c1ccc(-c2cccc(N(c3cc(-c4ccccc4)cc(-c4ccccc4)c3)c3cccc(-c4ccc5ccccc5c4)c3-c3cccc4ccccc34)c2)cc1 ; c1ccc(-c2cccc(-c3ccc(N(c4cccc(-c5ccccc5)c4)c4cccc(-c5ccc6ccccc6c5)c4-c4cccc5ccccc45)cc3)c2)cc1 ; c1ccc(-c2ccc(N(c3ccccc3-c3ccccc3)c3cccc(-c4ccc5ccccc5c4)c3-c3cccc4ccccc34)cc2)cc1]

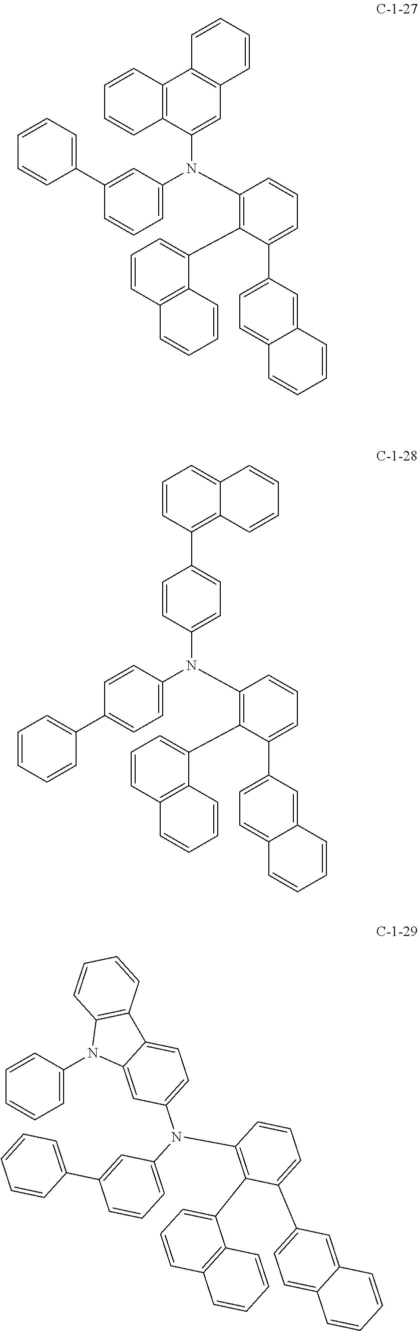 [SMILES: c1ccc(-c2cccc(N(c3cccc(-c4ccc5ccccc5c4)c3-c3cccc4ccccc34)c3cc4ccccc4c4ccccc34)c2)cc1 ; c1ccc(-c2ccc(N(c3ccc(-c4cccc5ccccc45)cc3)c3cccc(-c4ccc5ccccc5c4)c3-c3cccc4ccccc34)cc2)cc1 ; c1ccc(-c2cccc(N(c3ccc4c5ccccc5n(-c5ccccc5)c4c3)c3cccc(-c4ccc5ccccc5c4)c3-c3cccc4ccccc34)c2)cc1]

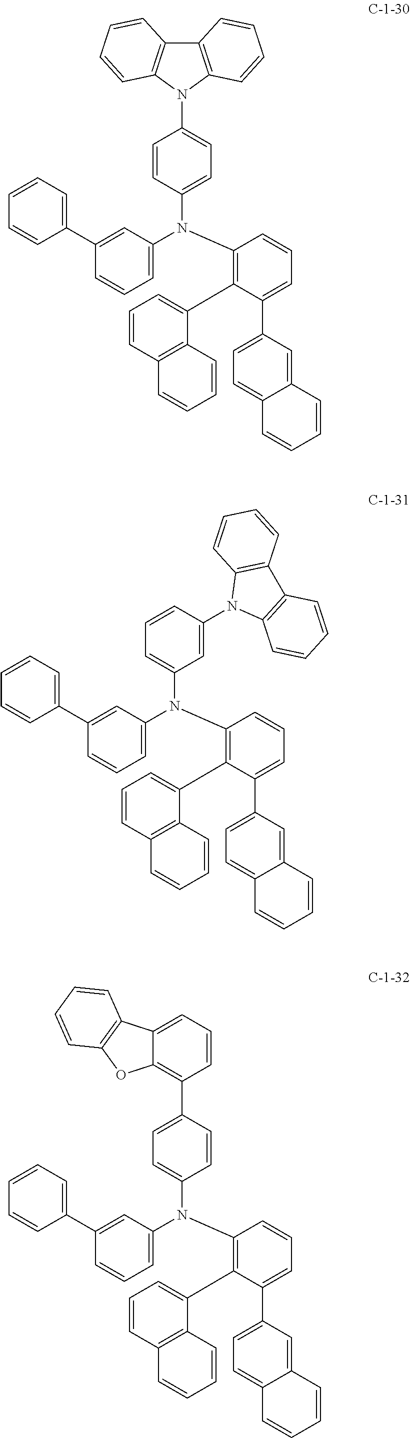 [SMILES: c1ccc(-c2cccc(N(c3ccc(-n4c5ccccc5c5ccccc54)cc3)c3cccc(-c4ccc5ccccc5c4)c3-c3cccc4ccccc34)c2)cc1 ; c1ccc(-c2cccc(N(c3cccc(-n4c5ccccc5c5ccccc54)c3)c3cccc(-c4ccc5ccccc5c4)c3-c3cccc4ccccc34)c2)cc1 ; c1ccc(-c2cccc(N(c3ccc(-c4cccc5c4oc4ccccc45)cc3)c3cccc(-c4ccc5ccccc5c4)c3-c3cccc4ccccc34)c2)cc1]

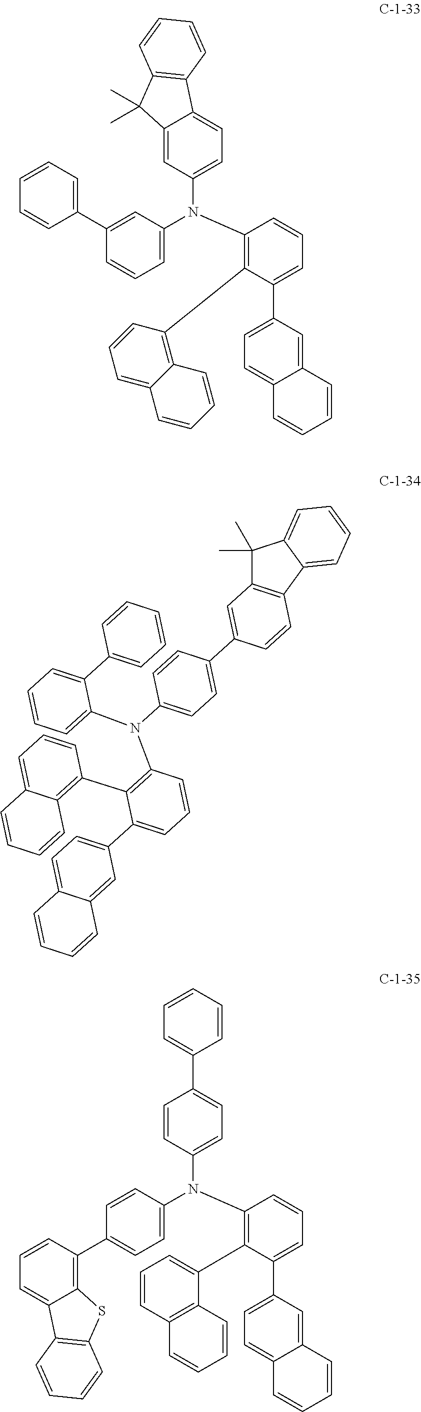 [SMILES: CC1(C)c2ccccc2-c2ccc(N(c3cccc(-c4ccccc4)c3)c3cccc(-c4ccc5ccccc5c4)c3-c3cccc4ccccc34)cc21 ; CC1(C)c2ccccc2-c2ccc(-c3ccc(N(c4ccccc4-c4ccccc4)c4cccc(-c5ccc6ccccc6c5)c4-c4cccc5ccccc45)cc3)cc21 ; c1ccc(-c2ccc(N(c3ccc(-c4cccc5c4sc4ccccc45)cc3)c3cccc(-c4ccc5ccccc5c4)c3-c3cccc4ccccc34)cc2)cc1]

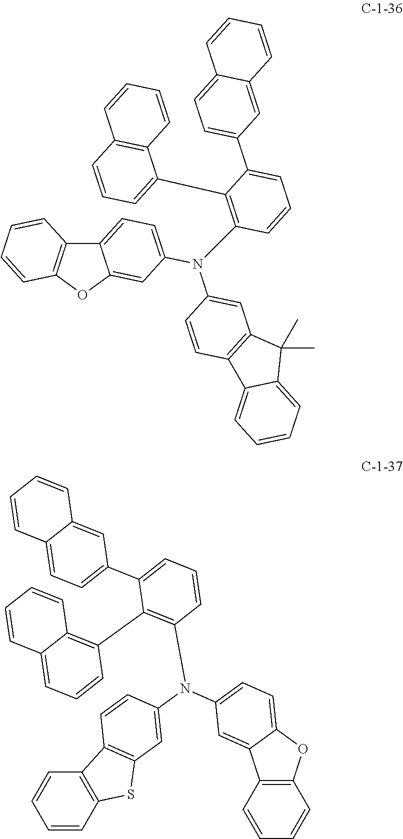 [SMILES: CC1(C)c2ccccc2-c2ccc(N(c3ccc4c(c3)oc3ccccc34)c3cccc(-c4ccc5ccccc5c4)c3-c3cccc4ccccc34)cc21 ; c1cc(-c2ccc3ccccc3c2)c(-c2cccc3ccccc23)c(N(c2ccc3c(c2)sc2ccccc23)c2ccc3oc4ccccc4c3c2)c1]

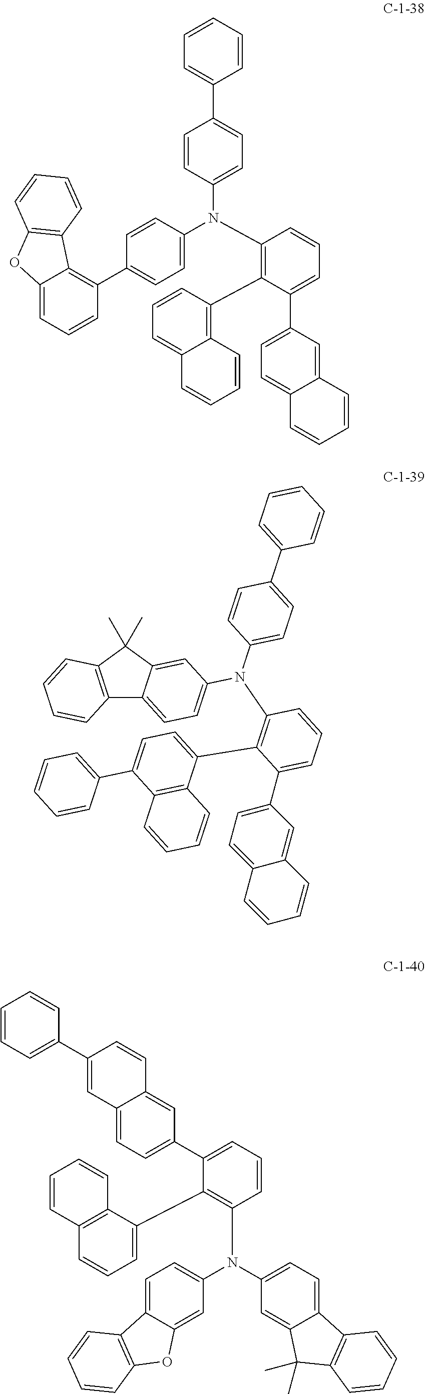

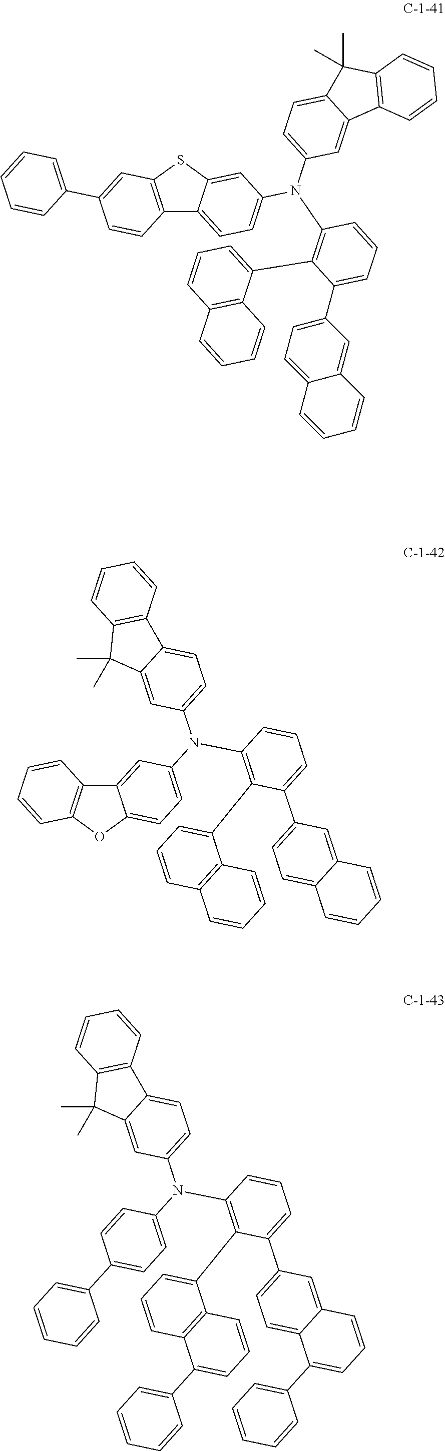 [SMILES: CC1(C)c2ccccc2-c2cc(N(c3ccc4c(c3)sc3cc(-c5ccccc5)ccc34)c3cccc(-c4ccc5ccccc5c4)c3-c3cccc4ccccc34)ccc21 ; CC1(C)c2ccccc2-c2ccc(N(c3ccc4oc5ccccc5c4c3)c3cccc(-c4ccc5ccccc5c4)c3-c3cccc4ccccc34)cc21 ; CC1(C)c2ccccc2-c2ccc(N(c3ccc(-c4ccccc4)cc3)c3cccc(-c4ccc5c(-c6ccccc6)cccc5c4)c3-c3cccc4c(-c5ccccc5)cccc34)cc21]

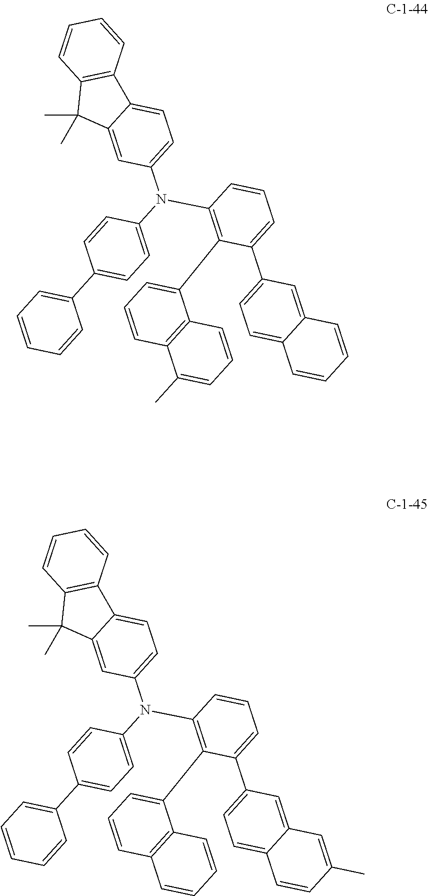 [SMILES: Cc1cccc2c(-c3c(-c4ccc5ccccc5c4)cccc3N(c3ccc(-c4ccccc4)cc3)c3ccc4c(c3)C(C)(C)c3ccccc3-4)cccc12 ; Cc1ccc2ccc(-c3cccc(N(c4ccc(-c5ccccc5)cc4)c4ccc5c(c4)C(C)(C)c4ccccc4-5)c3-c3cccc4ccccc34)cc2c1]

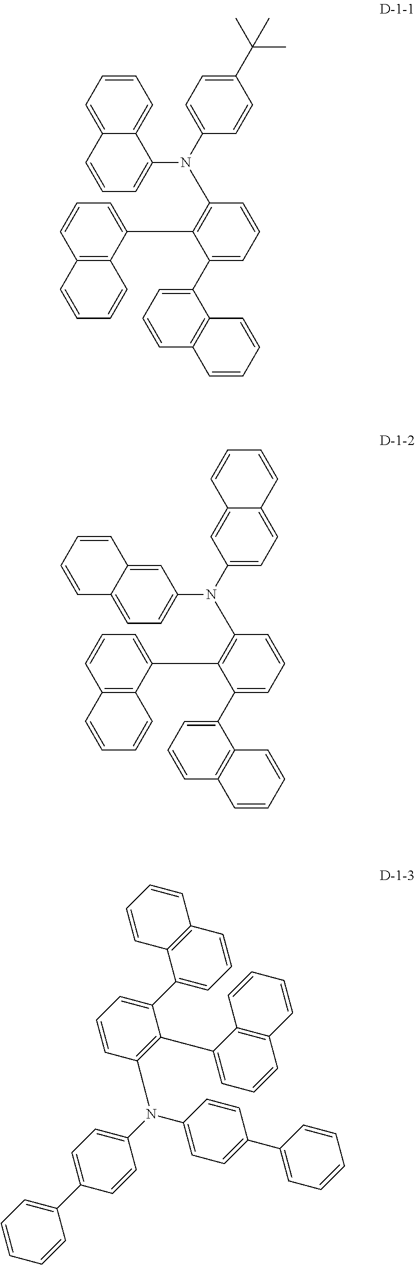 [SMILES: CC(C)(C)c1ccc(N(c2cccc(-c3cccc4ccccc34)c2-c2cccc3ccccc23)c2cccc3ccccc23)cc1 ; c1cc(-c2cccc3ccccc23)c(-c2cccc3ccccc23)c(N(c2ccc3ccccc3c2)c2ccc3ccccc3c2)c1 ; c1ccc(-c2ccc(N(c3ccc(-c4ccccc4)cc3)c3cccc(-c4cccc5ccccc45)c3-c3cccc4ccccc34)cc2)cc1]

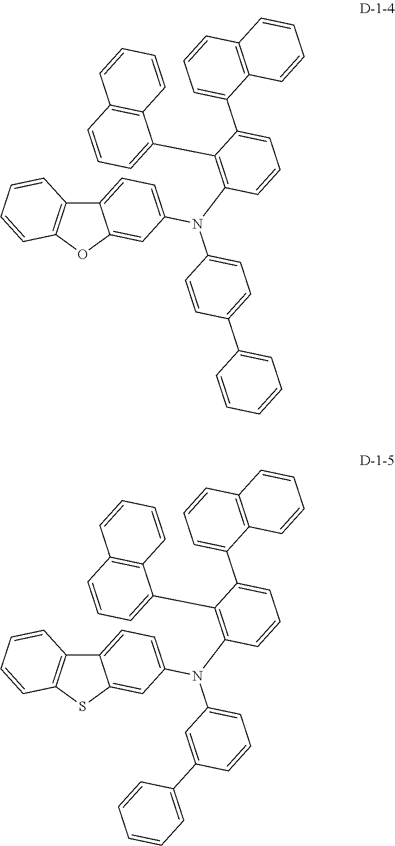 [SMILES: c1ccc(-c2ccc(N(c3ccc4c(c3)oc3ccccc34)c3cccc(-c4cccc5ccccc45)c3-c3cccc4ccccc34)cc2)cc1 ; c1ccc(-c2cccc(N(c3ccc4c(c3)sc3ccccc34)c3cccc(-c4cccc5ccccc45)c3-c3cccc4ccccc34)c2)cc1]

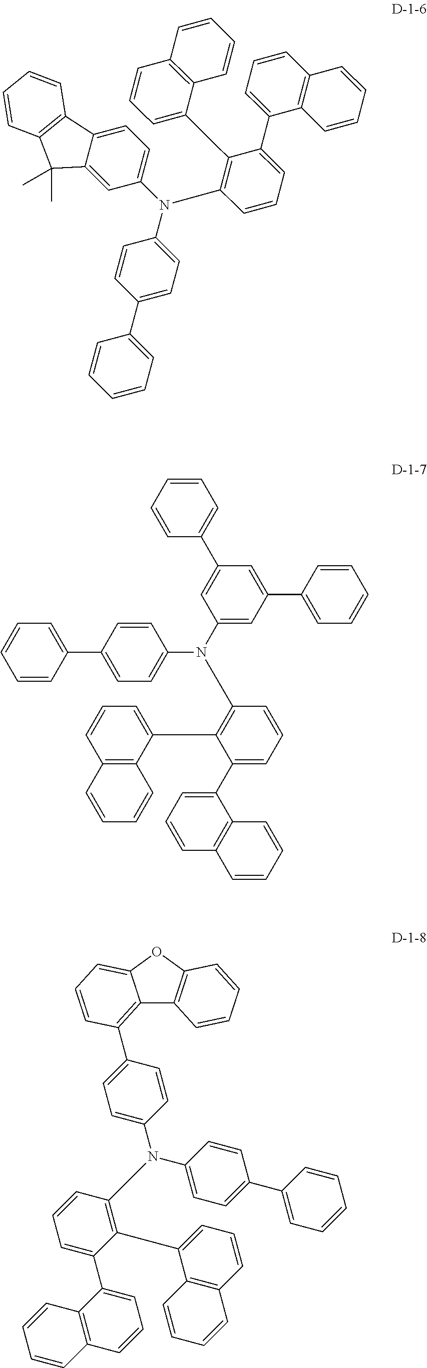 [SMILES: CC1(C)c2ccccc2-c2ccc(N(c3ccc(-c4ccccc4)cc3)c3cccc(-c4cccc5ccccc45)c3-c3cccc4ccccc34)cc21 ; c1ccc(-c2ccc(N(c3cc(-c4ccccc4)cc(-c4ccccc4)c3)c3cccc(-c4cccc5ccccc45)c3-c3cccc4ccccc34)cc2)cc1 ; c1ccc(-c2ccc(N(c3ccc(-c4cccc5oc6ccccc6c45)cc3)c3cccc(-c4cccc5ccccc45)c3-c3cccc4ccccc34)cc2)cc1]

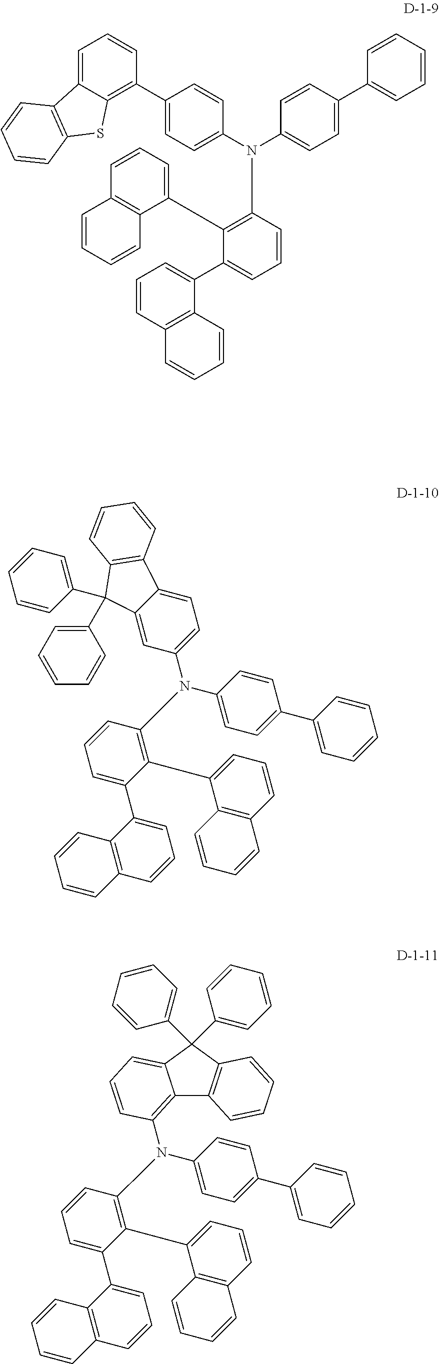 [SMILES: c1ccc(-c2ccc(N(c3ccc(-c4cccc5c4sc4ccccc45)cc3)c3cccc(-c4cccc5ccccc45)c3-c3cccc4ccccc34)cc2)cc1 ; c1ccc(-c2ccc(N(c3ccc4c(c3)C(c3ccccc3)(c3ccccc3)c3ccccc3-4)c3cccc(-c4cccc5ccccc45)c3-c3cccc4ccccc34)cc2)cc1 ; c1ccc(-c2ccc(N(c3cccc(-c4cccc5ccccc45)c3-c3cccc4ccccc34)c3cccc4c3-c3ccccc3C4(c3ccccc3)c3ccccc3)cc2)cc1]

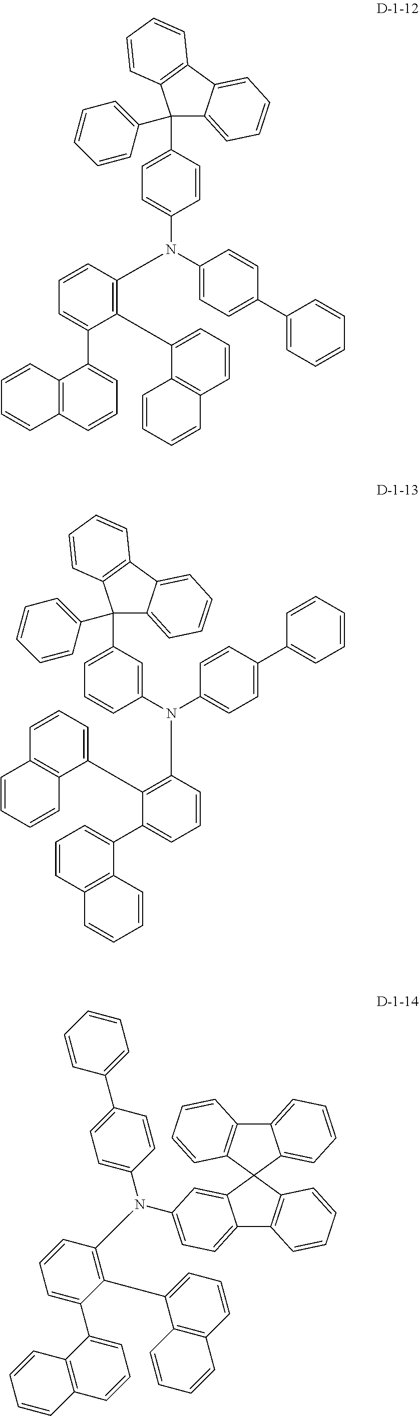 [SMILES: c1ccc(-c2ccc(N(c3ccc(C4(c5ccccc5)c5ccccc5-c5ccccc54)cc3)c3cccc(-c4cccc5ccccc45)c3-c3cccc4ccccc34)cc2)cc1 ; c1ccc(-c2ccc(N(c3cccc(C4(c5ccccc5)c5ccccc5-c5ccccc54)c3)c3cccc(-c4cccc5ccccc45)c3-c3cccc4ccccc34)cc2)cc1 ; c1ccc(-c2ccc(N(c3ccc4c(c3)C3(c5ccccc5-c5ccccc53)c3ccccc3-4)c3cccc(-c4cccc5ccccc45)c3-c3cccc4ccccc34)cc2)cc1]

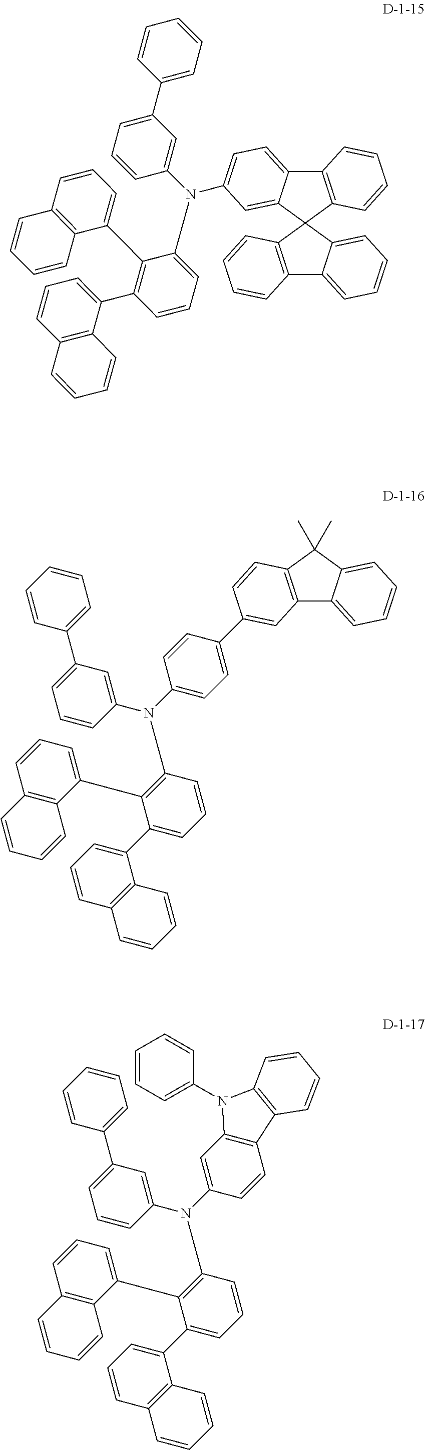 [SMILES: c1ccc(-c2cccc(N(c3ccc4c(c3)C3(c5ccccc5-c5ccccc53)c3ccccc3-4)c3cccc(-c4cccc5ccccc45)c3-c3cccc4ccccc34)c2)cc1 ; CC1(C)c2ccccc2-c2cc(-c3ccc(N(c4cccc(-c5ccccc5)c4)c4cccc(-c5cccc6ccccc56)c4-c4cccc5ccccc45)cc3)ccc21 ; c1ccc(-c2cccc(N(c3ccc4c5ccccc5n(-c5ccccc5)c4c3)c3cccc(-c4cccc5ccccc45)c3-c3cccc4ccccc34)c2)cc1]

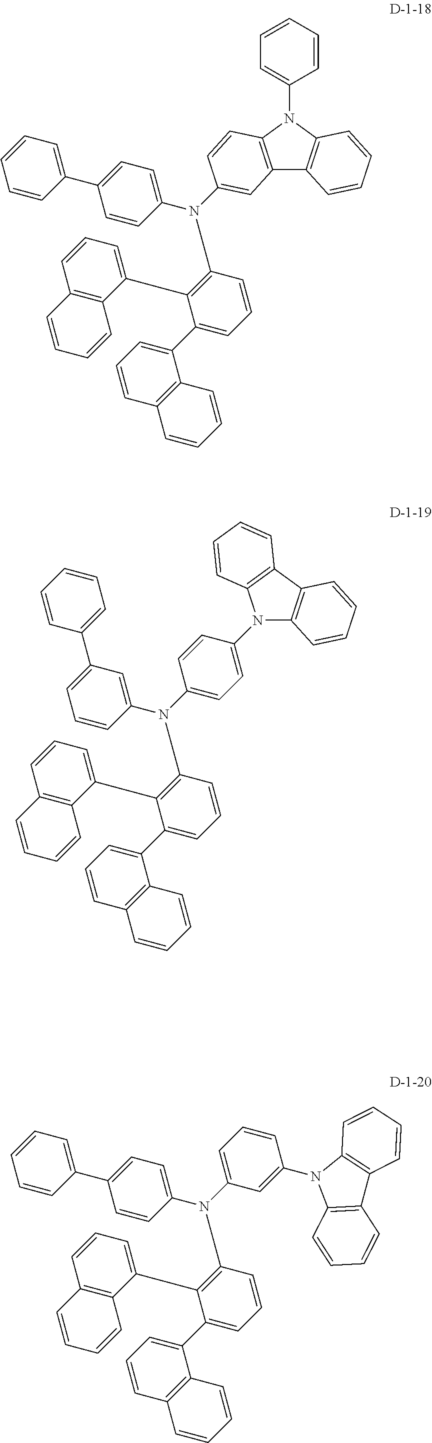 [SMILES: c1ccc(-c2ccc(N(c3ccc4c(c3)c3ccccc3n4-c3ccccc3)c3cccc(-c4cccc5ccccc45)c3-c3cccc4ccccc34)cc2)cc1 ; c1ccc(-c2cccc(N(c3ccc(-n4c5ccccc5c5ccccc54)cc3)c3cccc(-c4cccc5ccccc45)c3-c3cccc4ccccc34)c2)cc1 ; c1ccc(-c2ccc(N(c3cccc(-n4c5ccccc5c5ccccc54)c3)c3cccc(-c4cccc5ccccc45)c3-c3cccc4ccccc34)cc2)cc1]

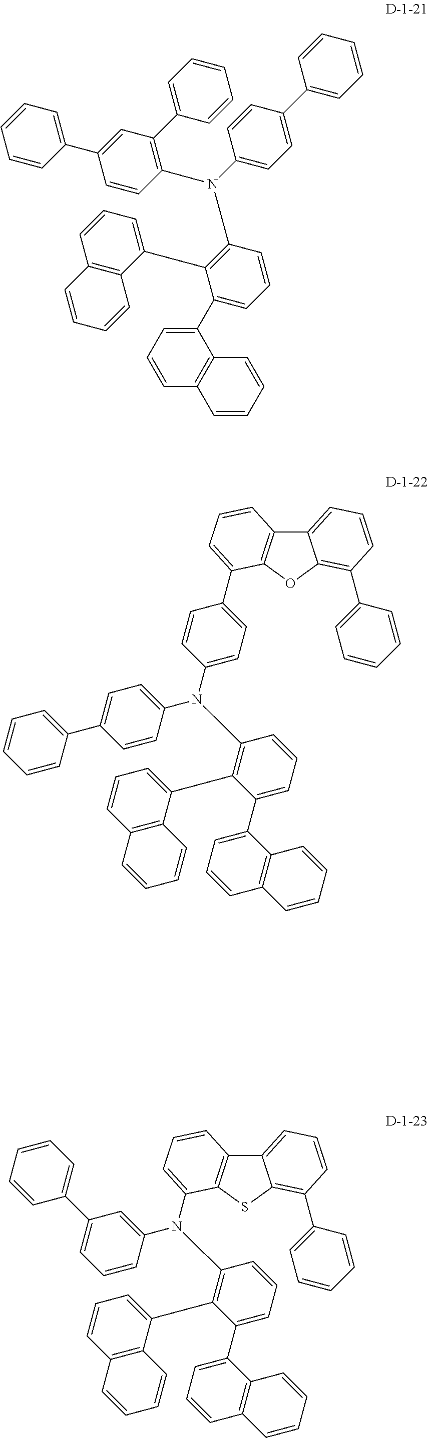 [SMILES: c1ccc(-c2ccc(N(c3ccc(-c4ccccc4)cc3-c3ccccc3)c3cccc(-c4cccc5ccccc45)c3-c3cccc4ccccc34)cc2)cc1 ; c1ccc(-c2ccc(N(c3ccc(-c4cccc5c4oc4c(-c6ccccc6)cccc45)cc3)c3cccc(-c4cccc5ccccc45)c3-c3cccc4ccccc34)cc2)cc1 ; c1ccc(-c2cccc(N(c3cccc(-c4cccc5ccccc45)c3-c3cccc4ccccc34)c3cccc4c3sc3c(-c5ccccc5)cccc34)c2)cc1]

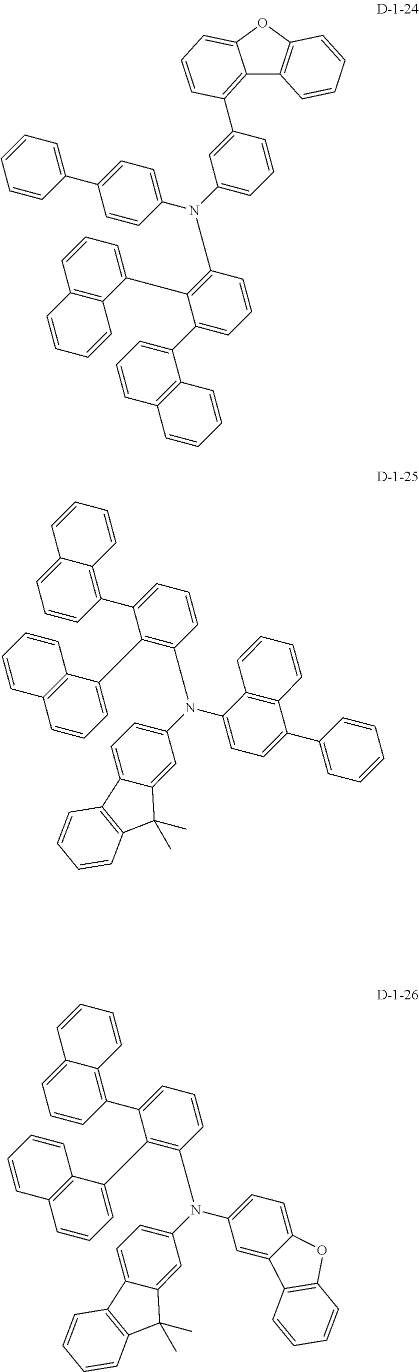 [SMILES: c1ccc(-c2ccc(N(c3cccc(-c4cccc5oc6ccccc6c45)c3)c3cccc(-c4cccc5ccccc45)c3-c3cccc4ccccc34)cc2)cc1 ; CC1(C)c2ccccc2-c2ccc(N(c3cccc(-c4cccc5ccccc45)c3-c3cccc4ccccc34)c3ccc(-c4ccccc4)c4ccccc34)cc21 ; CC1(C)c2ccccc2-c2ccc(N(c3ccc4oc5ccccc5c4c3)c3cccc(-c4cccc5ccccc45)c3-c3cccc4ccccc34)cc21]

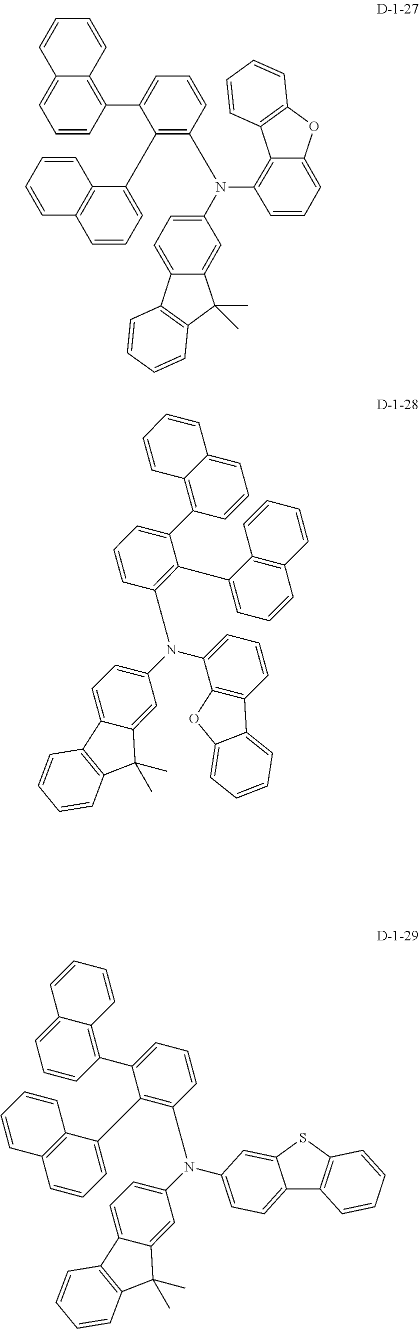

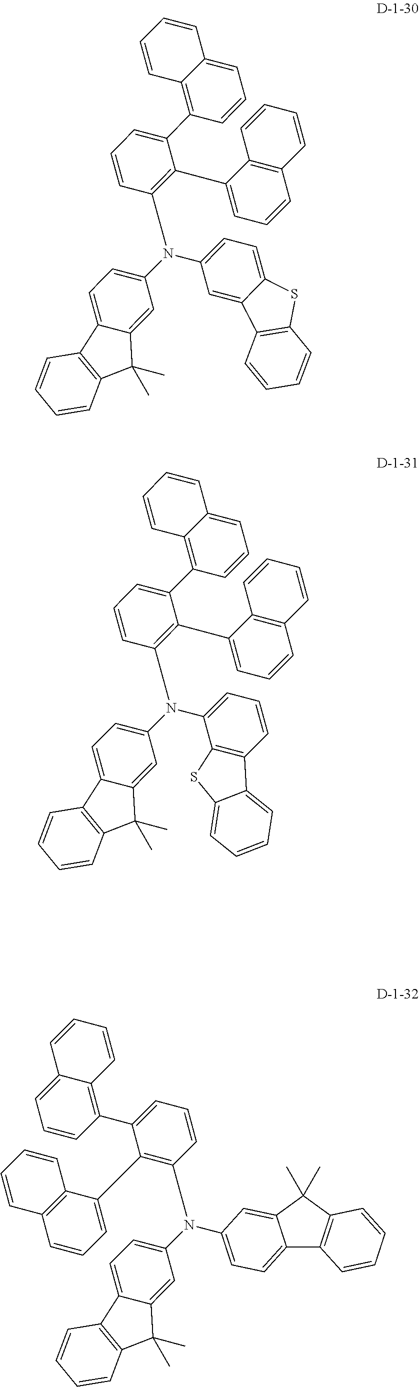 [SMILES: CC1(C)c2ccccc2-c2ccc(N(c3ccc4sc5ccccc5c4c3)c3cccc(-c4cccc5ccccc45)c3-c3cccc4ccccc34)cc21 ; CC1(C)c2ccccc2-c2ccc(N(c3cccc(-c4cccc5ccccc45)c3-c3cccc4ccccc34)c3cccc4c3sc3ccccc34)cc21 ; CC1(C)c2ccccc2-c2ccc(N(c3ccc4c(c3)C(C)(C)c3ccccc3-4)c3cccc(-c4cccc5ccccc45)c3-c3cccc4ccccc34)cc21]

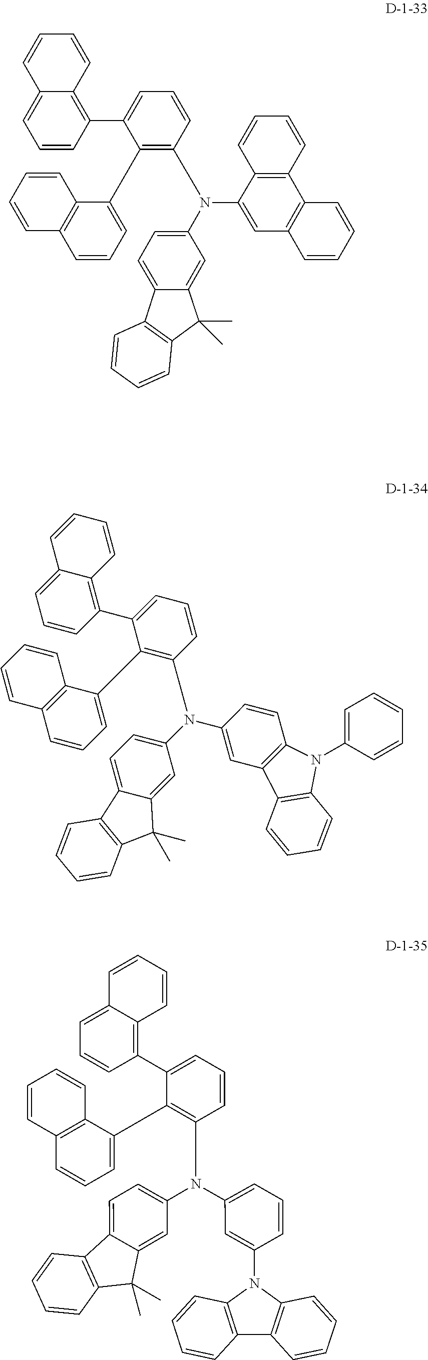 [SMILES: CC1(C)c2ccccc2-c2ccc(N(c3cccc(-c4cccc5ccccc45)c3-c3cccc4ccccc34)c3cc4ccccc4c4ccccc34)cc21 ; CC1(C)c2ccccc2-c2ccc(N(c3ccc4c(c3)c3ccccc3n4-c3ccccc3)c3cccc(-c4cccc5ccccc45)c3-c3cccc4ccccc34)cc21 ; CC1(C)c2ccccc2-c2ccc(N(c3cccc(-n4c5ccccc5c5ccccc54)c3)c3cccc(-c4cccc5ccccc45)c3-c3cccc4ccccc34)cc21]

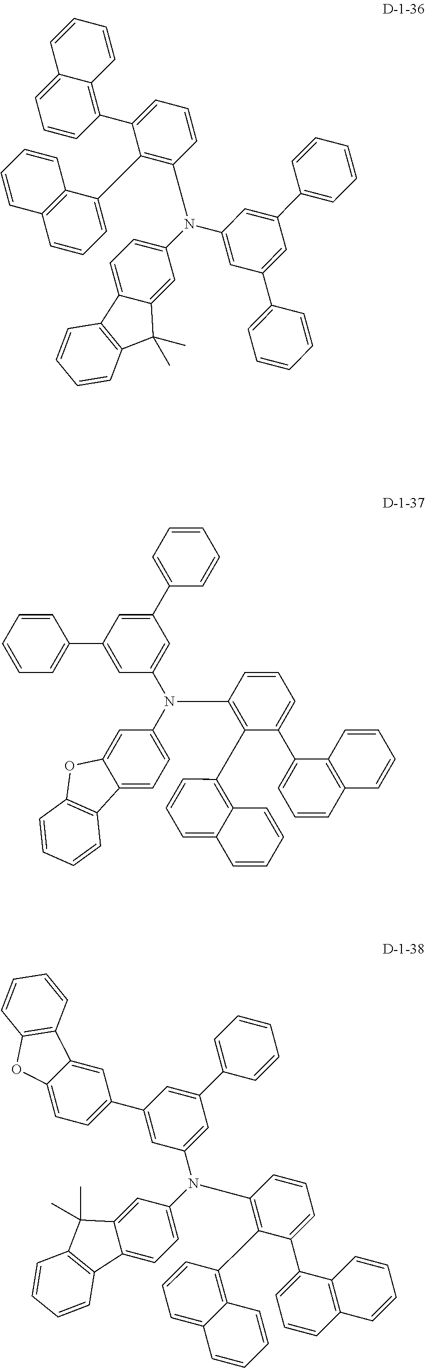 [SMILES: CC1(C)c2ccccc2-c2ccc(N(c3cc(-c4ccccc4)cc(-c4ccccc4)c3)c3cccc(-c4cccc5ccccc45)c3-c3cccc4ccccc34)cc21 ; c1ccc(-c2cc(-c3ccccc3)cc(N(c3ccc4c(c3)oc3ccccc34)c3cccc(-c4cccc5ccccc45)c3-c3cccc4ccccc34)c2)cc1 ; CC1(C)c2ccccc2-c2ccc(N(c3cc(-c4ccccc4)cc(-c4ccc5oc6ccccc6c5c4)c3)c3cccc(-c4cccc5ccccc45)c3-c3cccc4ccccc34)cc21]

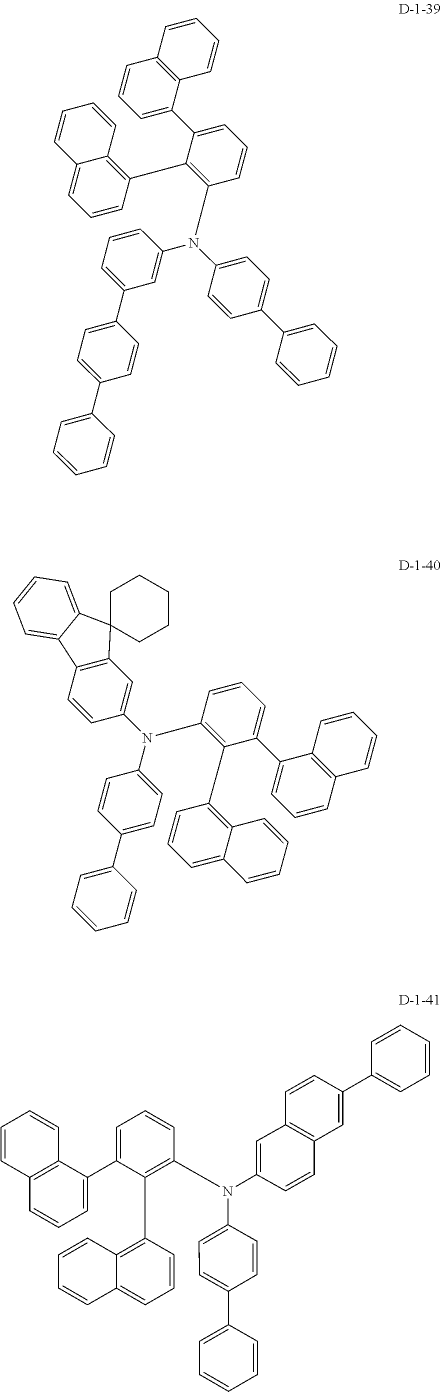 [SMILES: c1ccc(-c2ccc(-c3cccc(N(c4ccc(-c5ccccc5)cc4)c4cccc(-c5cccc6ccccc56)c4-c4cccc5ccccc45)c3)cc2)cc1 ; c1ccc(-c2ccc(N(c3ccc4c(c3)C3(CCCCC3)c3ccccc3-4)c3cccc(-c4cccc5ccccc45)c3-c3cccc4ccccc34)cc2)cc1 ; c1ccc(-c2ccc(N(c3ccc4cc(-c5ccccc5)ccc4c3)c3cccc(-c4cccc5ccccc45)c3-c3cccc4ccccc34)cc2)cc1]

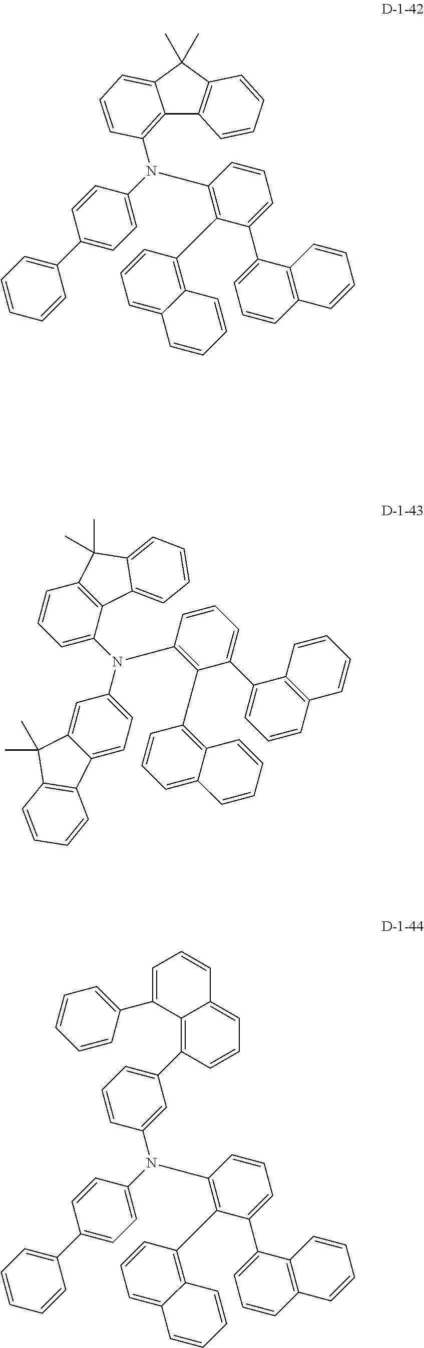 [SMILES: CC1(C)c2ccccc2-c2c(N(c3ccc(-c4ccccc4)cc3)c3cccc(-c4cccc5ccccc45)c3-c3cccc4ccccc34)cccc21 ; CC1(C)c2ccccc2-c2ccc(N(c3cccc(-c4cccc5ccccc45)c3-c3cccc4ccccc34)c3cccc4c3-c3ccccc3C4(C)C)cc21 ; c1ccc(-c2ccc(N(c3cccc(-c4cccc5cccc(-c6ccccc6)c45)c3)c3cccc(-c4cccc5ccccc45)c3-c3cccc4ccccc34)cc2)cc1]

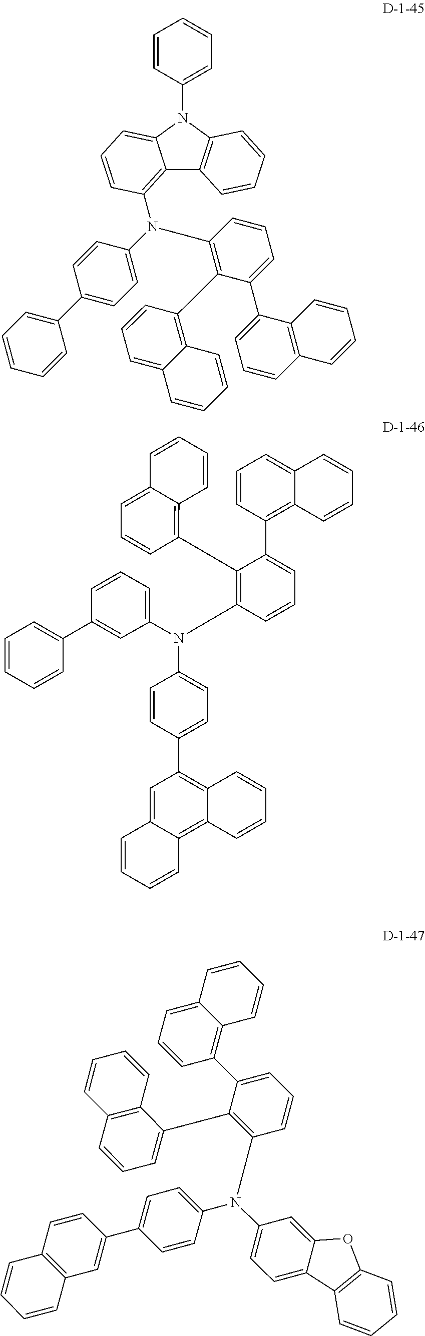

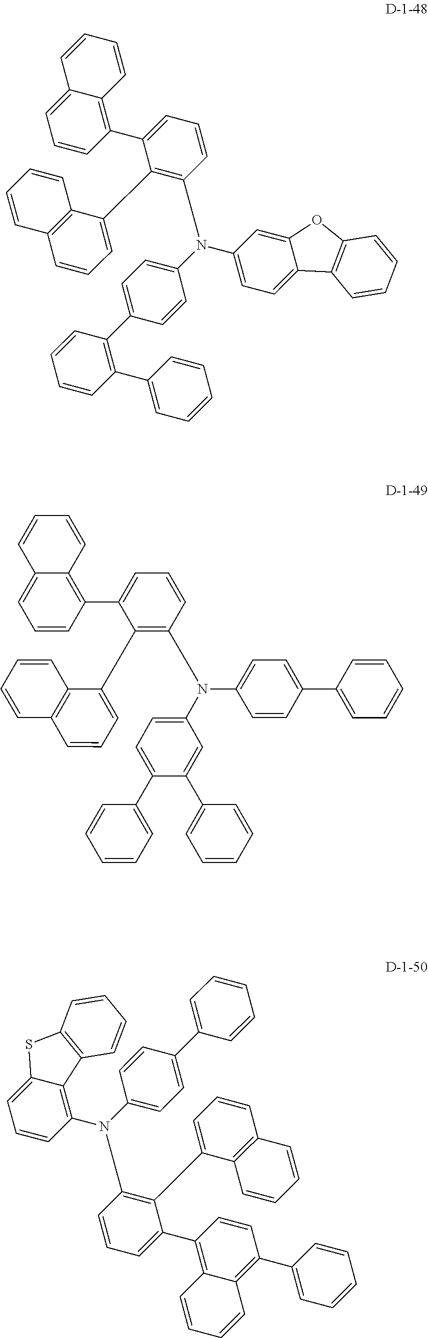 [SMILES: c1ccc(-c2ccccc2-c2ccc(N(c3ccc4c(c3)oc3ccccc34)c3cccc(-c4cccc5ccccc45)c3-c3cccc4ccccc34)cc2)cc1 ; c1ccc(-c2ccc(N(c3ccc(-c4ccccc4)c(-c4ccccc4)c3)c3cccc(-c4cccc5ccccc45)c3-c3cccc4ccccc34)cc2)cc1 ; c1ccc(-c2ccc(N(c3cccc(-c4ccc(-c5ccccc5)c5ccccc45)c3-c3cccc4ccccc34)c3cccc4sc5ccccc5c34)cc2)cc1]

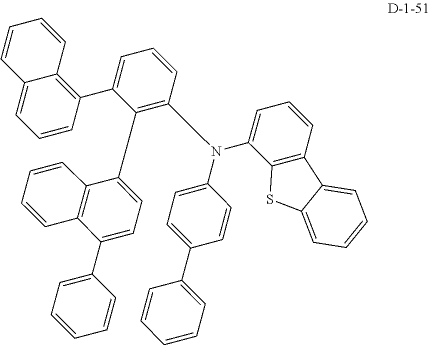 [SMILES: c1ccc(-c2ccc(N(c3cccc(-c4cccc5ccccc45)c3-c3ccc(-c4ccccc4)c4ccccc34)c3cccc4c3sc3ccccc34)cc2)cc1]

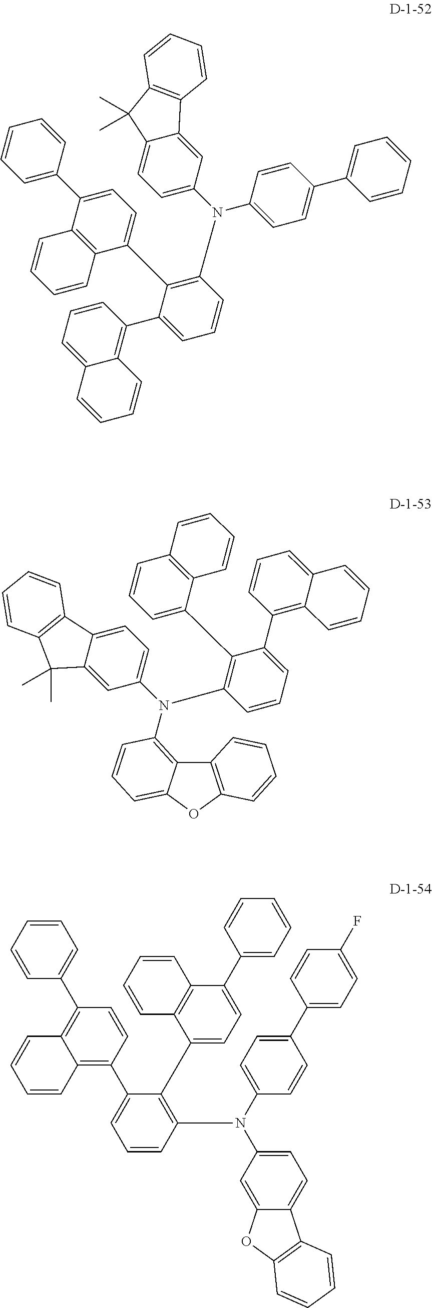 [SMILES: CC1(C)c2ccccc2-c2cc(N(c3ccc(-c4ccccc4)cc3)c3cccc(-c4cccc5ccccc45)c3-c3ccc(-c4ccccc4)c4ccccc34)ccc21 ; CC1(C)c2ccccc2-c2ccc(N(c3cccc(-c4cccc5ccccc45)c3-c3cccc4ccccc34)c3cccc4oc5ccccc5c34)cc21 ; Fc1ccc(-c2ccc(N(c3ccc4c(c3)oc3ccccc34)c3cccc(-c4ccc(-c5ccccc5)c5ccccc45)c3-c3ccc(-c4ccccc4)c4ccccc34)cc2)cc1]

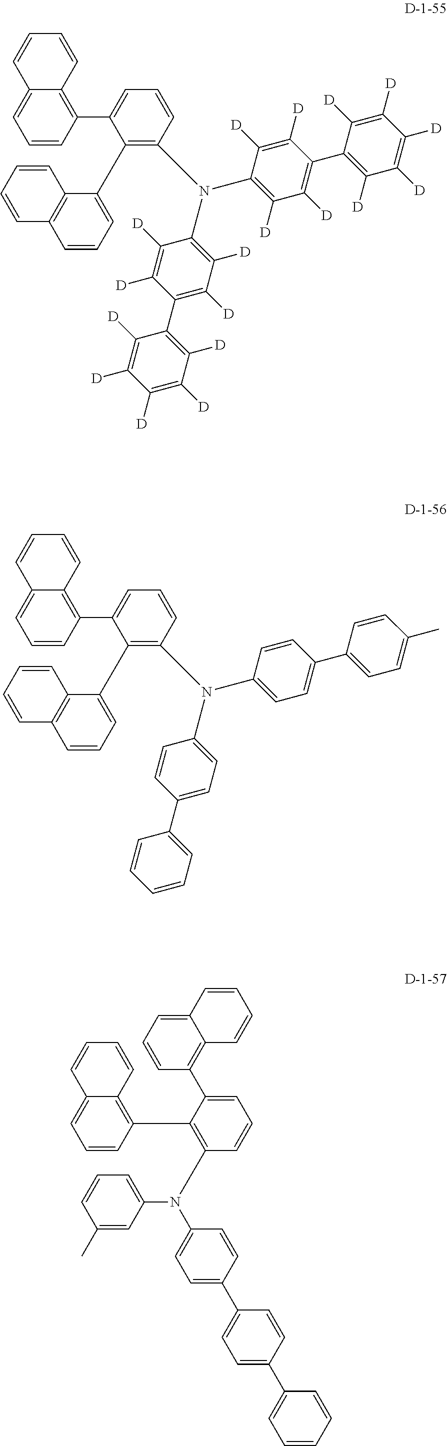 [SMILES: [2H]c1c([2H])c([2H])c(-c2c([2H])c([2H])c(N(c3cccc(-c4cccc5ccccc45)c3-c3cccc4ccccc34)c3c([2H])c([2H])c(-c4c([2H])c([2H])c([2H])c([2H])c4[2H])c([2H])c3[2H])c([2H])c2[2H])c([2H])c1[2H] ; Cc1ccc(-c2ccc(N(c3ccc(-c4ccccc4)cc3)c3cccc(-c4cccc5ccccc45)c3-c3cccc4ccccc34)cc2)cc1 ; Cc1cccc(N(c2ccc(-c3ccc(-c4ccccc4)cc3)cc2)c2cccc(-c3cccc4ccccc34)c2-c2cccc3ccccc23)c1]

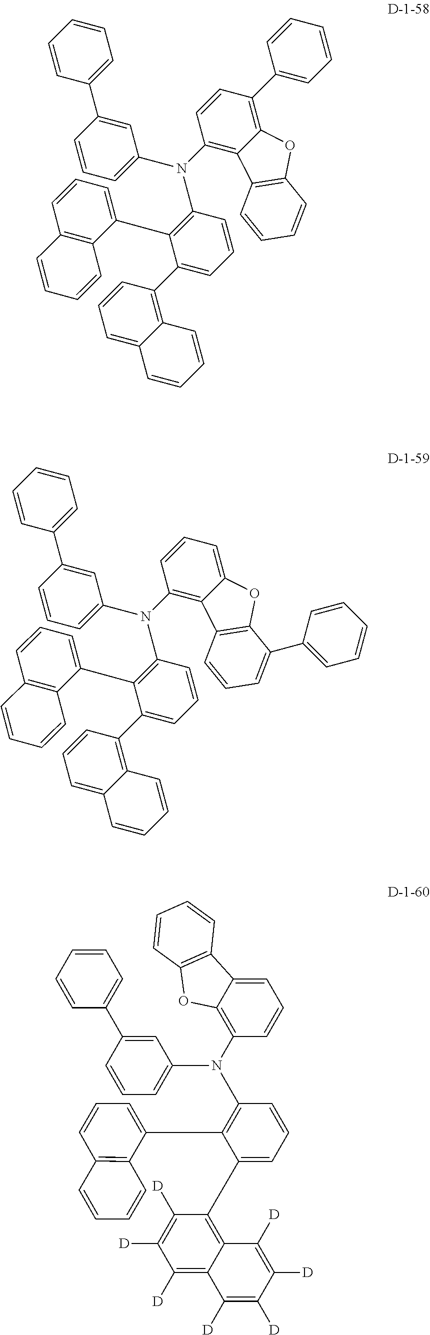 [SMILES: c1ccc(-c2cccc(N(c3cccc(-c4cccc5ccccc45)c3-c3cccc4ccccc34)c3ccc(-c4ccccc4)c4oc5ccccc5c34)c2)cc1 ; c1ccc(-c2cccc(N(c3cccc(-c4cccc5ccccc45)c3-c3cccc4ccccc34)c3cccc4oc5c(-c6ccccc6)cccc5c34)c2)cc1 ; [2H]c1c([2H])c([2H])c2c(-c3cccc(N(c4cccc(-c5ccccc5)c4)c4cccc5c4oc4ccccc45)c3-c3cccc4ccccc34)c([2H])c([2H])c([2H])c2c1[2H]]

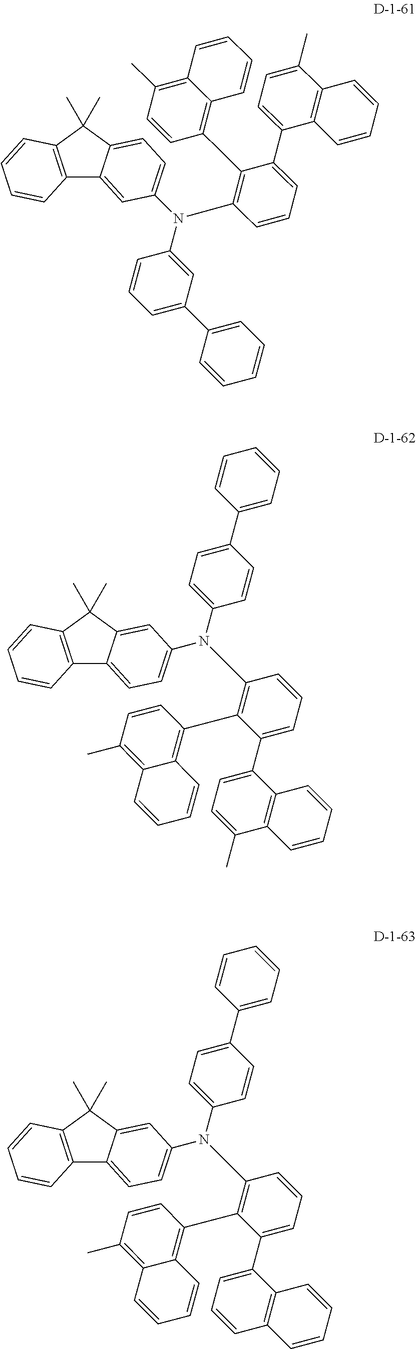 [SMILES: Cc1ccc(-c2cccc(N(c3cccc(-c4ccccc4)c3)c3ccc4c(c3)-c3ccccc3C4(C)C)c2-c2ccc(C)c3ccccc23)c2ccccc12 ; Cc1ccc(-c2cccc(N(c3ccc(-c4ccccc4)cc3)c3ccc4c(c3)C(C)(C)c3ccccc3-4)c2-c2ccc(C)c3ccccc23)c2ccccc12 ; Cc1ccc(-c2c(-c3cccc4ccccc34)cccc2N(c2ccc(-c3ccccc3)cc2)c2ccc3c(c2)C(C)(C)c2ccccc2-3)c2ccccc12]

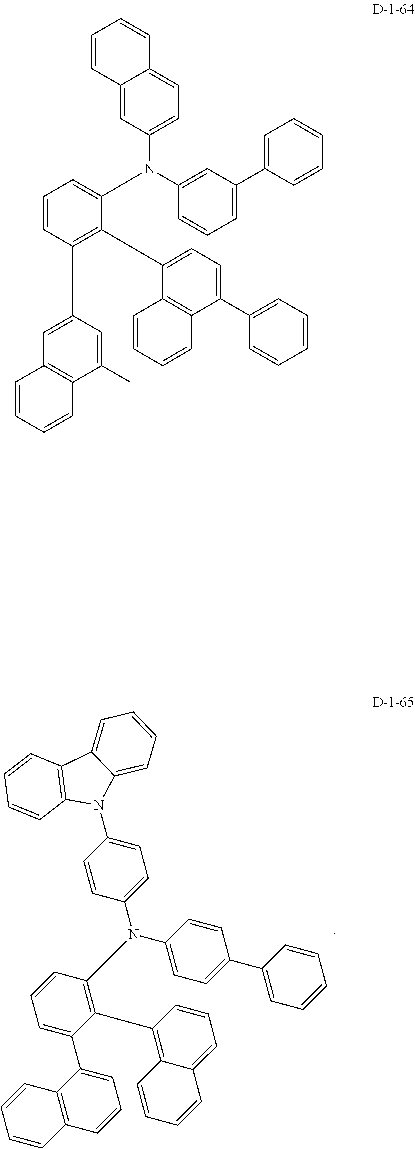 [SMILES: Cc1cc(-c2cccc(N(c3cccc(-c4ccccc4)c3)c3ccc4ccccc4c3)c2-c2ccc(-c3ccccc3)c3ccccc23)cc2ccccc12 ; c1ccc(-c2ccc(N(c3ccc(-n4c5ccccc5c5ccccc54)cc3)c3cccc(-c4cccc5ccccc45)c3-c3cccc4ccccc34)cc2)cc1]

The present application, in a second aspect, provides an electronic element, comprising an anode, a cathode, and a functional layer disposed between the anode and the cathode, wherein the functional layer comprises the nitrogen-containing compound described in the first aspect.

Optionally, the functional layer comprises a hole transport layer, and the hole transport layer comprises the nitrogen-containing compound described in the present application.

In the present application, the electronic element is an organic electroluminescent device or a photoelectric conversion device.

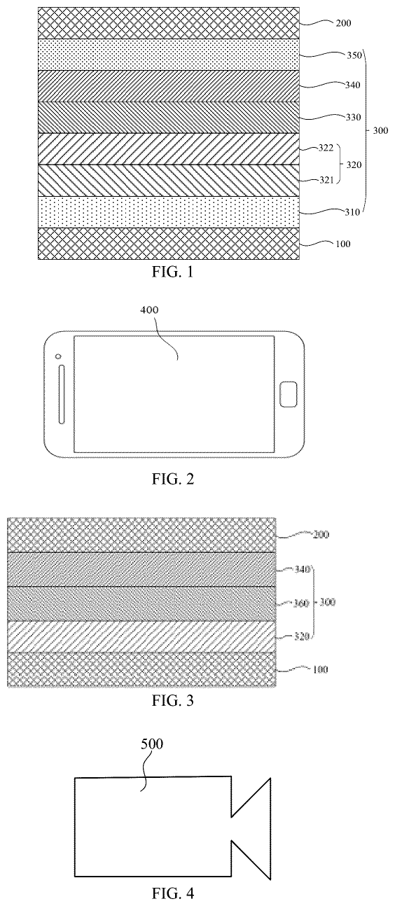

According to a specific embodiment, the electronic element is an organic electroluminescent device. As shown in FIG. 1, the organic electroluminescent device comprises an anode 100, a hole transport layer 320, an organic emissive layer 330, an electron transport layer 340, and a cathode 200 that are stacked in sequence.

Optionally, the hole transport layer 320 comprises the nitrogen-containing compound in the present application.

Optionally, the hole transport layer 320 comprises a first hole transport layer 321 and a second hole transport layer 322 that are stacked in layers. The first hole transport layer 321 is closer to the anode 100 than the second hole transport layer 322.

In the present application, the anode 100 comprises the following anode material, which is preferably a high work function material contributing to injection of holes into the functional layer. Specific examples of the anode material include, but are not limited to: metals such as nickel, platinum, vanadium, chromium, copper, zinc, gold, and alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly[3,4-(ethylene-1,2-dioxy)thiophene](PEDT), polypyrrole, and polyaniline. Preferably, a transparent electrode comprising indium tin oxide (ITO) is included as the anode.

Optionally, the material of the first hole transport layer 321 is selected from a variety of existing hole transport layer materials, which are not specifically qualified in the present application. The hole transport layer materials may be selected from carbazole polymers, carbazole-linked aromatic amine compounds, dibenzofuran-linked aromatic amine compounds, substituted fluorene-linked triarylamine compounds or other types of compounds, and specific examples include but are not limited to at least one of the following compounds:

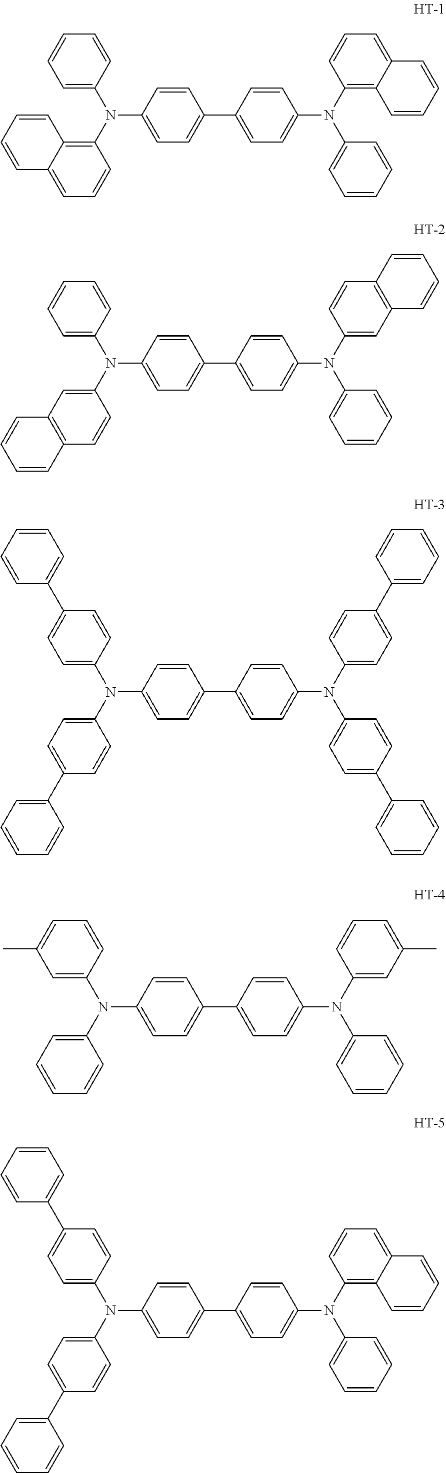

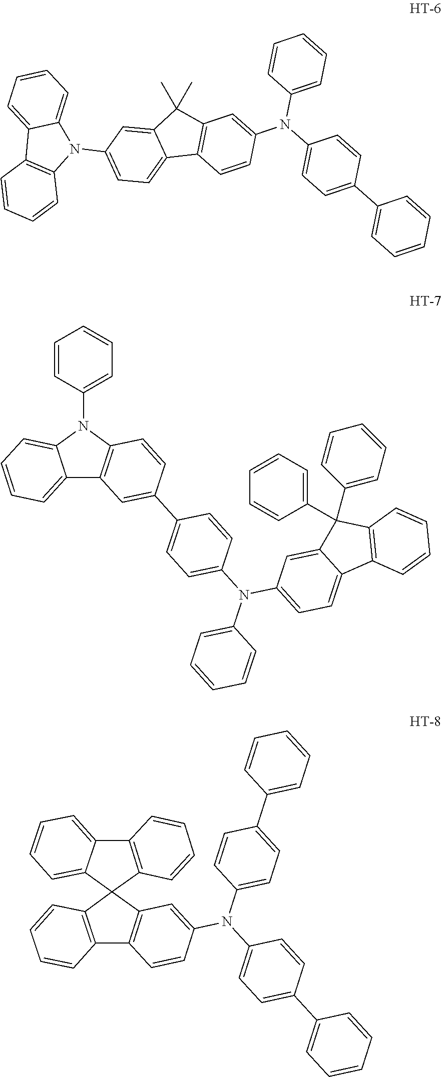 [SMILES: CC1(C)c2cc(N(c3ccccc3)c3ccc(-c4ccccc4)cc3)ccc2-c2ccc(-n3c4ccccc4c4ccccc43)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccccc4)cc3)c3ccc4c(c3)C3(c5ccccc5-c5ccccc53)c3ccccc3-4)cc2)cc1]

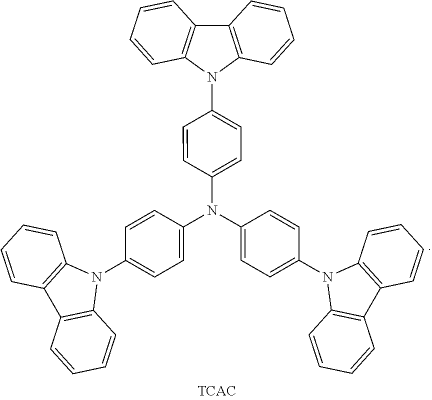 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

In a specific embodiment, the material of the first hole transport layer 321 is HT-1.

Optionally, the material of the second hole transport layer 322 comprises the nitrogen-containing compound in the present application.

The organic emissive layer 330 may be composed of a single emissive material, or may include a host material and a guest material (i.e. dopant). Optionally, the organic emissive layer 330 is composed of a host material and a guest material. Holes injected into the organic emissive layer 330 and electrons injected into the organic emissive layer 330 can recombine in the organic emissive layer 330 to form excitons. The excitons transmit energy to the host material, and the host material transmits the energy to the guest material, thereby enabling the guest material to emit light.

The host material of the organic emissive layer 330 may be a metal chelating compound, a stilbene derivative, an aromatic amine derivative, a dibenzofuran derivative, an anthracene derivative or other types of materials, and the present application is not particularly restricted in this respect. For example, the host material is selected from one or more of the following compounds:

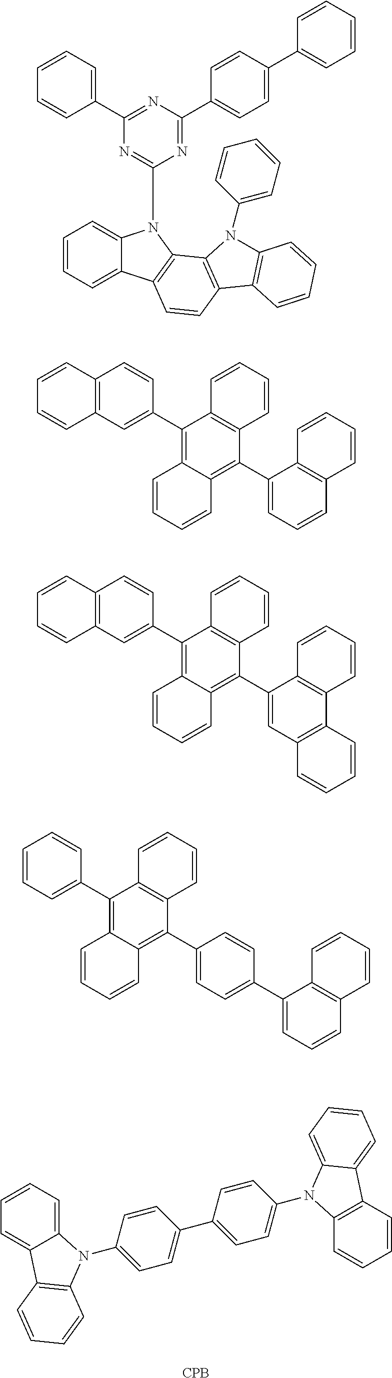

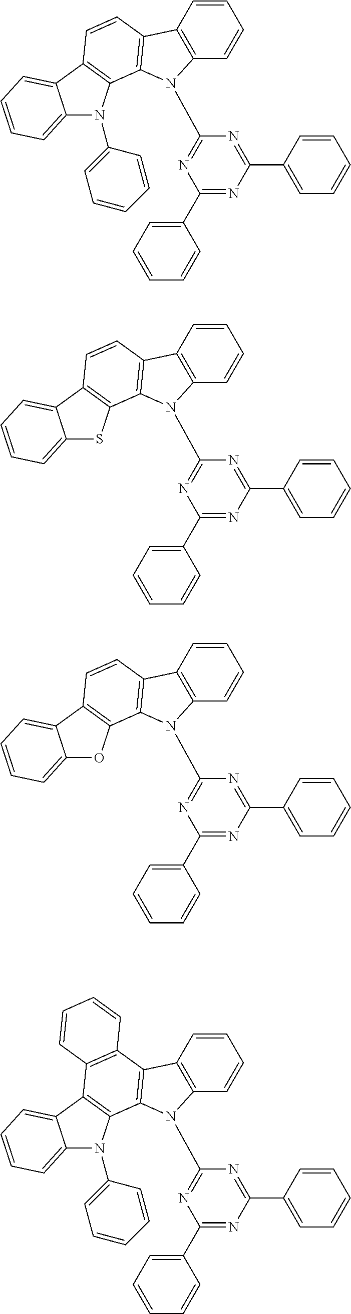

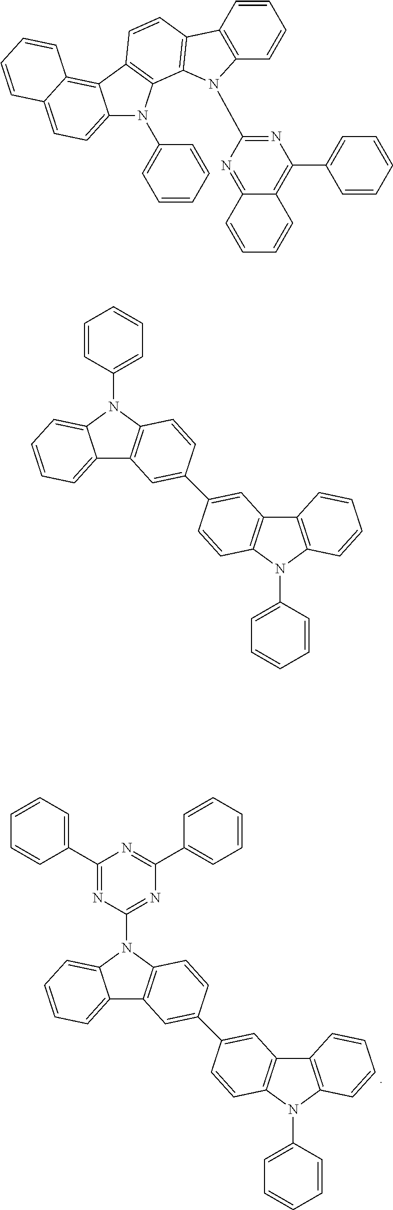 [SMILES: c1ccc(-c2nc(-n3c4ccccc4c4ccc5c6c7ccccc7ccc6n(-c6ccccc6)c5c43)nc3ccccc23)cc1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

In a specific embodiment, the host material is CPB.

The guest material of the organic emissive layer 330 may be a compound having a condensed aryl ring or a derivative thereof, a compound having a heteroaryl ring or a derivative thereof, a bisarylamine derivative thereof with fused arylene, or other materials, and the present application is not particularly restricted in this respect. For example, the guest material is at least one of the following compounds:

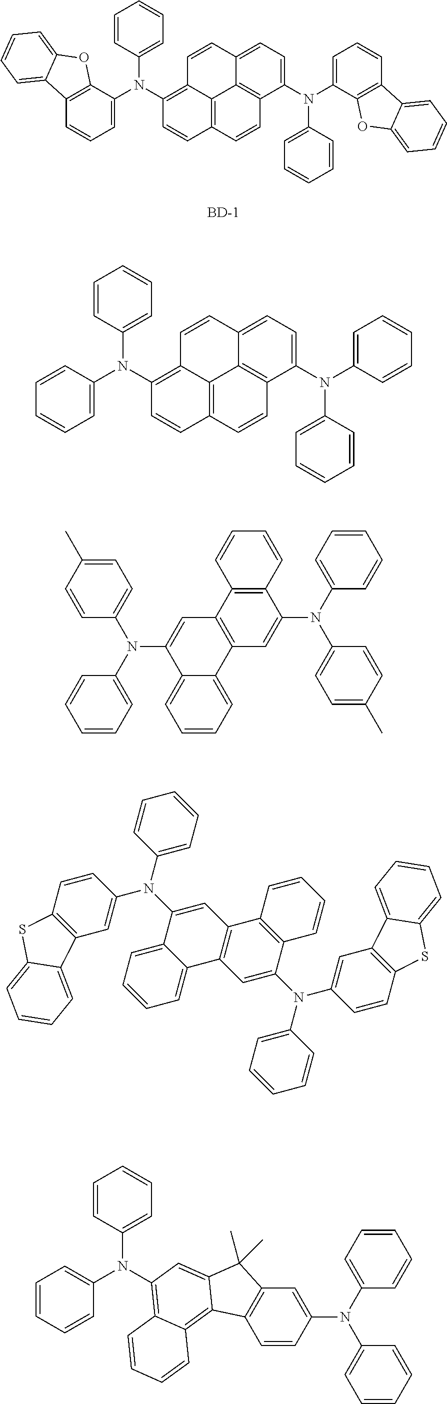 [SMILES: c1ccc(N(c2ccc3ccc4c(N(c5ccccc5)c5cccc6c5oc5ccccc56)ccc5ccc2c3c54)c2cccc3c2oc2ccccc23)cc1 ; "BD-1" ; c1ccc(N(c2ccccc2)c2ccc3ccc4c(N(c5ccccc5)c5ccccc5)ccc5ccc2c3c54)cc1 ; Cc1ccc(N(c2ccccc2)c2cc3c4ccccc4c(N(c4ccccc4)c4ccc(C)cc4)cc3c3ccccc23)cc1 ; c1ccc(N(c2ccc3sc4ccccc4c3c2)c2cc3c4ccccc4c(N(c4ccccc4)c4ccc5sc6ccccc6c5c4)cc3c3ccccc23)cc1 ; CC1(C)c2cc(N(c3ccccc3)c3ccccc3)ccc2-c2c1cc(N(c1ccccc1)c1ccccc1)c1ccccc21]

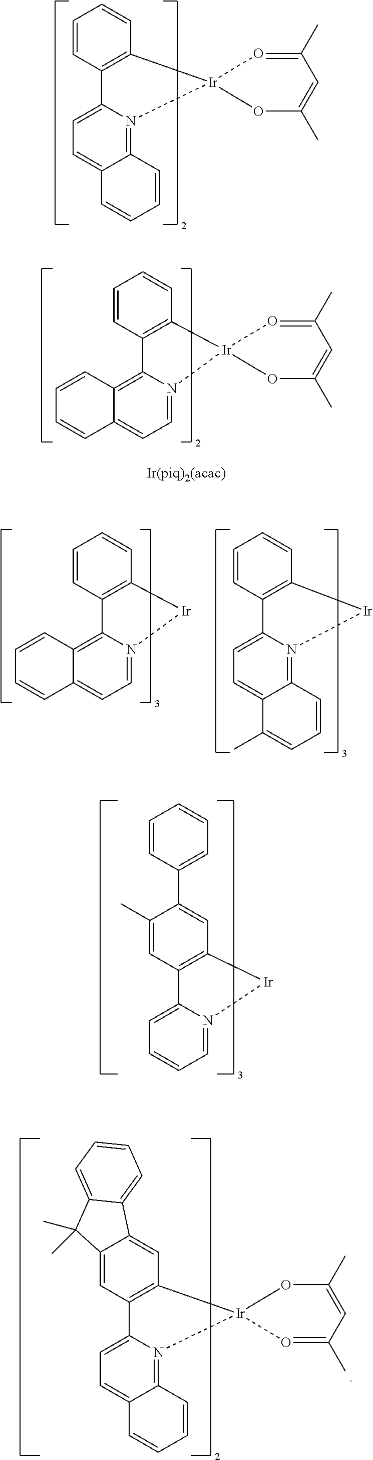

In a specific embodiment, the guest material is Ir(piq)₂(acac).

The electron transport layer 340 may be a single-layer structure, or a multi-layer structure, and may comprise the nitrogen-containing compound in the present application and optionally one or more other electron transport materials. The other electron transport materials may typically comprise metal complexes and/or nitrogen-containing heterocyclic derivatives, wherein the metal complex material may, for example, be selected from LiQ, Alq₃, Bepq₂, etc. The nitrogen-containing heterocyclic derivative may be an aromatic ring having a nitrogen-containing 6-membered ring or 5-membered ring skeleton, a fused aromatic ring compound having a nitrogen-containing 6-membered ring or 5-membered ring skeleton, such as DBimiBphen.

In a specific embodiment, the electron transport layer 340 is composed of LiQ and DBimiBphen.

In the present application, the cathode 200 comprises a cathode material, which is a low-work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multilayer materials such as LiF/Al, Liq/Al, LiO₂/Al, LiF/Ca, LiF/Al, and BaF₂/Ca. Preferably, a metal electrode comprising magnesium and silver is used as the cathode.

Optionally, as shown in FIG. 1, a hole injection layer 310 is further provided between the anode 100 and the hole transport layer 320 to enhance the ability to inject holes into the hole transport layer 320. The hole injection layer 310 may be composed of a material selected from benzidine derivatives, starburst arylamine compounds, phthalocyanine derivatives, or other materials, and the present application is not particularly limited in this respect. For example, the hole injection layer 310 is selected from the group consisting of the following compounds:

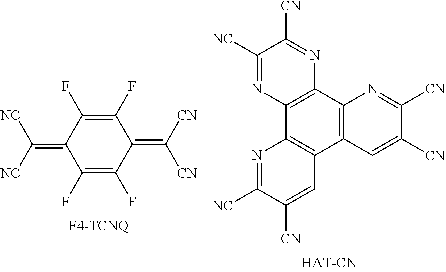 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2nc1C#N]

In a specific embodiment, the hole injection layer 310 is m-MTDATA.

Optionally, as shown in FIG. 1, an electron injection layer 350 is further provided between the cathode 200 and the electron transport layer 340 to enhance the ability to inject electrons into the electron transport layer 340. The electron injection layer 350 may comprise an inorganic material such as alkali metal sulfides, alkali metal halides, or may comprise a complex of an alkali metal and an organic substance.

In a specific embodiment, the electron injection layer 350 comprises LiQ.

In the present application, the organic electroluminescent device may be a blue light-emitting device, a red light-emitting device, a green light-emitting device, preferably a red light-emitting device.

According to another embodiment, the electronic component is a photoelectric conversion device. As shown in FIG. 3, the photoelectric conversion device may comprise an anode 100, a hole transport layer 320, a photoelectric conversion layer 360, an electron transport layer 340, and a cathode 200 that are stacked in sequence, wherein the hole transport layer 320 comprises the nitrogen-containing compound provided in the present application.

Optionally, the photoelectric conversion device is a solar cell, such as an organic thin-film solar cell.

The present application, in a third aspect, provides an electronic device, comprising the electronic element as described in the second aspect.

According to an embodiment, as shown in FIG. 2, the electronic device is a first electronic device 400. The first electronic device 400 comprises the organic electroluminescent device described above. The first electronic device 400 may be, for example, a display device, a lighting device, an optical communication device, or other type of electronic devices, which, for example, may include, but are not limited to, computer screens, mobile phone screens, televisions, electronic paper, emergency lamps, optical modules, etc.

According to another embodiment, as shown in FIG. 4, the electronic device is a second electronic device 500. The second electronic device 500 comprises the photoelectric conversion device described above. The second electronic device 500 may be a solar power generation device, a light detector, a fingerprint recognition device, an optical module, a CCD camera, or other types of electronic devices.

The present application is further described below by way of examples. However, the following examples are only examples of the present application, and not limit to, the present application in any way.

Compounds for which a synthesis method is not mentioned in the present application are raw material products obtained commercially.

### Synthesis of IM-a-1

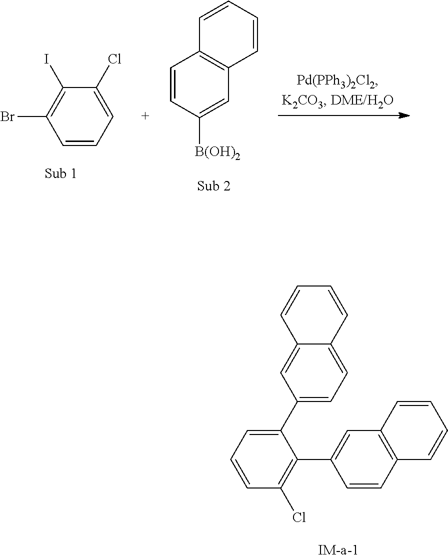 [SMILES: Clc1cccc(Br)c1I ; OB(O)c1ccc2ccccc2c1 ; Clc1cccc(-c2ccc3ccccc3c2)c1-c1ccc2ccccc2c1]

To a three-necked flask equipped with a mechanical stirrer, a thermometer and a spherical condensing tube was purged with nitrogen gas (0.100 L/min) for 15 min to replace the air therein. Sub 1 (3-chloro-2-bromo-1-iodobenzene, 100.0 g, 315.1 mmol), Sub 2 (2-naphthylboric acid, 113.81 g, 661.7 mmol), bis(triphenylphosphine)palladium dichloride (Pd(PPh₃)₂Cl₂, 2.2 g, 3.15 mmol), and potassium carbonate (K₂CO₃, 174.21 g, 1260.44 mmol) were added sequentially to the three-neck flask, followed by addition of a solvent mixture of ethylene glycol dimethyl ether (DME, 1600 mL) and water (400 mL). The resulting mixture was stirred and heated to 78° C. to 80° C. to react for 48 h. After the reaction was completed, the reaction solution was cooled to room temperature. The reaction solution was washed with water and separated to obtain an organic phase. The organic phase was dried with anhydrous magnesium sulfate and filtered. The resulting filtrate was concentrated under reduced pressure to remove the solvent, obtaining a crude product. The crude product was purified by silica gel column chromatography with a mixture of dichloromethane/n-heptane as the eluent, to give IM-a-1 as a white solid (74.73 g, yield 65%).

The intermediates listed in Table 1 were prepared by the same method for preparing IM-a-1, except that Sub 2 was replaced with Raw material A. The main raw materials used, the intermediates prepared and yields thereof are shown in Table 1.

TABLE 1

| Raw material A | Intermediate | Yield (%) |
|---|---|---|
| 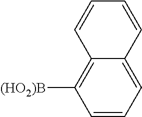 | 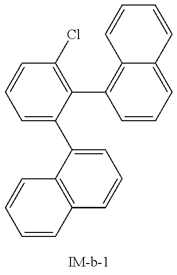 [SMILES: Clc1cccc(-c2cccc3ccccc23)c1-c1cccc2ccccc12] | 50 |
| 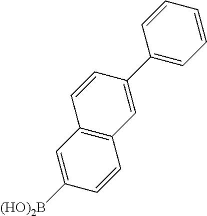 [SMILES: OB(O)c1ccc2cc(-c3ccccc3)ccc2c1] | 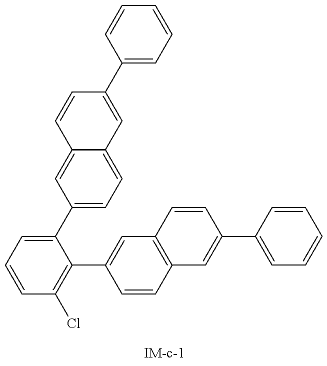 [SMILES: Clc1cccc(-c2ccc3cc(-c4ccccc4)ccc3c2)c1-c1ccc2cc(-c3ccccc3)ccc2c1] | 53 |
| 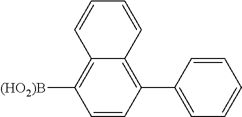 | 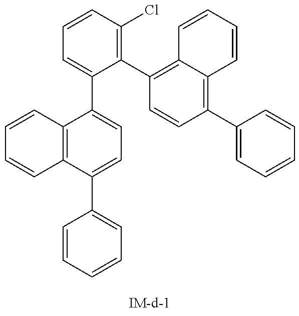 [SMILES: Clc1cccc(-c2ccc(-c3ccccc3)c3ccccc23)c1-c1ccc(-c2ccccc2)c2ccccc12] | 48 |
| 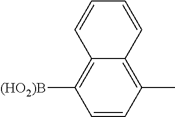 | 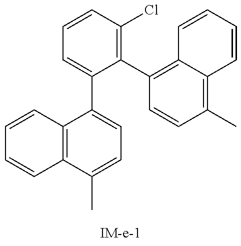 [SMILES: Cc1ccc(-c2cccc(Cl)c2-c2ccc(C)c3ccccc23)c2ccccc12] | 41 |
| 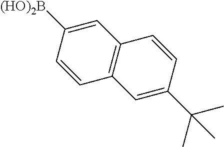 [SMILES: CC(C)(C)c1ccc2cc(B(O)O)ccc2c1] | 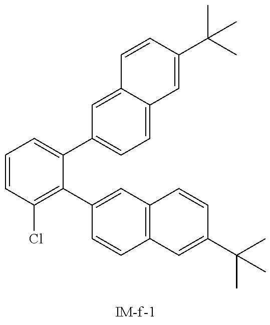 [SMILES: CC(C)(C)c1ccc2cc(-c3cccc(Cl)c3-c3ccc4cc(C(C)(C)C)ccc4c3)ccc2c1] | 43 |

### 2. Synthesis of IM-e-1-1

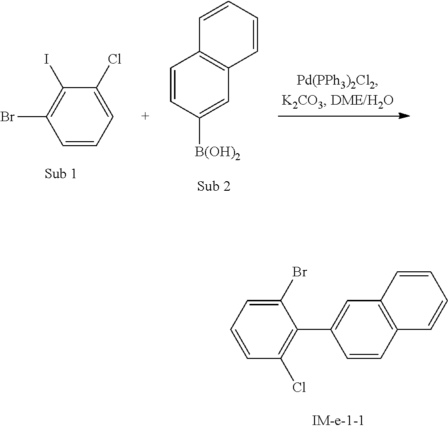 [SMILES: Clc1cccc(Br)c1I ; OB(O)c1ccc2ccccc2c1 ; Clc1cccc(Br)c1-c1ccc2ccccc2c1]

To a three-necked flask equipped with a mechanical stirrer, a thermometer and a spherical condensing tube was purged with nitrogen gas (0.100 L/min) for 15 min to replace the air therein. Sub 1 (100.0 g, 315.1 mmol), Sub 2 (54.20 g, 315.1 mmol), bis(triphenylphosphine) palladium dichloride (1.11 g, 1.58 mmol), and potassium carbonate (108.88 g, 787.78 mmol) were added sequentially to the three-neck flask, followed by addition of a solvent mixture of ethylene glycol dimethyl ether (800 mL) and water (400 mL). The resulting mixture was stirred and heated to 70° C. to 75° C., to react for 48 h. After the reaction was completed, the reaction solution was cooled to room temperature. The reaction solution was washed with water and separated to obtain an organic phase. The organic phase was dried with anhydrous magnesium sulfate and filtered. The resulting filtrate was concentrated under reduced pressure to remove the solvent, obtaining a crude product. The crude product was purified by silica gel column chromatography with a mixture of dichloromethane/n-heptane as the eluent, to give IM-e-1-1 as a white solid (49.04 g, yield 49%).

The intermediates listed in Table 2 were prepared by the same method for preparing IM-e-1-1, except that Sub 2 was replaced with Raw material B. The main raw materials used, the intermediates prepared and yields thereof are shown in Table 2.

TABLE 2

| Raw material B | Intermediate | Yield (%) |
|---|---|---|
| 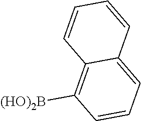 [SMILES: OB(O)c1cccc2ccccc12] | 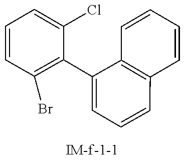 [SMILES: Clc1cccc(Br)c1-c1cccc2ccccc12] | 48 |
| 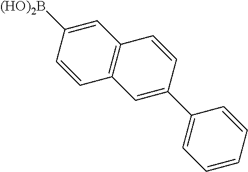 [SMILES: OB(O)c1ccc2cc(-c3ccccc3)ccc2c1] | 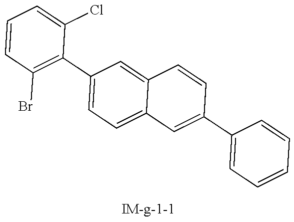 [SMILES: Clc1cccc(Br)c1-c1ccc2cc(-c3ccccc3)ccc2c1] | 51 |
| 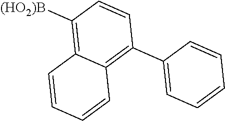 | 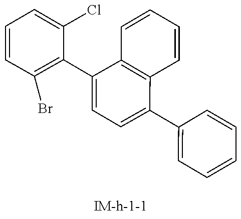 [SMILES: Clc1cccc(Br)c1-c1ccc(-c2ccccc2)c2ccccc12] | 53 |
| 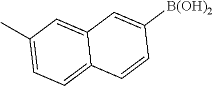 | 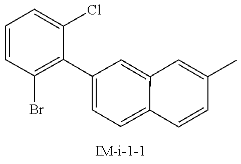 [SMILES: Cc1ccc2ccc(-c3c(Cl)cccc3Br)cc2c1] | 43 |

### 3. Synthesis of IM-g-1

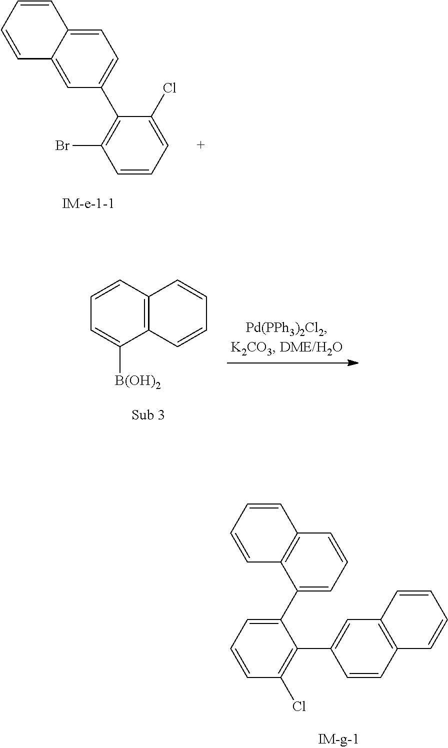 [SMILES: Clc1cccc(Br)c1-c1ccc2ccccc2c1 ; OB(O)c1cccc2ccccc12 ; Clc1cccc(-c2cccc3ccccc23)c1-c1ccc2ccccc2c1]

To a three-necked flask equipped with a mechanical stirrer, a thermometer and a spherical condensing tube was purged with nitrogen gas (0.100 L/min) for 15 min to replace the air therein. IM-e-1-1 (45.00 g, 141.68 mmol), Sub 3 (24.37 g, 141.68 mmol), bis(triphenylphosphine) palladium dichloride (0.99 g, 1.42 mmol), and potassium carbonate (48.95 g, 354.21 mmol) were added sequentially to the three-neck flask, followed by addition of a solvent mixture of ethylene glycol dimethyl ether (360 mL) and water (90 mL). The resulting mixture was stirred, and heated to 75° C. to 78° C., to react for 12 h. After the reaction was completed, the reaction solution was cooled to room temperature. The reaction solution was washed with water and separated to obtain an organic phase. The organic phase was dried with anhydrous magnesium sulfate and filtrated. The resulting filtrate was concentrated under reduced pressure to remove the solvent, obtaining a crude product. The crude product was purified by silica gel column chromatography with a mixture of dichloromethane/n-heptane as the eluent, to give IM-g-1 as a white solid (35.15 g, yield 68%).

The intermediates listed in Table 3 were prepared by the same method for preparing IM-g-1, except that IM-e-1-1 was replaced with Raw material C, Sub 3 was replaced with Raw material D. The main raw materials used, the intermediates prepared and yields thereof are shown in Table 3.

TABLE 3

| Raw material C | Raw material D | Intermediate | Yield (%) |
|---|---|---|---|
| 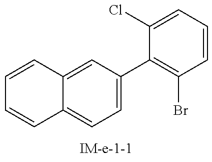 [SMILES: Clc1cccc(Br)c1-c1ccc2ccccc2c1] | 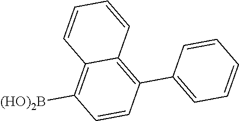 [SMILES: OB(O)c1ccc(-c2ccccc2)c2ccccc12] | 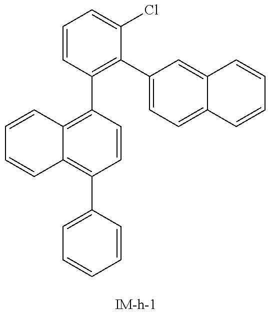 [SMILES: Clc1cccc(-c2ccc(-c3ccccc3)c3ccccc23)c1-c1ccc2ccccc2c1] | 65 |
| | 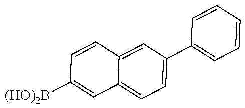 [SMILES: OB(O)c1ccc2cc(-c3ccccc3)ccc2c1] | 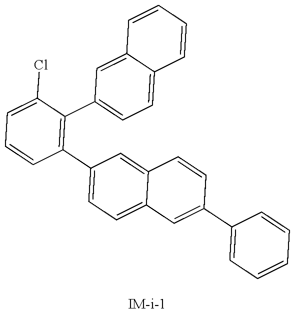 [SMILES: Clc1cccc(-c2ccc3cc(-c4ccccc4)ccc3c2)c1-c1ccc2ccccc2c1] | 68 |
| 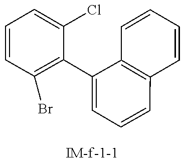 [SMILES: Clc1cccc(Br)c1-c1cccc2ccccc12] | 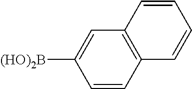 [SMILES: OB(O)c1ccc2ccccc2c1] | 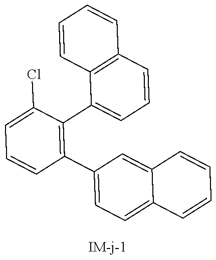 [SMILES: Clc1cccc(-c2ccc3ccccc3c2)c1-c1cccc2ccccc12] | 73 |
| | 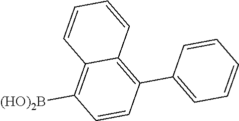 [SMILES: OB(O)c1ccc(-c2ccccc2)c2ccccc12] | 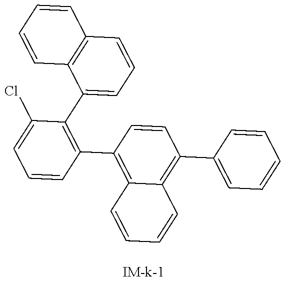 [SMILES: Clc1cccc(-c2ccc(-c3ccccc3)c3ccccc23)c1-c1cccc2ccccc12] | 71 |
| | 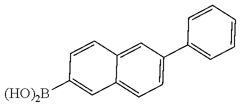 [SMILES: OB(O)c1ccc2cc(-c3ccccc3)ccc2c1] | 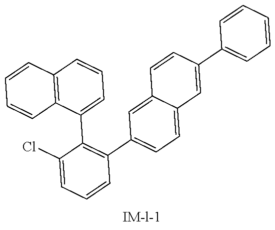 [SMILES: Clc1cccc(-c2ccc3cc(-c4ccccc4)ccc3c2)c1-c1cccc2ccccc12] | 75 |
| 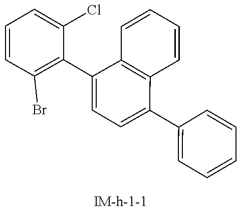 [SMILES: Clc1cccc(Br)c1-c1ccc(-c2ccccc2)c2ccccc12] | 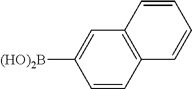 [SMILES: OB(O)c1ccc2ccccc2c1] | 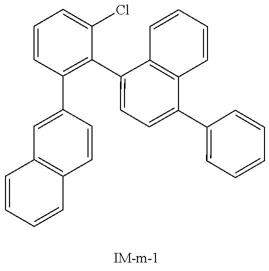 [SMILES: Clc1cccc(-c2ccc3ccccc3c2)c1-c1ccc(-c2ccccc2)c2ccccc12] | 63 |
| | 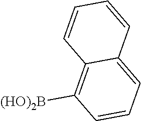 [SMILES: OB(O)c1cccc2ccccc12] | 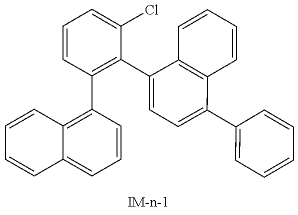 [SMILES: Clc1cccc(-c2cccc3ccccc23)c1-c1ccc(-c2ccccc2)c2ccccc12] | 76 |
| | 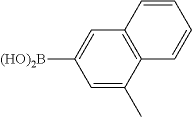 | 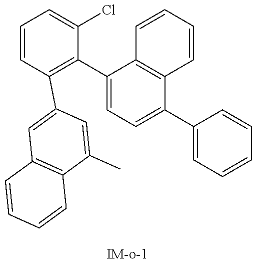 [SMILES: Cc1cc(-c2cccc(Cl)c2-c2ccc(-c3ccccc3)c3ccccc23)cc2ccccc12] | 68 |
| 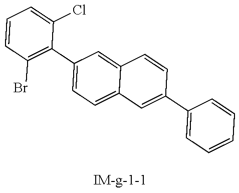 [SMILES: Clc1cccc(Br)c1-c1ccc2cc(-c3ccccc3)ccc2c1] | 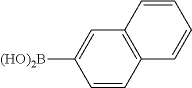 [SMILES: OB(O)c1ccc2ccccc2c1] | 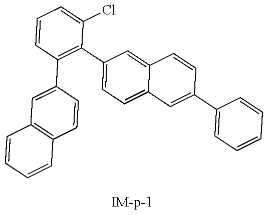 [SMILES: Clc1cccc(-c2ccc3ccccc3c2)c1-c1ccc2cc(-c3ccccc3)ccc2c1] | 70 |
| 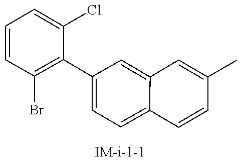 [SMILES: Cc1ccc2ccc(-c3c(Cl)cccc3Br)cc2c1] | 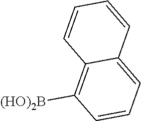 [SMILES: OB(O)c1cccc2ccccc12] | 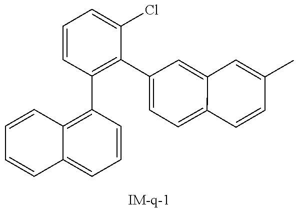 [SMILES: Cc1ccc2ccc(-c3c(Cl)cccc3-c3cccc4ccccc34)cc2c1] | 62 |

### 4. Synthesis of Compound A-1-3

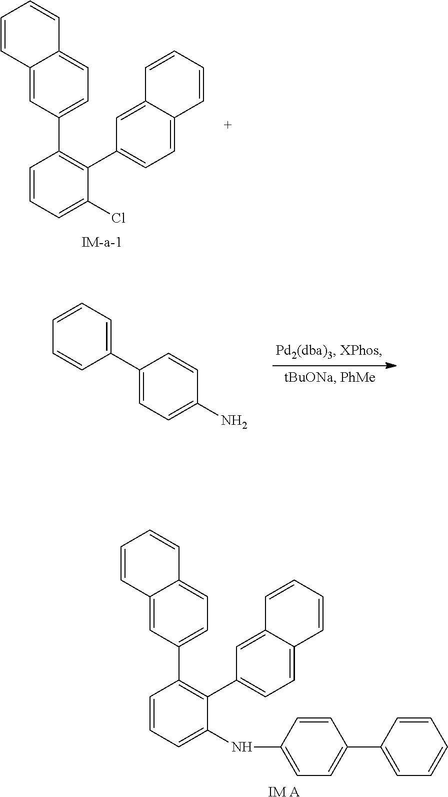

To a three-necked flask equipped with a mechanical stirrer, a thermometer and a spherical condensing tube was purged with nitrogen gas (0.200 L/min) for 15 min to replace the air therein. IM-a-1 (15 g, 41.11 mmol), 4-aminobiphenyl (6.96 g, 41.11 mmol), tris(dibenzylideneacetone)bis-palladium (Pd₂(dba)₃, 0.37 g, 0.41 mmol), 2-dicyclohexylphosphino-2′,4′,6′-triisopropylbiphenyl (XPhos, 0.39 g, 0.82 mmol), sodium tert-butoxide (tBuONa, 5.93 g, 61.66 mmol) and toluene (PhMe, 150 mL) were added sequentially to the three-neck flask. The resulting mixture was heated to reflux, to react for 4 h. After the reaction was completed, the reaction solution was cooled to room temperature. The reaction solution was washed with water and separated to obtain an organic phase, and the organic phase was dried with anhydrous magnesium sulfate, and filtered. The resulting filtrate was concentrated under reduced pressure to remove the solvent, obtaining a crude product. The crude product was purified by silica gel column chromatography with a mixture of dichloromethane/n-heptane system as the eluent, to give IM A as a white solid (14.93 g, yield 73%).

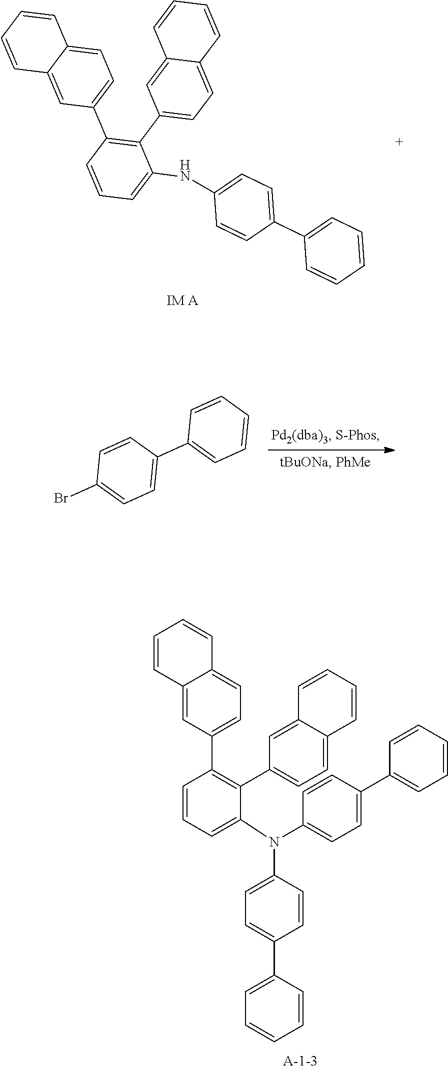

To a three-necked flask equipped with a mechanical stirrer, a thermometer and a spherical condensing tube was purged with nitrogen gas (0.100 L/min) for 15 min to replace the air therein. IM A (10 g, 20.09 mmol), 4-bromobiphenyl (4.68 g, 20.09 mmol), tris(dibenzylideneacetone) bis-palladium (0.184 g, 0.20 mmol), 2-dicyclohexylphosphino-2′,6′-dimethoxybiphenyl (S-Phos, 0.165 g, 0.40 mmol), sodium tert-butoxide (2.90 g, 30.14 mmol) and toluene (100 mL) were added sequentially to the three-neck flask. The resulting mixture was heated to reflux, to react for 30 h. After the reaction was completed, the reaction solution was cooled to room temperature after reaction. The reaction solution was washed with water and separated to obtain an organic phase, and the organic phase was dried with anhydrous magnesium sulfate and filtered. The resulting filtrate was concentrated under reduced pressure to remove the solvent, obtaining a crude product. The crude product was purified by silica gel column chromatography with a mixture of dichloromethane/n-heptane as the eluent, to give Compound A-1-3 as a white solid (6.65 g, yield 51%), mass spectrometry(m/z)=650.3[M+H]⁺.

The compounds listed in Table 4 were prepared by the same method for preparing Compound A-1-3, except that IM-a-1 was replaced with Raw material E, 4-aminobenzyl biphenyl was replaced with Raw material F, 4-bromobiphenyl was replaced with Raw material G. The main raw materials used, the compounds prepared and yields and mass spectrometry thereof are shown in Table 4.

TABLE 4

| | | | | | MS |
|---|---|---|---|---|---|
| | | | | | (m/z)/ |
| | | | | Yield | [M + |
| Raw material E | Raw material F | Raw material G | Compound | (%) | H]⁺ |
| 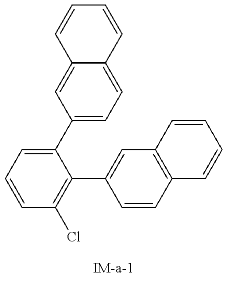 [SMILES: Clc1cccc(-c2ccc3ccccc3c2)c1-c1ccc2ccccc2c1] | 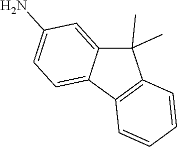 [SMILES: CC1(C)c2ccccc2-c2ccc(N)cc21] | 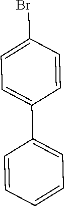 [SMILES: Brc1ccc(-c2ccccc2)cc1] | 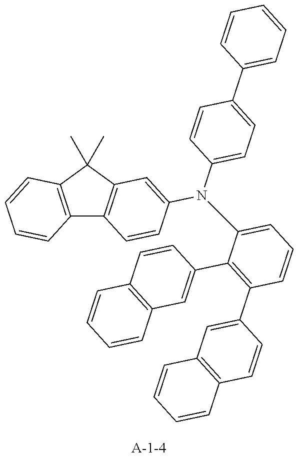 [SMILES: CC1(C)c2ccccc2-c2ccc(N(c3ccc(-c4ccccc4)cc3)c3cccc(-c4ccc5ccccc5c4)c3-c3ccc4ccccc4c3)cc21] | 34 | 690.3 |
| 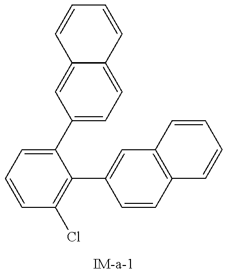 [SMILES: Clc1cccc(-c2ccc3ccccc3c2)c1-c1ccc2ccccc2c1] | 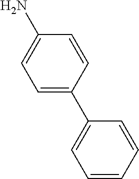 [SMILES: Nc1ccc(-c2ccccc2)cc1] | 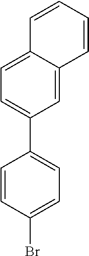 [SMILES: Brc1ccc(-c2ccc3ccccc3c2)cc1] | 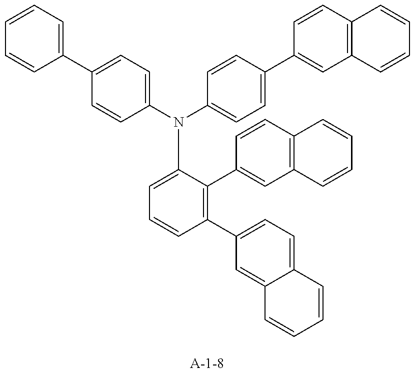 [SMILES: c1ccc(-c2ccc(N(c3ccc(-c4ccc5ccccc5c4)cc3)c3cccc(-c4ccc5ccccc5c4)c3-c3ccc4ccccc4c3)cc2)cc1] | 39 | 700.3 |
| 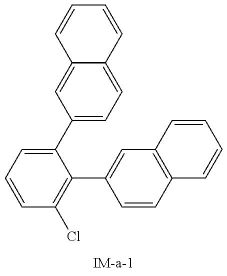 [SMILES: Clc1cccc(-c2ccc3ccccc3c2)c1-c1ccc2ccccc2c1] | 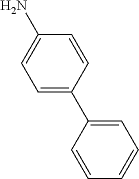 [SMILES: Nc1ccc(-c2ccccc2)cc1] | 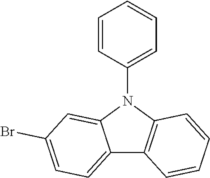 [SMILES: Brc1ccc2c3ccccc3n(-c3ccccc3)c2c1] | 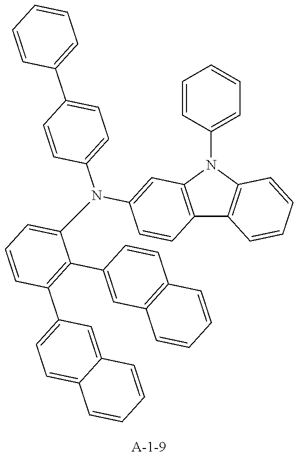 [SMILES: c1ccc(-c2ccc(N(c3ccc4c5ccccc5n(-c5ccccc5)c4c3)c3cccc(-c4ccc5ccccc5c4)c3-c3ccc4ccccc4c3)cc2)cc1] | 37 | 739.3 |
| 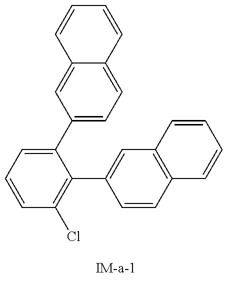 [SMILES: Clc1cccc(-c2ccc3ccccc3c2)c1-c1ccc2ccccc2c1] | 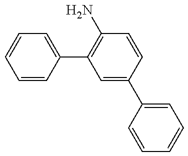 [SMILES: Nc1ccc(-c2ccccc2)cc1-c1ccccc1] | 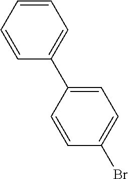 [SMILES: Brc1ccc(-c2ccccc2)cc1] | 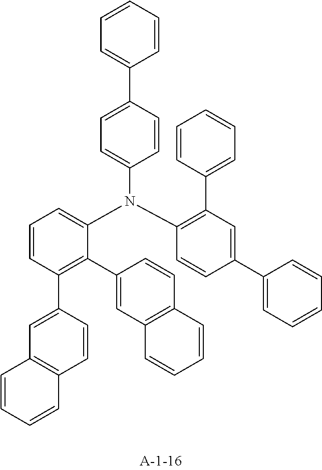 [SMILES: c1ccc(-c2ccc(N(c3ccc(-c4ccccc4)cc3-c3ccccc3)c3cccc(-c4ccc5ccccc5c4)c3-c3ccc4ccccc4c3)cc2)cc1] | 40 | 726.3 |
| 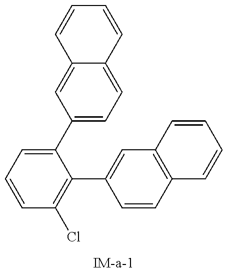 [SMILES: Clc1cccc(-c2ccc3ccccc3c2)c1-c1ccc2ccccc2c1] | 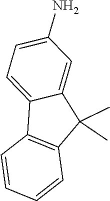 [SMILES: CC1(C)c2ccccc2-c2ccc(N)cc21] | 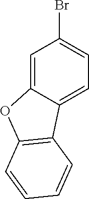 [SMILES: Brc1ccc2c(c1)oc1ccccc12] | 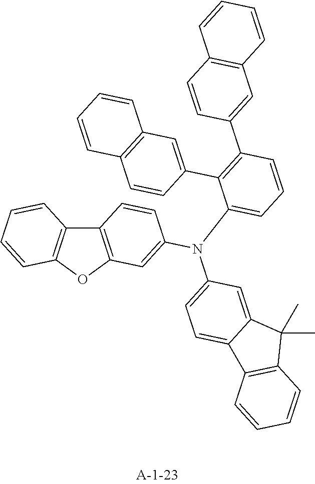 [SMILES: CC1(C)c2ccccc2-c2ccc(N(c3ccc4c(c3)oc3ccccc34)c3cccc(-c4ccc5ccccc5c4)c3-c3ccc4ccccc4c3)cc21] | 42 | 704.3 |
| 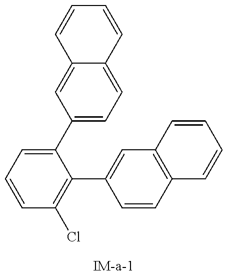 [SMILES: Clc1cccc(-c2ccc3ccccc3c2)c1-c1ccc2ccccc2c1] | 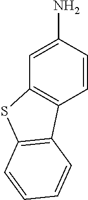 [SMILES: Nc1ccc2c(c1)sc1ccccc12] | 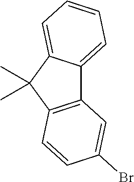 [SMILES: CC1(C)c2ccccc2-c2cc(Br)ccc21] | 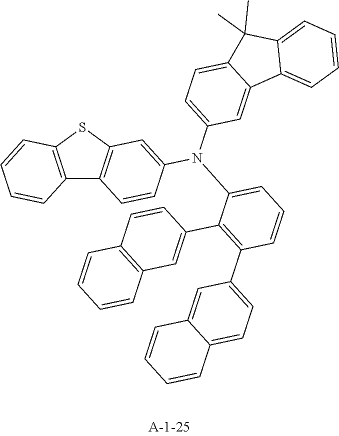 [SMILES: CC1(C)c2ccccc2-c2cc(N(c3ccc4c(c3)sc3ccccc34)c3cccc(-c4ccc5ccccc5c4)c3-c3ccc4ccccc4c3)ccc21] | 39 | 720.3 |
| 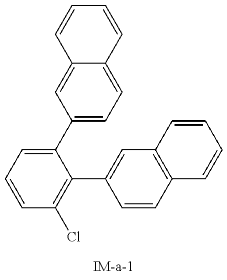 [SMILES: Clc1cccc(-c2ccc3ccccc3c2)c1-c1ccc2ccccc2c1] | 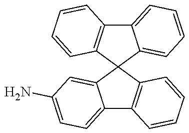 [SMILES: Nc1ccc2c(c1)C1(c3ccccc3-c3ccccc31)c1ccccc1-2] | 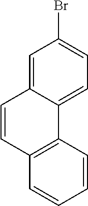 [SMILES: Brc1ccc2c(ccc3ccccc32)c1] | 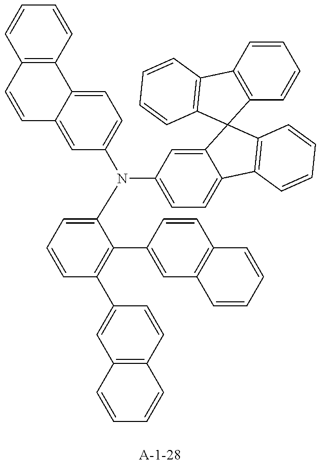 | 34 | 836.3 |
| 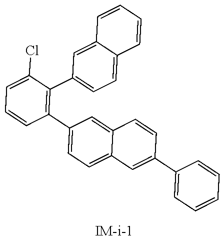 [SMILES: Clc1cccc(-c2ccc3cc(-c4ccccc4)ccc3c2)c1-c1ccc2ccccc2c1] | 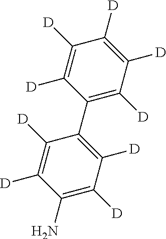 [SMILES: [2H]c1c([2H])c([2H])c(-c2c([2H])c([2H])c(N)c([2H])c2[2H])c([2H])c1[2H]] | 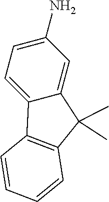 [SMILES: CC1(C)c2ccccc2-c2ccc(N)cc21] | 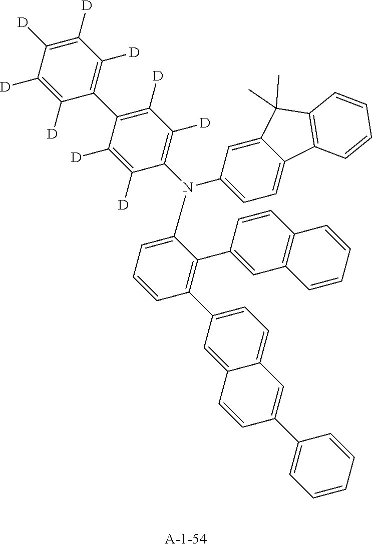 [SMILES: [2H]c1c([2H])c([2H])c(-c2c([2H])c([2H])c(N(c3ccc4c(c3)C(C)(C)c3ccccc3-4)c3cccc(-c4ccc5cc(-c6ccccc6)ccc5c4)c3-c3ccc4ccccc4c3)c([2H])c2[2H])c([2H])c1[2H]] | 29 | 775.4 |
| 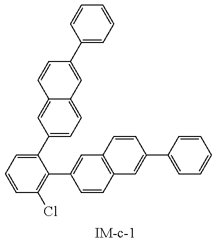 [SMILES: Clc1cccc(-c2ccc3cc(-c4ccccc4)ccc3c2)c1-c1ccc2cc(-c3ccccc3)ccc2c1] | 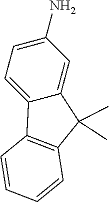 [SMILES: CC1(C)c2ccccc2-c2ccc(N)cc21] | 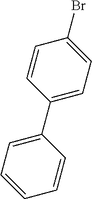 [SMILES: Brc1ccc(-c2ccccc2)cc1] | 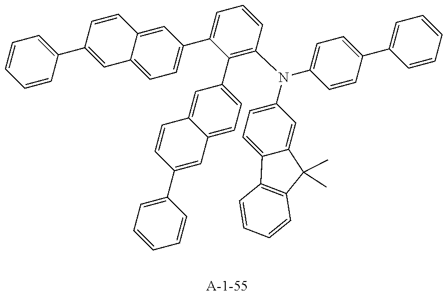 [SMILES: CC1(C)c2ccccc2-c2ccc(N(c3ccc(-c4ccccc4)cc3)c3cccc(-c4ccc5cc(-c6ccccc6)ccc5c4)c3-c3ccc4cc(-c5ccccc5)ccc4c3)cc21] | 32 | 842.4 |
| 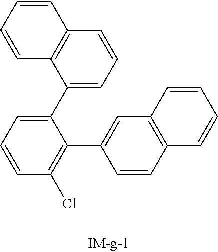 [SMILES: Clc1cccc(-c2cccc3ccccc23)c1-c1ccc2ccccc2c1] | 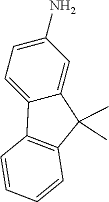 [SMILES: CC1(C)c2ccccc2-c2ccc(N)cc21] | 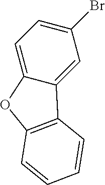 [SMILES: Brc1ccc2oc3ccccc3c2c1] | 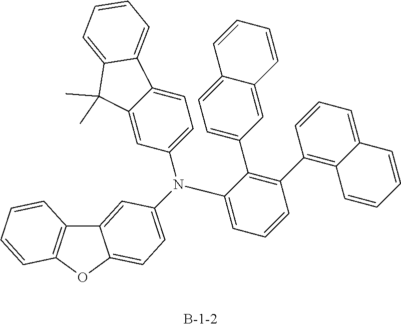 [SMILES: CC1(C)c2ccccc2-c2ccc(N(c3ccc4oc5ccccc5c4c3)c3cccc(-c4cccc5ccccc45)c3-c3ccc4ccccc4c3)cc21] | 39 | 704.3 |
| 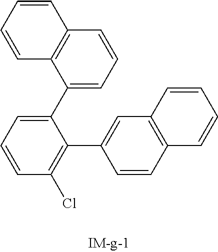 [SMILES: Clc1cccc(-c2cccc3ccccc23)c1-c1ccc2ccccc2c1] | 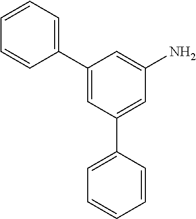 [SMILES: Nc1cc(-c2ccccc2)cc(-c2ccccc2)c1] | 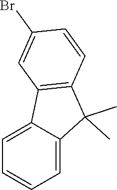 [SMILES: CC1(C)c2ccccc2-c2cc(Br)ccc21] | 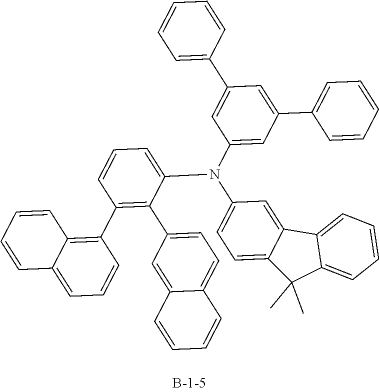 [SMILES: CC1(C)c2ccccc2-c2cc(N(c3cc(-c4ccccc4)cc(-c4ccccc4)c3)c3cccc(-c4cccc5ccccc45)c3-c3ccc4ccccc4c3)ccc21] | 31 | 766.3 |
| 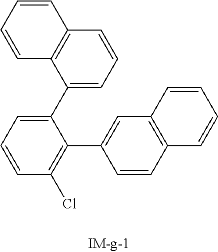 [SMILES: Clc1cccc(-c2cccc3ccccc23)c1-c1ccc2ccccc2c1] | 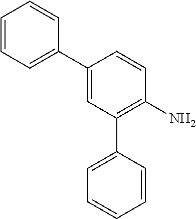 [SMILES: Nc1ccc(-c2ccccc2)cc1-c1ccccc1] | 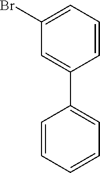 [SMILES: Brc1cccc(-c2ccccc2)c1] | 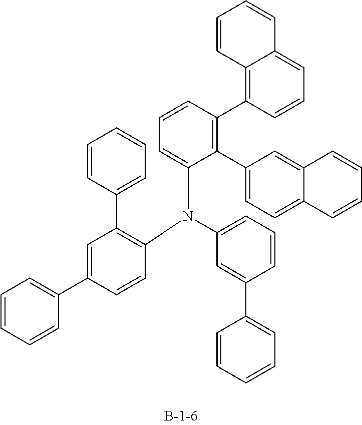 [SMILES: c1ccc(-c2cccc(N(c3ccc(-c4ccccc4)cc3-c3ccccc3)c3cccc(-c4cccc5ccccc45)c3-c3ccc4ccccc4c3)c2)cc1] | 38 | 726.3 |
| 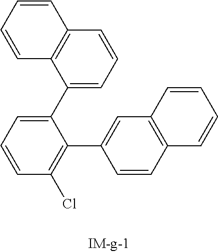 [SMILES: Clc1cccc(-c2cccc3ccccc23)c1-c1ccc2ccccc2c1] | 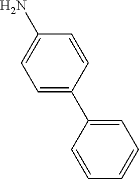 [SMILES: Nc1ccc(-c2ccccc2)cc1] | 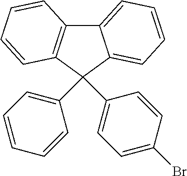 [SMILES: Brc1ccc(C2(c3ccccc3)c3ccccc3-c3ccccc32)cc1] | 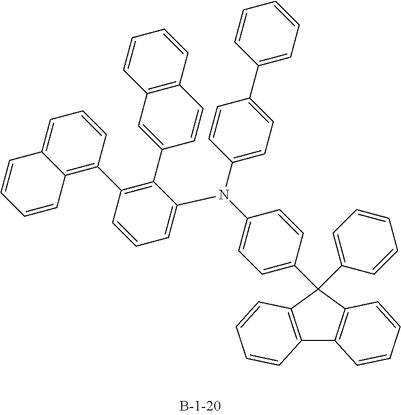 [SMILES: c1ccc(-c2ccc(N(c3ccc(C4(c5ccccc5)c5ccccc5-c5ccccc54)cc3)c3cccc(-c4cccc5ccccc45)c3-c3ccc4ccccc4c3)cc2)cc1] | 32 | 814.3 |
| 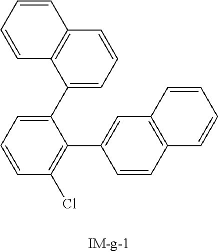 [SMILES: Clc1cccc(-c2cccc3ccccc23)c1-c1ccc2ccccc2c1] | 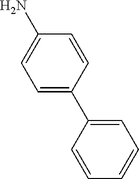 [SMILES: Nc1ccc(-c2ccccc2)cc1] | 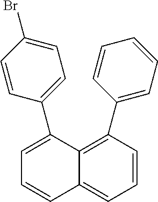 [SMILES: Brc1ccc(-c2cccc3cccc(-c4ccccc4)c23)cc1] | 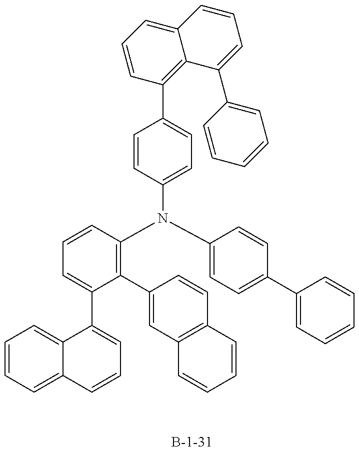 [SMILES: c1ccc(-c2ccc(N(c3ccc(-c4cccc5cccc(-c6ccccc6)c45)cc3)c3cccc(-c4cccc5ccccc45)c3-c3ccc4ccccc4c3)cc2)cc1] | 34 | 776.3 |
| 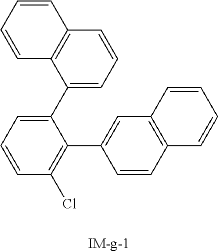 [SMILES: Clc1cccc(-c2cccc3ccccc23)c1-c1ccc2ccccc2c1] | 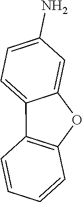 [SMILES: Nc1ccc2c(c1)oc1ccccc12] | 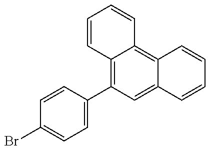 [SMILES: Brc1ccc(-c2cc3ccccc3c3ccccc23)cc1] | 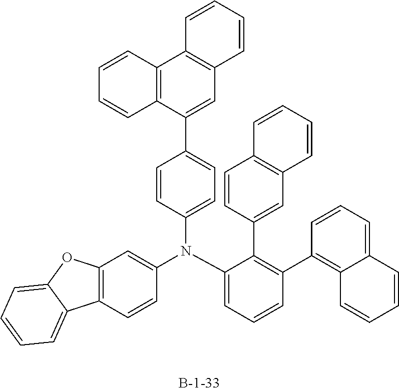 [SMILES: c1cc(-c2cccc3ccccc23)c(-c2ccc3ccccc3c2)c(N(c2ccc(-c3cc4ccccc4c4ccccc34)cc2)c2ccc3c(c2)oc2ccccc23)c1] | 37 | 764.3 |
| 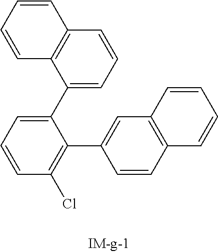 [SMILES: Clc1cccc(-c2cccc3ccccc23)c1-c1ccc2ccccc2c1] | 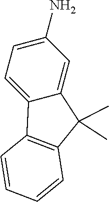 [SMILES: CC1(C)c2ccccc2-c2ccc(N)cc21] | 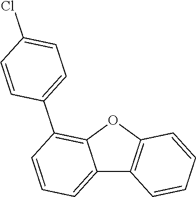 [SMILES: Clc1ccc(-c2cccc3c2oc2ccccc23)cc1] | 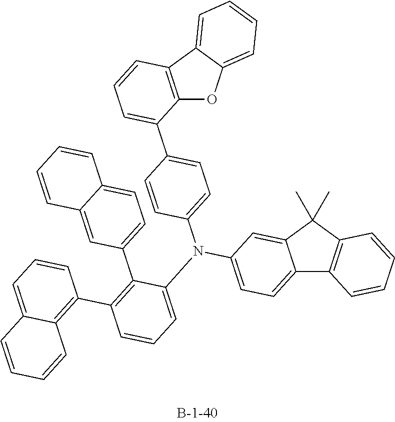 [SMILES: CC1(C)c2ccccc2-c2ccc(N(c3ccc(-c4cccc5c4oc4ccccc45)cc3)c3cccc(-c4cccc5ccccc45)c3-c3ccc4ccccc4c3)cc21] | 29 | 780.3 |
| 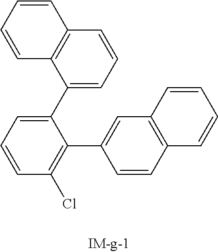 [SMILES: Clc1cccc(-c2cccc3ccccc23)c1-c1ccc2ccccc2c1] | 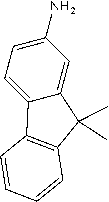 [SMILES: CC1(C)c2ccccc2-c2ccc(N)cc21] | 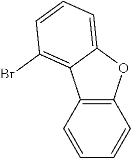 [SMILES: Brc1cccc2oc3ccccc3c12] | 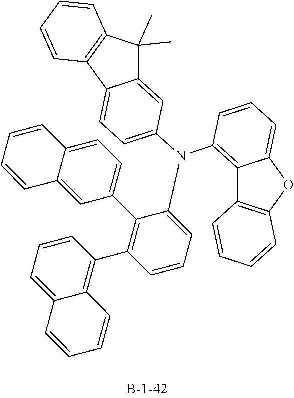 [SMILES: CC1(C)c2ccccc2-c2ccc(N(c3cccc(-c4cccc5ccccc45)c3-c3ccc4ccccc4c3)c3cccc4oc5ccccc5c34)cc21] | 42 | 704.3 |
| 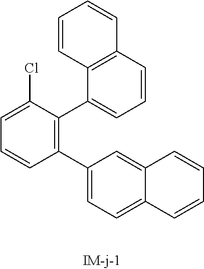 [SMILES: Clc1cccc(-c2ccc3ccccc3c2)c1-c1cccc2ccccc12] | 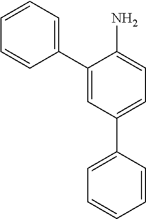 [SMILES: Nc1ccc(-c2ccccc2)cc1-c1ccccc1] | 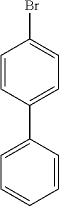 [SMILES: Brc1ccc(-c2ccccc2)cc1] | 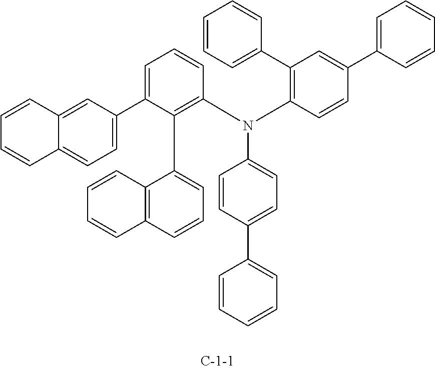 [SMILES: c1ccc(-c2ccc(N(c3ccc(-c4ccccc4)cc3-c3ccccc3)c3cccc(-c4ccc5ccccc5c4)c3-c3cccc4ccccc34)cc2)cc1] | 40 | 726.3 |
| 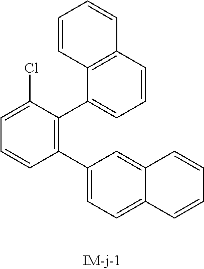 [SMILES: Clc1cccc(-c2ccc3ccccc3c2)c1-c1cccc2ccccc12] | 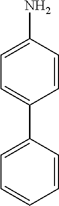 [SMILES: Nc1ccc(-c2ccccc2)cc1] | 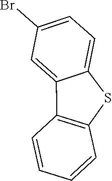 [SMILES: Brc1ccc2sc3ccccc3c2c1] | 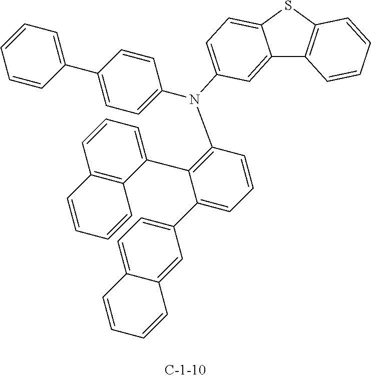 [SMILES: c1ccc(-c2ccc(N(c3ccc4sc5ccccc5c4c3)c3cccc(-c4ccc5ccccc5c4)c3-c3cccc4ccccc34)cc2)cc1] | 32 | 680.2 |
| 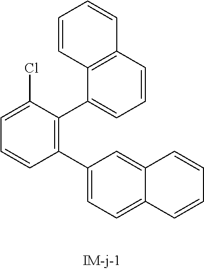 [SMILES: Clc1cccc(-c2ccc3ccccc3c2)c1-c1cccc2ccccc12] | 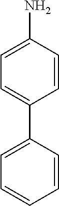 [SMILES: Nc1ccc(-c2ccccc2)cc1] | 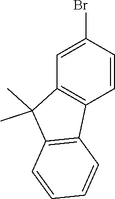 [SMILES: CC1(C)c2ccccc2-c2ccc(Br)cc21] | 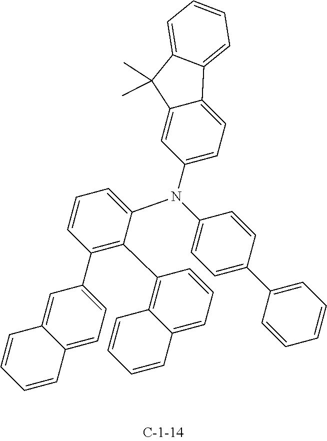 [SMILES: CC1(C)c2ccccc2-c2ccc(N(c3ccc(-c4ccccc4)cc3)c3cccc(-c4ccc5ccccc5c4)c3-c3cccc4ccccc34)cc21] | 42 | 690.3 |
| 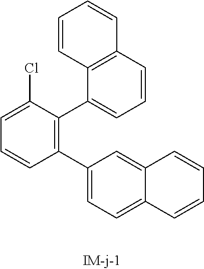 [SMILES: Clc1cccc(-c2ccc3ccccc3c2)c1-c1cccc2ccccc12] | 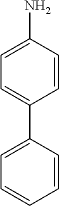 [SMILES: Nc1ccc(-c2ccccc2)cc1] | 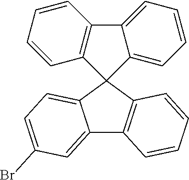 [SMILES: Brc1ccc2c(c1)-c1ccccc1C21c2ccccc2-c2ccccc21] | 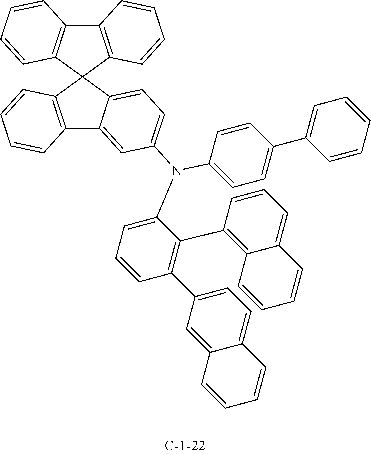 [SMILES: c1ccc(-c2ccc(N(c3ccc4c(c3)-c3ccccc3C43c4ccccc4-c4ccccc43)c3cccc(-c4ccc5ccccc5c4)c3-c3cccc4ccccc34)cc2)cc1] | 29 | 812.3 |
| 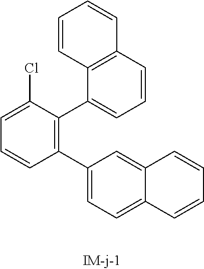 [SMILES: Clc1cccc(-c2ccc3ccccc3c2)c1-c1cccc2ccccc12] | 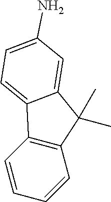 [SMILES: CC1(C)c2ccccc2-c2ccc(N)cc21] | 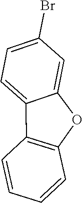 [SMILES: Brc1ccc2c(c1)oc1ccccc12] | 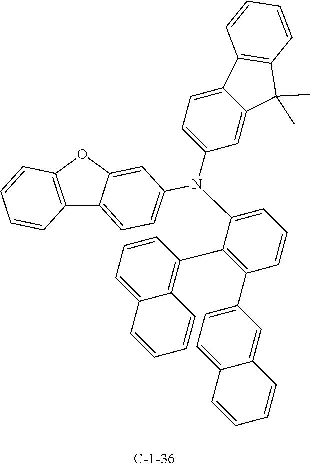 [SMILES: CC1(C)c2ccccc2-c2ccc(N(c3ccc4c(c3)oc3ccccc34)c3cccc(-c4ccc5ccccc5c4)c3-c3cccc4ccccc34)cc21] | 33 | 704.3 |
| 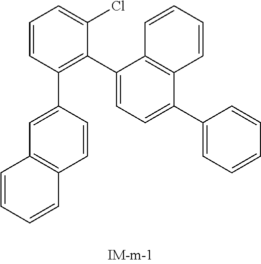 [SMILES: Clc1cccc(-c2ccc3ccccc3c2)c1-c1ccc(-c2ccccc2)c2ccccc12] | 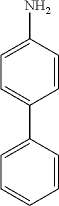 [SMILES: Nc1ccc(-c2ccccc2)cc1] | 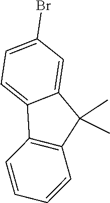 [SMILES: CC1(C)c2ccccc2-c2ccc(Br)cc21] | 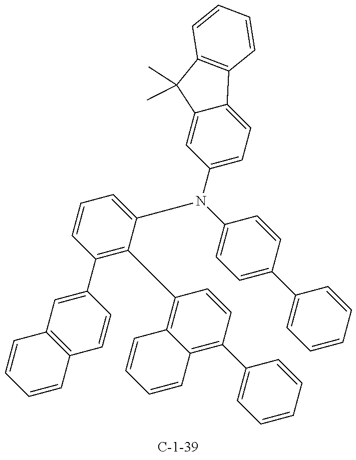 [SMILES: CC1(C)c2ccccc2-c2ccc(N(c3ccc(-c4ccccc4)cc3)c3cccc(-c4ccc5ccccc5c4)c3-c3ccc(-c4ccccc4)c4ccccc34)cc21] | 40 | 766.3 |
| 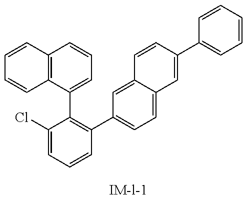 [SMILES: Clc1cccc(-c2ccc3cc(-c4ccccc4)ccc3c2)c1-c1cccc2ccccc12] | 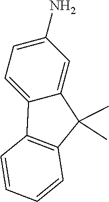 [SMILES: CC1(C)c2ccccc2-c2ccc(N)cc21] | 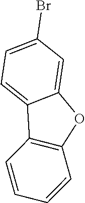 [SMILES: Brc1ccc2c(c1)oc1ccccc12] | 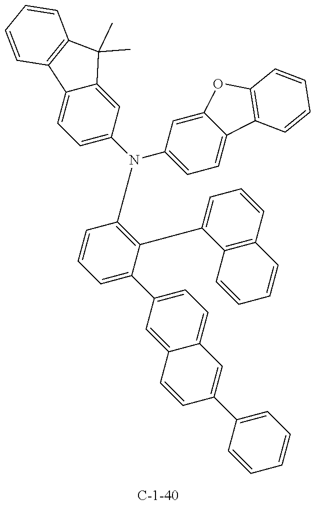 [SMILES: CC1(C)c2ccccc2-c2ccc(N(c3ccc4c(c3)oc3ccccc34)c3cccc(-c4ccc5cc(-c6ccccc6)ccc5c4)c3-c3cccc4ccccc34)cc21] | 31 | 780.3 |
| 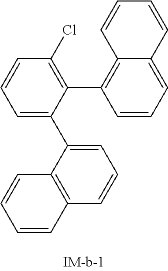 [SMILES: Clc1cccc(-c2cccc3ccccc23)c1-c1cccc2ccccc12] | 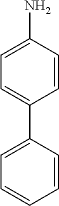 [SMILES: Nc1ccc(-c2ccccc2)cc1] | 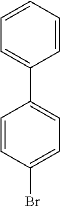 [SMILES: Brc1ccc(-c2ccccc2)cc1] | 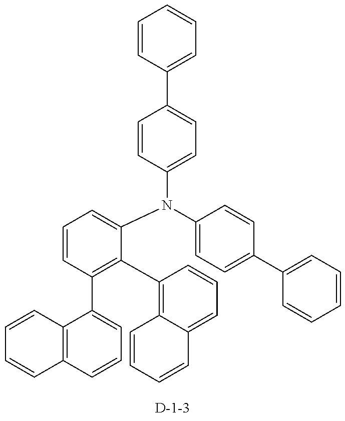 [SMILES: c1ccc(-c2ccc(N(c3ccc(-c4ccccc4)cc3)c3cccc(-c4cccc5ccccc45)c3-c3cccc4ccccc34)cc2)cc1] | 42 | 650.3 |
| 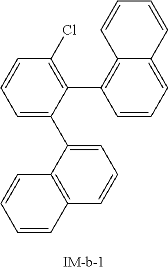 [SMILES: Clc1cccc(-c2cccc3ccccc23)c1-c1cccc2ccccc12] | 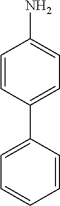 [SMILES: Nc1ccc(-c2ccccc2)cc1] | 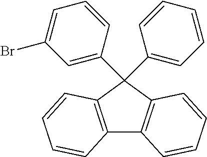 [SMILES: Brc1cccc(C2(c3ccccc3)c3ccccc3-c3ccccc32)c1] | 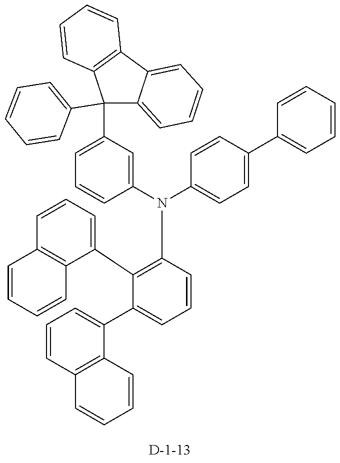 [SMILES: c1ccc(-c2ccc(N(c3cccc(C4(c5ccccc5)c5ccccc5-c5ccccc54)c3)c3cccc(-c4cccc5ccccc45)c3-c3cccc4ccccc34)cc2)cc1] | 35 | 814.3 |
| 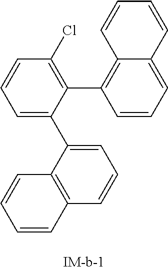 [SMILES: Clc1cccc(-c2cccc3ccccc23)c1-c1cccc2ccccc12] | 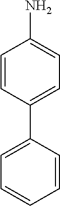 [SMILES: Nc1ccc(-c2ccccc2)cc1] | 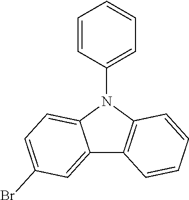 [SMILES: Brc1ccc2c(c1)c1ccccc1n2-c1ccccc1] | 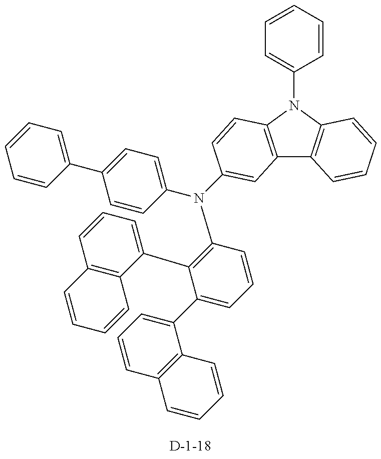 [SMILES: c1ccc(-c2ccc(N(c3ccc4c(c3)c3ccccc3n4-c3ccccc3)c3cccc(-c4cccc5ccccc45)c3-c3cccc4ccccc34)cc2)cc1] | 33 | 739.3 |
| 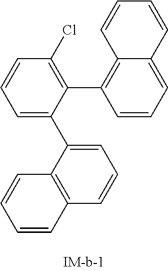 [SMILES: Clc1cccc(-c2cccc3ccccc23)c1-c1cccc2ccccc12] | 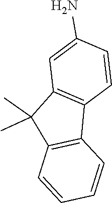 [SMILES: CC1(C)c2ccccc2-c2ccc(N)cc21] | 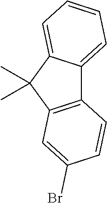 [SMILES: CC1(C)c2ccccc2-c2ccc(Br)cc21] | 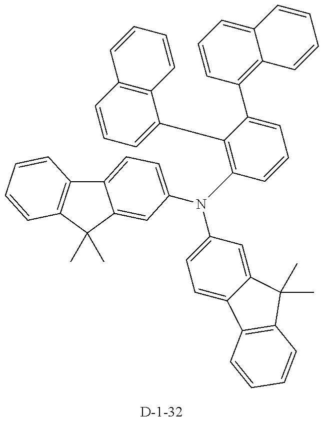 [SMILES: CC1(C)c2ccccc2-c2ccc(N(c3ccc4c(c3)C(C)(C)c3ccccc3-4)c3cccc(-c4cccc5ccccc45)c3-c3cccc4ccccc34)cc21] | 40 | 730.3 |
| 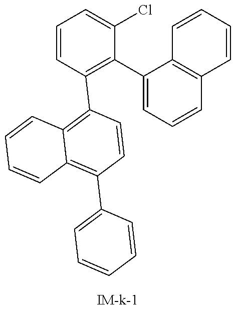 [SMILES: Clc1cccc(-c2ccc(-c3ccccc3)c3ccccc23)c1-c1cccc2ccccc12] | 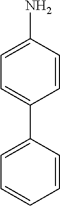 [SMILES: Nc1ccc(-c2ccccc2)cc1] | 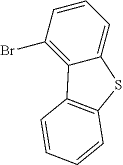 [SMILES: Brc1cccc2sc3ccccc3c12] | 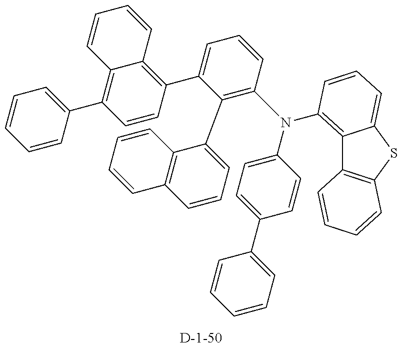 [SMILES: c1ccc(-c2ccc(N(c3cccc(-c4ccc(-c5ccccc5)c5ccccc45)c3-c3cccc4ccccc34)c3cccc4sc5ccccc5c34)cc2)cc1] | 33 | 756.3 |
| 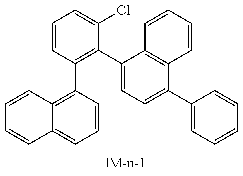 [SMILES: Clc1cccc(-c2cccc3ccccc23)c1-c1ccc(-c2ccccc2)c2ccccc12] | 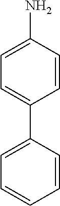 [SMILES: Nc1ccc(-c2ccccc2)cc1] | 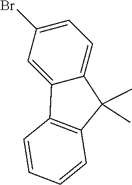 [SMILES: CC1(C)c2ccccc2-c2cc(Br)ccc21] | 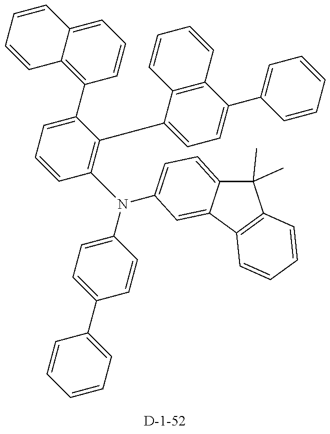 [SMILES: CC1(C)c2ccccc2-c2cc(N(c3ccc(-c4ccccc4)cc3)c3cccc(-c4cccc5ccccc45)c3-c3ccc(-c4ccccc4)c4ccccc34)ccc21] | 34 | 766.3 |
| 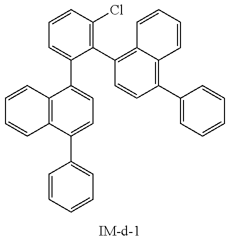 [SMILES: Clc1cccc(-c2ccc(-c3ccccc3)c3ccccc23)c1-c1ccc(-c2ccccc2)c2ccccc12] | 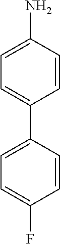 [SMILES: Nc1ccc(-c2ccc(F)cc2)cc1] | 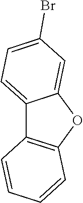 [SMILES: Brc1ccc2c(c1)oc1ccccc12] | 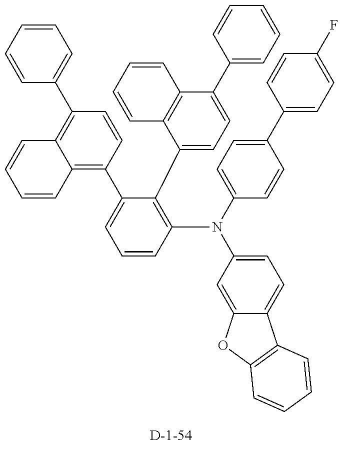 [SMILES: Fc1ccc(-c2ccc(N(c3ccc4c(c3)oc3ccccc34)c3cccc(-c4ccc(-c5ccccc5)c5ccccc45)c3-c3ccc(-c4ccccc4)c4ccccc34)cc2)cc1] | 32 | 834.3 |
| 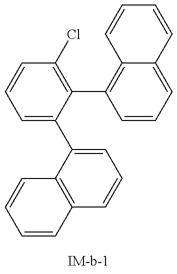 [SMILES: Clc1cccc(-c2cccc3ccccc23)c1-c1cccc2ccccc12] | 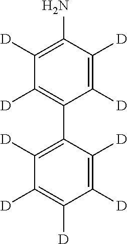 [SMILES: [2H]c1c([2H])c([2H])c(-c2c([2H])c([2H])c(N)c([2H])c2[2H])c([2H])c1[2H]] | 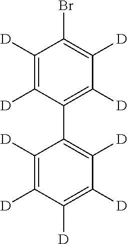 [SMILES: [2H]c1c([2H])c([2H])c(-c2c([2H])c([2H])c(Br)c([2H])c2[2H])c([2H])c1[2H]] | 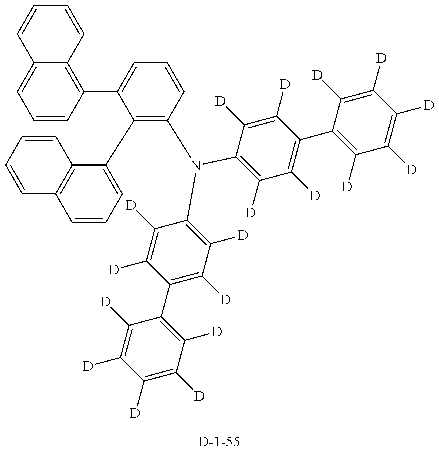 [SMILES: [2H]c1c([2H])c([2H])c(-c2c([2H])c([2H])c(N(c3cccc(-c4cccc5ccccc45)c3-c3cccc4ccccc34)c3c([2H])c([2H])c(-c4c([2H])c([2H])c([2H])c([2H])c4[2H])c([2H])c3[2H])c([2H])c2[2H])c([2H])c1[2H]] | 41 | 668.4 |
| 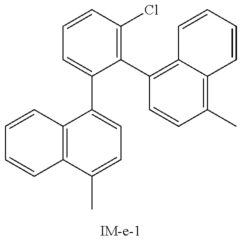 [SMILES: Cc1ccc(-c2cccc(Cl)c2-c2ccc(C)c3ccccc23)c2ccccc12] | 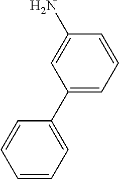 [SMILES: Nc1cccc(-c2ccccc2)c1] | 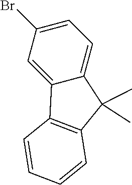 [SMILES: CC1(C)c2ccccc2-c2cc(Br)ccc21] | 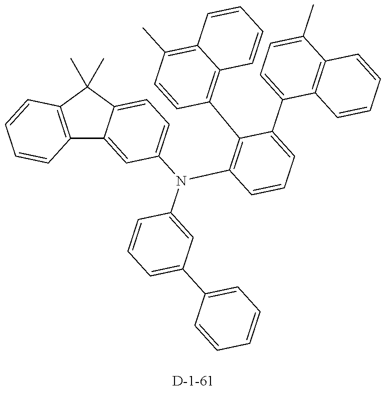 [SMILES: Cc1ccc(-c2cccc(N(c3cccc(-c4ccccc4)c3)c3ccc4c(c3)-c3ccccc3C4(C)C)c2-c2ccc(C)c3ccccc23)c2ccccc12] | 30 | 718.3 |
| 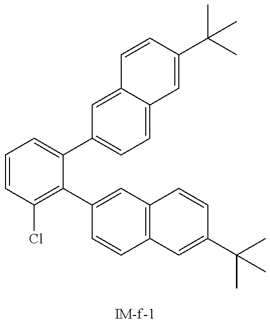 [SMILES: CC(C)(C)c1ccc2cc(-c3cccc(Cl)c3-c3ccc4cc(C(C)(C)C)ccc4c3)ccc2c1] | 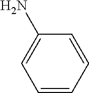 | 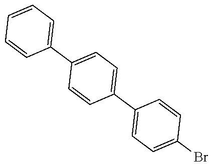 [SMILES: Brc1ccc(-c2ccc(-c3ccccc3)cc2)cc1] | 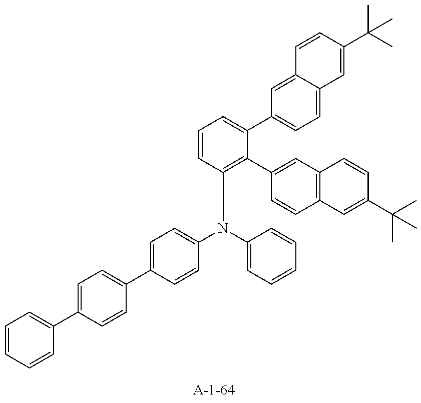 [SMILES: CC(C)(C)c1ccc2cc(-c3cccc(N(c4ccccc4)c4ccc(-c5ccc(-c6ccccc6)cc5)cc4)c3-c3ccc4cc(C(C)(C)C)ccc4c3)ccc2c1] | 31 | 762.4 |
| 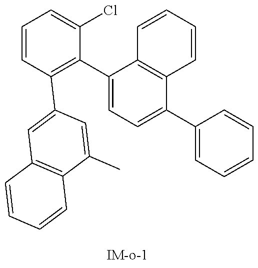 [SMILES: Cc1cc(-c2cccc(Cl)c2-c2ccc(-c3ccccc3)c3ccccc23)cc2ccccc12] | 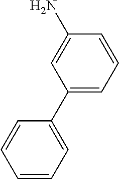 [SMILES: Nc1cccc(-c2ccccc2)c1] | 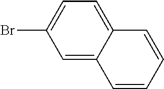 [SMILES: Brc1ccc2ccccc2c1] | 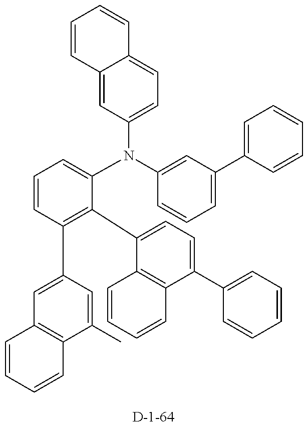 [SMILES: Cc1cc(-c2cccc(N(c3cccc(-c4ccccc4)c3)c3ccc4ccccc4c3)c2-c2ccc(-c3ccccc3)c3ccccc23)cc2ccccc12] | 32 | 714.3 |
| 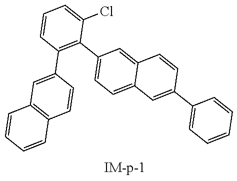 [SMILES: Clc1cccc(-c2ccc3ccccc3c2)c1-c1ccc2cc(-c3ccccc3)ccc2c1] | 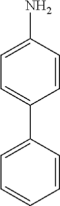 [SMILES: Nc1ccc(-c2ccccc2)cc1] | 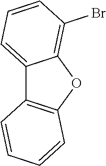 [SMILES: Brc1cccc2c1oc1ccccc12] | 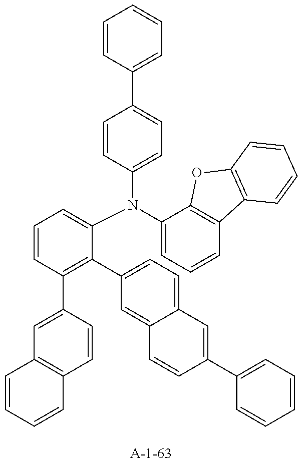 [SMILES: c1ccc(-c2ccc(N(c3cccc(-c4ccc5ccccc5c4)c3-c3ccc4cc(-c5ccccc5)ccc4c3)c3cccc4c3oc3ccccc34)cc2)cc1] | 36 | 740.3 |
| 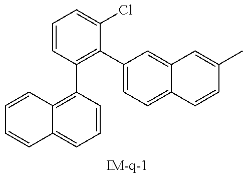 [SMILES: Cc1ccc2ccc(-c3c(Cl)cccc3-c3cccc4ccccc34)cc2c1] | 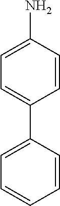 [SMILES: Nc1ccc(-c2ccccc2)cc1] | 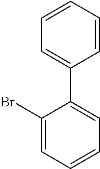 [SMILES: Brc1ccccc1-c1ccccc1] | 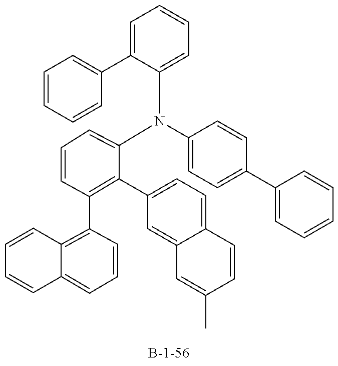 [SMILES: Cc1ccc2ccc(-c3c(-c4cccc5ccccc45)cccc3N(c3ccc(-c4ccccc4)cc3)c3ccccc3-c3ccccc3)cc2c1] | 39 | 664.3 |
| 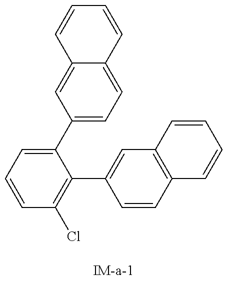 [SMILES: Clc1cccc(-c2ccc3ccccc3c2)c1-c1ccc2ccccc2c1] | 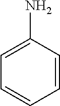 | 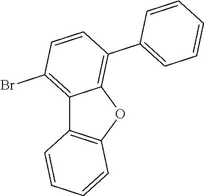 [SMILES: Brc1ccc(-c2ccccc2)c2oc3ccccc3c12] | 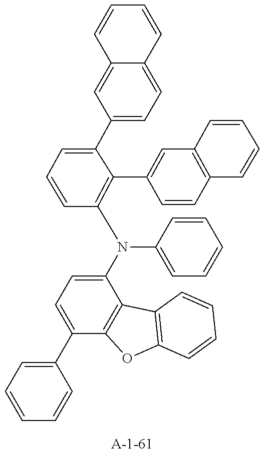 [SMILES: c1ccc(-c2ccc(N(c3ccccc3)c3cccc(-c4ccc5ccccc5c4)c3-c3ccc4ccccc4c3)c3c2oc2ccccc23)cc1] | 37 | 664.3 |
| 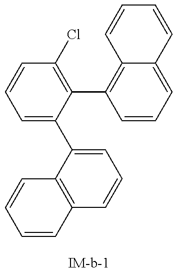 [SMILES: Clc1cccc(-c2cccc3ccccc23)c1-c1cccc2ccccc12] | 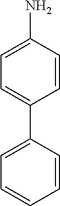 [SMILES: Nc1ccc(-c2ccccc2)cc1] | 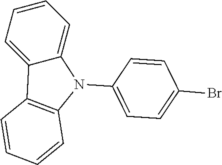 [SMILES: Brc1ccc(-n2c3ccccc3c3ccccc32)cc1] | 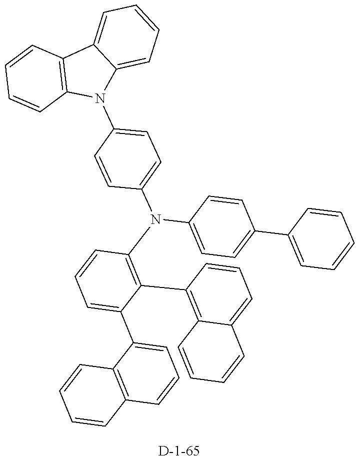 [SMILES: c1ccc(-c2ccc(N(c3ccc(-n4c5ccccc5c5ccccc54)cc3)c3cccc(-c4cccc5ccccc45)c3-c3cccc4ccccc34)cc2)cc1] | 31 | 739.3 |
| 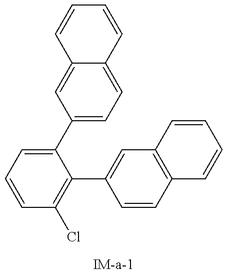 [SMILES: Clc1cccc(-c2ccc3ccccc3c2)c1-c1ccc2ccccc2c1] | 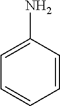 | 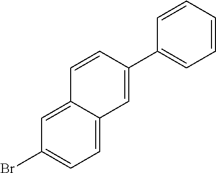 [SMILES: Brc1ccc2cc(-c3ccccc3)ccc2c1] | 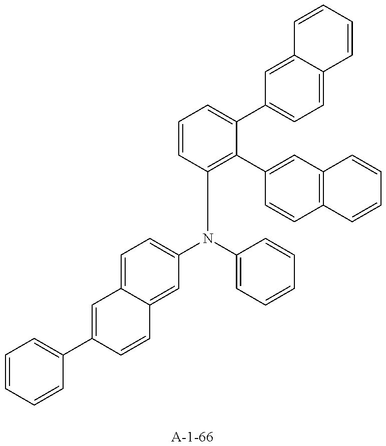 [SMILES: c1ccc(-c2ccc3cc(N(c4ccccc4)c4cccc(-c5ccc6ccccc6c5)c4-c4ccc5ccccc5c4)ccc3c2)cc1 ; "A-1-66"] | 32 | 624.3 |
| 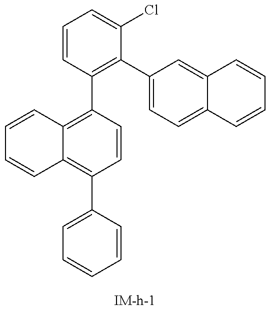 [SMILES: Clc1cccc(-c2ccc(-c3ccccc3)c3ccccc23)c1-c1ccc2ccccc2c1] | 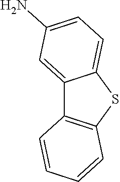 [SMILES: Nc1ccc2sc3ccccc3c2c1] | 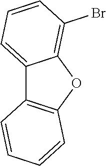 [SMILES: Brc1cccc2c1oc1ccccc12] | 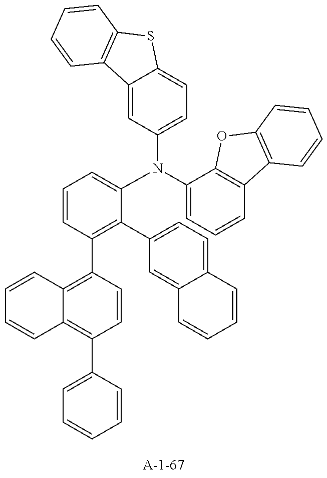 [SMILES: c1ccc(-c2ccc(-c3cccc(N(c4ccc5sc6ccccc6c5c4)c4cccc5c4oc4ccccc45)c3-c3ccc4ccccc4c3)c3ccccc23)cc1] | 29 | 770.2 |
| 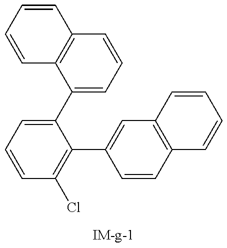 [SMILES: Clc1cccc(-c2cccc3ccccc23)c1-c1ccc2ccccc2c1] | 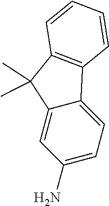 [SMILES: CC1(C)c2ccccc2-c2ccc(N)cc21] | 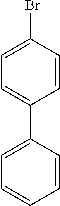 [SMILES: Brc1ccc(-c2ccccc2)cc1] | 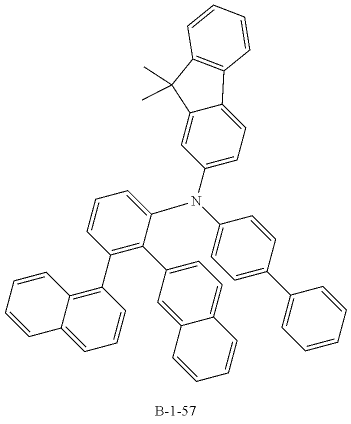 [SMILES: CC1(C)c2ccccc2-c2ccc(N(c3ccc(-c4ccccc4)cc3)c3cccc(-c4cccc5ccccc45)c3-c3ccc4ccccc4c3)cc21] | 40 | 690.3 |
| 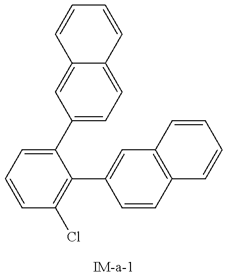 [SMILES: Clc1cccc(-c2ccc3ccccc3c2)c1-c1ccc2ccccc2c1] | 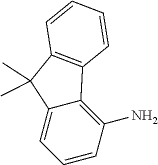 [SMILES: CC1(C)c2ccccc2-c2c(N)cccc21] | 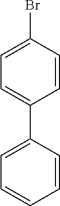 [SMILES: Brc1ccc(-c2ccccc2)cc1] | 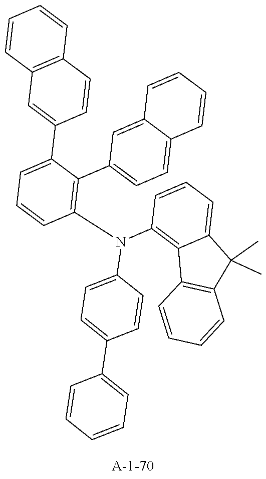 [SMILES: CC1(C)c2ccccc2-c2c(N(c3ccc(-c4ccccc4)cc3)c3cccc(-c4ccc5ccccc5c4)c3-c3ccc4ccccc4c3)cccc21] | 35 | 690.3 |

NMR data for some of the compounds are shown in Table 5.

TABLE 5

| Compound | NMR data ¹H-NMR(400 MHz, CD₂Cl₂) δ ppm |
|---|---|
| Compound | 7.75(s, 1H), 7.72-7.61(m, 2H), 7.59-7.54(m, 1H), |
| A-1-4 | 7.51-7.48(m, 6H), 7.30-7.46(m, 9H), 7.11-7.29(m, 10H), |
| | 6.98(d, 2H), 6.82(m, 2H), 1.18(s, 6H). |
| Compound | 7.81(d, 1H), 7.78(s, 1H), 7.76-7.62(m, 3H), 7.60-7.53(m, |
| A-1-23 | 1H), 7.49-7.41(m, 3H), 7.40-7.38(m, 3H), 7.37-7.31(m, |
| | 5H), 7.29-7.06(m, 11H), 6.96(s, 1H), 6.88(d, 1H), |
| | 6.78(m, 1H), 1.18(s, 6H). |

### Example of Fabricating and Evaluating of Organic Electroluminescent Devices

### Example 1: Fabrication of a Red Light-Emitting Organic Electroluminescent Device

An ITO/Ag/ITO substrate, with thicknesses of ITO/Ag/ITO being 70 Å, 1000 Å, and 100 Å, respectively, was cut to have dimensions of 40 mm×40 mm×0.7 mm, and then fabricated by a photoetching process, into an experimental substrate with patterns of a cathode, and of an insulation layer, firstly cleaning its surface using ultrapure water and isopropyl to clean the pollutants on the substrate, then followed by treatment of its surface using O₂:N₂ plasma.

M-MTDATA was deposited by vacuum evaporation on the experimental substrate (anode) to form a hole injection layer with a thickness of 100 Å, and then NPB (HT-1) was deposited by vacuum evaporation on the hole injection layer to form a first hole transport layer with a thickness of 1130 Å.

Compound A-1-3 was deposited by vacuum evaporation on the first hole transport layer to form a second hole transport layer with a thickness of 270 Å.

CBP and Ir(pig)₂(acac) were co-deposited by evaporation on the second hole transport layer at a film thickness ratio of 100:3 to form an organic emissive layer with a thickness of 350 Å.

DBimiBphen and LiQ were mixed at a weight ratio of 1:1 and co-deposited by evaporation on the organic emissive layer to form an electron transport layer with a thickness of 300 Å;

LiQ was deposited by evaporation on the electron transport layer to form an electron injection layer with a thickness of 12 Å. And then magnesium (Mg) and silver (Ag) were mixed at an evaporation rate ratio of 1:9 and deposited by vacuum evaporation on the electron injection layer to form a cathode with a thickness of 120 Å.

In the end, CP-1 was deposited by evaporation on the above cathode to form an organic capping layer (CPL) with a thickness of 650 Å, thereby completing the fabrication of the organic electroluminescent device.

### Examples 2 to 44

Organic electroluminescent devices were fabricated by the same method as used in Example 1, except that Compound A-1-3 in Example 1 was replaced with a compound shown in Table 6 (Line “HTL-2”) when a second hole transport layer was formed.

### Comparative Examples 1 to 3

Organic electroluminescent devices were fabricated by the same method as used in Example 1, except that Compound A-1-3 in Example 1 was replaced with a respective one of Compound A, Compound B, and Compound C when a second hole transport layer was formed.

Structures of the materials used in the Examples and the Comparative Examples during the fabrication of the organic electroluminescent devices are as follows:

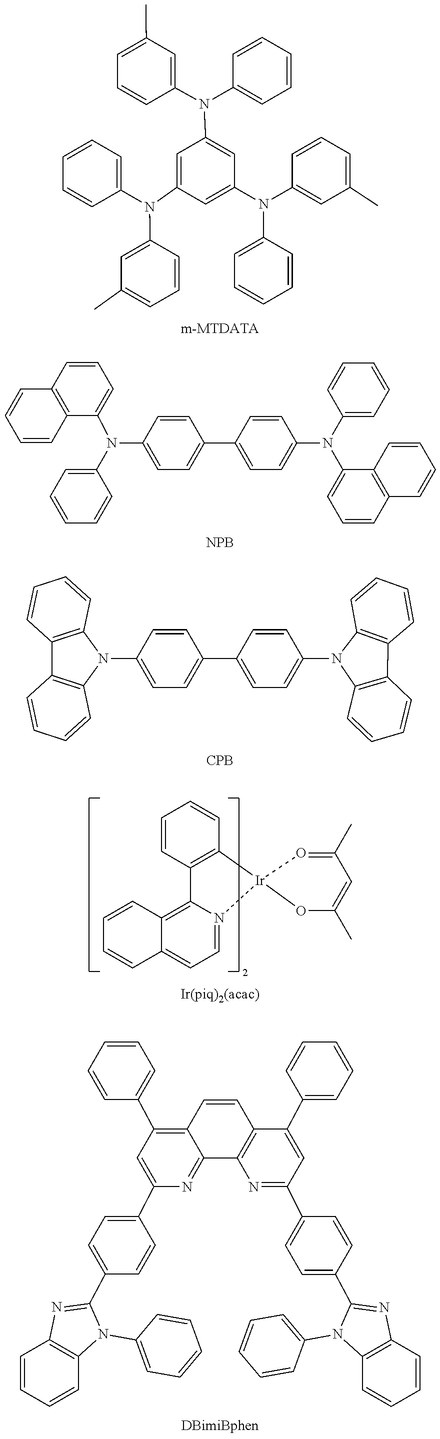

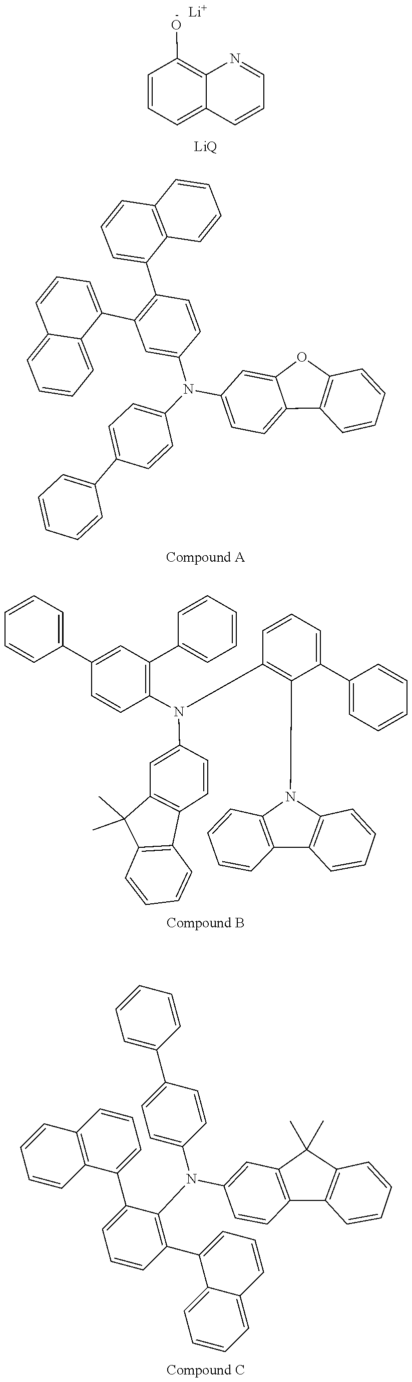

The organic electroluminescent devices fabricated in the Examples and Comparative Examples were tested for their performance, test results are shown in Table 6. Specifically, the IVL (driving voltage, current efficiency) characteristics of the devices were tested under the condition of 10 mA/cm², and the T95 lifetime was tested under the condition of 15 mA/cm².

TABLE 6

| | | Driving | Current | | |
|---|---|---|---|---|---|
| | | volt- | Effi- | Color | Life- |
| Example | | age | ciency | coordinate | time |
| No. | HTL-2 | (V) | (Cd/A) | CIE_{X}, CIEy | T95(h) |
| Example1 | A-1-3 | 3.86 | 38.9 | 0.680, 0.320 | 531 |
| Example 2 | A-1-4 | 3.97 | 38.5 | 0.680, 0.320 | 560 |
| Example 3 | A-1-8 | 3.99 | 39.1 | 0.680, 0.320 | 528 |
| Example 4 | A-1-9 | 3.85 | 39.3 | 0.680, 0.320 | 564 |
| Example 5 | A-1-16 | 3.87 | 38.1 | 0.680, 0.320 | 529 |
| Example 6 | A-1-23 | 3.88 | 38.6 | 0.680, 0.320 | 561 |
| Example 7 | A-1-25 | 3.86 | 38.8 | 0.680, 0.320 | 558 |
| Example 8 | A-1-28 | 3.81 | 37.8 | 0.680, 0.320 | 570 |
| Example 9 | A-1-54 | 3.89 | 36.9 | 0.680, 0.320 | 566 |
| Example 10 | A-1-55 | 3.92 | 38.1 | 0.680, 0.320 | 557 |
| Example 11 | B-1-2 | 3.93 | 39.3 | 0.680, 0.320 | 562 |
| Example 12 | B-1-5 | 3.91 | 38.6 | 0.680, 0.320 | 559 |
| Example 13 | B-1-6 | 3.88 | 38.1 | 0.680, 0.320 | 533 |
| Example 14 | B-1-20 | 3.92 | 38.3 | 0.680, 0.320 | 567 |
| Example 15 | B-1-31 | 3.93 | 38.8 | 0.680, 0.320 | 530 |
| Example 16 | B-1-33 | 3.91 | 39.3 | 0.680, 0.320 | 561 |
| Example 17 | B-1-40 | 3.92 | 37.6 | 0.680, 0.320 | 565 |
| Example 18 | B-1-42 | 3.89 | 38.9 | 0.680, 0.320 | 570 |
| Example 19 | C-1-1 | 3.94 | 38.2 | 0.680, 0.320 | 535 |
| Example 20 | C-1-10 | 3.89 | 37.8 | 0.680, 0.320 | 573 |
| Example 21 | C-1-14 | 3.84 | 37.5 | 0.680, 0.320 | 559 |
| Example 22 | C-1-22 | 3.86 | 38.6 | 0.680, 0.320 | 565 |
| Example 23 | C-1-36 | 3.93 | 37.1 | 0.680, 0.320 | 568 |
| Example 24 | C-1-39 | 3.91 | 38.2 | 0.680, 0.320 | 556 |
| Example 25 | C-1-40 | 3.90 | 39.1 | 0.680, 0.320 | 579 |
| Example 26 | D-1-3 | 3.95 | 37.3 | 0.680, 0.320 | 532 |
| Example 27 | D-1-13 | 3.87 | 38.8 | 0.680, 0.320 | 568 |
| Example 28 | D-1-18 | 3.89 | 37.5 | 0.680, 0.320 | 564 |
| Example 29 | D-1-32 | 3.92 | 38.9 | 0.680, 0.320 | 576 |
| Example 30 | D-1-50 | 3.96 | 37.1 | 0.680, 0.320 | 562 |
| Example 31 | D-1-52 | 3.89 | 38.9 | 0.680, 0.320 | 571 |
| Example 32 | D-1-54 | 3.96 | 38.3 | 0.680, 0.320 | 568 |
| Example 33 | D-1-55 | 3.84 | 37.9 | 0.680, 0.320 | 583 |
| Example 34 | D-1-61 | 3.90 | 38.5 | 0.680, 0.320 | 555 |
| Example 35 | A-1-64 | 3.95 | 38.1 | 0.680, 0.320 | 527 |
| Example 36 | D-1-64 | 3.86 | 38.5 | 0.680, 0.320 | 530 |
| Example 37 | A-1-63 | 3.82 | 37.0 | 0.680, 0.320 | 566 |
| Example 38 | B-1-56 | 3.94 | 37.3 | 0.680, 0.320 | 536 |
| Example 39 | A-1-61 | 3.90 | 37.4 | 0.680, 0.320 | 572 |
| Example 40 | D-1-65 | 3.92 | 37.0 | 0.680, 0.320 | 563 |
| Example 41 | A-1-65 | 3.81 | 39.6 | 0.680, 0.320 | 534 |
| Example 42 | A-1-67 | 3.97 | 37.8 | 0.680, 0.320 | 569 |
| Example 43 | B-1-57 | 3.85 | 38.4 | 0.680, 0.320 | 574 |
| Example 44 | A-1-70 | 3.94 | 38.2 | 0.680, 0.320 | 569 |
| Comparative | Compound A | 4.06 | 30.5 | 0.680, 0.320 | 426 |
| Example 1 | | | | | |
| Comparative | Compound B | 4.05 | 32.8 | 0.680, 0.320 | 431 |
| Example 2 | | | | | |
| Comparative | Compound C | 4.10 | 31.2 | 0.680, 0.320 | 449 |
| Example 3 | | | | | |

As can be seen from Table 6 above, compared with the organic electroluminescent devices fabricated in Comparative Examples 1 to 3, the luminescence efficiency of the organic electroluminescent devices fabricated in Examples 1 to 44, using the nitrogen-containing compound in the present application as the second hole transport layer materials, is increased by at least 12.5%, the service lifetime is increased by at least 17.3%, and the driving voltage is relatively low.

The preferred embodiments of the present application are described above, however, the present application is not limited to the specific details of the embodiments above, within the scope of the technical concept of the present application, a variety of simple variations of the technical schemes of the present application can be carried out, and these simple variations are within the scope of protection of the present application.

1. A nitrogen-containing compound, having a structure shown in Formula 1: 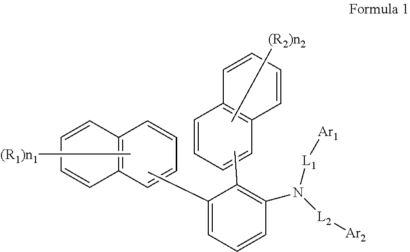
wherein Ar₁ is selected from a substituted or unsubstituted group W, and the unsubstituted group W is selected from the group consisting of the following groups: 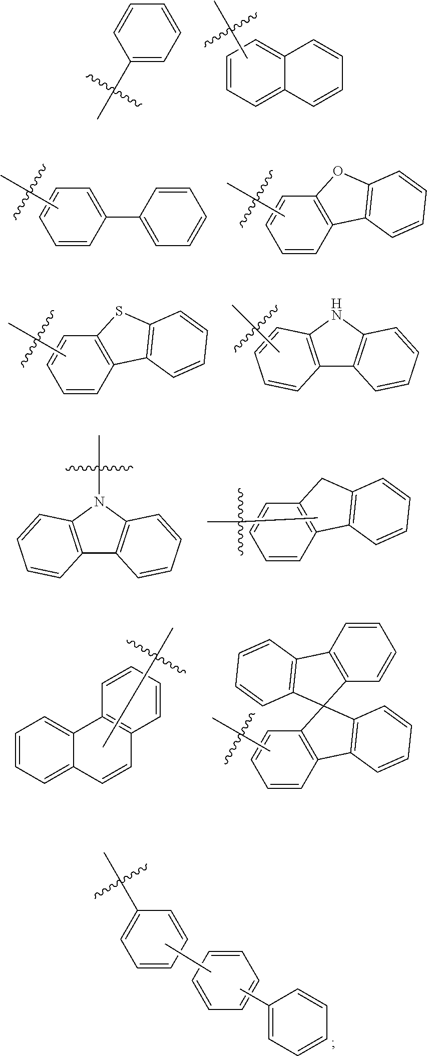
the substituent(s) of the substituted group W are each independently selected from deuterium, a fluorine, a cyano, a methyl, an ethyl, an isopropyl, a tert-butyl, or a phenyl, and when the number of the substituents is greater than 1, each substituent is identical or different;
Ar₂ is selected from a substituted or unsubstituted group W, and the unsubstituted group W is selected from the group consisting of the following groups: 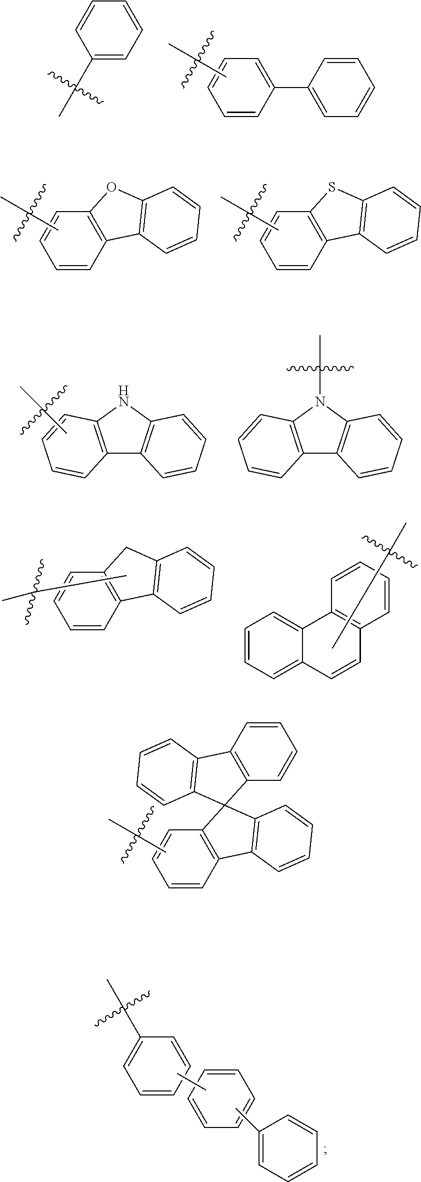
the substituent(s) of the substituted group W are each independently selected from deuterium, a fluorine, a cyano, a methyl, an ethyl, an isopropyl, a tert-butyl, or a phenyl, and when the number of the substituents is greater than 1, each substituent is identical or different;
L₁ is selected from a single bond, a substituted or unsubstituted phenylene, a substituted or unsubstituted naphthylene, a substituted or unsubstituted biphenylene;
L₂ is selected from a single bond, a substituted or unsubstituted phenylene, a substituted or unsubstituted biphenylene;
the substituent(s) of L₁ and L₂ are each independently selected from deuterium, a fluorine, a cyano, a methyl, an ethyl, an isopropyl, a tert-butyl, or a phenyl;
R₁ and R₂ are identical or different, and are each independently selected from deuterium a methyl, an ethyl, a n-propyl, an isopropyl, a tert-butyl, or a phenyl;
n₁ represents the number of R₁, n₁ is selected from 0, 1, 2, 3, 4, 5, 6 or 7, wherein n₁ is greater than 1, each R₁ is identical or different;
n₂ represents the number of R₂, n₂ is selected from 0, 1, 2, 3, 4, 5, 6 or 7, wherein when n₂ is greater than 1, each R₂ is identical or different.

2. The nitrogen-containing compound according to claim 1, wherein the structure of the nitrogen-containing compound is selected from Formula 1-1, Formula 1-2, Formula 2-1 or Formula 2-2:

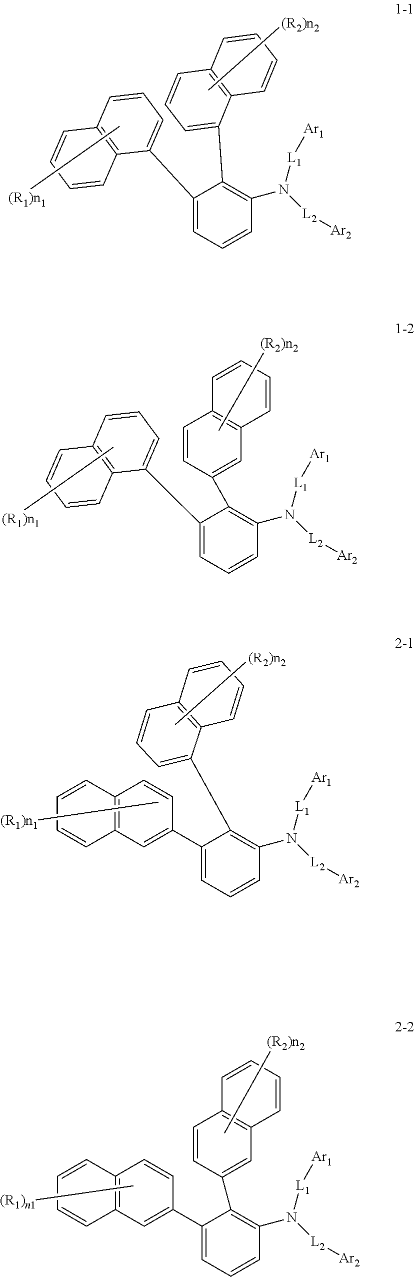

3-4. (canceled)

5. The nitrogen-containing compound according to claim 1, wherein L₁ is selected from a single bond and the group consisting of the following groups: 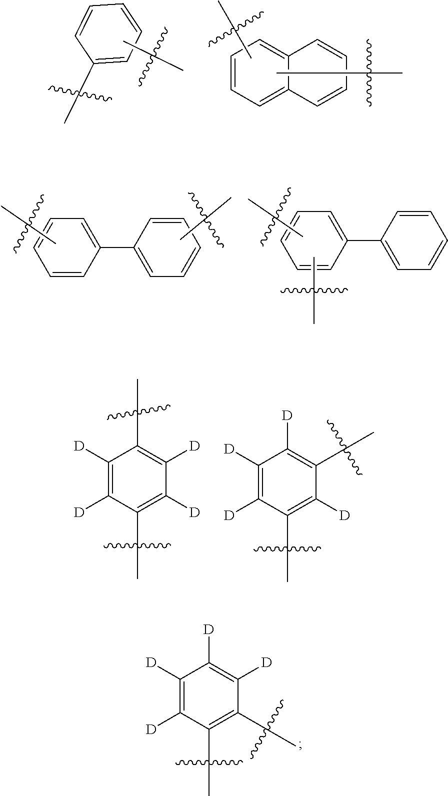
L₂ is selected from a single bond and the group consisting of the following groups:

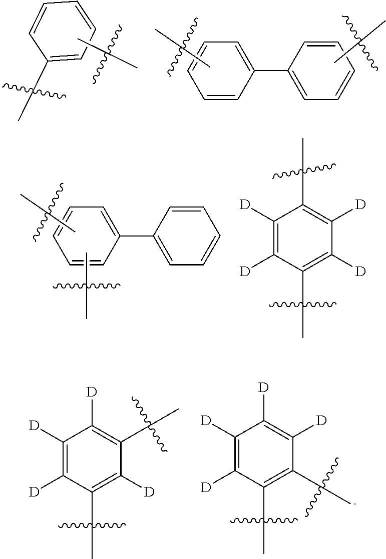

6. (canceled)

7. The nitrogen-containing compound according to claim 1, wherein Ar₁ is selected from a substituted or unsubstituted phenyl, a substituted or unsubstituted naphthyl, a substituted or unsubstituted biphenyl, a substituted or unsubstituted phenanthryl, a substituted or unsubstituted fluorenyl, a substituted or unsubstituted terphenyl, a substituted or unsubstituted dibenzofuranyl, a substituted or unsubstituted dibenzothienyl, or a substituted or unsubstituted carbazolyl;
Ar₂ is selected from a substituted or unsubstituted phenyl, a substituted or unsubstituted biphenyl, a substituted or unsubstituted phenanthryl, a substituted or unsubstituted fluorenyl, a substituted or unsubstituted terphenyl, a substituted or unsubstituted dibenzofuranyl, a substituted or unsubstituted dibenzothienyl, or a substituted or unsubstituted carbazolyl.

8. (canceled)

9. The nitrogen-containing compound according to claim 1, wherein Ar₁ is selected from the group consisting of the following groups: 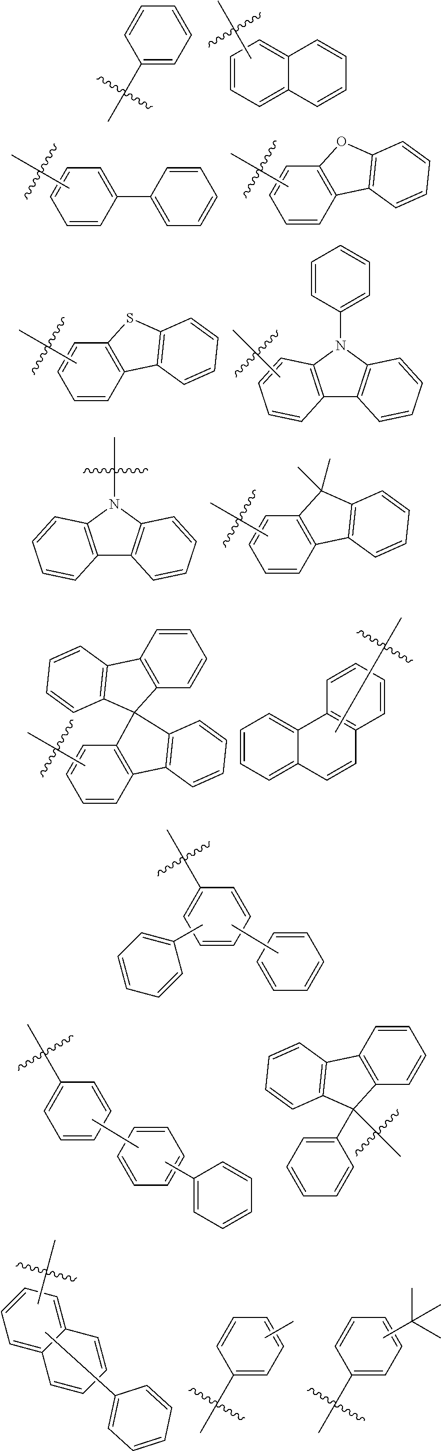 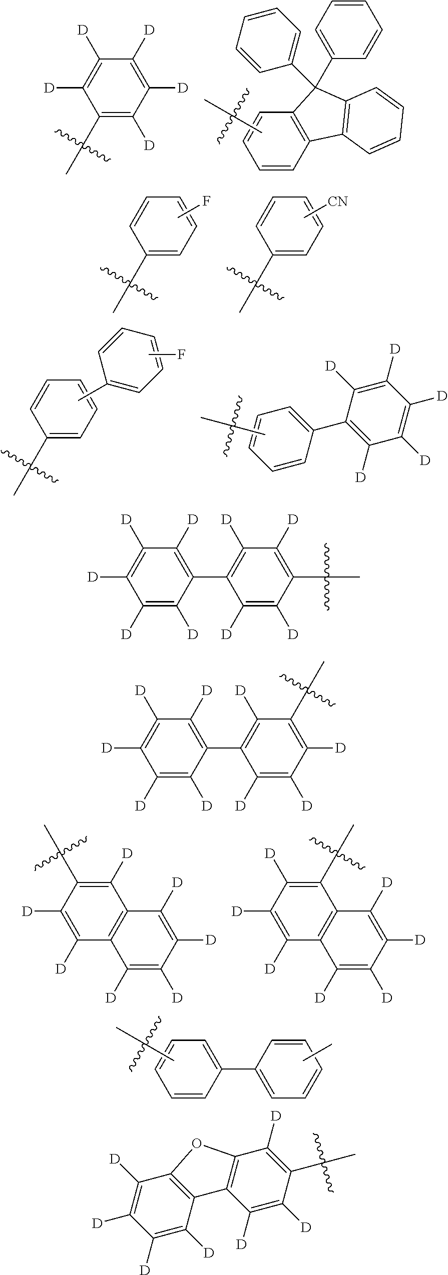 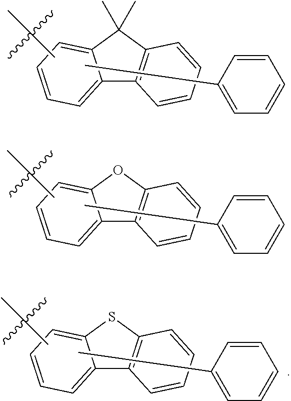
Ar₂ is selected from the group consisting of the following groups:

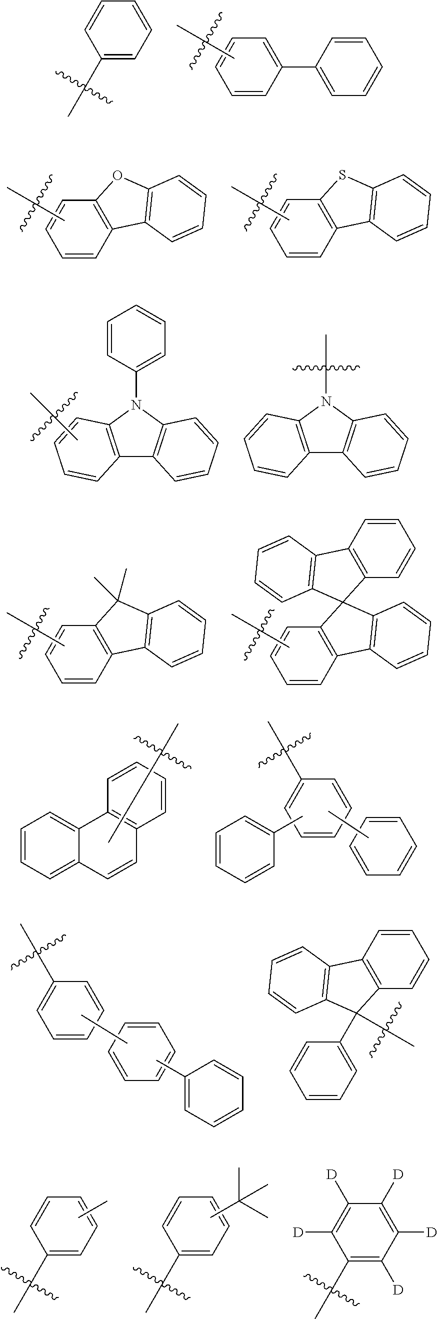

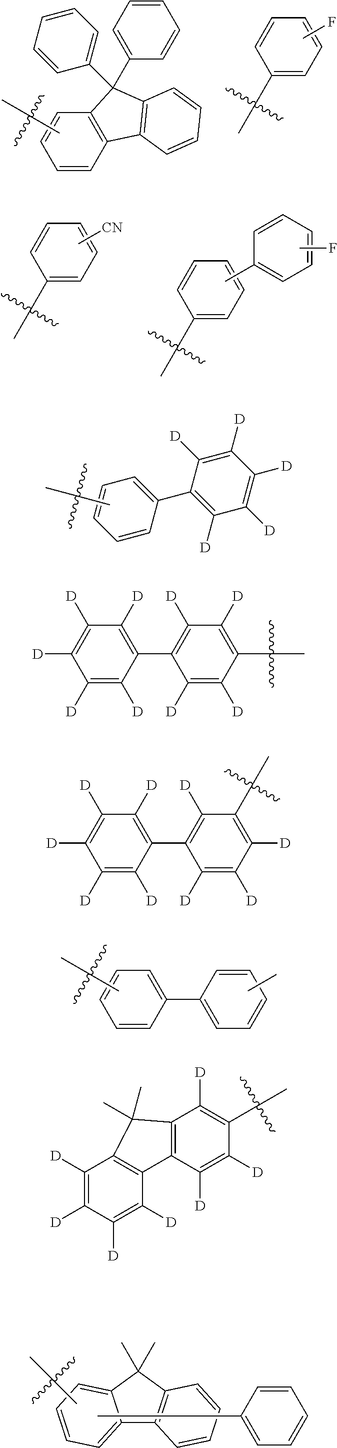

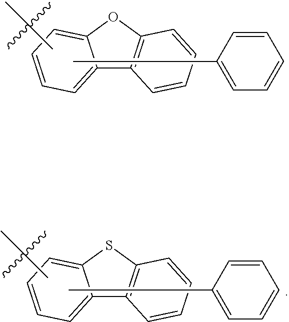

10. The nitrogen-containing compound according to claim 1, wherein 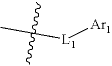
is selected from the group consisting of the following groups: 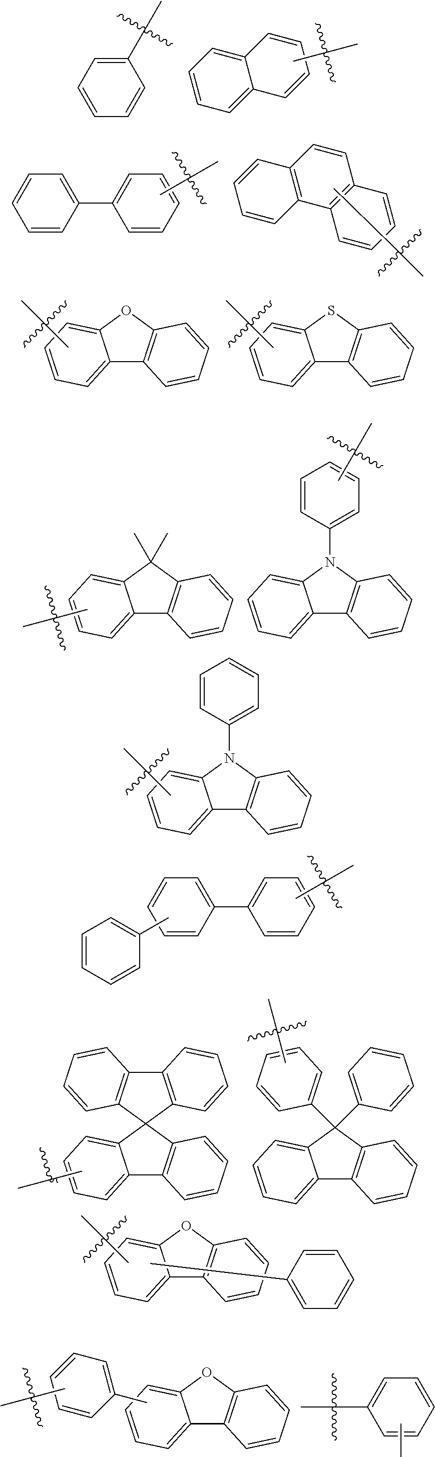 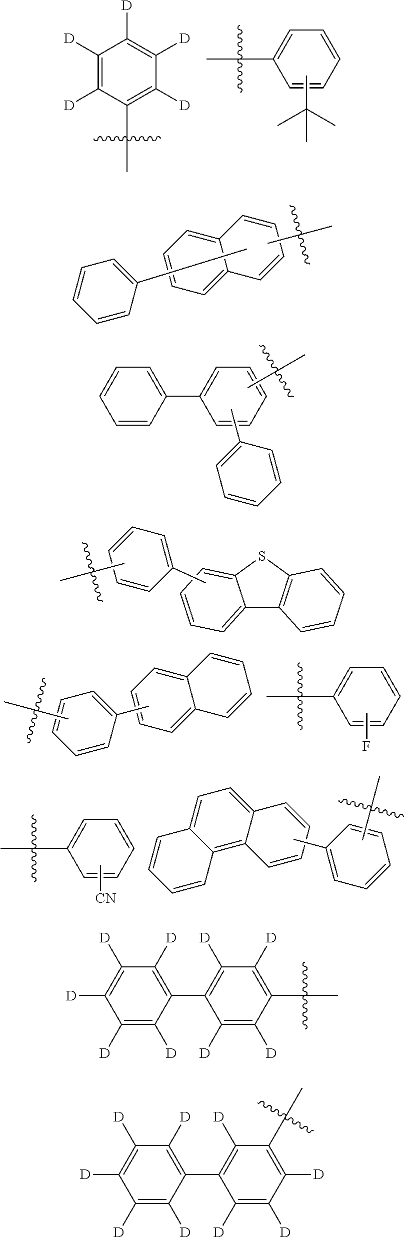 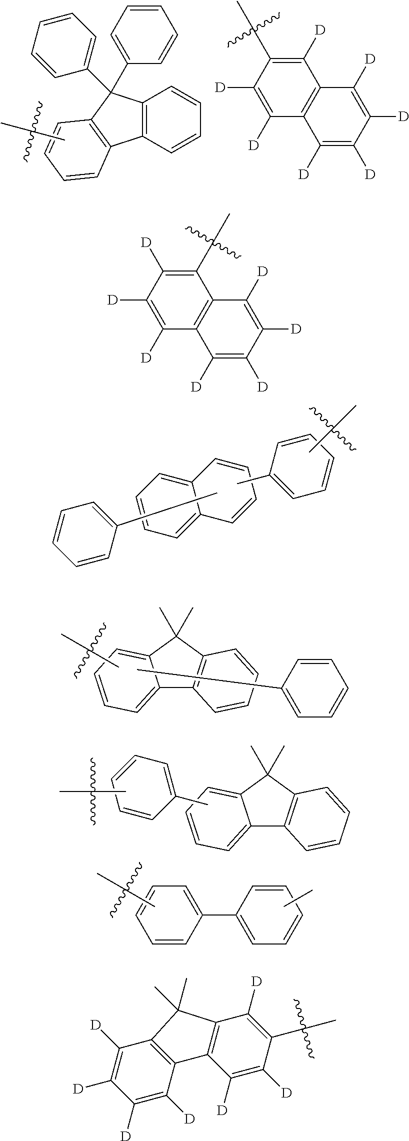
is selected from the group consisting of the following groups:

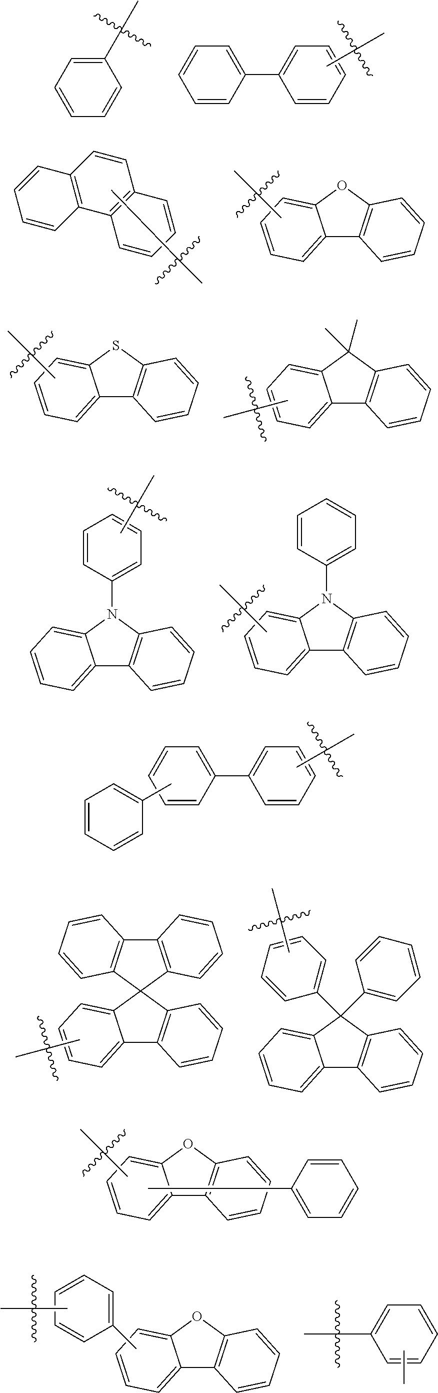

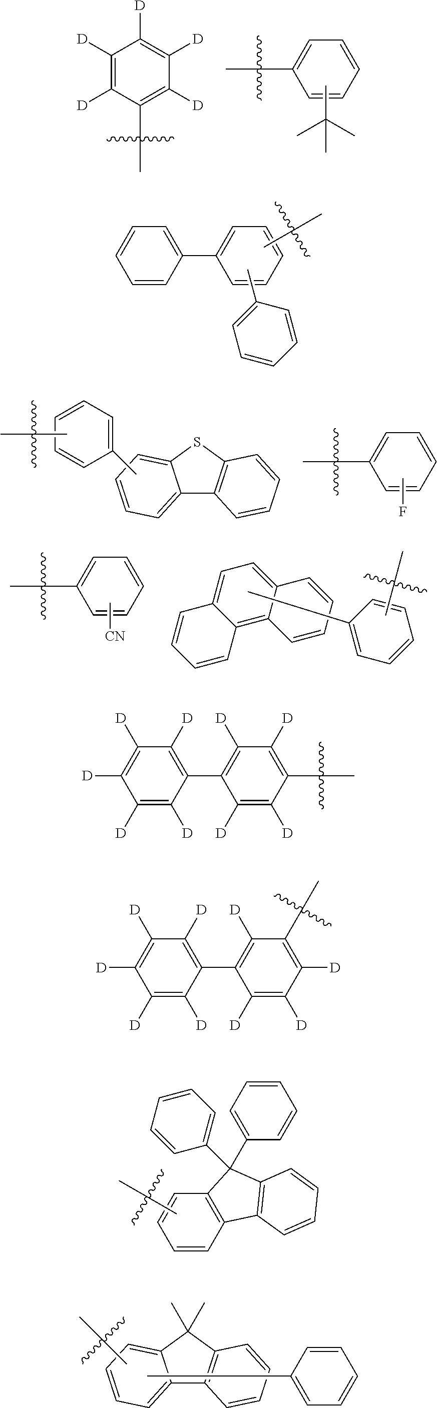

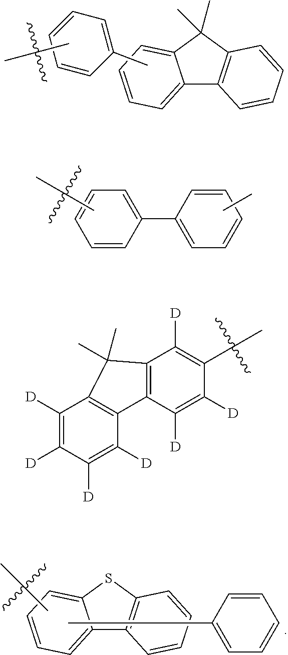

11. The nitrogen-containing compound according to claim 1, wherein the nitrogen-containing compound is selected from the group consisting of the following compounds:

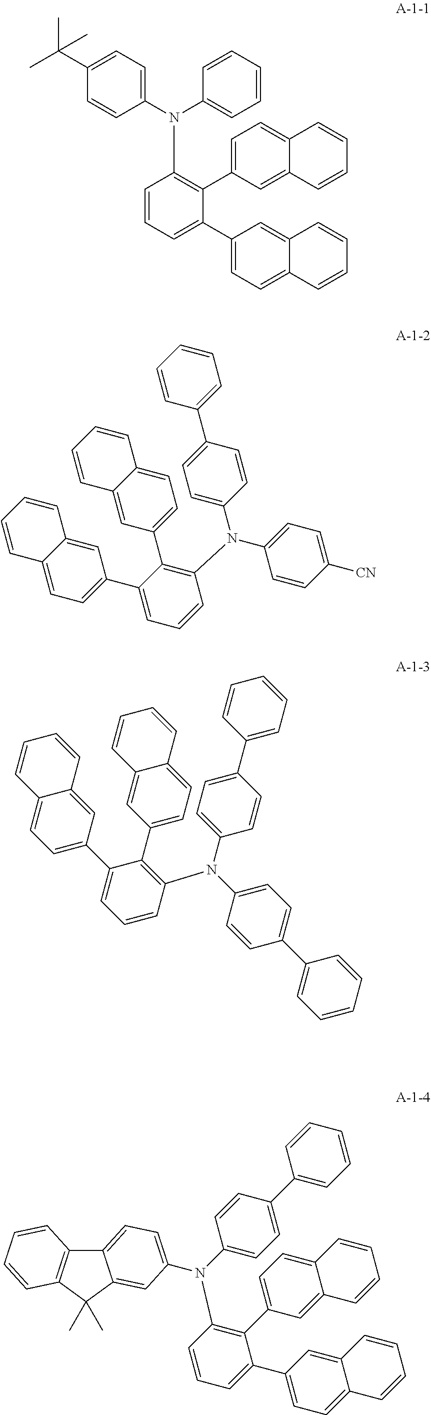 [SMILES: CC(C)(C)c1ccc(N(c2ccccc2)c2cccc(-c3ccc4ccccc4c3)c2-c2ccc3ccccc3c2)cc1 ; N#Cc1ccc(N(c2ccc(-c3ccccc3)cc2)c2cccc(-c3ccc4ccccc4c3)c2-c2ccc3ccccc3c2)cc1 ; c1ccc(-c2ccc(N(c3ccc(-c4ccccc4)cc3)c3cccc(-c4ccc5ccccc5c4)c3-c3ccc4ccccc4c3)cc2)cc1 ; CC1(C)c2ccccc2-c2ccc(N(c3ccc(-c4ccccc4)cc3)c3cccc(-c4ccc5ccccc5c4)c3-c3ccc4ccccc4c3)cc21]

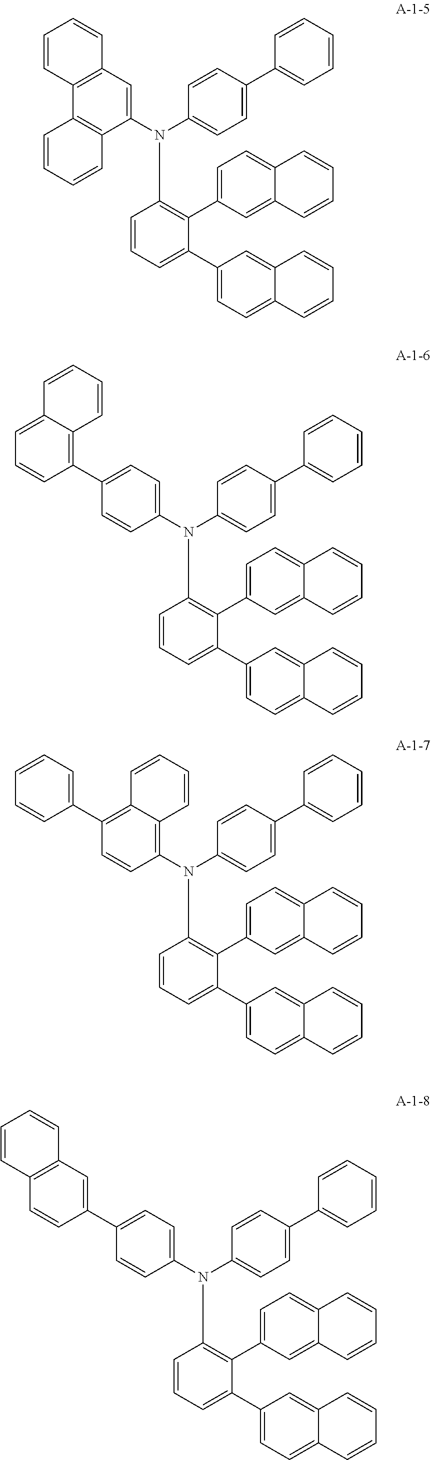

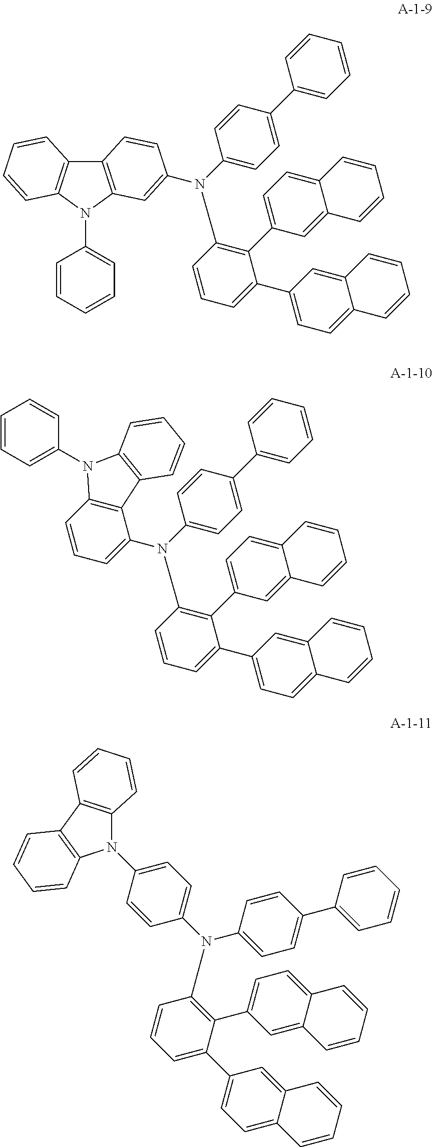 [SMILES: c1ccc(-c2ccc(N(c3ccc4c5ccccc5n(-c5ccccc5)c4c3)c3cccc(-c4ccc5ccccc5c4)c3-c3ccc4ccccc4c3)cc2)cc1 ; c1ccc(-c2ccc(N(c3cccc(-c4ccc5ccccc5c4)c3-c3ccc4ccccc4c3)c3cccc4c3c3ccccc3n4-c3ccccc3)cc2)cc1 ; c1ccc(-c2ccc(N(c3ccc(-n4c5ccccc5c5ccccc54)cc3)c3cccc(-c4ccc5ccccc5c4)c3-c3ccc4ccccc4c3)cc2)cc1]

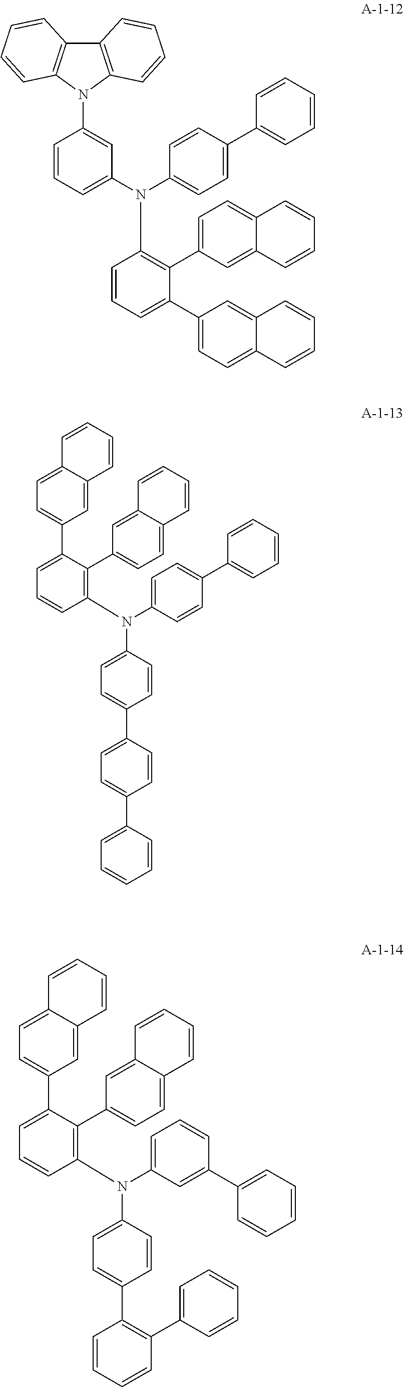 [SMILES: c1ccc(-c2ccc(N(c3cccc(-n4c5ccccc5c5ccccc54)c3)c3cccc(-c4ccc5ccccc5c4)c3-c3ccc4ccccc4c3)cc2)cc1 ; c1ccc(-c2ccc(-c3ccc(N(c4ccc(-c5ccccc5)cc4)c4cccc(-c5ccc6ccccc6c5)c4-c4ccc5ccccc5c4)cc3)cc2)cc1 ; c1ccc(-c2cccc(N(c3ccc(-c4ccccc4-c4ccccc4)cc3)c3cccc(-c4ccc5ccccc5c4)c3-c3ccc4ccccc4c3)c2)cc1]

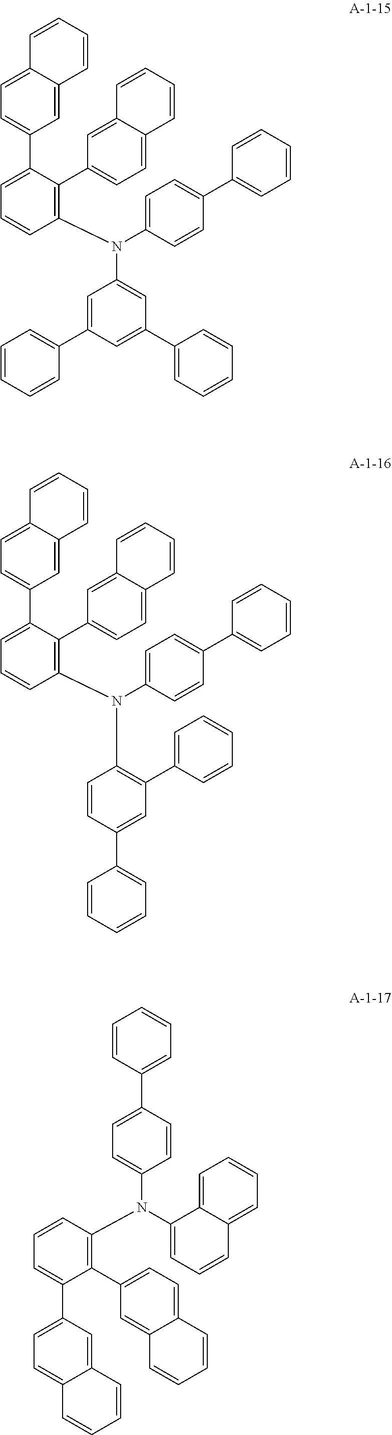 [SMILES: c1ccc(-c2ccc(N(c3cc(-c4ccccc4)cc(-c4ccccc4)c3)c3cccc(-c4ccc5ccccc5c4)c3-c3ccc4ccccc4c3)cc2)cc1 ; c1ccc(-c2ccc(N(c3ccc(-c4ccccc4)cc3-c3ccccc3)c3cccc(-c4ccc5ccccc5c4)c3-c3ccc4ccccc4c3)cc2)cc1 ; c1ccc(-c2ccc(N(c3cccc(-c4ccc5ccccc5c4)c3-c3ccc4ccccc4c3)c3cccc4ccccc34)cc2)cc1]

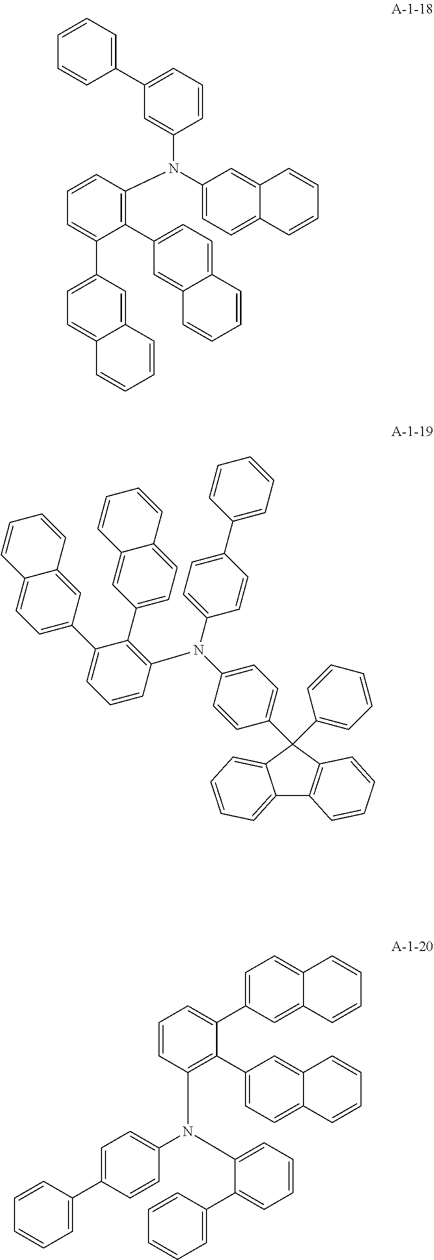 [SMILES: c1ccc(-c2cccc(N(c3ccc4ccccc4c3)c3cccc(-c4ccc5ccccc5c4)c3-c3ccc4ccccc4c3)c2)cc1 ; c1ccc(-c2ccc(N(c3ccc(C4(c5ccccc5)c5ccccc5-c5ccccc54)cc3)c3cccc(-c4ccc5ccccc5c4)c3-c3ccc4ccccc4c3)cc2)cc1 ; c1ccc(-c2ccc(N(c3ccccc3-c3ccccc3)c3cccc(-c4ccc5ccccc5c4)c3-c3ccc4ccccc4c3)cc2)cc1]

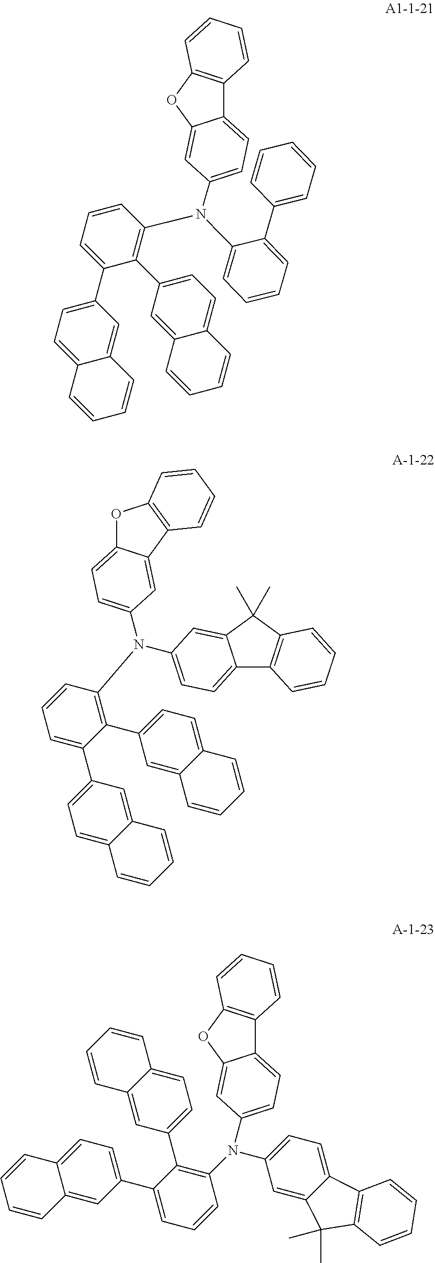 [SMILES: c1ccc(-c2ccccc2N(c2ccc3c(c2)oc2ccccc23)c2cccc(-c3ccc4ccccc4c3)c2-c2ccc3ccccc3c2)cc1 ; CC1(C)c2ccccc2-c2ccc(N(c3ccc4oc5ccccc5c4c3)c3cccc(-c4ccc5ccccc5c4)c3-c3ccc4ccccc4c3)cc21 ; CC1(C)c2ccccc2-c2ccc(N(c3ccc4c(c3)oc3ccccc34)c3cccc(-c4ccc5ccccc5c4)c3-c3ccc4ccccc4c3)cc21]

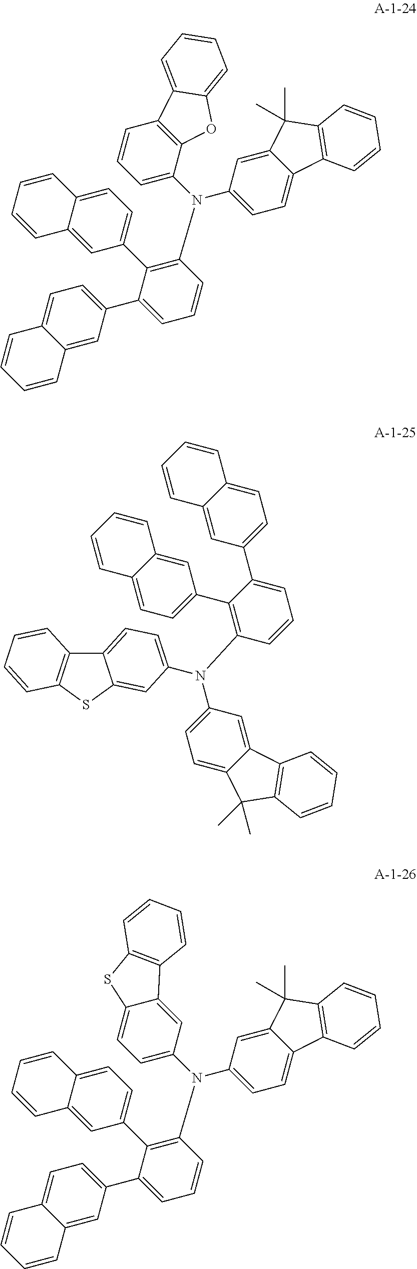 [SMILES: CC1(C)c2ccccc2-c2ccc(N(c3cccc(-c4ccc5ccccc5c4)c3-c3ccc4ccccc4c3)c3cccc4c3oc3ccccc34)cc21 ; CC1(C)c2ccccc2-c2cc(N(c3ccc4c(c3)sc3ccccc34)c3cccc(-c4ccc5ccccc5c4)c3-c3ccc4ccccc4c3)ccc21 ; CC1(C)c2ccccc2-c2ccc(N(c3ccc4sc5ccccc5c4c3)c3cccc(-c4ccc5ccccc5c4)c3-c3ccc4ccccc4c3)cc21]

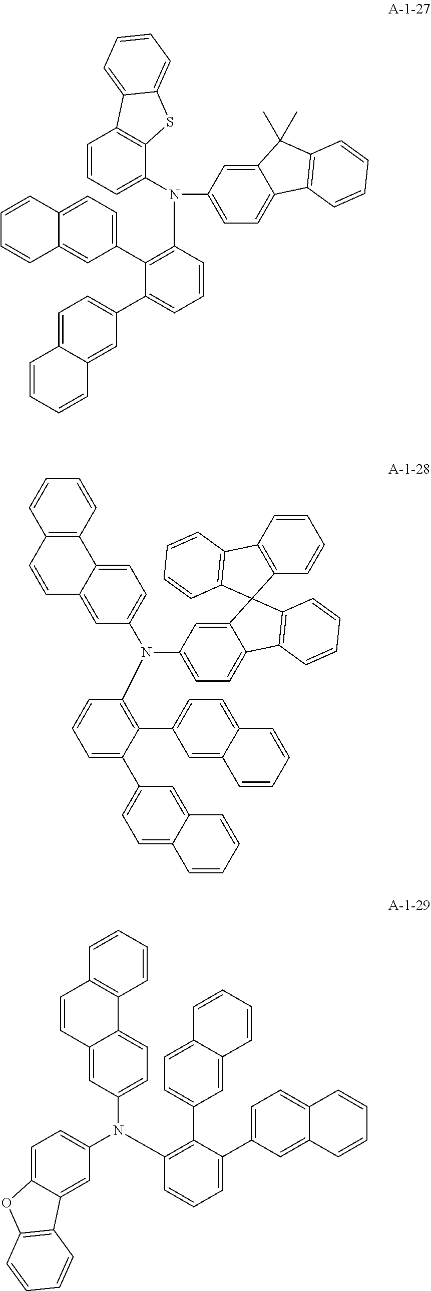 [SMILES: CC1(C)c2ccccc2-c2ccc(N(c3cccc(-c4ccc5ccccc5c4)c3-c3ccc4ccccc4c3)c3cccc4c3sc3ccccc34)cc21 ; c1ccc2c(c1)-c1ccccc1C21c2ccccc2-c2ccc(N(c3ccc4c(ccc5ccccc54)c3)c3cccc(-c4ccc5ccccc5c4)c3-c3ccc4ccccc4c3)cc21 ; c1cc(-c2ccc3ccccc3c2)c(-c2ccc3ccccc3c2)c(N(c2ccc3c(ccc4ccccc43)c2)c2ccc3oc4ccccc4c3c2)c1]

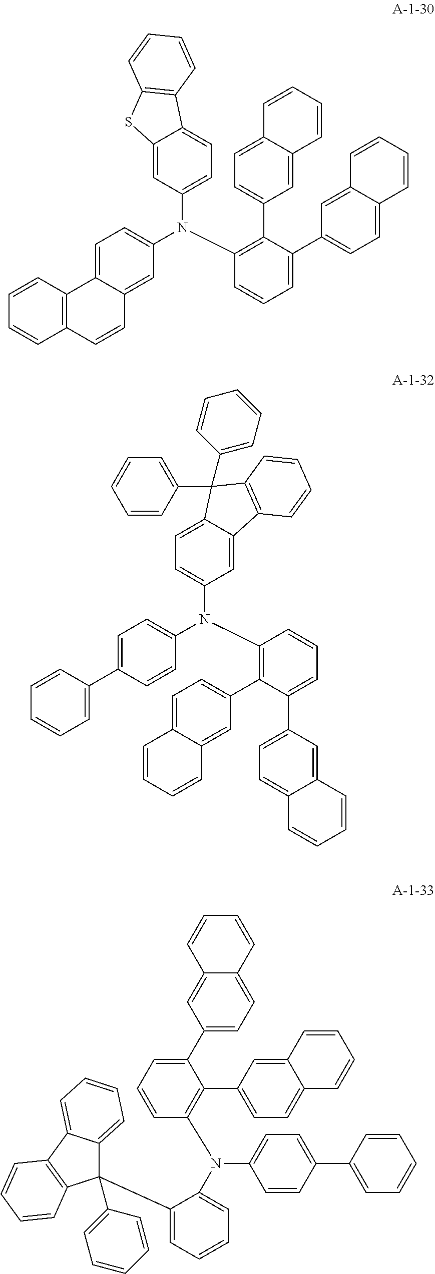 [SMILES: c1cc(-c2ccc3ccccc3c2)c(-c2ccc3ccccc3c2)c(N(c2ccc3c(ccc4ccccc43)c2)c2ccc3c(c2)sc2ccccc23)c1 ; c1ccc(-c2ccc(N(c3ccc4c(c3)-c3ccccc3C4(c3ccccc3)c3ccccc3)c3cccc(-c4ccc5ccccc5c4)c3-c3ccc4ccccc4c3)cc2)cc1 ; c1ccc(-c2ccc(N(c3ccccc3C3(c4ccccc4)c4ccccc4-c4ccccc43)c3cccc(-c4ccc5ccccc5c4)c3-c3ccc4ccccc4c3)cc2)cc1]

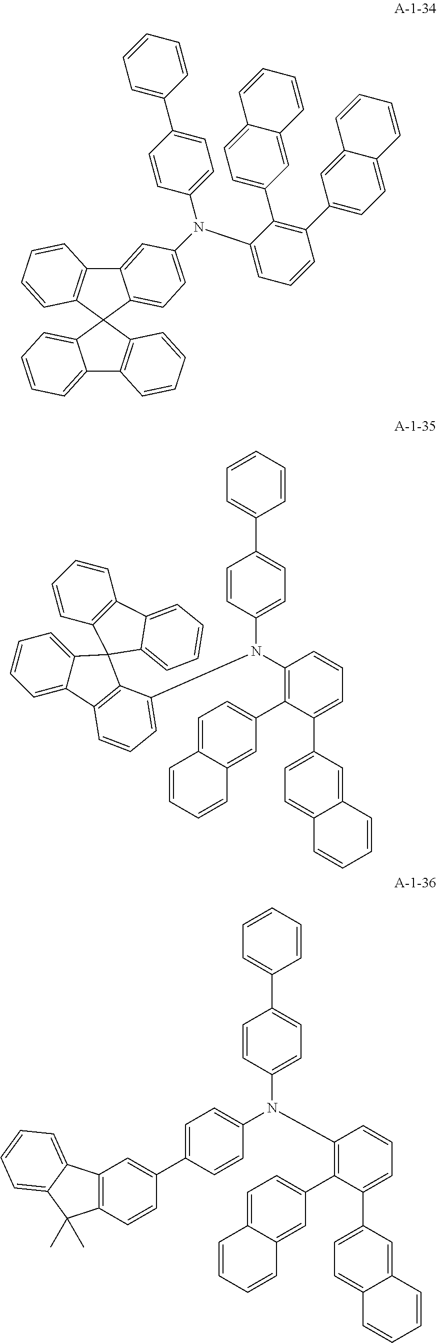 [SMILES: c1ccc(-c2ccc(N(c3ccc4c(c3)-c3ccccc3C43c4ccccc4-c4ccccc43)c3cccc(-c4ccc5ccccc5c4)c3-c3ccc4ccccc4c3)cc2)cc1 ; c1ccc(-c2ccc(N(c3cccc(-c4ccc5ccccc5c4)c3-c3ccc4ccccc4c3)c3cccc4c3C3(c5ccccc5-c5ccccc53)c3ccccc3-4)cc2)cc1 ; CC1(C)c2ccccc2-c2cc(-c3ccc(N(c4ccc(-c5ccccc5)cc4)c4cccc(-c5ccc6ccccc6c5)c4-c4ccc5ccccc5c4)cc3)ccc21]

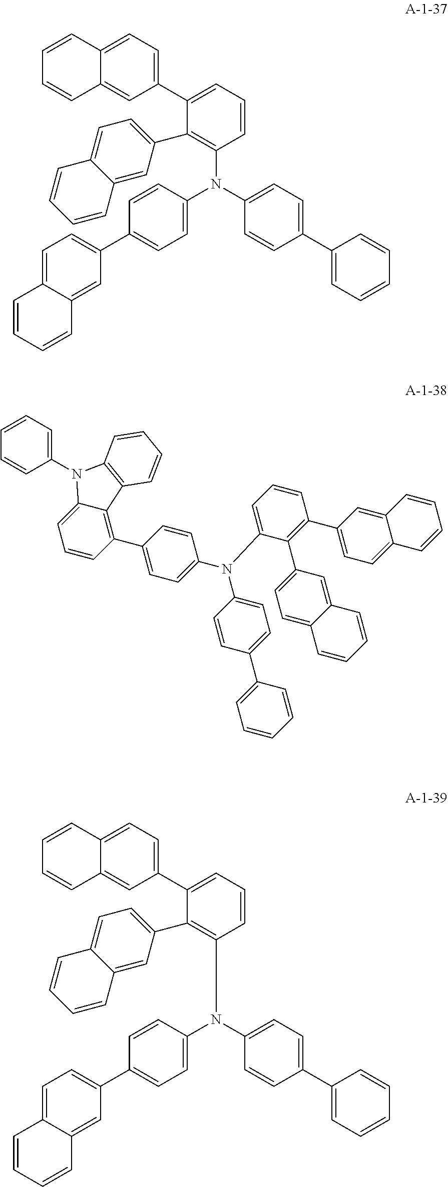 [SMILES: c1ccc(-c2ccc(N(c3ccc(-c4ccc5ccccc5c4)cc3)c3cccc(-c4ccc5ccccc5c4)c3-c3ccc4ccccc4c3)cc2)cc1 ; c1ccc(-c2ccc(N(c3ccc(-c4cccc5c4c4ccccc4n5-c4ccccc4)cc3)c3cccc(-c4ccc5ccccc5c4)c3-c3ccc4ccccc4c3)cc2)cc1 ; c1ccc(-c2ccc(N(c3ccc(-c4ccc5ccccc5c4)cc3)c3cccc(-c4ccc5ccccc5c4)c3-c3ccc4ccccc4c3)cc2)cc1]

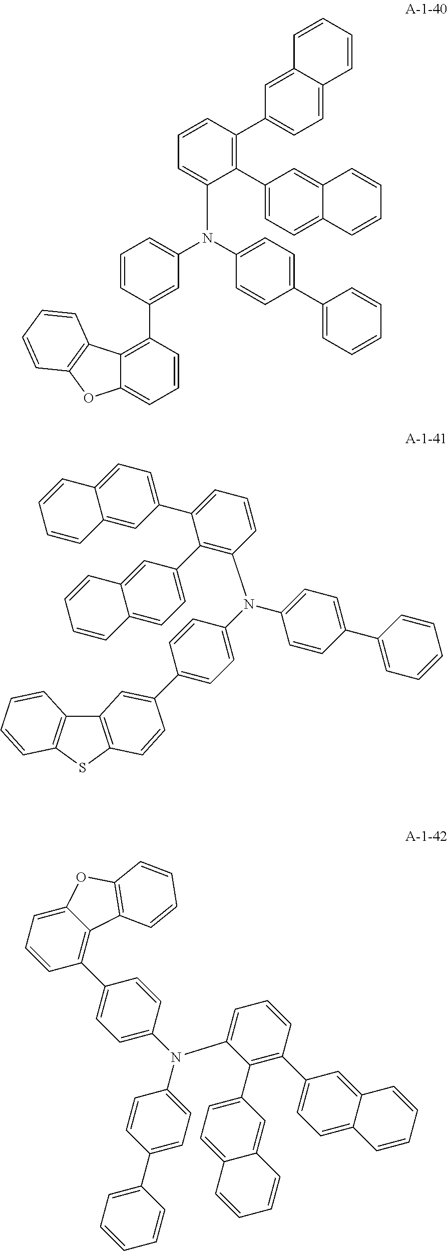 [SMILES: c1ccc(-c2ccc(N(c3cccc(-c4cccc5oc6ccccc6c45)c3)c3cccc(-c4ccc5ccccc5c4)c3-c3ccc4ccccc4c3)cc2)cc1 ; c1ccc(-c2ccc(N(c3ccc(-c4ccc5sc6ccccc6c5c4)cc3)c3cccc(-c4ccc5ccccc5c4)c3-c3ccc4ccccc4c3)cc2)cc1 ; c1ccc(-c2ccc(N(c3ccc(-c4cccc5oc6ccccc6c45)cc3)c3cccc(-c4ccc5ccccc5c4)c3-c3ccc4ccccc4c3)cc2)cc1]

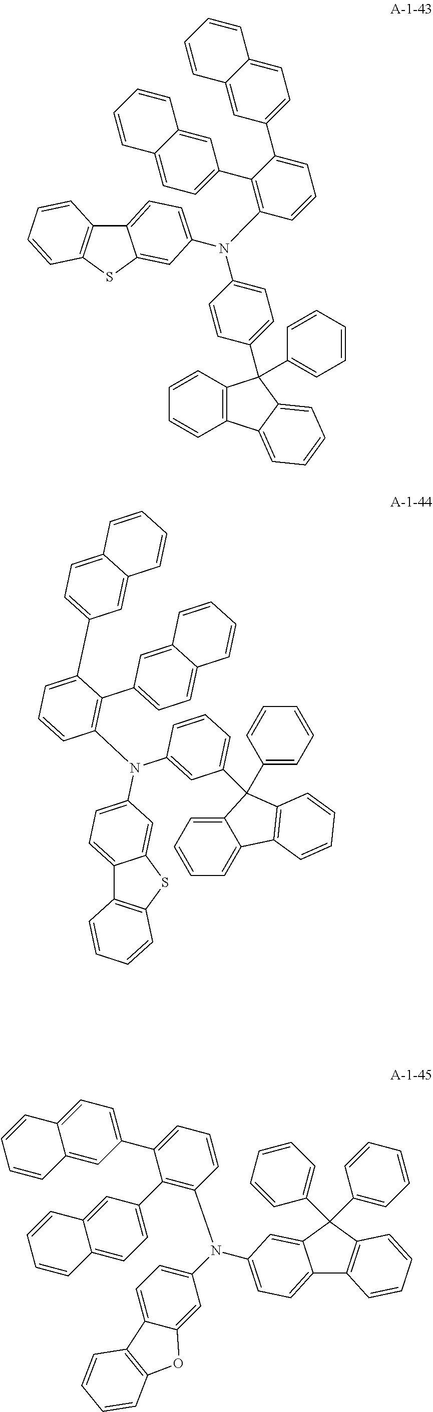 [SMILES: c1ccc(C2(c3ccc(N(c4ccc5c(c4)sc4ccccc45)c4cccc(-c5ccc6ccccc6c5)c4-c4ccc5ccccc5c4)cc3)c3ccccc3-c3ccccc32)cc1 ; c1ccc(C2(c3cccc(N(c4ccc5c(c4)sc4ccccc45)c4cccc(-c5ccc6ccccc6c5)c4-c4ccc5ccccc5c4)c3)c3ccccc3-c3ccccc32)cc1 ; c1ccc(C2(c3ccccc3)c3ccccc3-c3ccc(N(c4ccc5c(c4)oc4ccccc45)c4cccc(-c5ccc6ccccc6c5)c4-c4ccc5ccccc5c4)cc32)cc1]

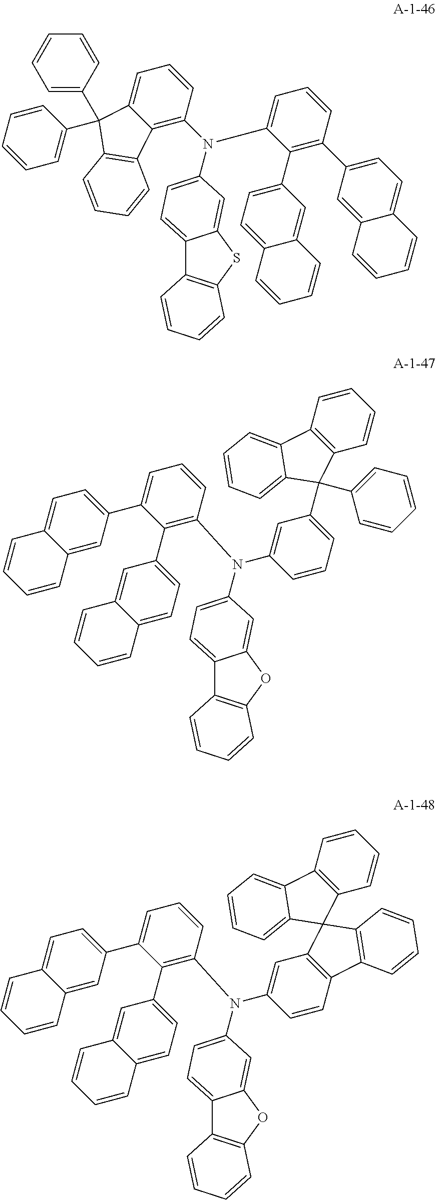 [SMILES: c1ccc(C2(c3ccccc3)c3ccccc3-c3c(N(c4ccc5c(c4)sc4ccccc45)c4cccc(-c5ccc6ccccc6c5)c4-c4ccc5ccccc5c4)cccc32)cc1 ; c1ccc(C2(c3cccc(N(c4ccc5c(c4)oc4ccccc45)c4cccc(-c5ccc6ccccc6c5)c4-c4ccc5ccccc5c4)c3)c3ccccc3-c3ccccc32)cc1 ; c1ccc2c(c1)-c1ccccc1C21c2ccccc2-c2ccc(N(c3ccc4c(c3)oc3ccccc34)c3cccc(-c4ccc5ccccc5c4)c3-c3ccc4ccccc4c3)cc21]

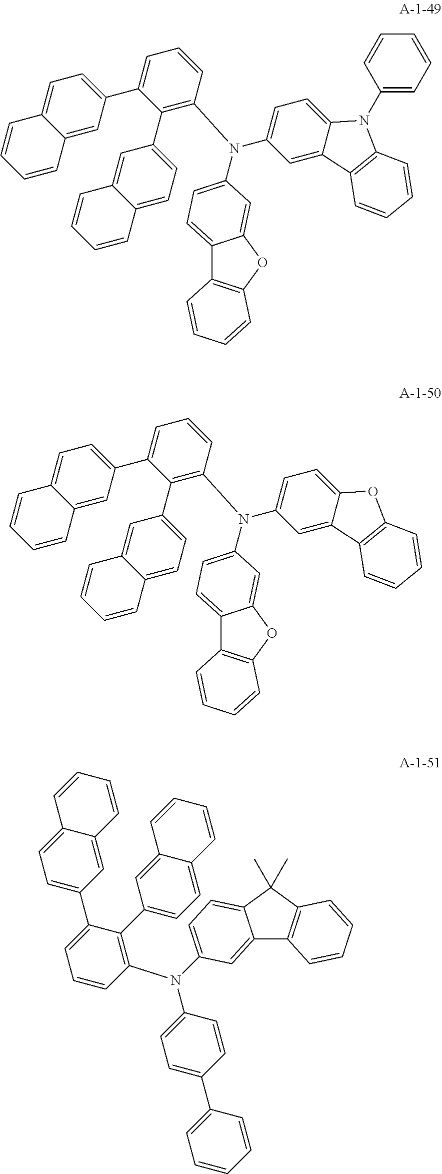 [SMILES: c1ccc(-n2c3ccccc3c3cc(N(c4ccc5c(c4)oc4ccccc45)c4cccc(-c5ccc6ccccc6c5)c4-c4ccc5ccccc5c4)ccc32)cc1 ; c1cc(-c2ccc3ccccc3c2)c(-c2ccc3ccccc3c2)c(N(c2ccc3c(c2)oc2ccccc23)c2ccc3oc4ccccc4c3c2)c1 ; CC1(C)c2ccccc2-c2cc(N(c3ccc(-c4ccccc4)cc3)c3cccc(-c4ccc5ccccc5c4)c3-c3ccc4ccccc4c3)ccc21]

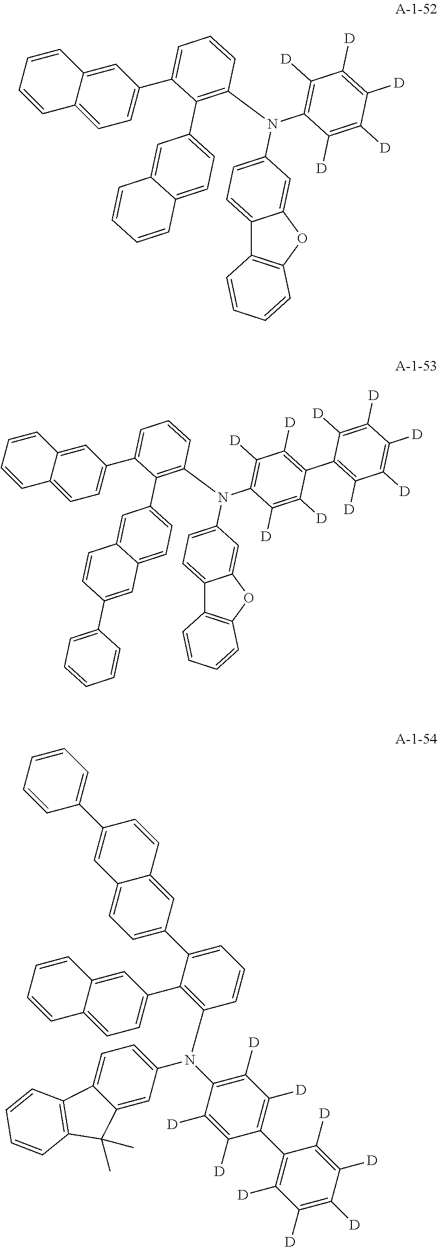 [SMILES: [2H]c1c([2H])c([2H])c(N(c2ccc3c(c2)oc2ccccc23)c2cccc(-c3ccc4ccccc4c3)c2-c2ccc3ccccc3c2)c([2H])c1[2H] ; [2H]c1c([2H])c([2H])c(-c2c([2H])c([2H])c(N(c3ccc4c(c3)oc3ccccc34)c3cccc(-c4ccc5ccccc5c4)c3-c3ccc4cc(-c5ccccc5)ccc4c3)c([2H])c2[2H])c([2H])c1[2H] ; [2H]c1c([2H])c([2H])c(-c2c([2H])c([2H])c(N(c3ccc4c(c3)C(C)(C)c3ccccc3-4)c3cccc(-c4ccc5cc(-c6ccccc6)ccc5c4)c3-c3ccc4ccccc4c3)c([2H])c2[2H])c([2H])c1[2H]]

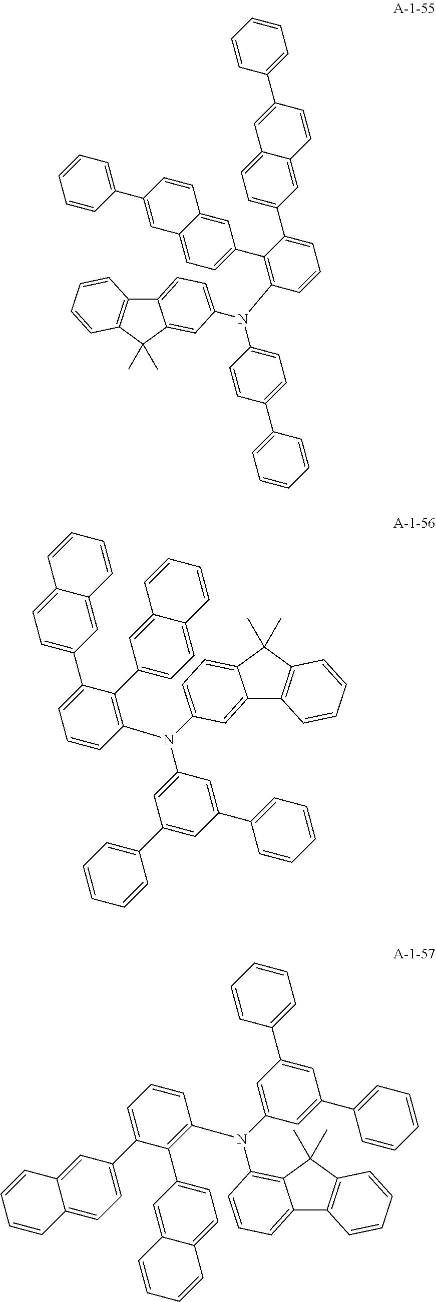 [SMILES: CC1(C)c2ccccc2-c2ccc(N(c3ccc(-c4ccccc4)cc3)c3cccc(-c4ccc5cc(-c6ccccc6)ccc5c4)c3-c3ccc4cc(-c5ccccc5)ccc4c3)cc21 ; CC1(C)c2ccccc2-c2cc(N(c3cc(-c4ccccc4)cc(-c4ccccc4)c3)c3cccc(-c4ccc5ccccc5c4)c3-c3ccc4ccccc4c3)ccc21 ; CC1(C)c2ccccc2-c2cccc(N(c3cc(-c4ccccc4)cc(-c4ccccc4)c3)c3cccc(-c4ccc5ccccc5c4)c3-c3ccc4ccccc4c3)c21]

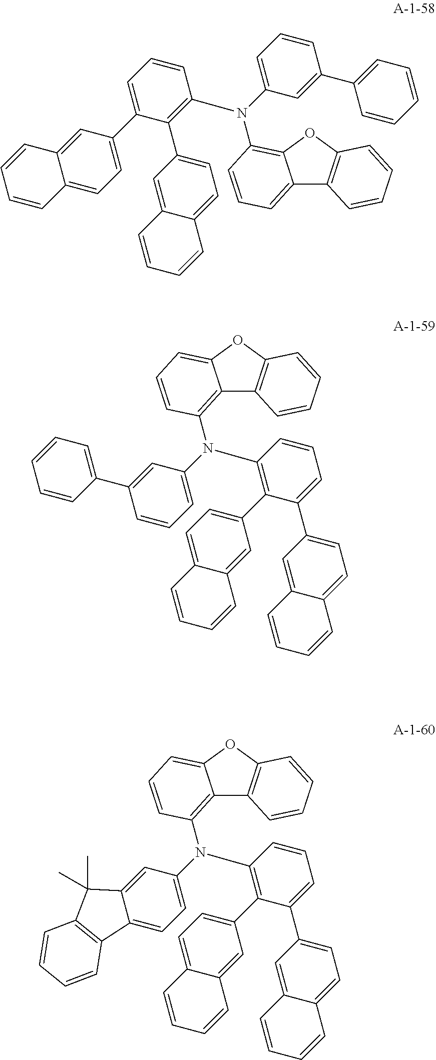 [SMILES: c1ccc(-c2cccc(N(c3cccc(-c4ccc5ccccc5c4)c3-c3ccc4ccccc4c3)c3cccc4c3oc3ccccc34)c2)cc1 ; c1ccc(-c2cccc(N(c3cccc(-c4ccc5ccccc5c4)c3-c3ccc4ccccc4c3)c3cccc4oc5ccccc5c34)c2)cc1 ; CC1(C)c2ccccc2-c2ccc(N(c3cccc(-c4ccc5ccccc5c4)c3-c3ccc4ccccc4c3)c3cccc4oc5ccccc5c34)cc21]

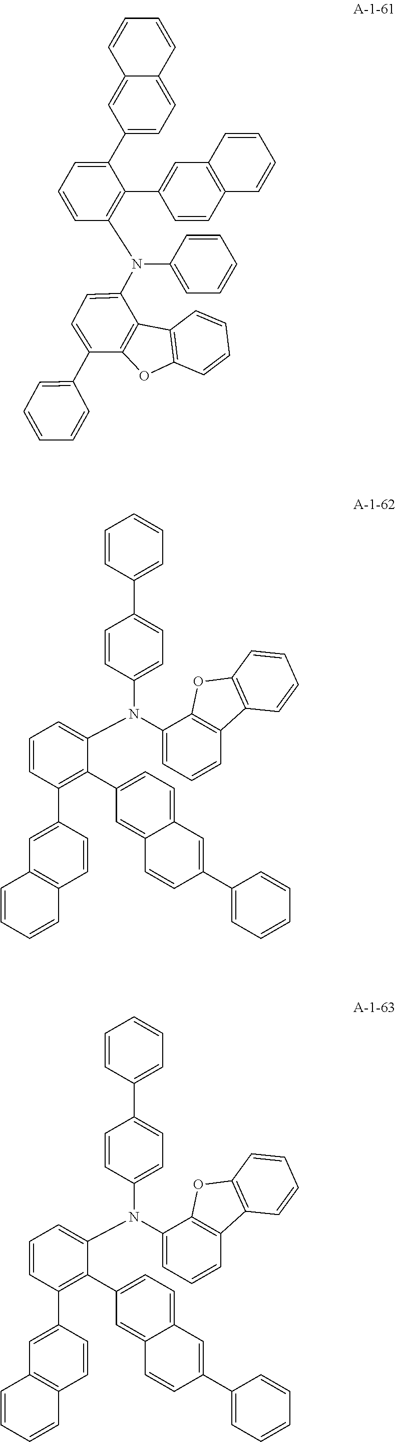 [SMILES: c1ccc(-c2ccc(N(c3ccccc3)c3cccc(-c4ccc5ccccc5c4)c3-c3ccc4ccccc4c3)c3c2oc2ccccc23)cc1 ; c1ccc(-c2ccc(N(c3cccc(-c4ccc5ccccc5c4)c3-c3ccc4cc(-c5ccccc5)ccc4c3)c3cccc4c3oc3ccccc34)cc2)cc1 ; c1ccc(-c2ccc(N(c3cccc(-c4ccc5ccccc5c4)c3-c3ccc4cc(-c5ccccc5)ccc4c3)c3cccc4c3oc3ccccc34)cc2)cc1]

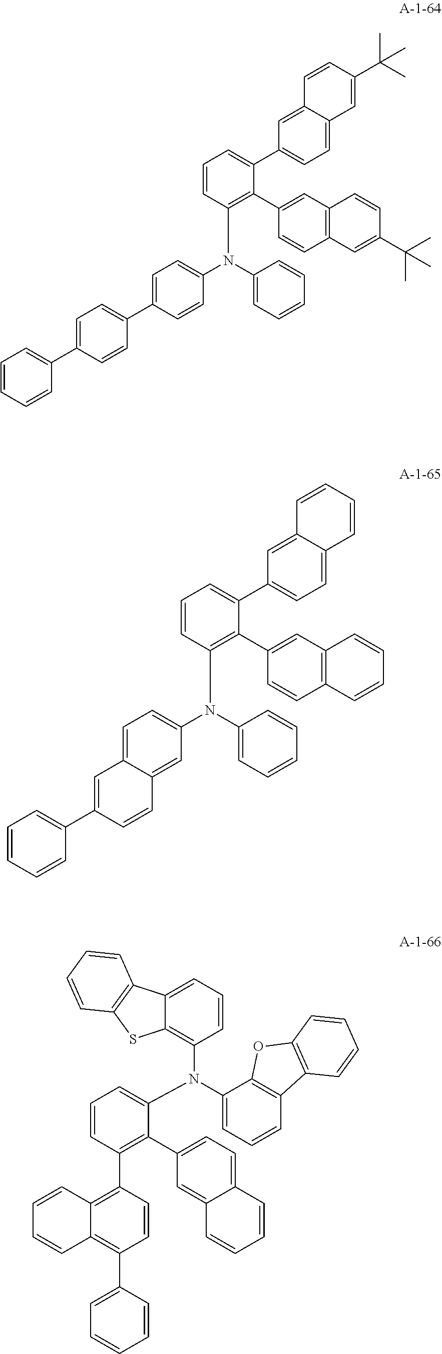 [SMILES: CC(C)(C)c1ccc2cc(-c3cccc(N(c4ccccc4)c4ccc(-c5ccc(-c6ccccc6)cc5)cc4)c3-c3ccc4cc(C(C)(C)C)ccc4c3)ccc2c1 ; c1ccc(-c2ccc3cc(N(c4ccccc4)c4cccc(-c5ccc6ccccc6c5)c4-c4ccc5ccccc5c4)ccc3c2)cc1 ; c1ccc(-c2ccc(-c3cccc(N(c4cccc5c4oc4ccccc45)c4cccc5c4sc4ccccc45)c3-c3ccc4ccccc4c3)c3ccccc23)cc1]

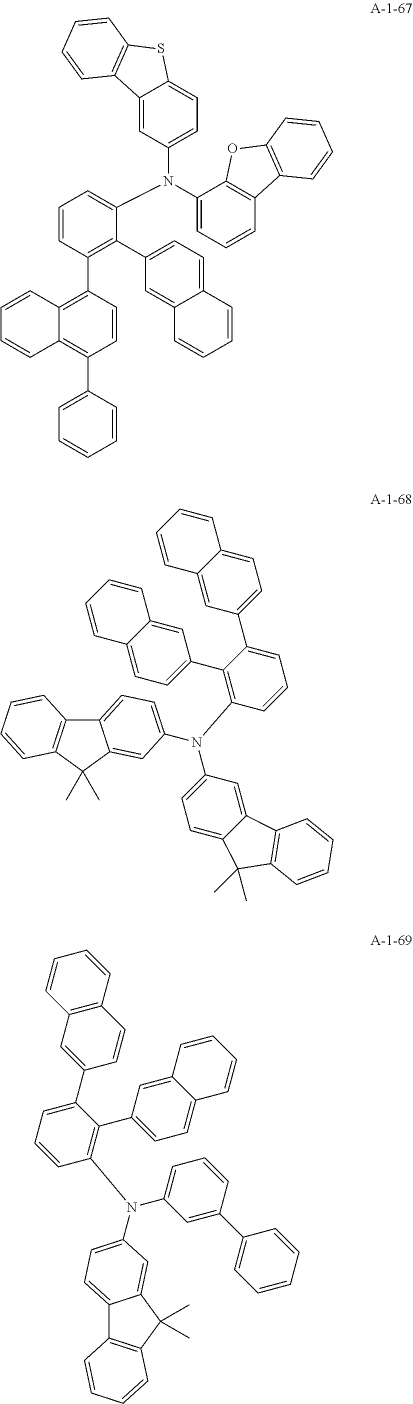 [SMILES: c1ccc(-c2ccc(-c3cccc(N(c4ccc5sc6ccccc6c5c4)c4cccc5c4oc4ccccc45)c3-c3ccc4ccccc4c3)c3ccccc23)cc1 ; CC1(C)c2ccccc2-c2cc(N(c3ccc4c(c3)C(C)(C)c3ccccc3-4)c3cccc(-c4ccc5ccccc5c4)c3-c3ccc4ccccc4c3)ccc21 ; CC1(C)c2ccccc2-c2ccc(N(c3cccc(-c4ccccc4)c3)c3cccc(-c4ccc5ccccc5c4)c3-c3ccc4ccccc4c3)cc21]

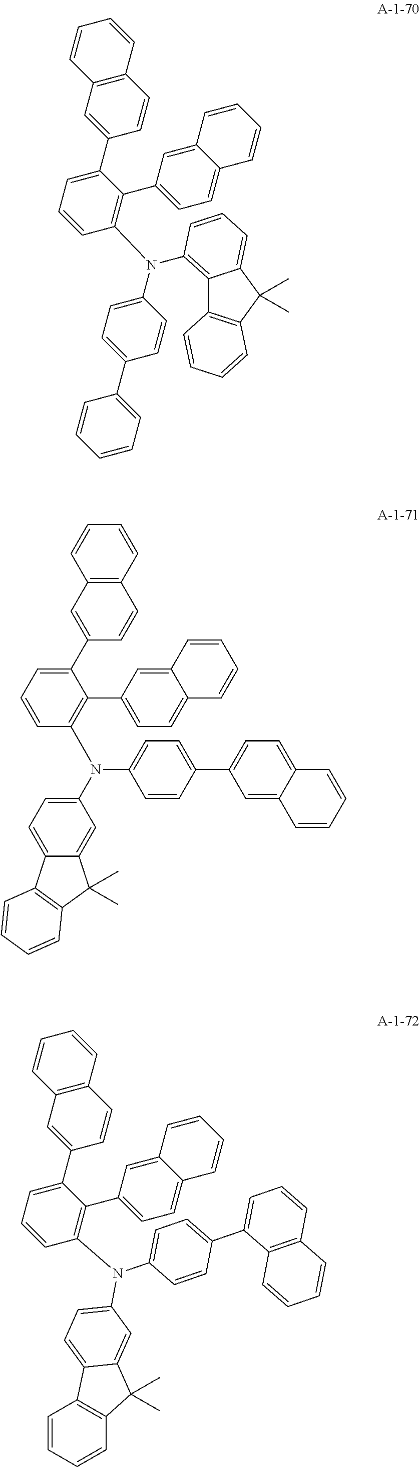 [SMILES: CC1(C)c2ccccc2-c2c(N(c3ccc(-c4ccccc4)cc3)c3cccc(-c4ccc5ccccc5c4)c3-c3ccc4ccccc4c3)cccc21 ; CC1(C)c2ccccc2-c2ccc(N(c3ccc(-c4ccc5ccccc5c4)cc3)c3cccc(-c4ccc5ccccc5c4)c3-c3ccc4ccccc4c3)cc21 ; CC1(C)c2ccccc2-c2ccc(N(c3ccc(-c4cccc5ccccc45)cc3)c3cccc(-c4ccc5ccccc5c4)c3-c3ccc4ccccc4c3)cc21]

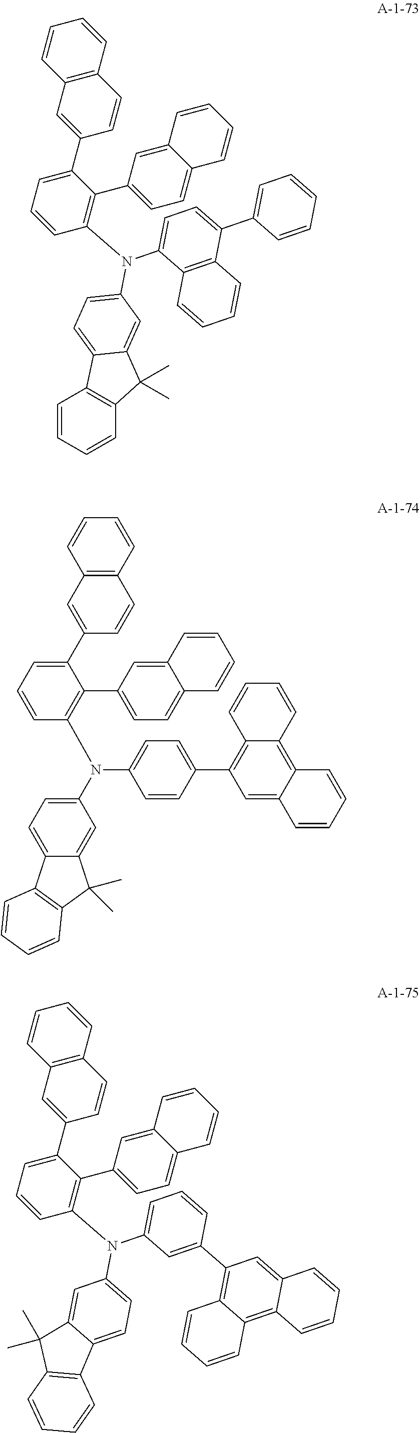 [SMILES: CC1(C)c2ccccc2-c2ccc(N(c3cccc(-c4ccc5ccccc5c4)c3-c3ccc4ccccc4c3)c3ccc(-c4ccccc4)c4ccccc34)cc21 ; CC1(C)c2ccccc2-c2ccc(N(c3ccc(-c4cc5ccccc5c5ccccc45)cc3)c3cccc(-c4ccc5ccccc5c4)c3-c3ccc4ccccc4c3)cc21 ; CC1(C)c2ccccc2-c2ccc(N(c3cccc(-c4cc5ccccc5c5ccccc45)c3)c3cccc(-c4ccc5ccccc5c4)c3-c3ccc4ccccc4c3)cc21]

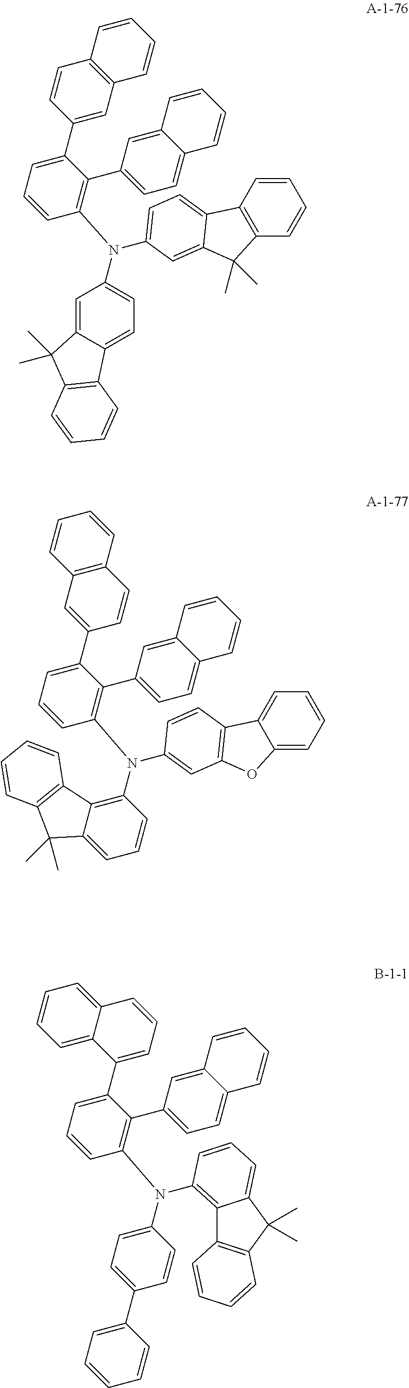 [SMILES: CC1(C)c2ccccc2-c2ccc(N(c3ccc4c(c3)C(C)(C)c3ccccc3-4)c3cccc(-c4ccc5ccccc5c4)c3-c3ccc4ccccc4c3)cc21 ; CC1(C)c2ccccc2-c2c(N(c3ccc4c(c3)oc3ccccc34)c3cccc(-c4ccc5ccccc5c4)c3-c3ccc4ccccc4c3)cccc21 ; CC1(C)c2ccccc2-c2c(N(c3ccc(-c4ccccc4)cc3)c3cccc(-c4cccc5ccccc45)c3-c3ccc4ccccc4c3)cccc21]

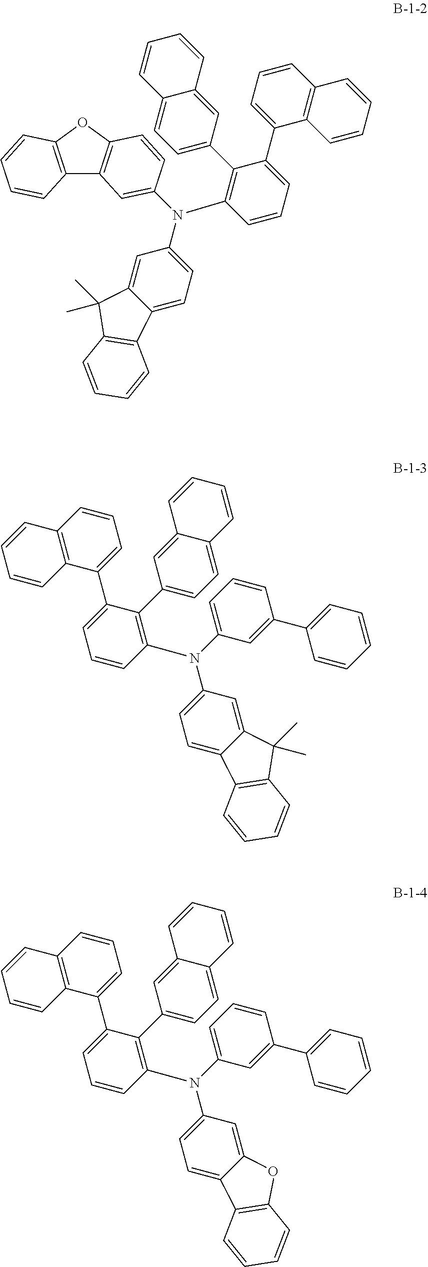 [SMILES: CC1(C)c2ccccc2-c2ccc(N(c3ccc4oc5ccccc5c4c3)c3cccc(-c4cccc5ccccc45)c3-c3ccc4ccccc4c3)cc21 ; CC1(C)c2ccccc2-c2ccc(N(c3cccc(-c4ccccc4)c3)c3cccc(-c4cccc5ccccc45)c3-c3ccc4ccccc4c3)cc21 ; c1ccc(-c2cccc(N(c3ccc4c(c3)oc3ccccc34)c3cccc(-c4cccc5ccccc45)c3-c3ccc4ccccc4c3)c2)cc1]

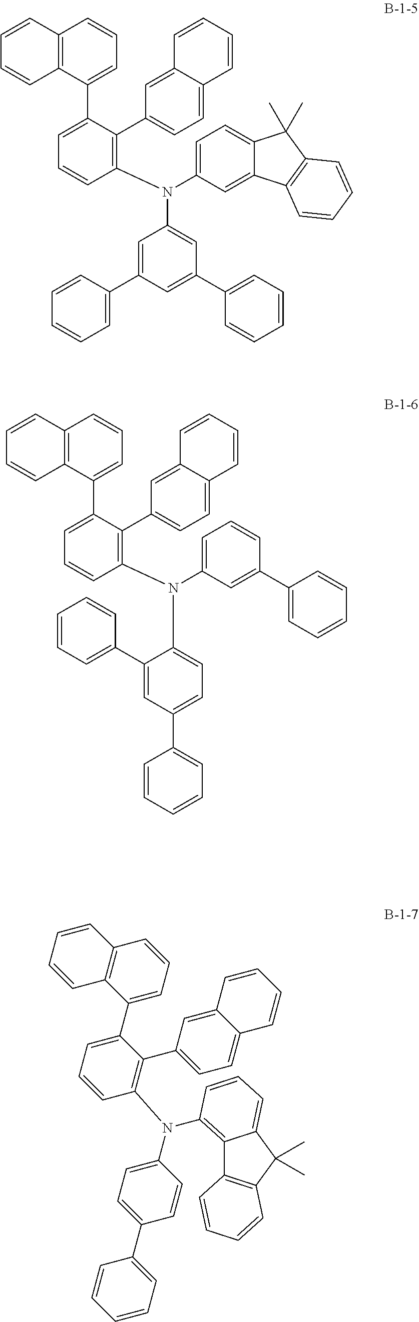 [SMILES: CC1(C)c2ccccc2-c2cc(N(c3cc(-c4ccccc4)cc(-c4ccccc4)c3)c3cccc(-c4cccc5ccccc45)c3-c3ccc4ccccc4c3)ccc21 ; c1ccc(-c2cccc(N(c3ccc(-c4ccccc4)cc3-c3ccccc3)c3cccc(-c4cccc5ccccc45)c3-c3ccc4ccccc4c3)c2)cc1 ; CC1(C)c2ccccc2-c2c(N(c3ccc(-c4ccccc4)cc3)c3cccc(-c4cccc5ccccc45)c3-c3ccc4ccccc4c3)cccc21]

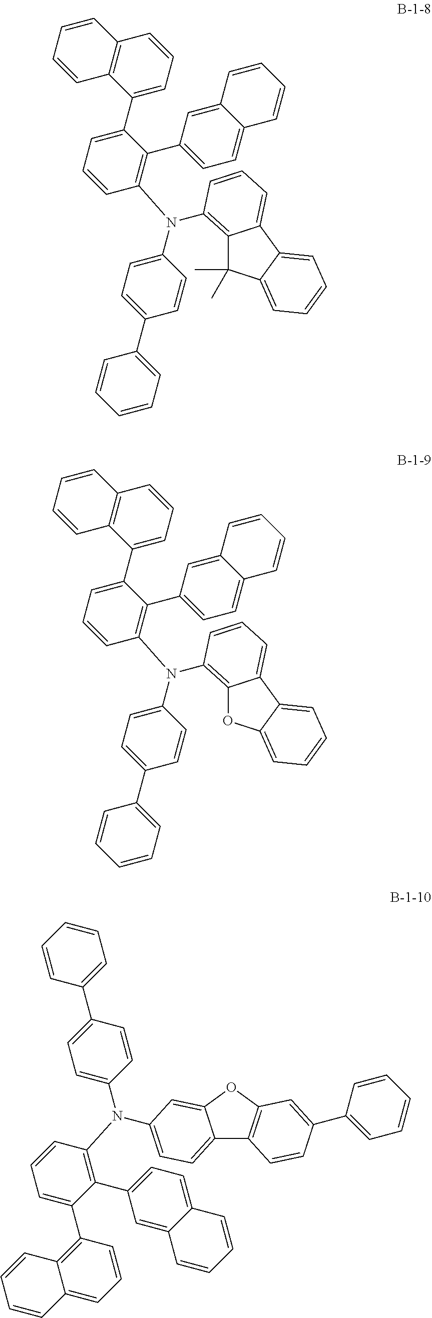 [SMILES: CC1(C)c2ccccc2-c2cccc(N(c3ccc(-c4ccccc4)cc3)c3cccc(-c4cccc5ccccc45)c3-c3ccc4ccccc4c3)c21 ; c1ccc(-c2ccc(N(c3cccc(-c4cccc5ccccc45)c3-c3ccc4ccccc4c3)c3cccc4c3oc3ccccc34)cc2)cc1 ; c1ccc(-c2ccc(N(c3ccc4c(c3)oc3cc(-c5ccccc5)ccc34)c3cccc(-c4cccc5ccccc45)c3-c3ccc4ccccc4c3)cc2)cc1]

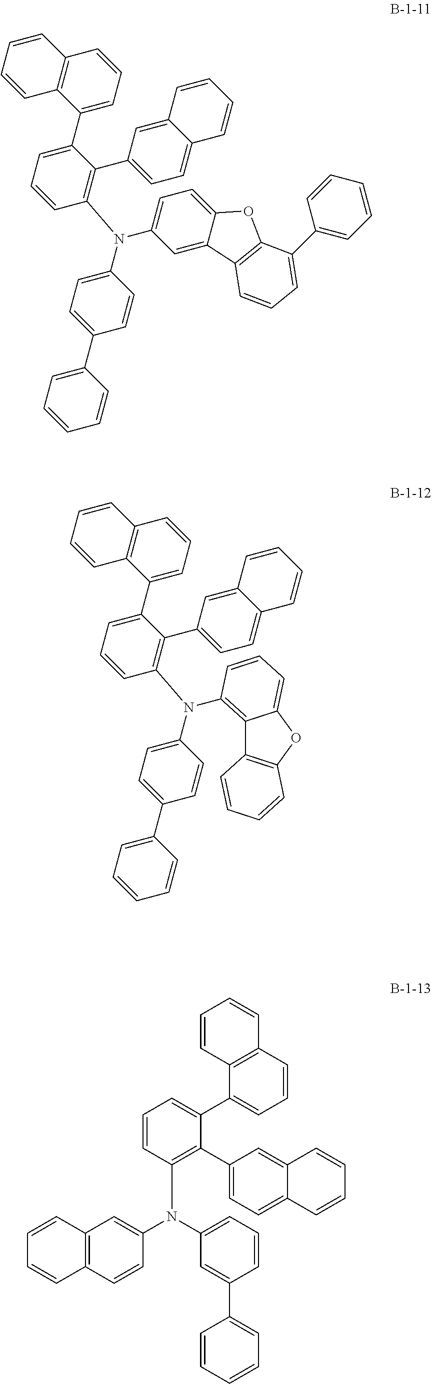 [SMILES: c1ccc(-c2ccc(N(c3ccc4oc5c(-c6ccccc6)cccc5c4c3)c3cccc(-c4cccc5ccccc45)c3-c3ccc4ccccc4c3)cc2)cc1 ; c1ccc(-c2ccc(N(c3cccc(-c4cccc5ccccc45)c3-c3ccc4ccccc4c3)c3cccc4oc5ccccc5c34)cc2)cc1 ; c1ccc(-c2cccc(N(c3ccc4ccccc4c3)c3cccc(-c4cccc5ccccc45)c3-c3ccc4ccccc4c3)c2)cc1]

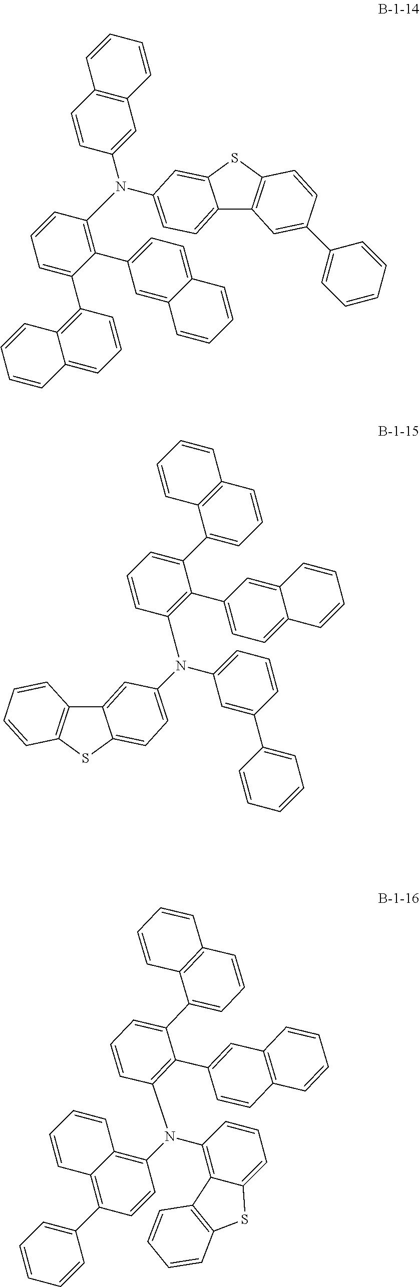 [SMILES: c1ccc(-c2ccc3sc4cc(N(c5ccc6ccccc6c5)c5cccc(-c6cccc7ccccc67)c5-c5ccc6ccccc6c5)ccc4c3c2)cc1 ; c1ccc(-c2cccc(N(c3ccc4sc5ccccc5c4c3)c3cccc(-c4cccc5ccccc45)c3-c3ccc4ccccc4c3)c2)cc1 ; c1ccc(-c2ccc(N(c3cccc(-c4cccc5ccccc45)c3-c3ccc4ccccc4c3)c3cccc4sc5ccccc5c34)c3ccccc23)cc1]

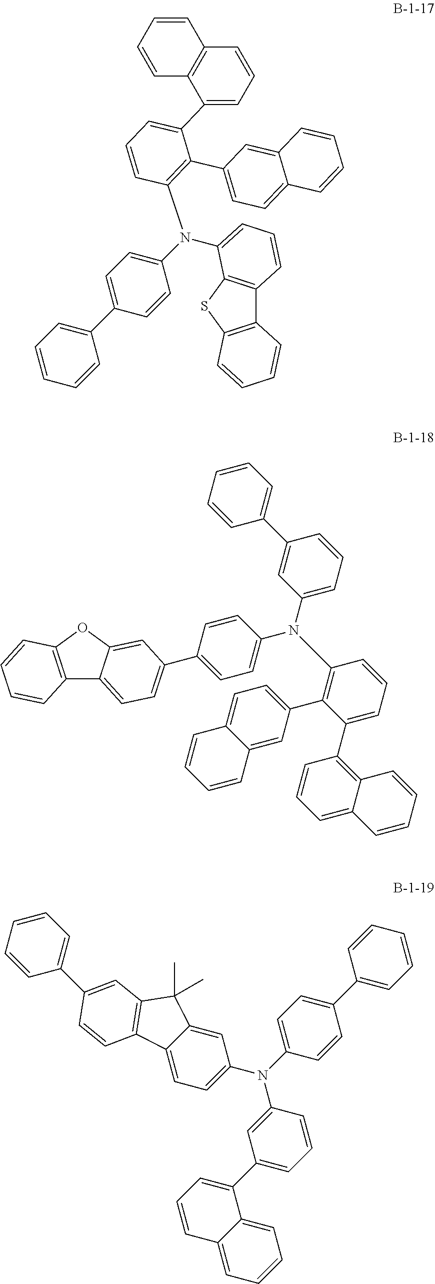 [SMILES: c1ccc(-c2ccc(N(c3cccc(-c4cccc5ccccc45)c3-c3ccc4ccccc4c3)c3cccc4c3sc3ccccc34)cc2)cc1 ; c1ccc(-c2cccc(N(c3ccc(-c4ccc5c(c4)oc4ccccc45)cc3)c3cccc(-c4cccc5ccccc45)c3-c3ccc4ccccc4c3)c2)cc1 ; CC1(C)c2cc(-c3ccccc3)ccc2-c2ccc(N(c3ccc(-c4ccccc4)cc3)c3cccc(-c4cccc5ccccc45)c3)cc21]

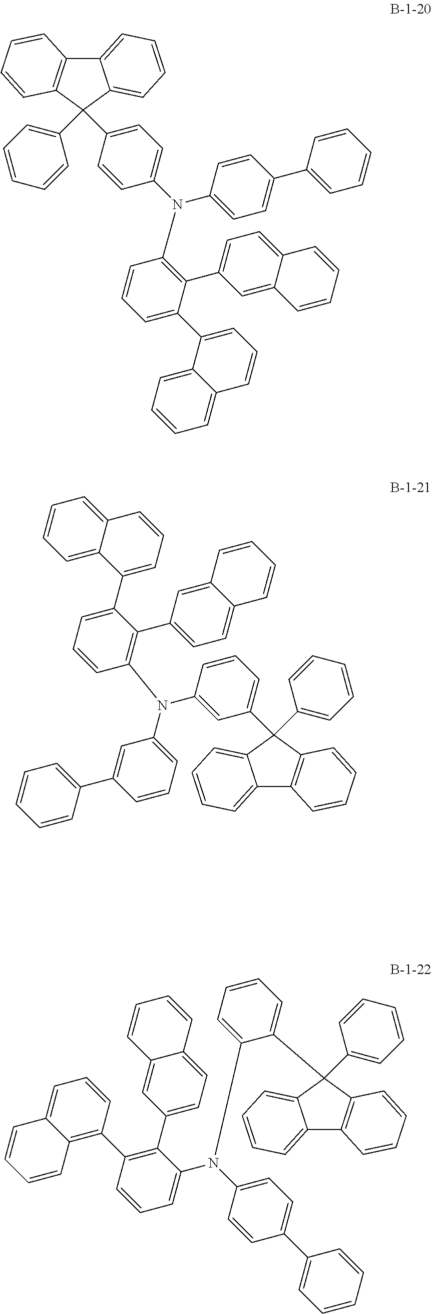 [SMILES: c1ccc(-c2ccc(N(c3ccc(C4(c5ccccc5)c5ccccc5-c5ccccc54)cc3)c3cccc(-c4cccc5ccccc45)c3-c3ccc4ccccc4c3)cc2)cc1 ; c1ccc(-c2cccc(N(c3cccc(C4(c5ccccc5)c5ccccc5-c5ccccc54)c3)c3cccc(-c4cccc5ccccc45)c3-c3ccc4ccccc4c3)c2)cc1 ; c1ccc(-c2ccc(N(c3ccccc3C3(c4ccccc4)c4ccccc4-c4ccccc43)c3cccc(-c4cccc5ccccc45)c3-c3ccc4ccccc4c3)cc2)cc1]

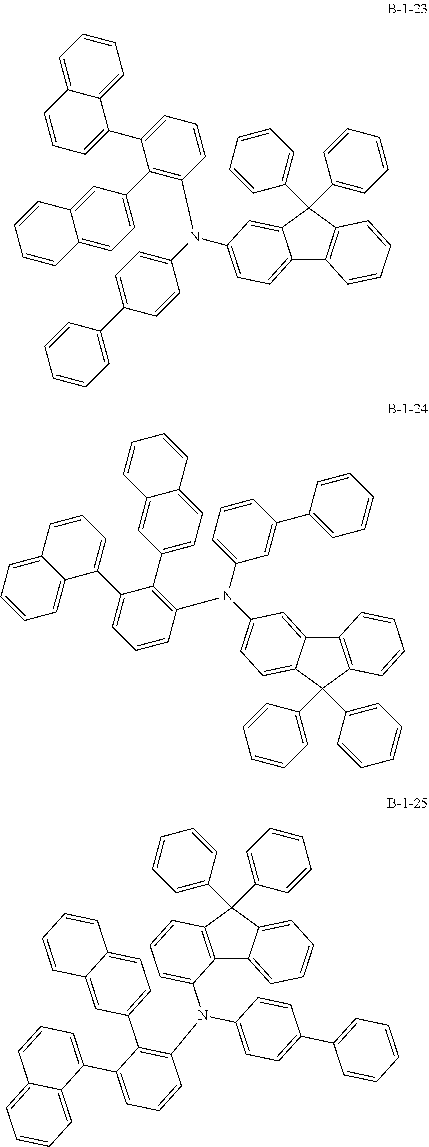 [SMILES: c1ccc(-c2ccc(N(c3ccc4c(c3)C(c3ccccc3)(c3ccccc3)c3ccccc3-4)c3cccc(-c4cccc5ccccc45)c3-c3ccc4ccccc4c3)cc2)cc1 ; c1ccc(-c2cccc(N(c3ccc4c(c3)-c3ccccc3C4(c3ccccc3)c3ccccc3)c3cccc(-c4cccc5ccccc45)c3-c3ccc4ccccc4c3)c2)cc1 ; c1ccc(-c2ccc(N(c3cccc(-c4cccc5ccccc45)c3-c3ccc4ccccc4c3)c3cccc4c3-c3ccccc3C4(c3ccccc3)c3ccccc3)cc2)cc1]

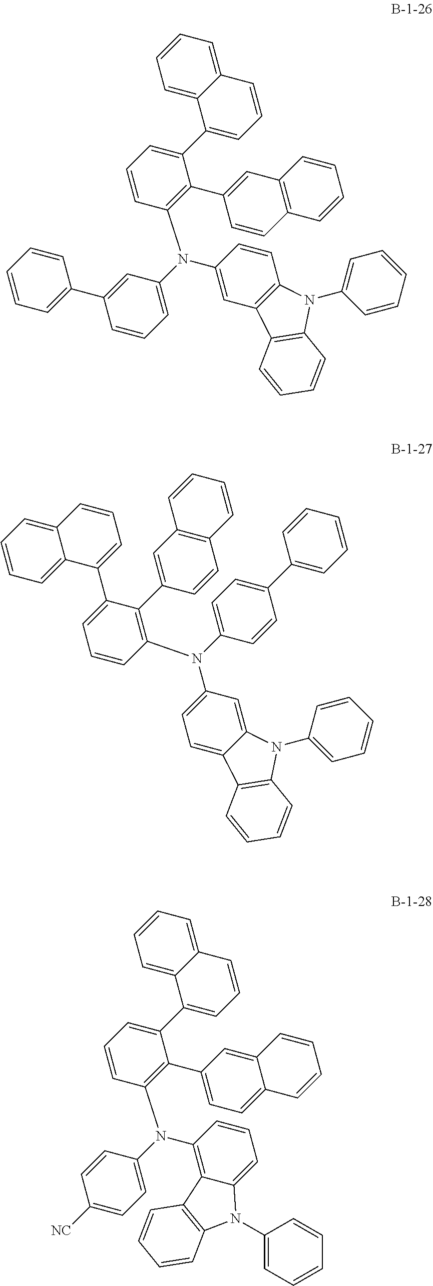 [SMILES: c1ccc(-c2cccc(N(c3ccc4c(c3)c3ccccc3n4-c3ccccc3)c3cccc(-c4cccc5ccccc45)c3-c3ccc4ccccc4c3)c2)cc1 ; c1ccc(-c2ccc(N(c3ccc4c5ccccc5n(-c5ccccc5)c4c3)c3cccc(-c4cccc5ccccc45)c3-c3ccc4ccccc4c3)cc2)cc1 ; N#Cc1ccc(N(c2cccc(-c3cccc4ccccc34)c2-c2ccc3ccccc3c2)c2cccc3c2c2ccccc2n3-c2ccccc2)cc1]

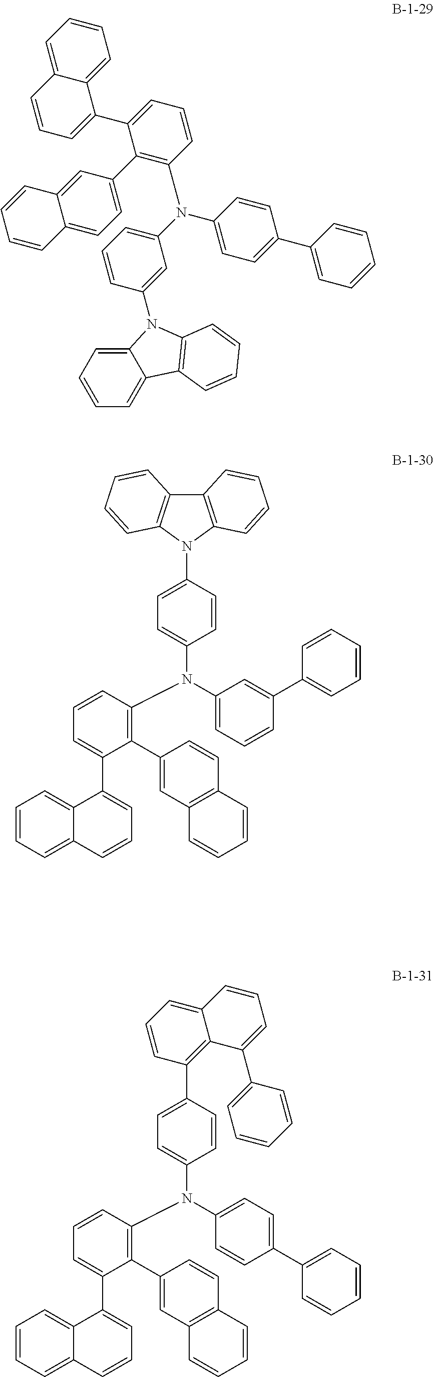 [SMILES: c1ccc(-c2ccc(N(c3cccc(-n4c5ccccc5c5ccccc54)c3)c3cccc(-c4cccc5ccccc45)c3-c3ccc4ccccc4c3)cc2)cc1 ; c1ccc(-c2cccc(N(c3ccc(-n4c5ccccc5c5ccccc54)cc3)c3cccc(-c4cccc5ccccc45)c3-c3ccc4ccccc4c3)c2)cc1 ; c1ccc(-c2ccc(N(c3ccc(-c4cccc5cccc(-c6ccccc6)c45)cc3)c3cccc(-c4cccc5ccccc45)c3-c3ccc4ccccc4c3)cc2)cc1]

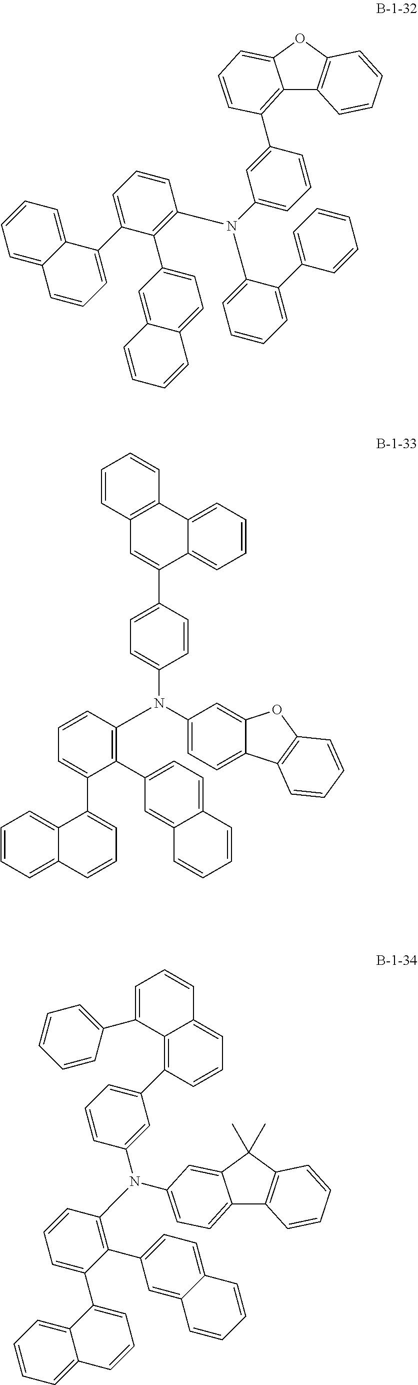 [SMILES: c1ccc(-c2ccccc2N(c2cccc(-c3cccc4oc5ccccc5c34)c2)c2cccc(-c3cccc4ccccc34)c2-c2ccc3ccccc3c2)cc1 ; c1cc(-c2cccc3ccccc23)c(-c2ccc3ccccc3c2)c(N(c2ccc(-c3cc4ccccc4c4ccccc34)cc2)c2ccc3c(c2)oc2ccccc23)c1 ; CC1(C)c2ccccc2-c2ccc(N(c3cccc(-c4cccc5cccc(-c6ccccc6)c45)c3)c3cccc(-c4cccc5ccccc45)c3-c3ccc4ccccc4c3)cc21]

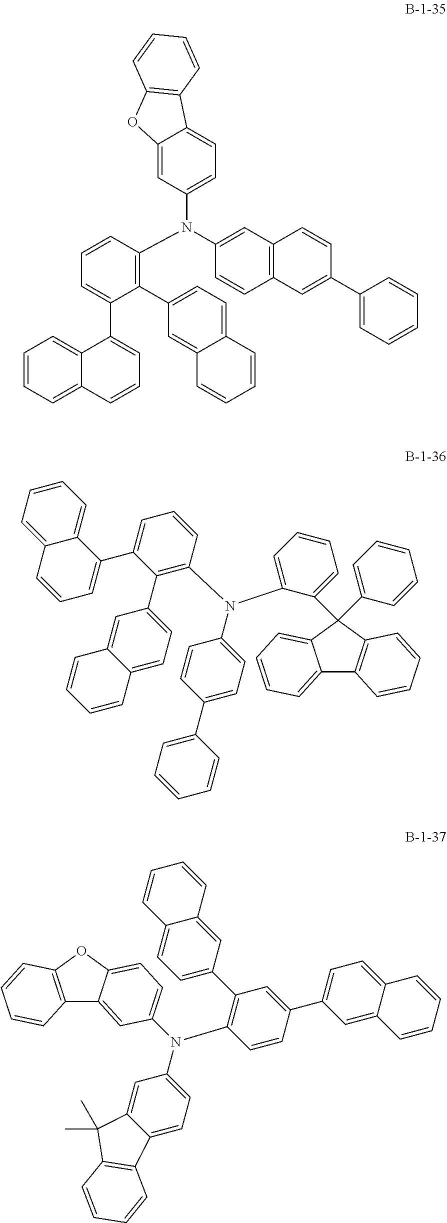 [SMILES: c1ccc(-c2ccc3cc(N(c4ccc5c(c4)oc4ccccc45)c4cccc(-c5cccc6ccccc56)c4-c4ccc5ccccc5c4)ccc3c2)cc1 ; c1ccc(-c2ccc(N(c3ccccc3C3(c4ccccc4)c4ccccc4-c4ccccc43)c3cccc(-c4cccc5ccccc45)c3-c3ccc4ccccc4c3)cc2)cc1 ; CC1(C)c2ccccc2-c2ccc(N(c3ccc4oc5ccccc5c4c3)c3ccc(-c4ccc5ccccc5c4)cc3-c3ccc4ccccc4c3)cc21]

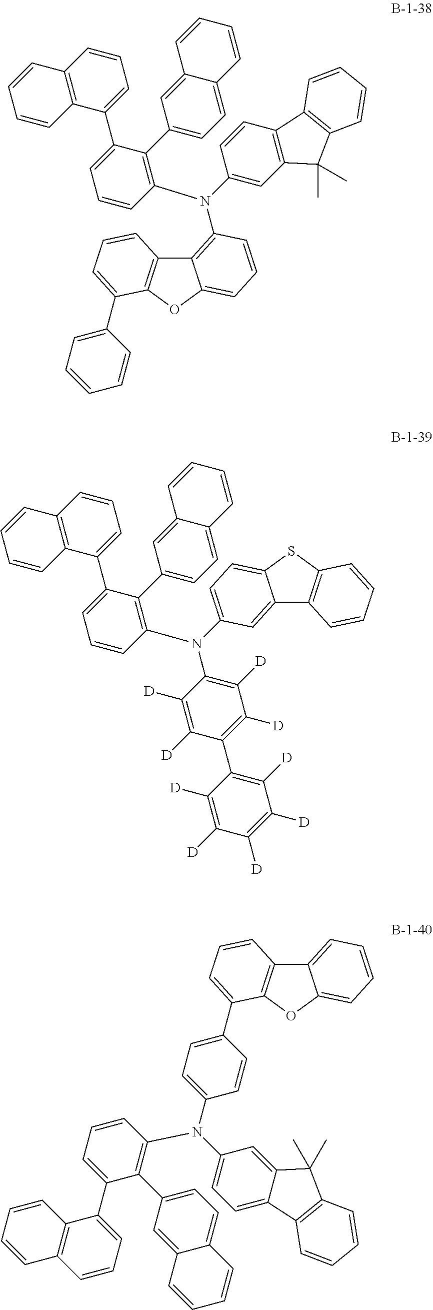 [SMILES: CC1(C)c2ccccc2-c2ccc(N(c3cccc(-c4cccc5ccccc45)c3-c3ccc4ccccc4c3)c3cccc4oc5c(-c6ccccc6)cccc5c34)cc21 ; [2H]c1c([2H])c([2H])c(-c2c([2H])c([2H])c(N(c3ccc4sc5ccccc5c4c3)c3cccc(-c4cccc5ccccc45)c3-c3ccc4ccccc4c3)c([2H])c2[2H])c([2H])c1[2H] ; CC1(C)c2ccccc2-c2ccc(N(c3ccc(-c4cccc5c4oc4ccccc45)cc3)c3cccc(-c4cccc5ccccc45)c3-c3ccc4ccccc4c3)cc21]

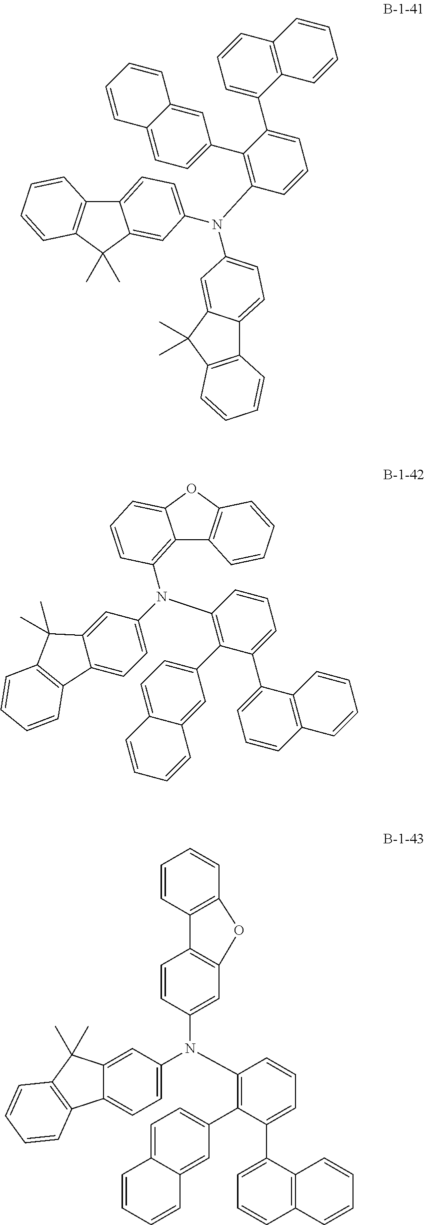 [SMILES: CC1(C)c2ccccc2-c2ccc(N(c3ccc4c(c3)C(C)(C)c3ccccc3-4)c3cccc(-c4cccc5ccccc45)c3-c3ccc4ccccc4c3)cc21 ; CC1(C)c2ccccc2-c2ccc(N(c3cccc(-c4cccc5ccccc45)c3-c3ccc4ccccc4c3)c3cccc4oc5ccccc5c34)cc21 ; CC1(C)c2ccccc2-c2ccc(N(c3ccc4c(c3)oc3ccccc34)c3cccc(-c4cccc5ccccc45)c3-c3ccc4ccccc4c3)cc21]

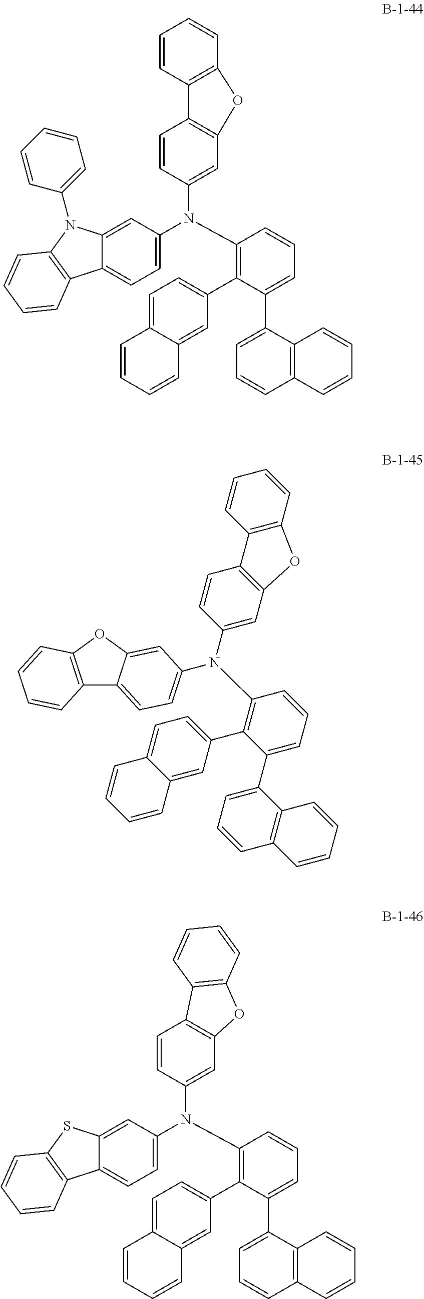 [SMILES: c1ccc(-n2c3ccccc3c3ccc(N(c4ccc5c(c4)oc4ccccc45)c4cccc(-c5cccc6ccccc56)c4-c4ccc5ccccc5c4)cc32)cc1 ; c1cc(-c2cccc3ccccc23)c(-c2ccc3ccccc3c2)c(N(c2ccc3c(c2)oc2ccccc23)c2ccc3c(c2)oc2ccccc23)c1 ; c1cc(-c2cccc3ccccc23)c(-c2ccc3ccccc3c2)c(N(c2ccc3c(c2)oc2ccccc23)c2ccc3c(c2)sc2ccccc23)c1]

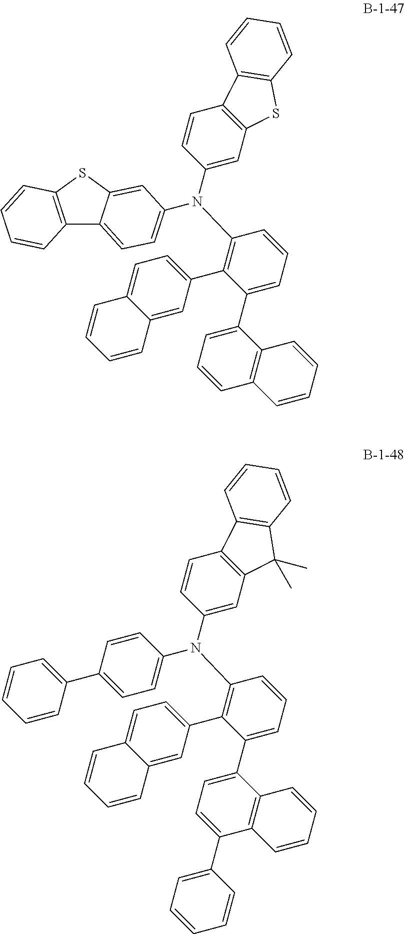 [SMILES: c1cc(-c2cccc3ccccc23)c(-c2ccc3ccccc3c2)c(N(c2ccc3c(c2)sc2ccccc23)c2ccc3c(c2)sc2ccccc23)c1 ; CC1(C)c2ccccc2-c2ccc(N(c3ccc(-c4ccccc4)cc3)c3cccc(-c4ccc(-c5ccccc5)c5ccccc45)c3-c3ccc4ccccc4c3)cc21]

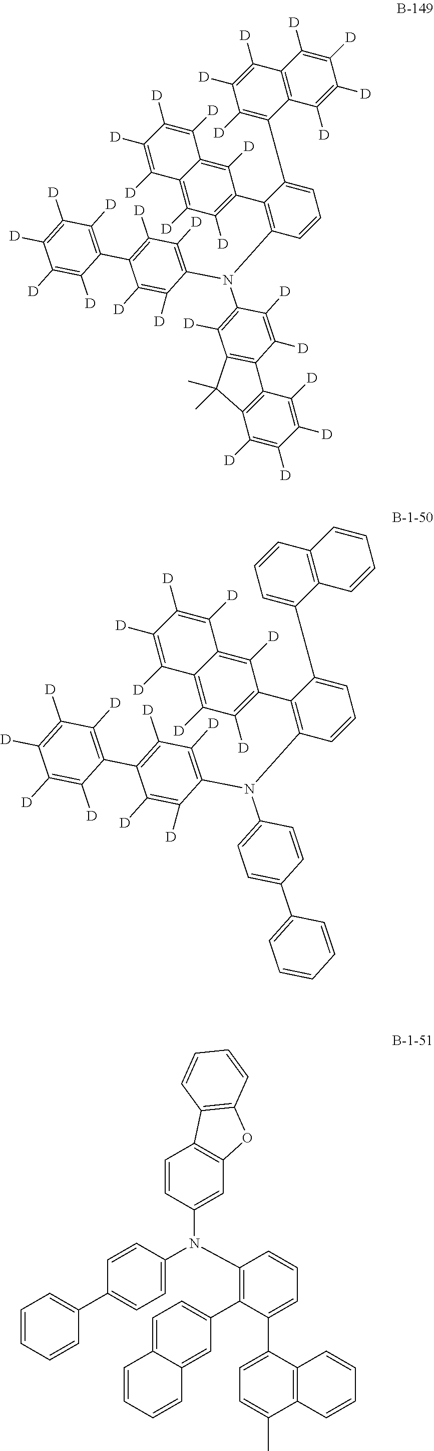

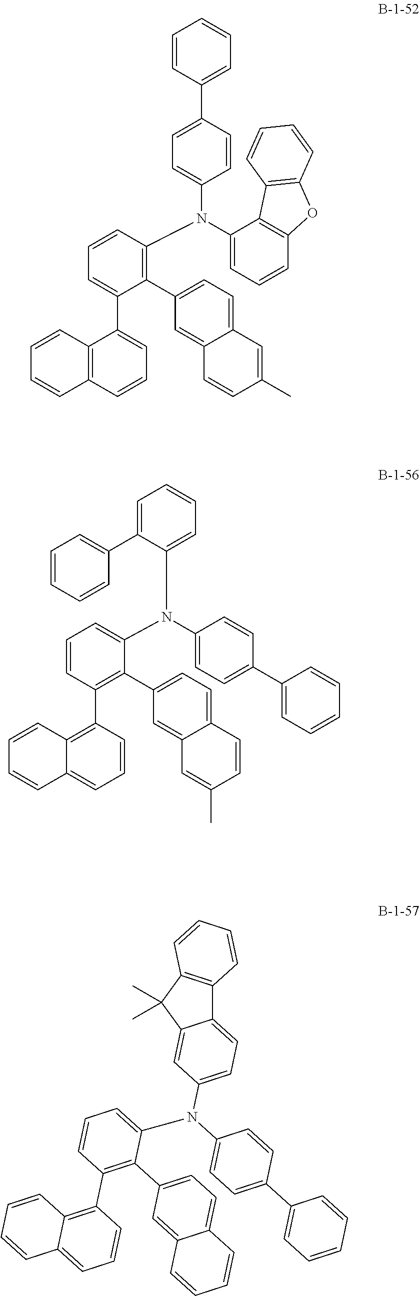 [SMILES: Cc1ccc2cc(-c3c(-c4cccc5ccccc45)cccc3N(c3ccc(-c4ccccc4)cc3)c3cccc4oc5ccccc5c34)ccc2c1 ; Cc1ccc2ccc(-c3c(-c4cccc5ccccc45)cccc3N(c3ccc(-c4ccccc4)cc3)c3ccccc3-c3ccccc3)cc2c1 ; CC1(C)c2ccccc2-c2ccc(N(c3ccc(-c4ccccc4)cc3)c3cccc(-c4cccc5ccccc45)c3-c3ccc4ccccc4c3)cc21]

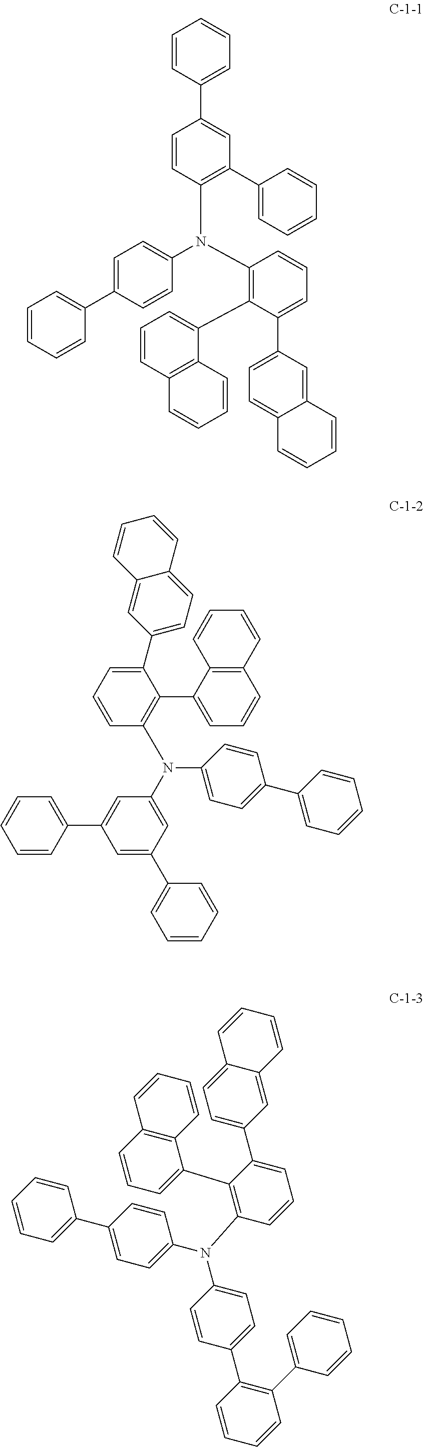 [SMILES: c1ccc(-c2ccc(N(c3ccc(-c4ccccc4)cc3-c3ccccc3)c3cccc(-c4ccc5ccccc5c4)c3-c3cccc4ccccc34)cc2)cc1 ; c1ccc(-c2ccc(N(c3cc(-c4ccccc4)cc(-c4ccccc4)c3)c3cccc(-c4ccc5ccccc5c4)c3-c3cccc4ccccc34)cc2)cc1 ; c1ccc(-c2ccc(N(c3ccc(-c4ccccc4-c4ccccc4)cc3)c3cccc(-c4ccc5ccccc5c4)c3-c3cccc4ccccc34)cc2)cc1]

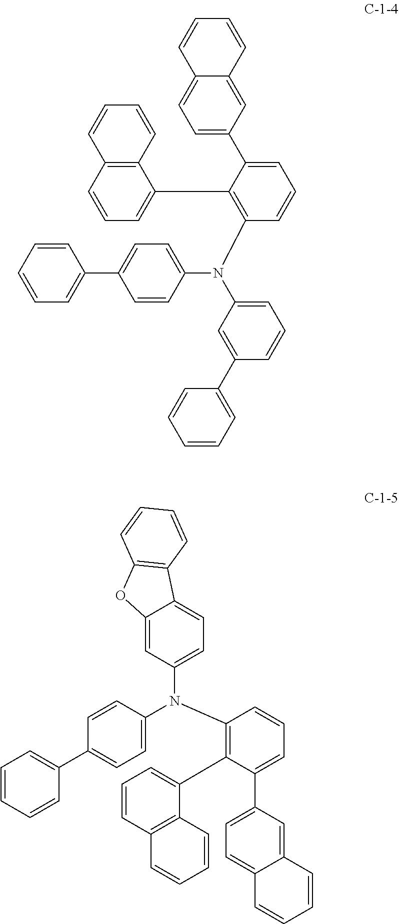 [SMILES: c1ccc(-c2ccc(N(c3cccc(-c4ccccc4)c3)c3cccc(-c4ccc5ccccc5c4)c3-c3cccc4ccccc34)cc2)cc1 ; c1ccc(-c2ccc(N(c3ccc4c(c3)oc3ccccc34)c3cccc(-c4ccc5ccccc5c4)c3-c3cccc4ccccc34)cc2)cc1]

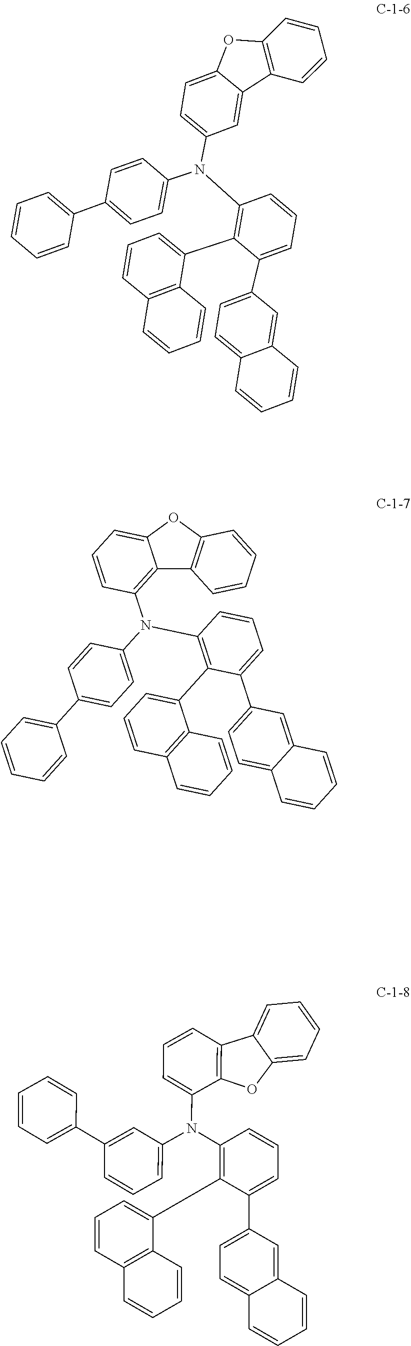 [SMILES: c1ccc(-c2ccc(N(c3ccc4oc5ccccc5c4c3)c3cccc(-c4ccc5ccccc5c4)c3-c3cccc4ccccc34)cc2)cc1 ; c1ccc(-c2ccc(N(c3cccc(-c4ccc5ccccc5c4)c3-c3cccc4ccccc34)c3cccc4oc5ccccc5c34)cc2)cc1 ; c1ccc(-c2cccc(N(c3cccc(-c4ccc5ccccc5c4)c3-c3cccc4ccccc34)c3cccc4c3oc3ccccc34)c2)cc1]

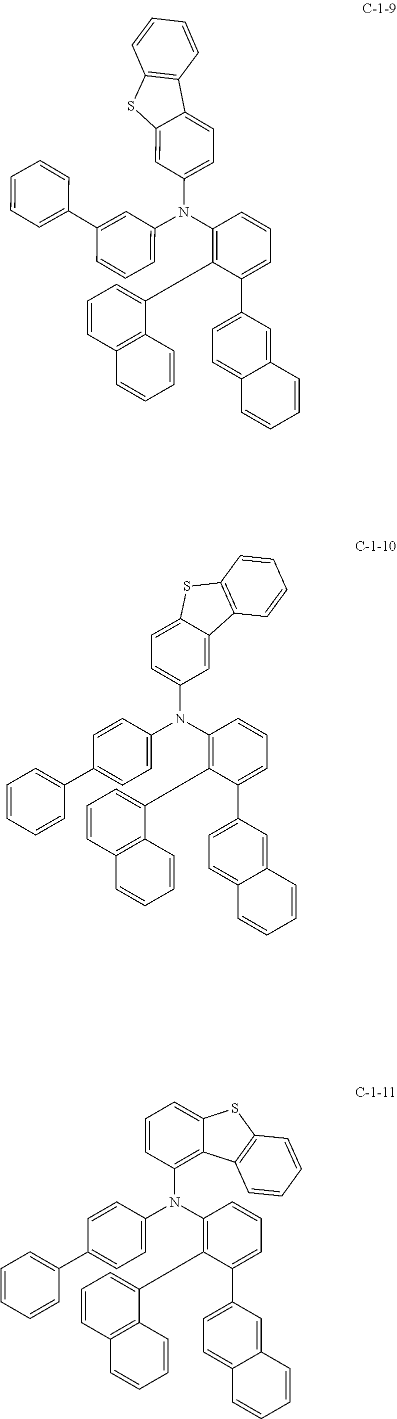 [SMILES: c1ccc(-c2cccc(N(c3ccc4c(c3)sc3ccccc34)c3cccc(-c4ccc5ccccc5c4)c3-c3cccc4ccccc34)c2)cc1 ; c1ccc(-c2ccc(N(c3ccc4sc5ccccc5c4c3)c3cccc(-c4ccc5ccccc5c4)c3-c3cccc4ccccc34)cc2)cc1 ; c1ccc(-c2ccc(N(c3cccc(-c4ccc5ccccc5c4)c3-c3cccc4ccccc34)c3cccc4sc5ccccc5c34)cc2)cc1]

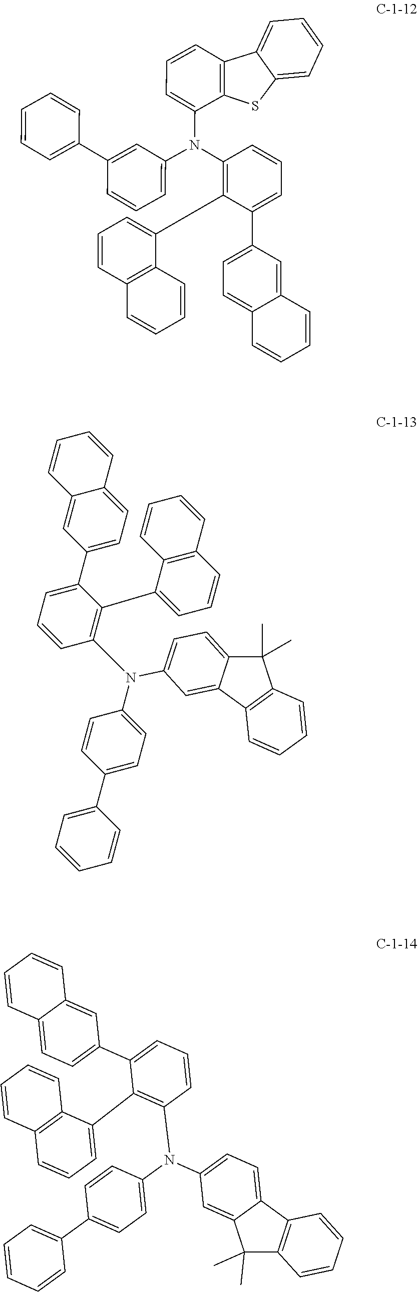 [SMILES: c1ccc(-c2cccc(N(c3cccc(-c4ccc5ccccc5c4)c3-c3cccc4ccccc34)c3cccc4c3sc3ccccc34)c2)cc1 ; CC1(C)c2ccccc2-c2cc(N(c3ccc(-c4ccccc4)cc3)c3cccc(-c4ccc5ccccc5c4)c3-c3cccc4ccccc34)ccc21 ; CC1(C)c2ccccc2-c2ccc(N(c3ccc(-c4ccccc4)cc3)c3cccc(-c4ccc5ccccc5c4)c3-c3cccc4ccccc34)cc21]

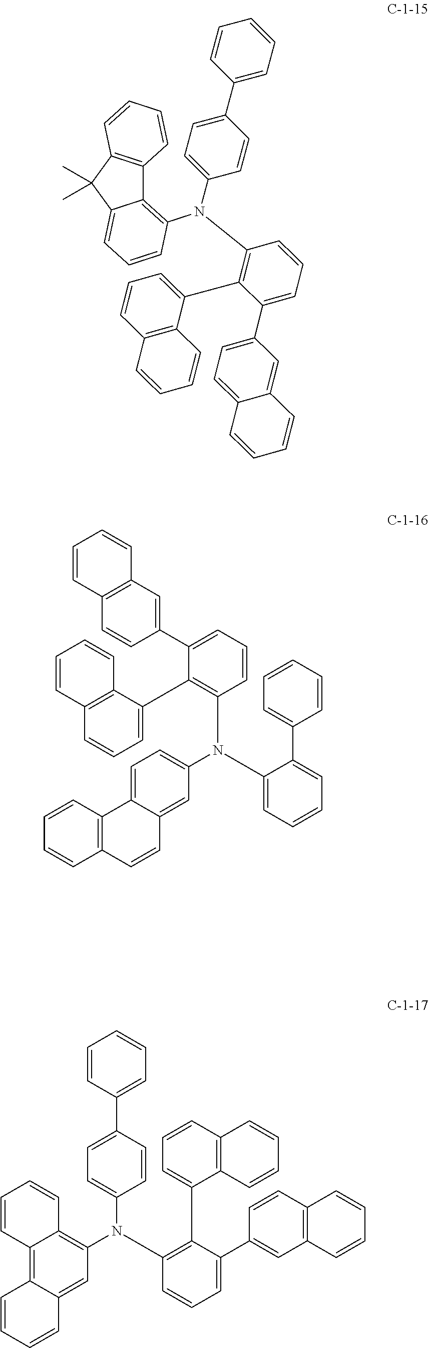 [SMILES: CC1(C)c2ccccc2-c2c(N(c3ccc(-c4ccccc4)cc3)c3cccc(-c4ccc5ccccc5c4)c3-c3cccc4ccccc34)cccc21 ; c1ccc(-c2ccccc2N(c2ccc3c(ccc4ccccc43)c2)c2cccc(-c3ccc4ccccc4c3)c2-c2cccc3ccccc23)cc1 ; c1ccc(-c2ccc(N(c3cccc(-c4ccc5ccccc5c4)c3-c3cccc4ccccc34)c3cc4ccccc4c4ccccc34)cc2)cc1]

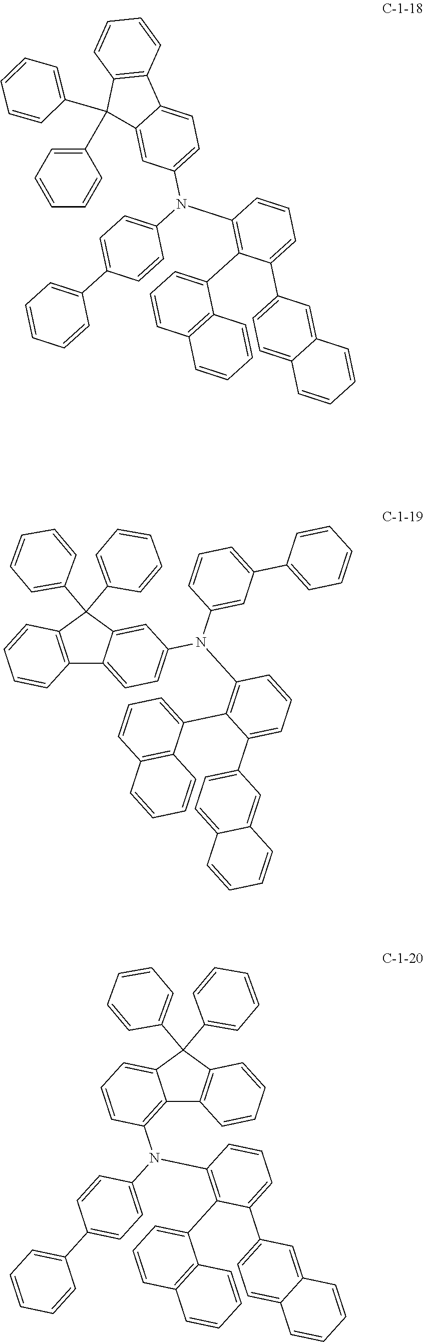 [SMILES: c1ccc(-c2ccc(N(c3ccc4c(c3)C(c3ccccc3)(c3ccccc3)c3ccccc3-4)c3cccc(-c4ccc5ccccc5c4)c3-c3cccc4ccccc34)cc2)cc1 ; c1ccc(-c2cccc(N(c3ccc4c(c3)C(c3ccccc3)(c3ccccc3)c3ccccc3-4)c3cccc(-c4ccc5ccccc5c4)c3-c3cccc4ccccc34)c2)cc1 ; c1ccc(-c2ccc(N(c3cccc(-c4ccc5ccccc5c4)c3-c3cccc4ccccc34)c3cccc4c3-c3ccccc3C4(c3ccccc3)c3ccccc3)cc2)cc1]

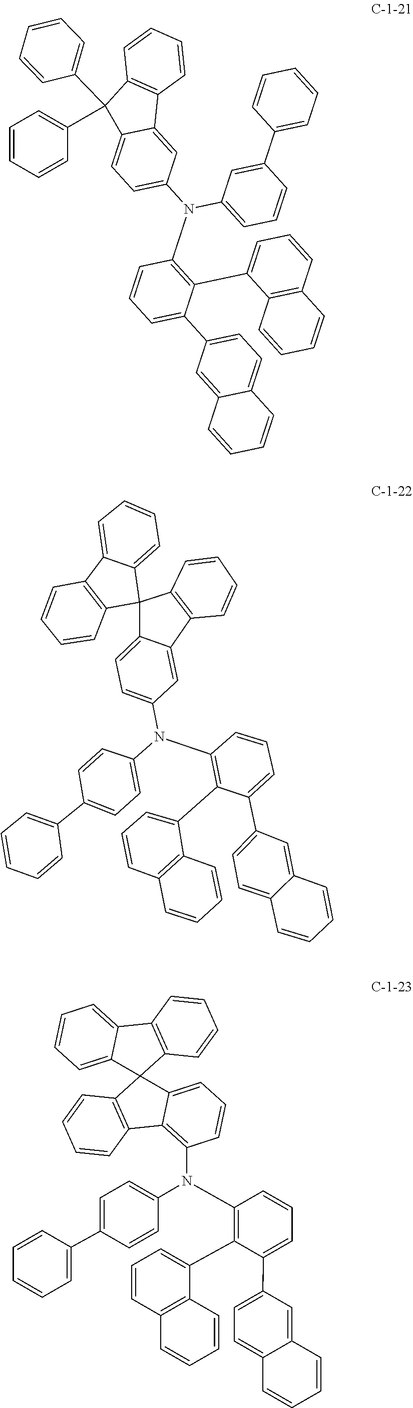 [SMILES: c1ccc(-c2cccc(N(c3ccc4c(c3)-c3ccccc3C4(c3ccccc3)c3ccccc3)c3cccc(-c4ccc5ccccc5c4)c3-c3cccc4ccccc34)c2)cc1 ; c1ccc(-c2ccc(N(c3ccc4c(c3)-c3ccccc3C43c4ccccc4-c4ccccc43)c3cccc(-c4ccc5ccccc5c4)c3-c3cccc4ccccc34)cc2)cc1 ; c1ccc(-c2ccc(N(c3cccc(-c4ccc5ccccc5c4)c3-c3cccc4ccccc34)c3cccc4c3-c3ccccc3C43c4ccccc4-c4ccccc43)cc2)cc1]

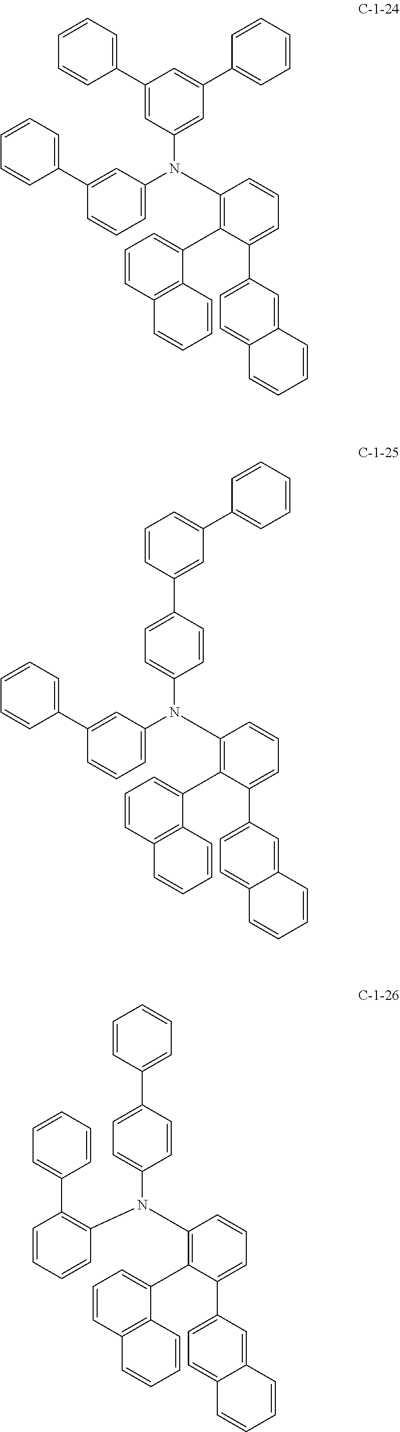 [SMILES: c1ccc(-c2cccc(N(c3cc(-c4ccccc4)cc(-c4ccccc4)c3)c3cccc(-c4ccc5ccccc5c4)c3-c3cccc4ccccc34)c2)cc1 ; c1ccc(-c2cccc(-c3ccc(N(c4cccc(-c5ccccc5)c4)c4cccc(-c5ccc6ccccc6c5)c4-c4cccc5ccccc45)cc3)c2)cc1 ; c1ccc(-c2ccc(N(c3ccccc3-c3ccccc3)c3cccc(-c4ccc5ccccc5c4)c3-c3cccc4ccccc34)cc2)cc1]

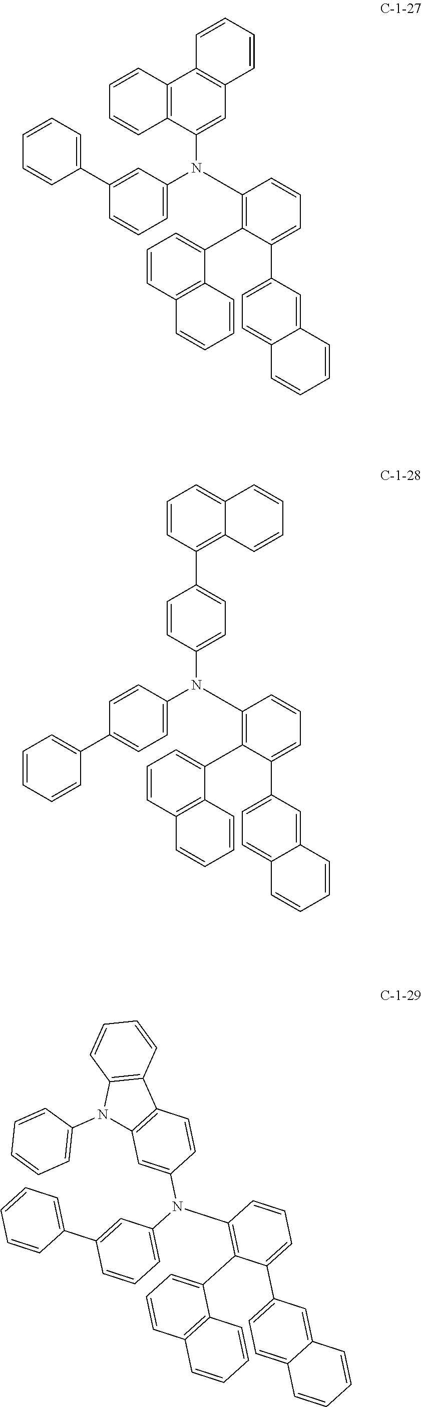 [SMILES: c1ccc(-c2cccc(N(c3cccc(-c4ccc5ccccc5c4)c3-c3cccc4ccccc34)c3cc4ccccc4c4ccccc34)c2)cc1 ; c1ccc(-c2ccc(N(c3ccc(-c4cccc5ccccc45)cc3)c3cccc(-c4ccc5ccccc5c4)c3-c3cccc4ccccc34)cc2)cc1 ; c1ccc(-c2cccc(N(c3ccc4c5ccccc5n(-c5ccccc5)c4c3)c3cccc(-c4ccc5ccccc5c4)c3-c3cccc4ccccc34)c2)cc1]

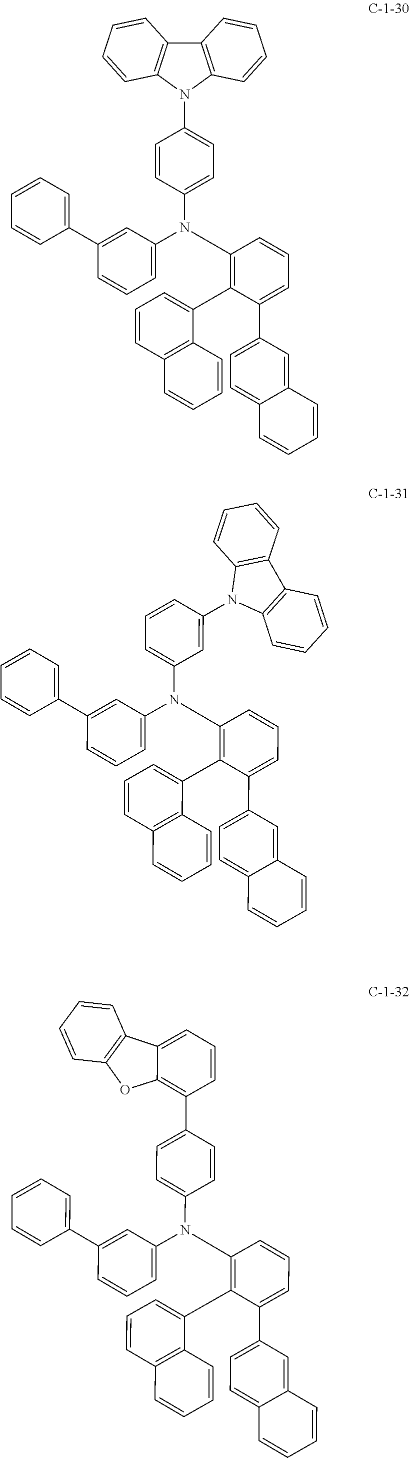 [SMILES: c1ccc(-c2cccc(N(c3ccc(-n4c5ccccc5c5ccccc54)cc3)c3cccc(-c4ccc5ccccc5c4)c3-c3cccc4ccccc34)c2)cc1 ; c1ccc(-c2cccc(N(c3cccc(-n4c5ccccc5c5ccccc54)c3)c3cccc(-c4ccc5ccccc5c4)c3-c3cccc4ccccc34)c2)cc1 ; c1ccc(-c2cccc(N(c3ccc(-c4cccc5c4oc4ccccc45)cc3)c3cccc(-c4ccc5ccccc5c4)c3-c3cccc4ccccc34)c2)cc1]

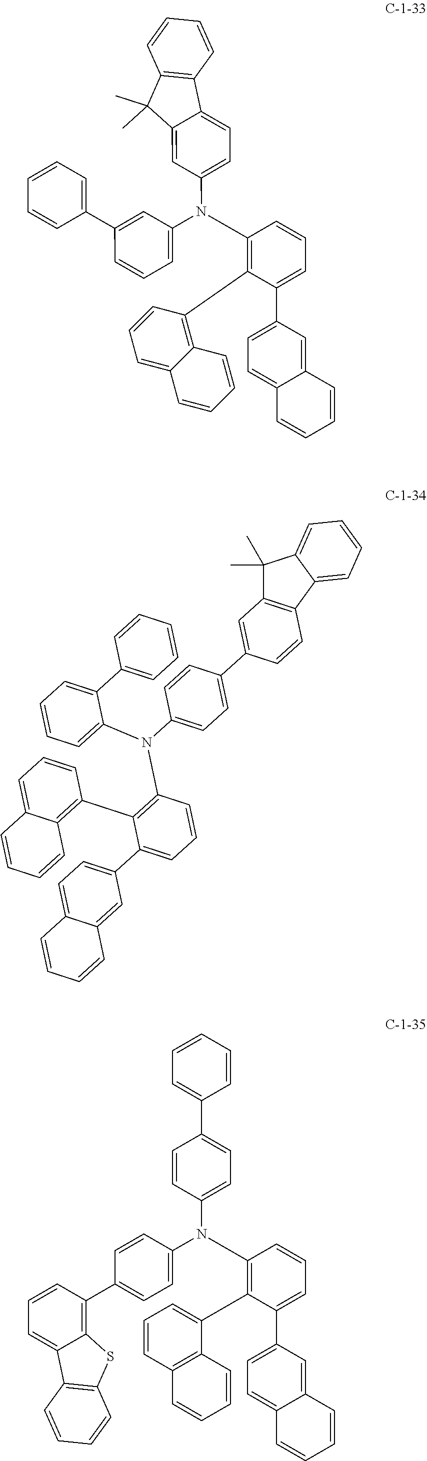 [SMILES: CC1(C)c2ccccc2-c2ccc(N(c3cccc(-c4ccccc4)c3)c3cccc(-c4ccc5ccccc5c4)c3-c3cccc4ccccc34)cc21 ; CC1(C)c2ccccc2-c2ccc(-c3ccc(N(c4ccccc4-c4ccccc4)c4cccc(-c5ccc6ccccc6c5)c4-c4cccc5ccccc45)cc3)cc21 ; c1ccc(-c2ccc(N(c3ccc(-c4cccc5c4sc4ccccc45)cc3)c3cccc(-c4ccc5ccccc5c4)c3-c3cccc4ccccc34)cc2)cc1]

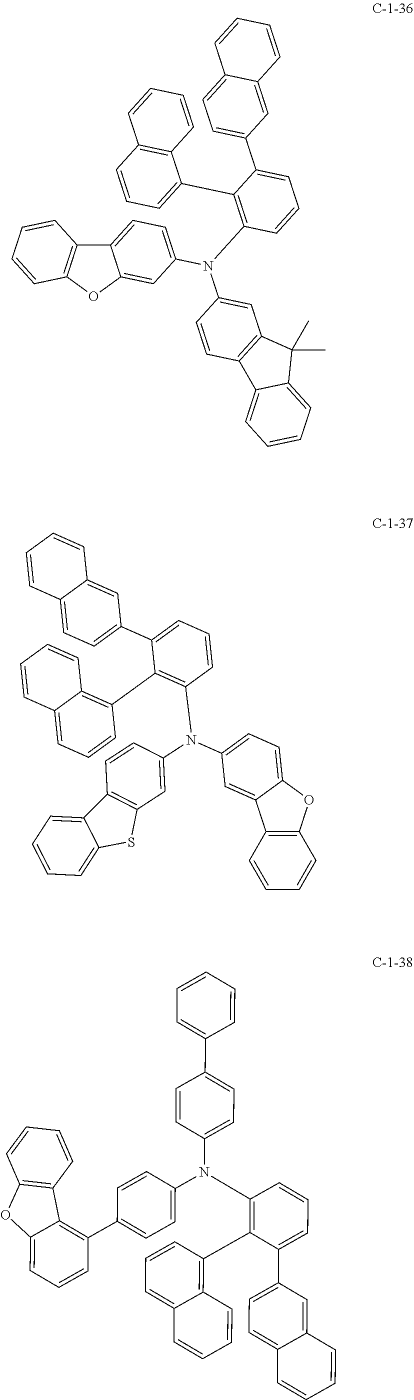 [SMILES: CC1(C)c2ccccc2-c2ccc(N(c3ccc4c(c3)oc3ccccc34)c3cccc(-c4ccc5ccccc5c4)c3-c3cccc4ccccc34)cc21 ; c1cc(-c2ccc3ccccc3c2)c(-c2cccc3ccccc23)c(N(c2ccc3c(c2)sc2ccccc23)c2ccc3oc4ccccc4c3c2)c1 ; c1ccc(-c2ccc(N(c3ccc(-c4cccc5oc6ccccc6c45)cc3)c3cccc(-c4ccc5ccccc5c4)c3-c3cccc4ccccc34)cc2)cc1]

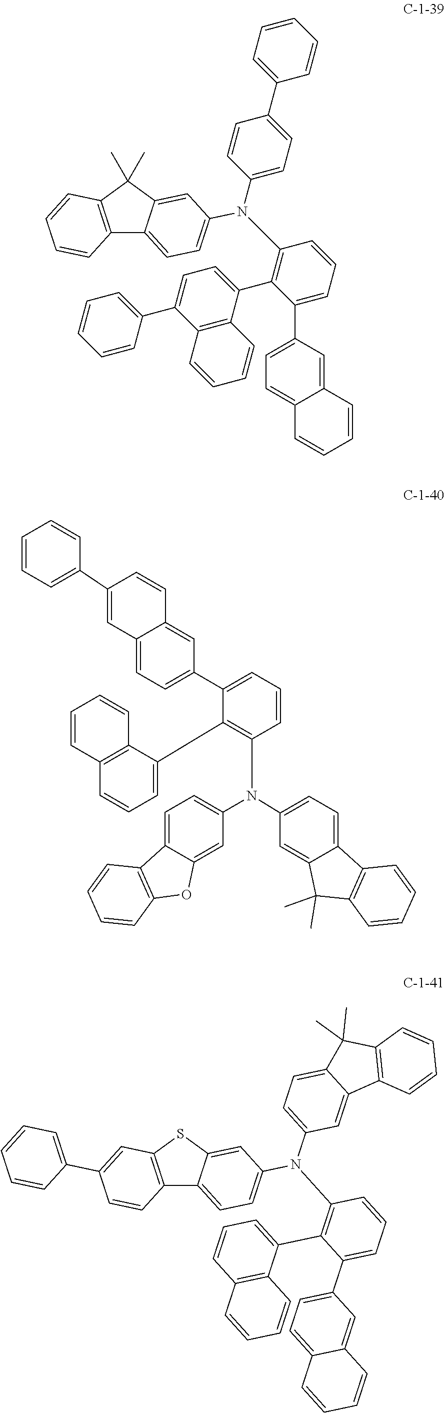 [SMILES: CC1(C)c2ccccc2-c2ccc(N(c3ccc(-c4ccccc4)cc3)c3cccc(-c4ccc5ccccc5c4)c3-c3ccc(-c4ccccc4)c4ccccc34)cc21 ; CC1(C)c2ccccc2-c2ccc(N(c3ccc4c(c3)oc3ccccc34)c3cccc(-c4ccc5cc(-c6ccccc6)ccc5c4)c3-c3cccc4ccccc34)cc21 ; CC1(C)c2ccccc2-c2cc(N(c3ccc4c(c3)sc3cc(-c5ccccc5)ccc34)c3cccc(-c4ccc5ccccc5c4)c3-c3cccc4ccccc34)ccc21]

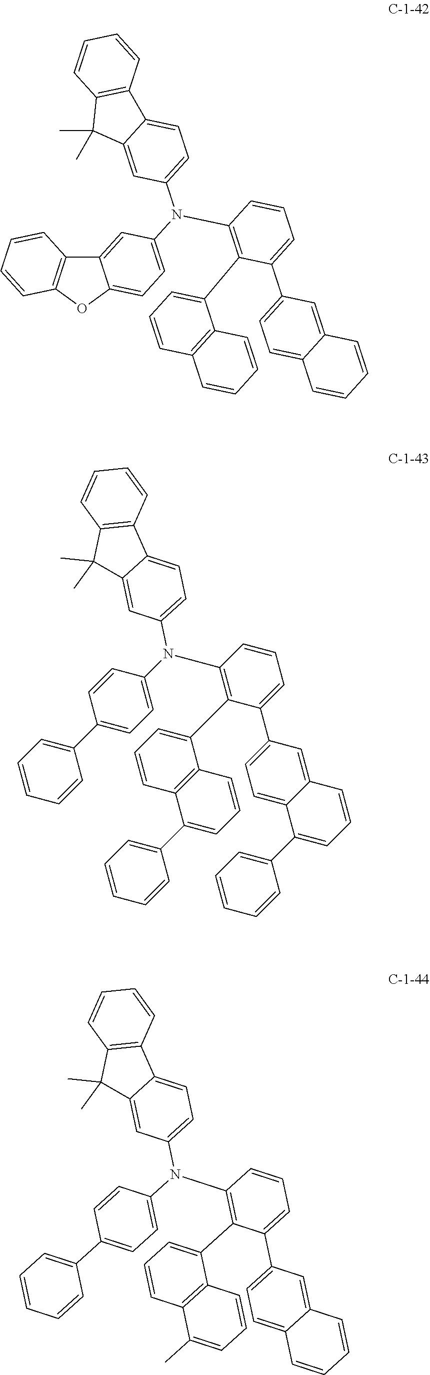 [SMILES: CC1(C)c2ccccc2-c2ccc(N(c3ccc4oc5ccccc5c4c3)c3cccc(-c4ccc5ccccc5c4)c3-c3cccc4ccccc34)cc21 ; CC1(C)c2ccccc2-c2ccc(N(c3ccc(-c4ccccc4)cc3)c3cccc(-c4ccc5c(-c6ccccc6)cccc5c4)c3-c3cccc4c(-c5ccccc5)cccc34)cc21 ; Cc1cccc2c(-c3c(-c4ccc5ccccc5c4)cccc3N(c3ccc(-c4ccccc4)cc3)c3ccc4c(c3)C(C)(C)c3ccccc3-4)cccc12]

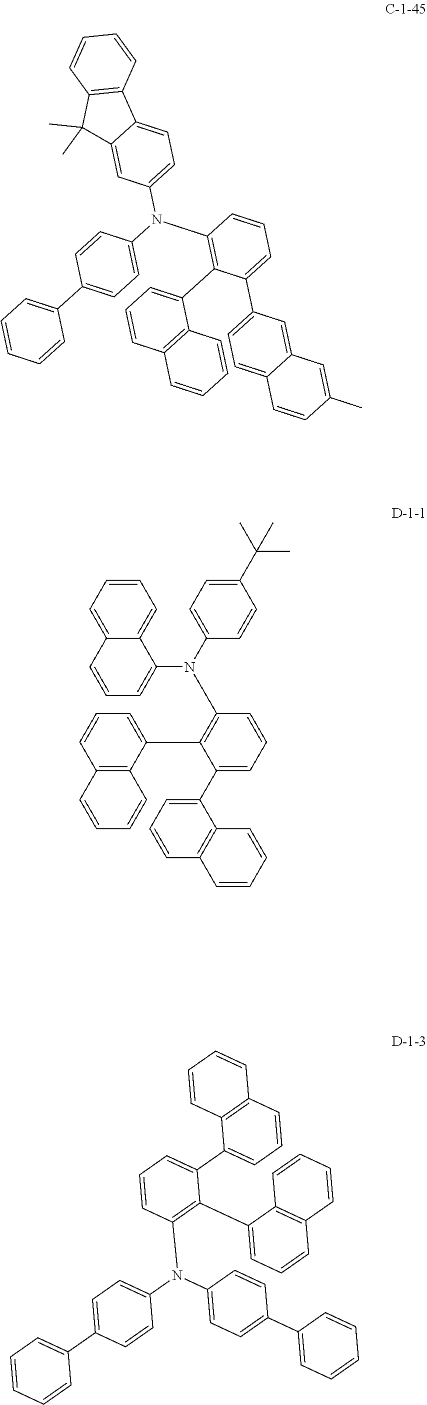 [SMILES: Cc1ccc2ccc(-c3cccc(N(c4ccc(-c5ccccc5)cc4)c4ccc5c(c4)C(C)(C)c4ccccc4-5)c3-c3cccc4ccccc34)cc2c1 ; CC(C)(C)c1ccc(N(c2cccc(-c3cccc4ccccc34)c2-c2cccc3ccccc23)c2cccc3ccccc23)cc1 ; c1ccc(-c2ccc(N(c3ccc(-c4ccccc4)cc3)c3cccc(-c4cccc5ccccc45)c3-c3cccc4ccccc34)cc2)cc1]

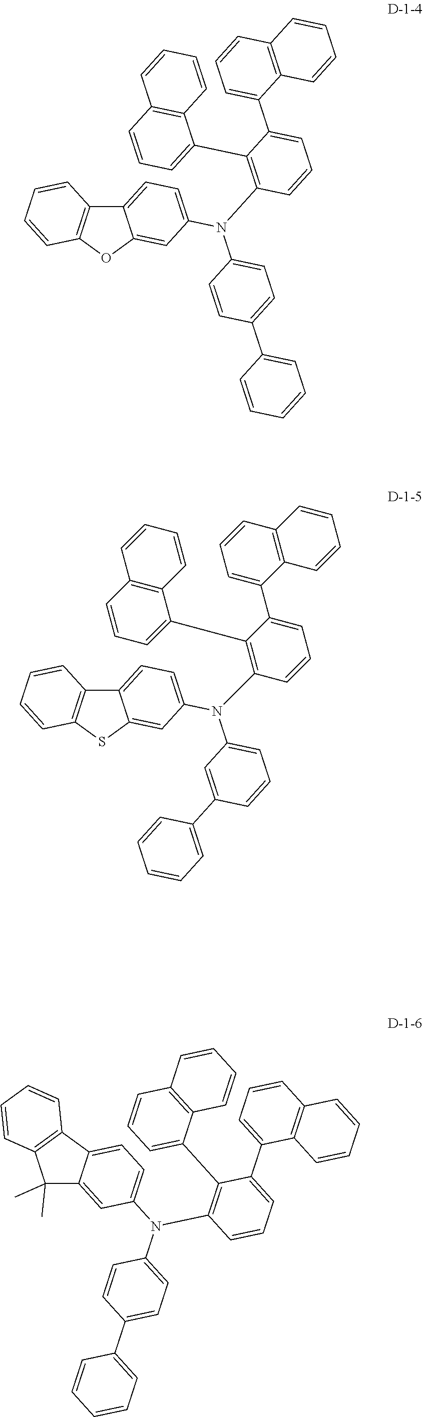 [SMILES: c1ccc(-c2ccc(N(c3ccc4c(c3)oc3ccccc34)c3cccc(-c4cccc5ccccc45)c3-c3cccc4ccccc34)cc2)cc1 ; c1ccc(-c2cccc(N(c3ccc4c(c3)sc3ccccc34)c3cccc(-c4cccc5ccccc45)c3-c3cccc4ccccc34)c2)cc1 ; CC1(C)c2ccccc2-c2ccc(N(c3ccc(-c4ccccc4)cc3)c3cccc(-c4cccc5ccccc45)c3-c3cccc4ccccc34)cc21]

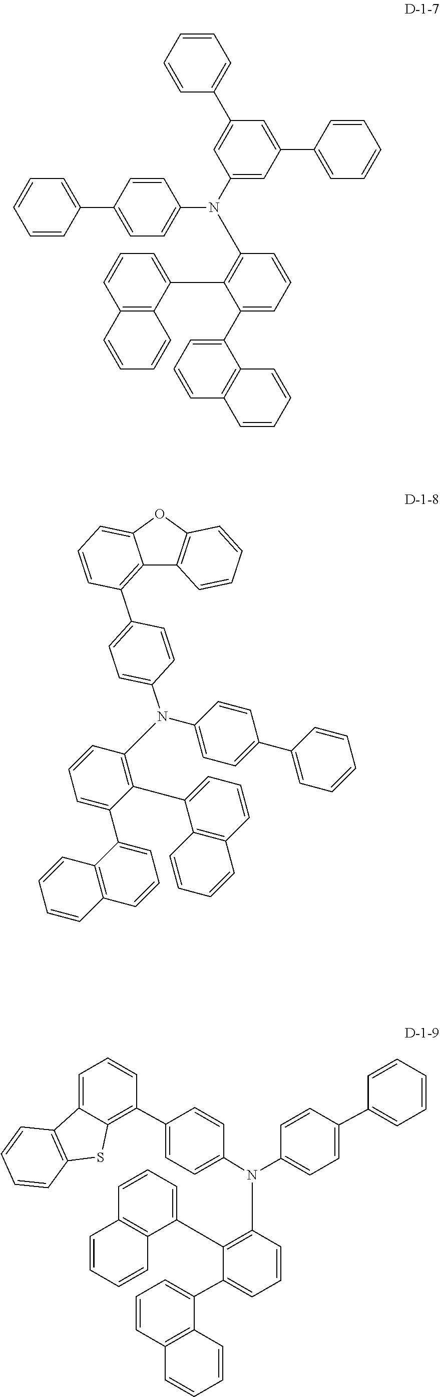 [SMILES: c1ccc(-c2ccc(N(c3cc(-c4ccccc4)cc(-c4ccccc4)c3)c3cccc(-c4cccc5ccccc45)c3-c3cccc4ccccc34)cc2)cc1 ; c1ccc(-c2ccc(N(c3ccc(-c4cccc5oc6ccccc6c45)cc3)c3cccc(-c4cccc5ccccc45)c3-c3cccc4ccccc34)cc2)cc1 ; c1ccc(-c2ccc(N(c3ccc(-c4cccc5c4sc4ccccc45)cc3)c3cccc(-c4cccc5ccccc45)c3-c3cccc4ccccc34)cc2)cc1]

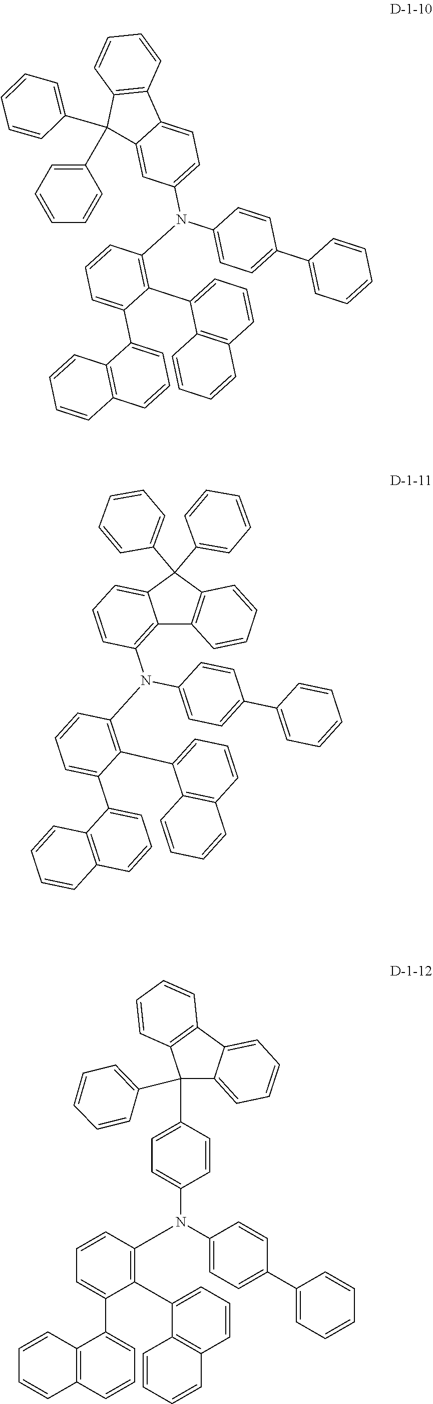 [SMILES: c1ccc(-c2ccc(N(c3ccc4c(c3)C(c3ccccc3)(c3ccccc3)c3ccccc3-4)c3cccc(-c4cccc5ccccc45)c3-c3cccc4ccccc34)cc2)cc1 ; c1ccc(-c2ccc(N(c3cccc(-c4cccc5ccccc45)c3-c3cccc4ccccc34)c3cccc4c3-c3ccccc3C4(c3ccccc3)c3ccccc3)cc2)cc1 ; c1ccc(-c2ccc(N(c3ccc(C4(c5ccccc5)c5ccccc5-c5ccccc54)cc3)c3cccc(-c4cccc5ccccc45)c3-c3cccc4ccccc34)cc2)cc1]

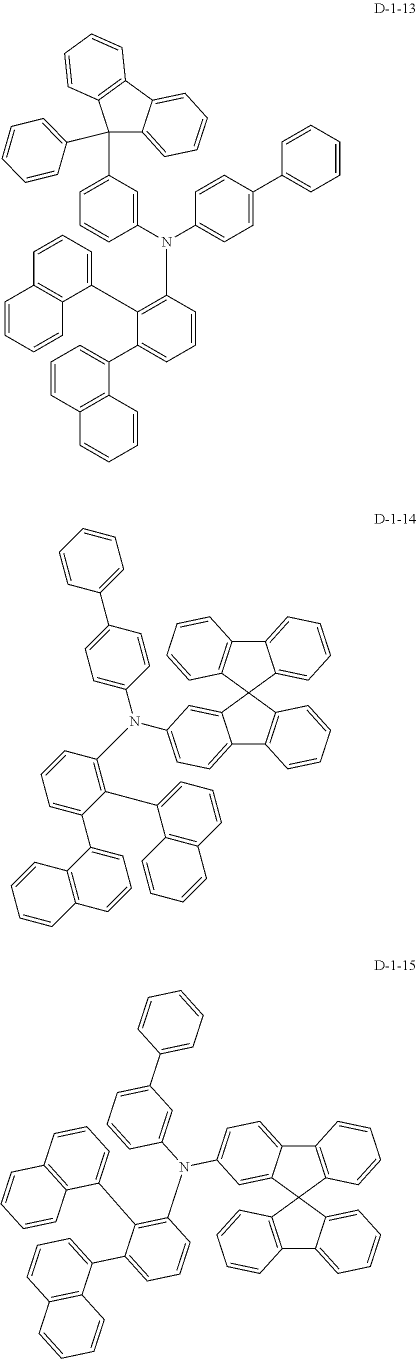 [SMILES: c1ccc(-c2ccc(N(c3cccc(C4(c5ccccc5)c5ccccc5-c5ccccc54)c3)c3cccc(-c4cccc5ccccc45)c3-c3cccc4ccccc34)cc2)cc1 ; c1ccc(-c2ccc(N(c3ccc4c(c3)C3(c5ccccc5-c5ccccc53)c3ccccc3-4)c3cccc(-c4cccc5ccccc45)c3-c3cccc4ccccc34)cc2)cc1 ; c1ccc(-c2cccc(N(c3ccc4c(c3)C3(c5ccccc5-c5ccccc53)c3ccccc3-4)c3cccc(-c4cccc5ccccc45)c3-c3cccc4ccccc34)c2)cc1]

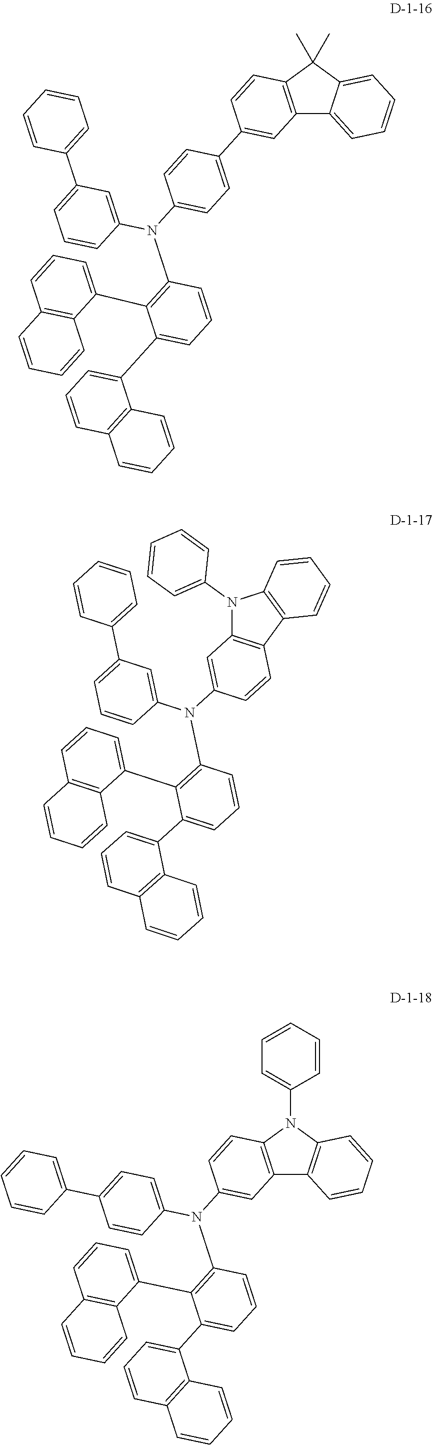 [SMILES: CC1(C)c2ccccc2-c2cc(-c3ccc(N(c4cccc(-c5ccccc5)c4)c4cccc(-c5cccc6ccccc56)c4-c4cccc5ccccc45)cc3)ccc21 ; c1ccc(-c2cccc(N(c3ccc4c5ccccc5n(-c5ccccc5)c4c3)c3cccc(-c4cccc5ccccc45)c3-c3cccc4ccccc34)c2)cc1 ; c1ccc(-c2ccc(N(c3ccc4c(c3)c3ccccc3n4-c3ccccc3)c3cccc(-c4cccc5ccccc45)c3-c3cccc4ccccc34)cc2)cc1]

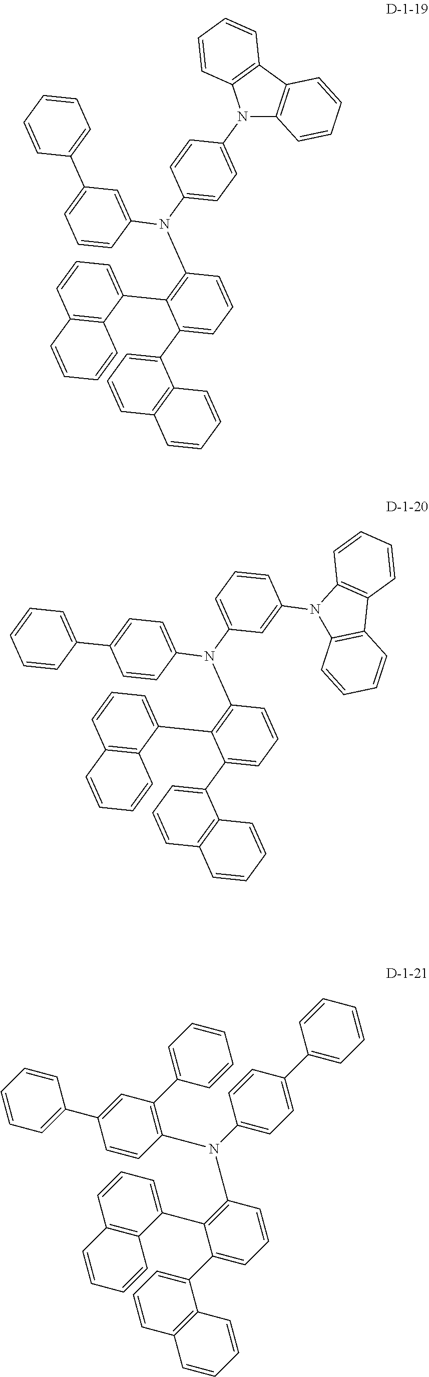 [SMILES: c1ccc(-c2cccc(N(c3ccc(-n4c5ccccc5c5ccccc54)cc3)c3cccc(-c4cccc5ccccc45)c3-c3cccc4ccccc34)c2)cc1 ; c1ccc(-c2ccc(N(c3cccc(-n4c5ccccc5c5ccccc54)c3)c3cccc(-c4cccc5ccccc45)c3-c3cccc4ccccc34)cc2)cc1 ; c1ccc(-c2ccc(N(c3ccc(-c4ccccc4)cc3-c3ccccc3)c3cccc(-c4cccc5ccccc45)c3-c3cccc4ccccc34)cc2)cc1]

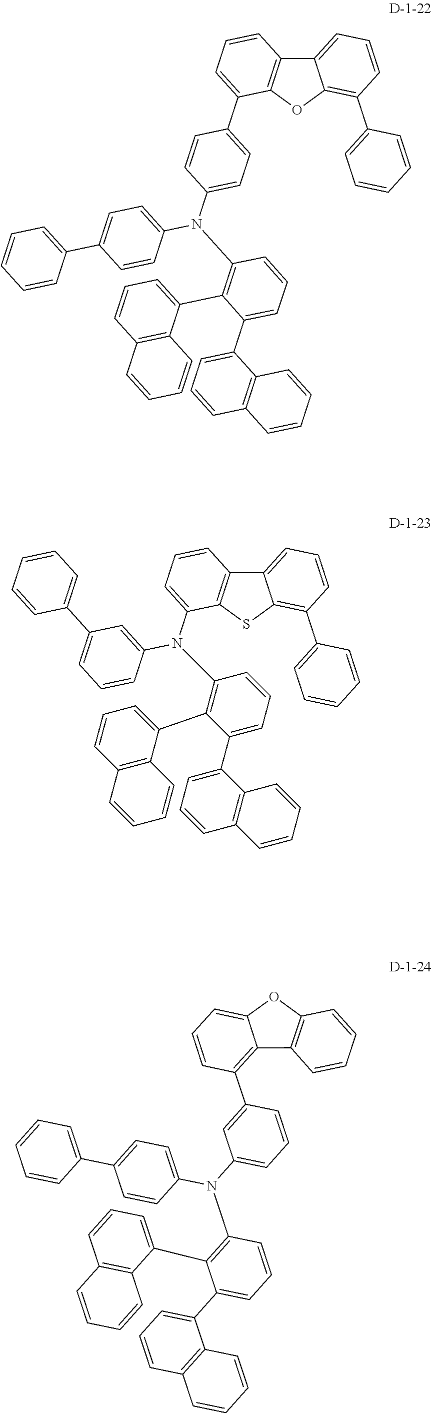 [SMILES: c1ccc(-c2ccc(N(c3ccc(-c4cccc5c4oc4c(-c6ccccc6)cccc45)cc3)c3cccc(-c4cccc5ccccc45)c3-c3cccc4ccccc34)cc2)cc1 ; c1ccc(-c2cccc(N(c3cccc(-c4cccc5ccccc45)c3-c3cccc4ccccc34)c3cccc4c3sc3c(-c5ccccc5)cccc34)c2)cc1 ; c1ccc(-c2ccc(N(c3cccc(-c4cccc5oc6ccccc6c45)c3)c3cccc(-c4cccc5ccccc45)c3-c3cccc4ccccc34)cc2)cc1]

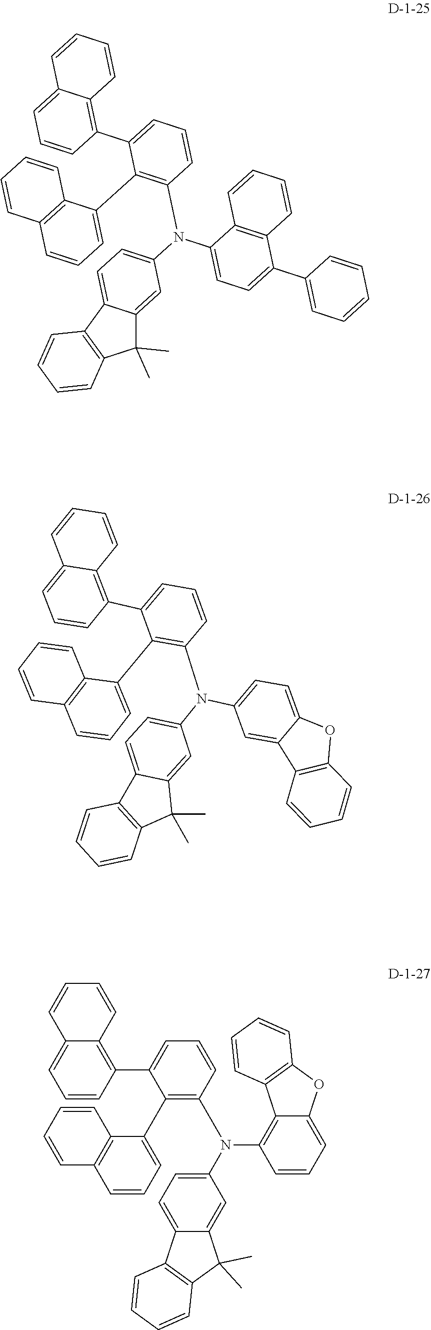 [SMILES: CC1(C)c2ccccc2-c2ccc(N(c3cccc(-c4cccc5ccccc45)c3-c3cccc4ccccc34)c3ccc(-c4ccccc4)c4ccccc34)cc21 ; CC1(C)c2ccccc2-c2ccc(N(c3ccc4oc5ccccc5c4c3)c3cccc(-c4cccc5ccccc45)c3-c3cccc4ccccc34)cc21 ; CC1(C)c2ccccc2-c2ccc(N(c3cccc(-c4cccc5ccccc45)c3-c3cccc4ccccc34)c3cccc4oc5ccccc5c34)cc21]

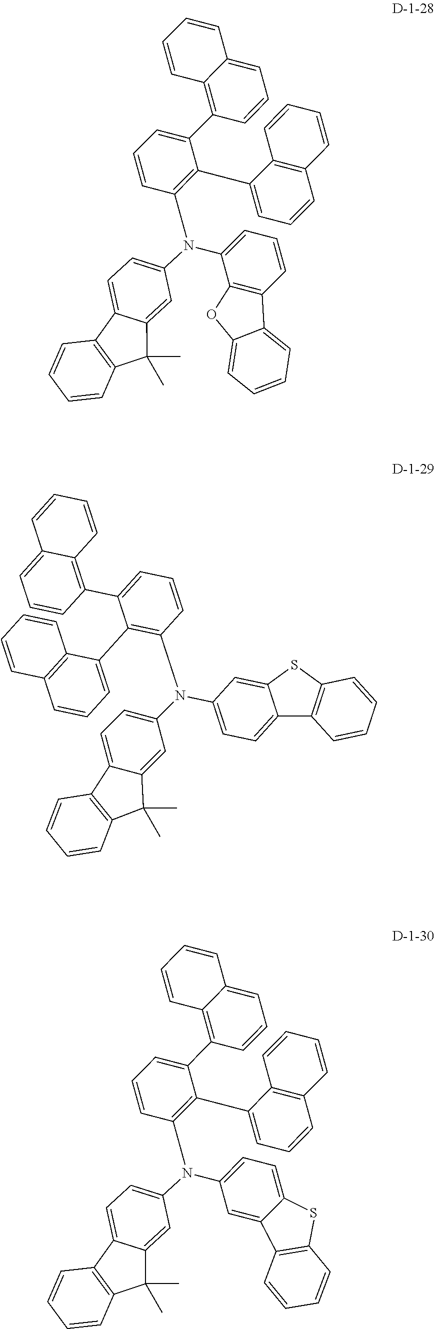 [SMILES: CC1(C)c2ccccc2-c2ccc(N(c3cccc(-c4cccc5ccccc45)c3-c3cccc4ccccc34)c3cccc4c3oc3ccccc34)cc21 ; CC1(C)c2ccccc2-c2ccc(N(c3ccc4c(c3)sc3ccccc34)c3cccc(-c4cccc5ccccc45)c3-c3cccc4ccccc34)cc21 ; CC1(C)c2ccccc2-c2ccc(N(c3ccc4sc5ccccc5c4c3)c3cccc(-c4cccc5ccccc45)c3-c3cccc4ccccc34)cc21]

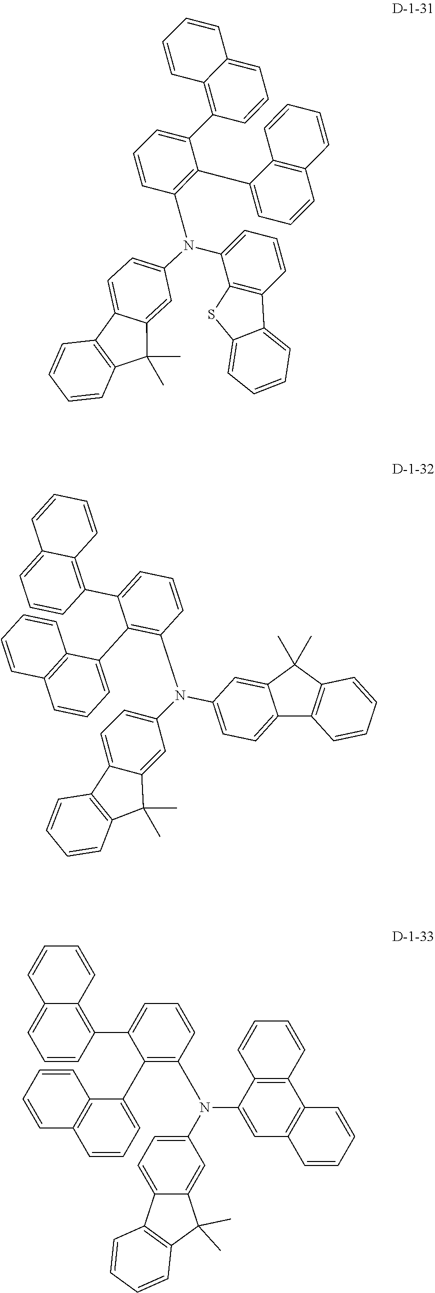 [SMILES: CC1(C)c2ccccc2-c2ccc(N(c3cccc(-c4cccc5ccccc45)c3-c3cccc4ccccc34)c3cccc4c3sc3ccccc34)cc21 ; CC1(C)c2ccccc2-c2ccc(N(c3ccc4c(c3)C(C)(C)c3ccccc3-4)c3cccc(-c4cccc5ccccc45)c3-c3cccc4ccccc34)cc21 ; CC1(C)c2ccccc2-c2ccc(N(c3cccc(-c4cccc5ccccc45)c3-c3cccc4ccccc34)c3cc4ccccc4c4ccccc34)cc21]

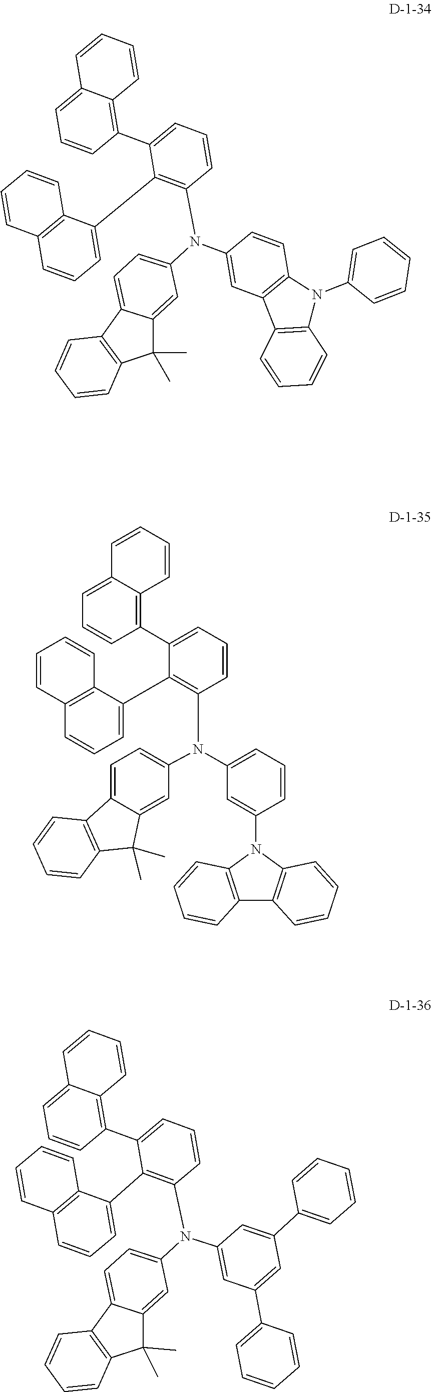 [SMILES: CC1(C)c2ccccc2-c2ccc(N(c3ccc4c(c3)c3ccccc3n4-c3ccccc3)c3cccc(-c4cccc5ccccc45)c3-c3cccc4ccccc34)cc21 ; CC1(C)c2ccccc2-c2ccc(N(c3cccc(-n4c5ccccc5c5ccccc54)c3)c3cccc(-c4cccc5ccccc45)c3-c3cccc4ccccc34)cc21 ; CC1(C)c2ccccc2-c2ccc(N(c3cc(-c4ccccc4)cc(-c4ccccc4)c3)c3cccc(-c4cccc5ccccc45)c3-c3cccc4ccccc34)cc21]

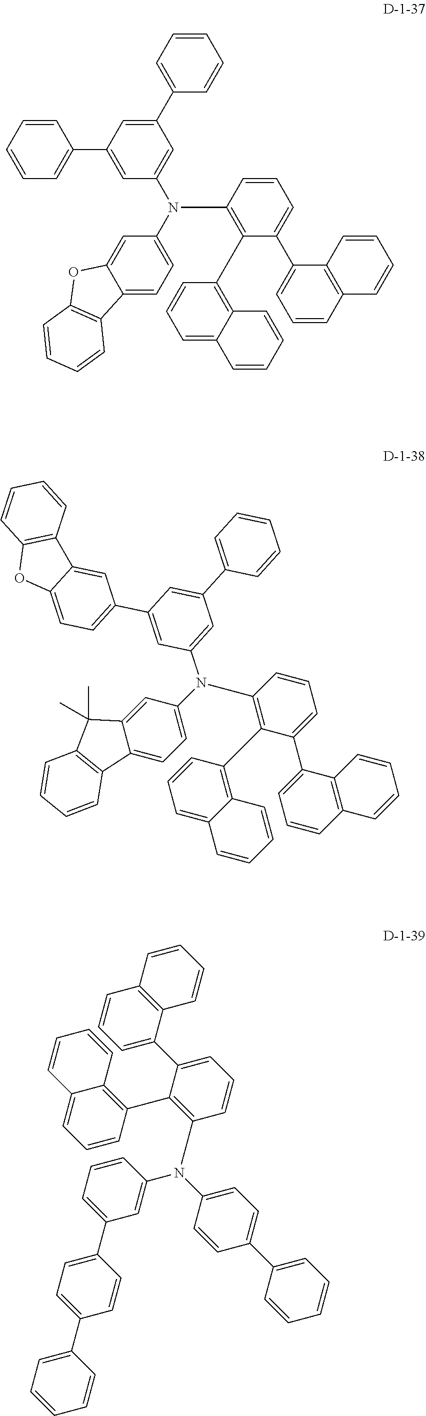 [SMILES: c1ccc(-c2cc(-c3ccccc3)cc(N(c3ccc4c(c3)oc3ccccc34)c3cccc(-c4cccc5ccccc45)c3-c3cccc4ccccc34)c2)cc1 ; CC1(C)c2ccccc2-c2ccc(N(c3cc(-c4ccccc4)cc(-c4ccc5oc6ccccc6c5c4)c3)c3cccc(-c4cccc5ccccc45)c3-c3cccc4ccccc34)cc21 ; c1ccc(-c2ccc(-c3cccc(N(c4ccc(-c5ccccc5)cc4)c4cccc(-c5cccc6ccccc56)c4-c4cccc5ccccc45)c3)cc2)cc1]

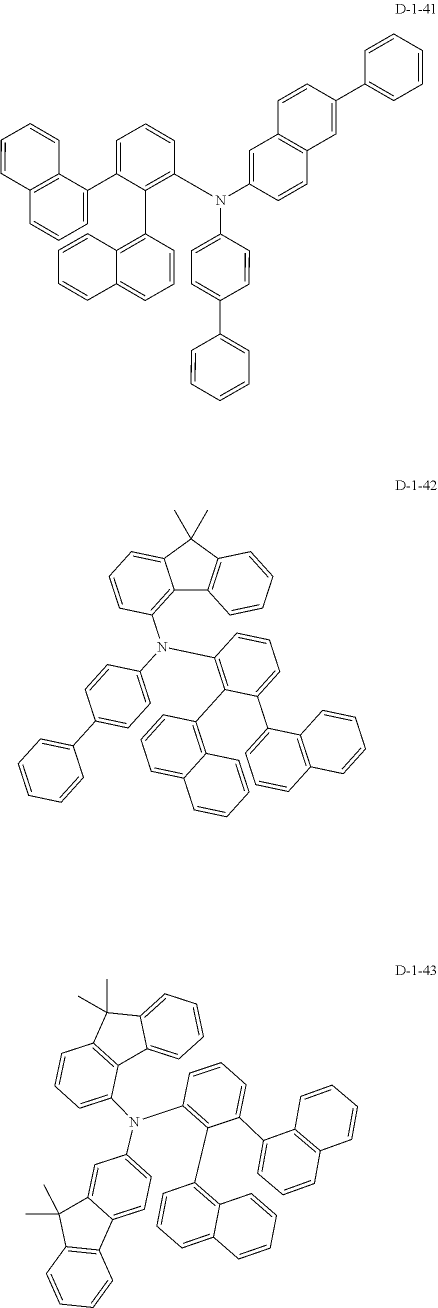 [SMILES: c1ccc(-c2ccc(N(c3ccc4cc(-c5ccccc5)ccc4c3)c3cccc(-c4cccc5ccccc45)c3-c3cccc4ccccc34)cc2)cc1 ; CC1(C)c2ccccc2-c2c(N(c3ccc(-c4ccccc4)cc3)c3cccc(-c4cccc5ccccc45)c3-c3cccc4ccccc34)cccc21 ; CC1(C)c2ccccc2-c2ccc(N(c3cccc(-c4cccc5ccccc45)c3-c3cccc4ccccc34)c3cccc4c3-c3ccccc3C4(C)C)cc21]

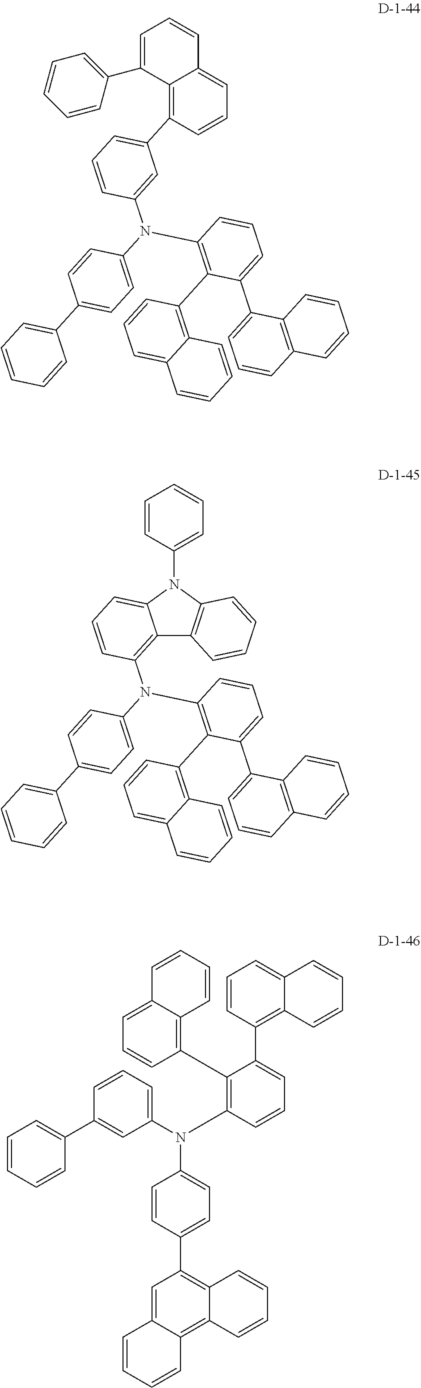 [SMILES: c1ccc(-c2ccc(N(c3cccc(-c4cccc5cccc(-c6ccccc6)c45)c3)c3cccc(-c4cccc5ccccc45)c3-c3cccc4ccccc34)cc2)cc1 ; c1ccc(-c2ccc(N(c3cccc(-c4cccc5ccccc45)c3-c3cccc4ccccc34)c3cccc4c3c3ccccc3n4-c3ccccc3)cc2)cc1 ; c1ccc(-c2cccc(N(c3ccc(-c4cc5ccccc5c5ccccc45)cc3)c3cccc(-c4cccc5ccccc45)c3-c3cccc4ccccc34)c2)cc1]

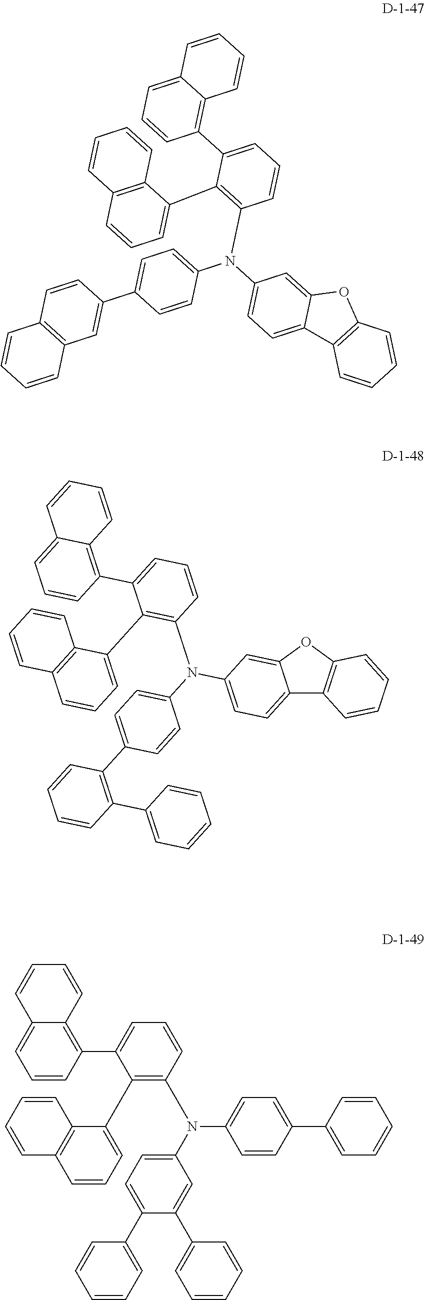 [SMILES: c1cc(-c2cccc3ccccc23)c(-c2cccc3ccccc23)c(N(c2ccc(-c3ccc4ccccc4c3)cc2)c2ccc3c(c2)oc2ccccc23)c1 ; c1ccc(-c2ccccc2-c2ccc(N(c3ccc4c(c3)oc3ccccc34)c3cccc(-c4cccc5ccccc45)c3-c3cccc4ccccc34)cc2)cc1 ; c1ccc(-c2ccc(N(c3ccc(-c4ccccc4)c(-c4ccccc4)c3)c3cccc(-c4cccc5ccccc45)c3-c3cccc4ccccc34)cc2)cc1]

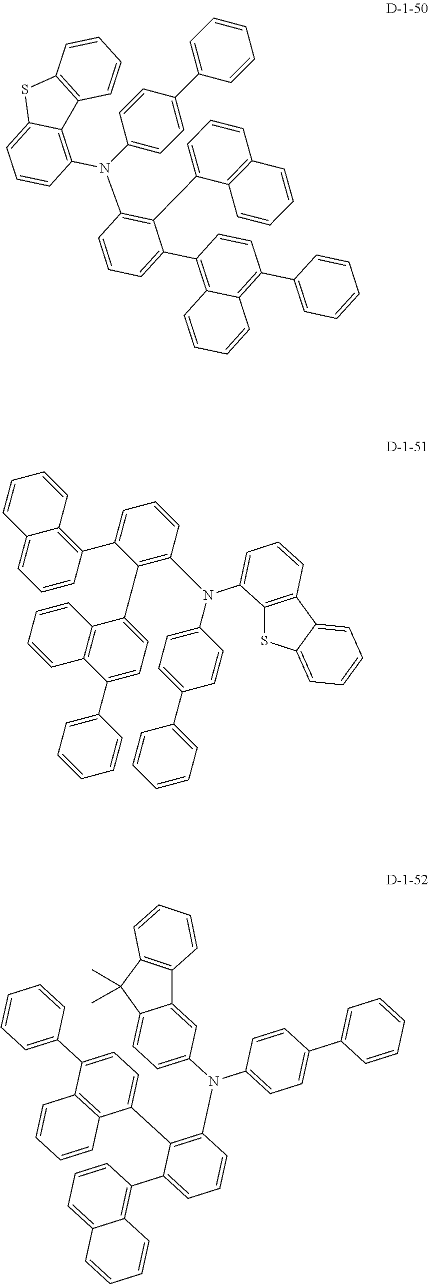 [SMILES: c1ccc(-c2ccc(N(c3cccc(-c4ccc(-c5ccccc5)c5ccccc45)c3-c3cccc4ccccc34)c3cccc4sc5ccccc5c34)cc2)cc1 ; c1ccc(-c2ccc(N(c3cccc(-c4cccc5ccccc45)c3-c3ccc(-c4ccccc4)c4ccccc34)c3cccc4c3sc3ccccc34)cc2)cc1 ; CC1(C)c2ccccc2-c2cc(N(c3ccc(-c4ccccc4)cc3)c3cccc(-c4cccc5ccccc45)c3-c3ccc(-c4ccccc4)c4ccccc34)ccc21]

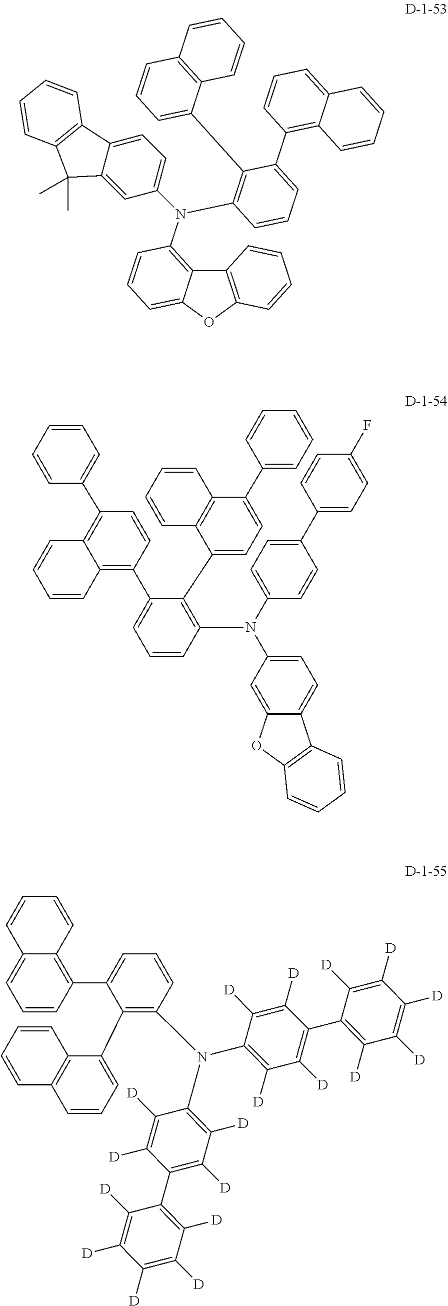 [SMILES: CC1(C)c2ccccc2-c2ccc(N(c3cccc(-c4cccc5ccccc45)c3-c3cccc4ccccc34)c3cccc4oc5ccccc5c34)cc21 ; Fc1ccc(-c2ccc(N(c3ccc4c(c3)oc3ccccc34)c3cccc(-c4ccc(-c5ccccc5)c5ccccc45)c3-c3ccc(-c4ccccc4)c4ccccc34)cc2)cc1 ; [2H]c1c([2H])c([2H])c(-c2c([2H])c([2H])c(N(c3cccc(-c4cccc5ccccc45)c3-c3cccc4ccccc34)c3c([2H])c([2H])c(-c4c([2H])c([2H])c([2H])c([2H])c4[2H])c([2H])c3[2H])c([2H])c2[2H])c([2H])c1[2H]]

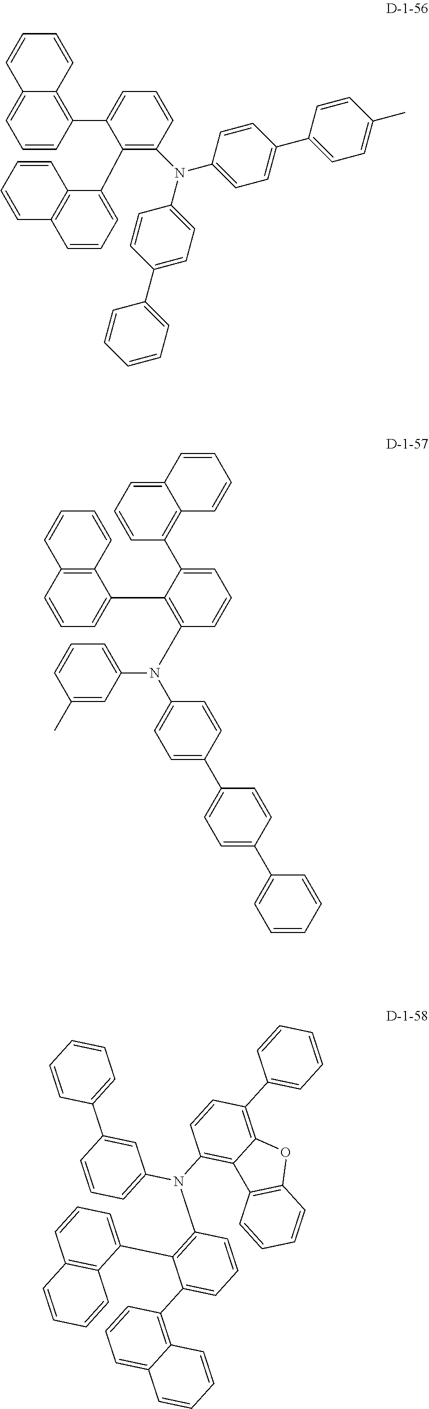 [SMILES: Cc1ccc(-c2ccc(N(c3ccc(-c4ccccc4)cc3)c3cccc(-c4cccc5ccccc45)c3-c3cccc4ccccc34)cc2)cc1 ; Cc1cccc(N(c2ccc(-c3ccc(-c4ccccc4)cc3)cc2)c2cccc(-c3cccc4ccccc34)c2-c2cccc3ccccc23)c1 ; c1ccc(-c2cccc(N(c3cccc(-c4cccc5ccccc45)c3-c3cccc4ccccc34)c3ccc(-c4ccccc4)c4oc5ccccc5c34)c2)cc1]

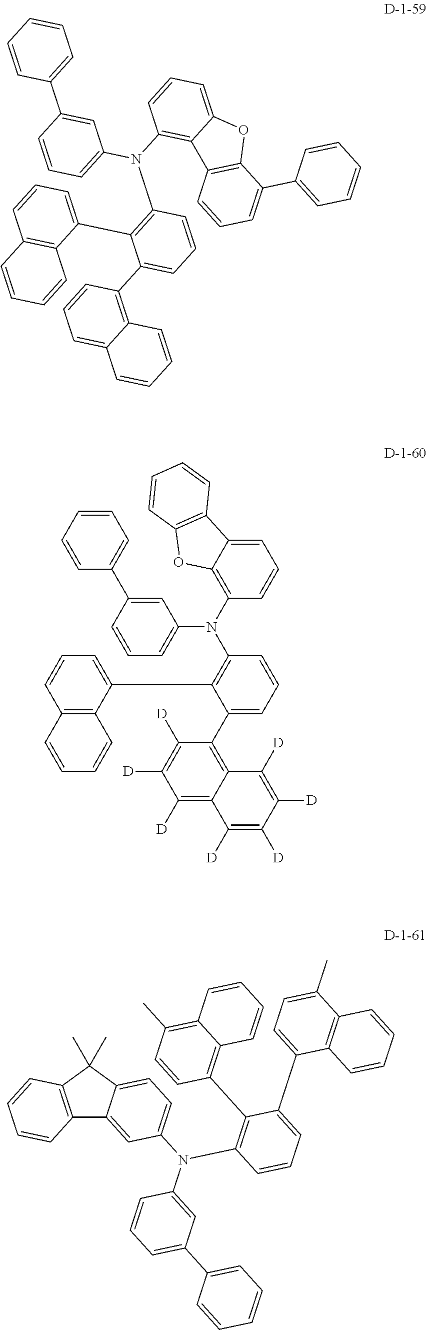 [SMILES: c1ccc(-c2cccc(N(c3cccc(-c4cccc5ccccc45)c3-c3cccc4ccccc34)c3cccc4oc5c(-c6ccccc6)cccc5c34)c2)cc1 ; [2H]c1c([2H])c([2H])c2c(-c3cccc(N(c4cccc(-c5ccccc5)c4)c4cccc5c4oc4ccccc45)c3-c3cccc4ccccc34)c([2H])c([2H])c([2H])c2c1[2H] ; Cc1ccc(-c2cccc(N(c3cccc(-c4ccccc4)c3)c3ccc4c(c3)-c3ccccc3C4(C)C)c2-c2ccc(C)c3ccccc23)c2ccccc12]

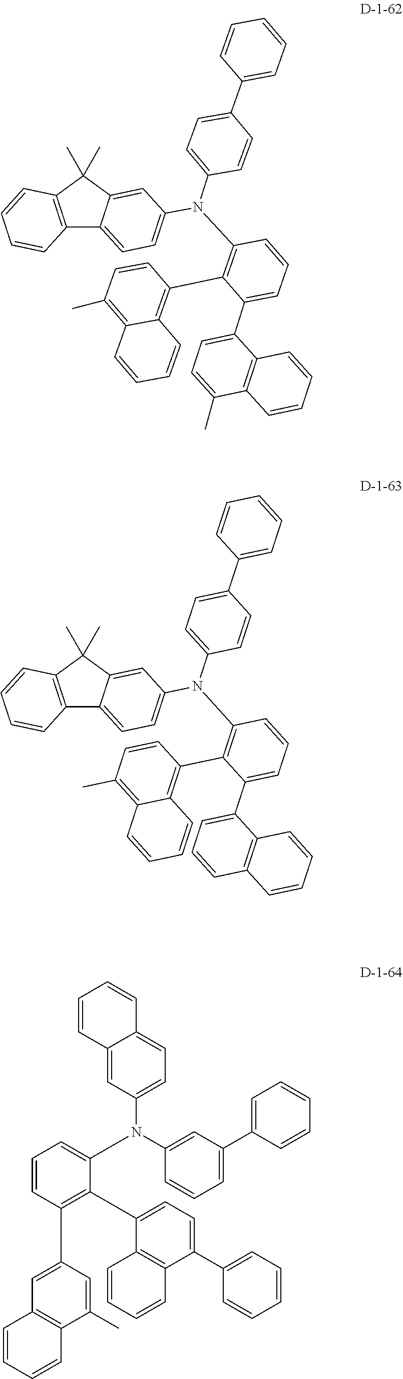 [SMILES: Cc1ccc(-c2cccc(N(c3ccc(-c4ccccc4)cc3)c3ccc4c(c3)C(C)(C)c3ccccc3-4)c2-c2ccc(C)c3ccccc23)c2ccccc12 ; Cc1ccc(-c2c(-c3cccc4ccccc34)cccc2N(c2ccc(-c3ccccc3)cc2)c2ccc3c(c2)C(C)(C)c2ccccc2-3)c2ccccc12 ; Cc1cc(-c2cccc(N(c3cccc(-c4ccccc4)c3)c3ccc4ccccc4c3)c2-c2ccc(-c3ccccc3)c3ccccc23)cc2ccccc12]

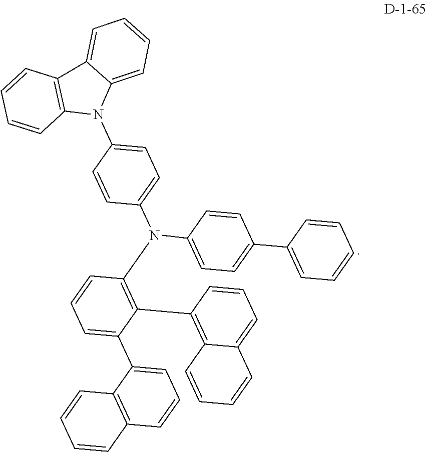 [SMILES: c1ccc(-c2ccc(N(c3ccc(-n4c5ccccc5c5ccccc54)cc3)c3cccc(-c4cccc5ccccc45)c3-c3cccc4ccccc34)cc2)cc1]

12. An electronic element, comprising an anode, a cathode, and a functional layer disposed between the anode and the cathode, wherein the functional layer comprises the nitrogen-containing compound according to claim 1.

13. The electronic element according to claim 12, wherein the functional layer comprises a hole transport layer, and the hole transport layer comprises the nitrogen-containing compound.

14. An electronic device, comprising the electronic element according to claim 12.

15. The nitrogen-containing compound according to claim 7, wherein the substituent(s) of Ar₁ and Ar₂ are each independently selected from deuterium, a fluorine, a cyano, a methyl, an ethyl, an isopropyl, a tert-butyl, or a phenyl.

16. The electronic element according to claim 13, wherein the electronic element is an organic electroluminescent device or a photoelectric conversion device.

17. The electronic element according to claim 13, wherein the electronic element is an organic electroluminescent device, and the hole transport layer comprises a first hole transport layer and a second hole transport layer, wherein the first hole transport layer is closer to the anode than the second hole transport layer, and the second hole transport layer comprises the nitrogen-containing compound.
